(19) United States
(12) Patent Application Publication
(10) Pub. No.: US 2023/0285310 A1
(43) Pub. Date: Sep. 14, 2023

(54) COMPOSITIONS FOR THE DELIVERY OF PAYLOAD MOLECULES TO AIRWAY EPITHELIUM

(71) Applicant: ModernaTX, Inc., Cambridge, MA (US)

(72) Inventors: Ana Cadete Pires, Arlington, MA (US); Jean C. Sung, Cambridge, MA (US); Mark Cornebise, Arlington, MA (US); Jeffrey Hrkach, Lexington, MA (US)

(21) Appl. No.: 18/040,485

(22) PCT Filed: Aug. 6, 2021
(86) PCT No.: PCT/US2021/045038

§ 371 Date: Feb. 3, 2023

Related U.S. Application Data
(60) Provisional application No. 63/062,367, filed on Aug. 6, 2020.

(51) Int. Cl. A61K 9/51; A61K 47/28; C07J 41/00; C07J 43/00

(52) U.S. Cl. CPC A61K 9/5123; A61K 9/5146; A61K 47/28; C07J 41/0005; C07J 41/0055; C07J 41/0088; C07J 43/003

(57) ABSTRACT

The present disclosure provides LNPs comprising payload molecules, e.g., mRNA therapeutics, for the treatment of diseases or disorders, which would benefit from delivery of payload molecules to airway cells.

### BACKGROUND

Respiratory epithelial cells line the respiratory tract. The primary functions of the respiratory epithelial cells are to moisten the respiratory tract, protect the airway tract from potential pathogens, infections and tissue injury, and/or facilitate gas exchange. Dysfunction in airway epithelial cells can lead to numerous disorders, including, for example, asthma, chronic obstructive pulmonary disease (COPD) and cystic fibrosis. Delivery of payloads to respiratory epithelial cells can be used to induce immunity to antigens of interest and to modulate the function of airway epithelial cells, e.g., to replace a missing or mutant protein or increase or decrease functionality of such cells.

For example, cystic fibrosis (“CF”) is an autosomal recessive disease characterized by the abnormal buildup of sticky and thick mucus in patients. CF is also known as cystic fibrosis of the pancreas, fibrocystic disease of the pancreas, or muscoviscidosis. Mucus is an important bodily fluid that lubricates and protects the lungs, reproductive system, digestive system, and other organs. However, CF patients produce thick and sticky mucus, which reduces the size of the airways leading to chronic coughing, wheezing, inflammation, bacterial infections, fibrosis, and cysts in the lungs. Additionally, most CF patients have mucus blocking the ducts in the pancreas, which prevents the release of insulin and digestive enzymes leading to diarrhea, malnutrition, poor growth, and weight loss. Gershman A. J. et al., Cleve Clin J Med. 73: 1065-1074 (2006). CF has an estimated incidence of 1 in 2,500 to 3,500 in Caucasian births, but is much more rare in other populations. Ratjen F. et al., Lancet 361: 681-689 (2003). Most current treatment for CF only controls the symptoms and does not cure the disease. Specifically, antibiotics, anti-inflammatory drugs, bronchodilators, decongestants, a diet high in protein and fat, and vitamin supplements are prescribed to control the symptoms. In advanced lung disease, lung transplants have also been performed to provide a patient with undamaged lungs. However, these treatments do not completely or reliably control the disease. New treatments have emerged that focus on the underlying cause of CF. These treatments modulate the cystic fibrosis transmembrane conductance regulator (CFTR) in patients with the Phe508del CFTR mutation. Middleton P. J. et al., N Engl J Med. 381: 1809-1819 (2019). However, about one in every one hundred CF patients does not have the Phe508del CFTR mutation, excluding them from this treatment.

As such, there is a need for improved therapy to treat disorders associated with airway epithelial cell dysfunction, e.g. CF, to target such airway epithelial cells for prophylactic therapy, e.g. immunization, or to treat other disorders that would benefit from therapeutic delivery of nucleic acid molecules or other payload molecules to airway epithelial cells.

### SUMMARY

The present disclosure provides LNP molecules for delivery of nucleic acid molecules, e.g., mRNA therapeutics, to airway epithelial cells for the treatment of disorders associated with airway epithelium dysfunction or for the prophylactic benefit of patients. In one embodiment, the subject LNP molecules can be used to treat disorders associated with epithelial cell dysfunction, such as cystic fibrosis (CF), COPD, or asthma as well as to administer vaccine payloads. The instant disclosure provides LNPs which have improved properties when administered to cells, e.g., in vitro and in vivo, for example, improved delivery of payloads to epithelial cells as measured, e.g., by cellular accumulation of LNP, expression of a desired protein, and/or mRNA expression.

In one aspect, provided herein is a nanoparticle comprising:
(a) a lipid nanoparticle core,
(b) a polynucleotide or polypeptide payload encapsulated within the core for delivery into a cell, and
(c) a cationic agent disposed primarily on the outer surface of the core, wherein the nanoparticle has a greater than neutral zeta potential at physiologic pH.

In one aspect, provided herein is a nanoparticle comprising:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payload encapsulated within the core for delivery into a cell, and
(c) a cationic agent.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% in epithelial cells and exhibits about 5% or greater expression in epithelial cells.

In one aspect, provided herein is a process of preparing a nanoparticle comprising contacting a lipid nanoparticle with a cationic agent, wherein the lipid nanoparticle comprises:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payload encapsulated within the core for delivery into a cell.

In one aspect, provided herein is a nanoparticle prepared by a process described herein.

In one aspect, provided herein is a method of delivering a polynucleotide or polypeptide payload into a cell comprising contacting the cell with a nanoparticle described herein.

In one aspect, provided herein is a method of treating or preventing a disease in a patient comprising administering to the patient a nanoparticle comprising a payload for treatment or prevention of the disease as described herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

FIG. 1 is a diagram of exemplary first generation post-hoc loading (PHL) process for preparing LNP.

FIG. 2 is a diagram of exemplary second generation PHL process (generic) for preparing LNP.

FIG. 3 is a diagram of exemplary second generation PHL process (specific) for preparing LNP.

FIG. 4 is a diagram of exemplary process of preparing an empty lipid nanoparticle prototype (“Neutral assembly”), where the empty LNP is mixed at pH 8.0 and the final formulation is pH 5.0.

FIG. 5 is a diagram of exemplary process of preparing an LNP with a sterol amine.

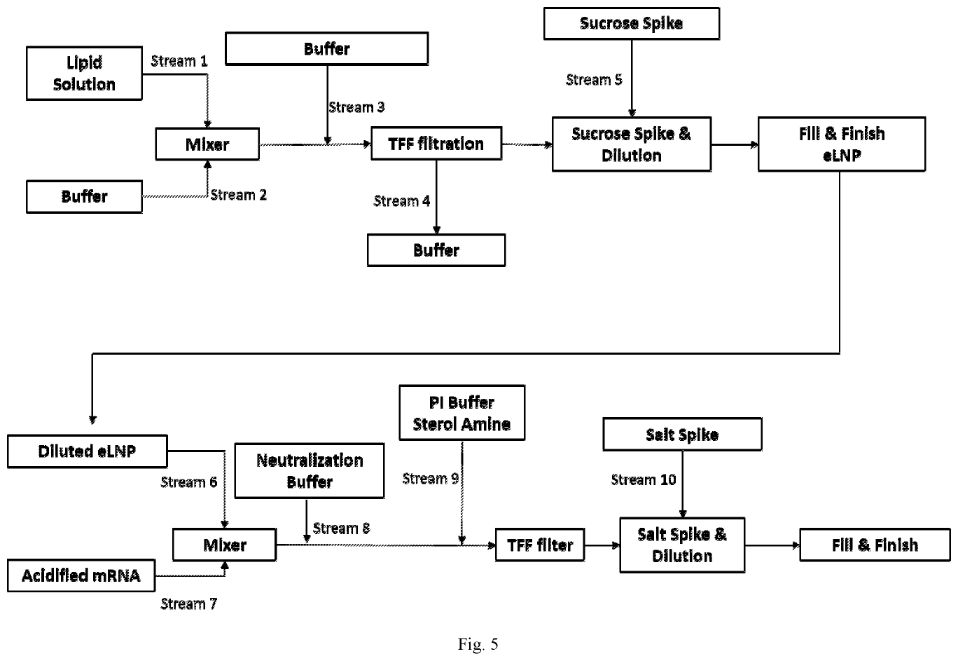

FIG. 6 is a small angle x-ray scatters (SAXS) analysis of LNP-1 and LNP-1a.

FIG. 7 is a graph showing the general polarity Laurdan (GLP) for LNP-1 and LNP-1a.

### DETAILED DESCRIPTION

The present disclosure provides LNP molecules for delivery of nucleic acid or payload molecules to airway epithelial cells. For example, such LNP molecules can be used to deliver payload molecules, e.g., mRNA therapeutics for the treatment of cystic fibrosis (CF) to airway epithelial cells. For example, cystic fibrosis (CF) is a progressive, genetic disease that causes persistent lung infections and limits the ability to breathe over time. This disease is characterized by the presence of mutations in both copies of the gene for the cystic fibrosis transmembrane conductance regulator (CFTR) protein. Without CFTR, which is involved in the production of sweat, digestive fluids and mucus, secretions that are usually thin instead become thick. mRNA therapeutics are particularly well-suited for the treatment of CF as the technology provides for the intracellular delivery of mRNA encoding CFTR followed by de novo synthesis of functional CFTR protein within target cells. After delivery of mRNA to the target cells, the desired CFTR protein is expressed by the cells' own translational machinery, and hence, fully functional CFTR protein replaces the defective or missing protein. In another embodiment, such LNPs can be used to deliver nucleic acid molecules for gene editing, small molecules, or other payloads to ameliorate epithelial cell dysfunction. In another embodiment, such LNPs can be used to deliver antigens to airway cells. In one embodiment, the antigen is in the form of an mRNA construct present in the LNP resulting in the expression of a polypeptide or peptide such that an immune response to the antigen is produced.

Lipid nanoparticles (LNPs) are an ideal platform for the safe and effective delivery of payload molecules, e.g., mRNAs to target cells. LNPs have the unique ability to deliver nucleic acids by a mechanism involving cellular uptake, intracellular transport and endosomal release or endosomal escape. Some embodiments provided herein feature LNPs that have improved properties. In some embodiments, the LNP provided herein comprises a lipid nanoparticle core, a polynucleotide or polypeptide payload encapsulated within the core for delivery into a cell, and a cationic agent disposed primarily on the outer surface of the nanoparticle. Without being bound by a particular theory, LNPs having a cationic agent disposed primarily on the outer surface of the core can improve accumulation of the LNP in cells such as human bronchial epithelial (HBE) and also improve function of the payload molecule, e.g., as measured by mRNA expression in cells, e.g., airway epithelial cells.

In some embodiments, provided herein is a nanoparticle comprising:
(a) a lipid nanoparticle core,
(b) a polynucleotide or polypeptide payload encapsulated within the core for delivery into a cell, and
(c) a cationic agent disposed primarily on the outer surface of the core, wherein the nanoparticle has a greater than neutral zeta potential at physiologic pH.

In some embodiments, provided herein is a nanoparticle comprising:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payload encapsulated within the core for delivery into a cell, and
(c) a cationic agent.

In some embodiments, provided herein is a nanoparticle comprising:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payload encapsulated within the core for delivery into a cell, and
(c) a cationic agent disposed primarily on the outer surface of the core.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% of cells and exhibits about 5% or greater expression in cells. In some embodiments, the nanoparticle exhibits a cellular accumulation of about 1% to about 75%, 5% to about 50%, about 10% to about 40%, or about 15% to about 25% of cells. In some embodiments, the nanoparticle exhibits about 0.5% to about 50%, about 1% to about 40%, about 3% to about 20%, or about 5% to about 15% expression in cells.

In one aspect, provided herein is a nanoparticle comprising:
(a) a lipid nanoparticle core,
(b) a polynucleotide or polypeptide payload encapsulated within the core for delivery into a cell, and
(c) a cationic agent disposed primarily on the outer surface of the core, wherein the nanoparticle exhibits a cellular accumulation of at least about 20% of cells and exhibits about 5% or greater expression in cells. In some embodiments, the nanoparticle exhibits a cellular accumulation of about 1% to about 75%, 5% to about 50%, about 10% to about 40%, or about 15% to about 25% of cells. In some embodiments, the nanoparticle exhibits about 0.5% to about 50%, about 1% to about 40%, about 3% to about 20%, or about 5% to about 15% expression in cells.

In one aspect, provided herein is a nanoparticle comprising:
(a) a lipid nanoparticle core,
(b) a polynucleotide or polypeptide payload encapsulated within the core for delivery into a cell, and
(c) a cationic agent disposed primarily on the outer surface of the core, wherein the nanoparticle exhibits protein expression of about 0.5% to 50% in cells. In some embodiments, the nanoparticle exhibits protein expression of about 0.1% to about 60%, about 0.5% to about 40%, about 1% to about 30%, or about 1% to about 20% in cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression of about 0.5% to 50% in cells. In some embodiments, the nanoparticle exhibits protein expression of about 0.1% to about 60%, about 0.5% to about 40%, about 1% to about 30%, or about 1% to about 20% in cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% in epithelial cells and exhibits about 5% or greater expression in epithelial cells. In some embodiments, the nanoparticle exhibits a cellular accumulation of about 1% to about 75%, 5% to about 50%, about 10% to about 40%, or about 15% to about 25% of epithelial cells. In some embodiments, the nanoparticle exhibits about 0.5% to about 50%, about 1% to about 40%, about 3% to about 20%, or about 5% to about 15% expression in epithelial cells. In some embodiments, the epithelial cells are HBE cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression of about 0.5% to 50% of epithelial cells. In some embodiments, the nanoparticle exhibits protein expression of about 0.1% to about 60%, about 0.5% to about 40%, about 1% to about 30%, or about 1% to about 20% of epithelial cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% of lung cells. In some embodiments, the nanoparticle exhibits protein expression of about 0.1% to about 60%, about 0.5% to about 40%, about 1% to about 30%, or about 1% to about 20% of lung cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% of nasal cells. In some embodiments, the nanoparticle exhibits protein expression of about 0.1% to about 60%, about 0.5% to about 40%, about 1% to about 30%, or about 1% to about 20% of nasal cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% of alveolar epithelial cells. In some embodiments, the nanoparticle exhibits protein expression of about 0.1% to about 60%, about 0.5% to about 40%, about 1% to about 30%, or about 1% to about 20% of alveolar epithelial cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% in respiratory epithelial cells and exhibits about 5% or greater expression in respiratory epithelial cells. In some embodiments, the nanoparticle exhibits a cellular accumulation of about 1% to about 75%, 5% to about 50%, about 10% to about 40%, or about 15% to about 25% of respiratory epithelial cells. In some embodiments, the nanoparticle exhibits about 0.5% to about 50%, about 1% to about 40%, about 3% to about 20%, or about 5% to about 15% expression in respiratory epithelial cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% respiratory epithelial cells. In some embodiments, the nanoparticle exhibits protein expression of about 0.1% to about 60%, about 0.5% to about 40%, about 1% to about 30%, or about 1% to about 20% of respiratory epithelial cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% of macrophages. In some embodiments, the nanoparticle exhibits protein expression of about 0.1% to about 60%, about 0.5% to about 40%, about 1% to about 30%, or about 1% to about 20% of macrophages.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% of HeLa cells. In some embodiments, the nanoparticle exhibits protein expression of about 0.1% to about 60%, about 0.5% to about 40%, about 1% to about 30%, or about 1% to about 20% of HeLa cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% in bronchial epithelial cells and exhibits about 5% or greater expression in bronchial epithelial cells. In some embodiments, the nanoparticle exhibits a cellular accumulation of about 1% to about 75%, 5% to about 50%, about 10% to about 40%, or about 15% to about 25% of respiratory epithelial cells. In some embodiments, the nanoparticle exhibits about 0.5% to about 50%, about 1% to about 40%, about 3% to about 20%, or about 5% to about 15% expression in bronchial epithelial cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits protein expression in about 0.5% to about 50% bronchial epithelial cells. In some embodiments, the nanoparticle exhibits protein expression of about 0.1% to about 60%, about 0.5% to about 40%, about 1% to about 30%, or about 1% to about 20% of bronchial epithelial cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% in HBE cells and exhibits about 5% or greater expression in HBE cells. In some embodiments, the nanoparticle exhibits a cellular accumulation of about 1% to about 75%, 5% to about 50%, about 10% to about 40%, or about 15% to about 25% in HBE. In some embodiments, the nanoparticle exhibits about 0.5% to about 50%, about 1% to about 40%, about 3% to about 20%, or about 5% to about 15% expression in HBE cells.

In one aspect, provided herein is a nanoparticle comprising:
(a) a lipid nanoparticle core,
(b) a polynucleotide or polypeptide payload encapsulated within the core for delivery into a cell, and
(c) a cationic agent,
   wherein the nanoparticle exhibits a cellular accumulation of at least about 20% in healthy HBE cells in vitro and exhibits about 5% or greater expression in healthy HBE cells in vitro. In some embodiments, the nanoparticle exhibits a cellular accumulation of about 1% to about 75%, 5% to about 50%, about 10% to about 40%, or about 15% to about 25% in healthy HBE cells in vitro. In some embodiments, the nanoparticle exhibits about 0.5% to about 50%, about 1% to about 40%, about 3% to about 20%, or about 5% to about 15% expression in healthy HBE cells in vitro.

In some embodiments, the cells referred to herein-above and herein-throughout can be in vitro cells or in vivo cells. In some embodiments, the cells are in vitro cells. In some embodiments, the cells are in vivo cells.

In some embodiments, the nanoparticles of the invention have increased cellular accumulation (e.g., in airway epithelial cells such as HBE) relative to nanoparticles of the substantially the same composition but prepared without post addition of the cationic agent (e.g., layering or contacting of the cationic agent with the pre-formed lipid nanoparticle). In some embodiments, the nanoparticles of the invention have increased cellular expression (e.g., in airway epithelial cells such as HBE) relative to nanoparticles of the substantially the same composition but prepared without post addition of the cationic agent (e.g., layering or contacting of the cationic agent with the pre-formed lipid nanoparticle).

In some embodiments, a weight ratio of the cationic agent to polynucleotide payload is about 0.1:1 to about 15:1. In some embodiments, a weight ratio of the cationic agent to polynucleotide payload is about 0.2:1 to about 10:1. In some embodiments, a weight ratio of the cationic agent to polynucleotide payload is about 1:1 to about 10:1. In some embodiments, a weight ratio of the cationic agent to polynucleotide payload is about 1:1 to about 8:1. In some embodiments, a weight ratio of the cationic agent to polynucleotide payload is about 1:1 to about 7:1. In some embodiments, a weight ratio of the cationic agent to polynucleotide payload is about 1:1 to about 6:1. In some embodiments, a weight ratio of the cationic agent to polynucleotide payload is about 1:1 to about 5:1. In some embodiments, a weight ratio of the cationic agent to polynucleotide payload is about 1:1 to about 4:1. In some embodiments, a weight ratio of the cationic agent to polynucleotide payload is about 1.25:1 to about 3.75:1. In some embodiments, a weight ratio of the cationic agent to polynucleotide payload is about 1.25:1. In some embodiments, a weight ratio of the cationic agent to polynucleotide payload is about 2.5:1. In some embodiments, a weight ratio of the cationic agent to polynucleotide payload is about 3.75:1.

In some embodiments, a molar ratio of the cationic agent to polynucleotide payload is about 0.1:1 to about 20:1. In some embodiments, a molar ratio of the cationic agent to polynucleotide payload is about 1.5:1 to about 10:1. In some embodiments, a molar ratio of the cationic agent to polynucleotide payload is about 1.5:1 to about 9:1. In some embodiments, a molar ratio of the cationic agent to polynucleotide payload is about 1.5:1 to about 8:1. In some embodiments, a molar ratio of the cationic agent to polynucleotide payload is about 1.5:1 to about 7:1. In some embodiments, a molar ratio of the cationic agent to polynucleotide payload is about 1.5:1 to about 6:1. In some embodiments, a molar ratio of the cationic agent to polynucleotide payload is about 1.5:1 to about 5:1. In some embodiments, a molar ratio of the cationic agent to polynucleotide payload is about 1.5:1. In some embodiments, a molar ratio of the cationic agent to polynucleotide payload is about 2:1. In some embodiments, a molar ratio of the cationic agent to polynucleotide payload is about 3:1. In some embodiments, a molar ratio of the cationic agent to polynucleotide payload is about 4:1. In some embodiments, a molar ratio of the cationic agent to polynucleotide payload is about 5:1.

In some embodiments, the nanoparticle of the invention has a zeta potential of about 5 mV to about 20 mV. In some embodiments, the nanoparticle has a zeta potential of about 5 mV to about 15 mV. In some embodiments, the nanoparticle has a zeta potential of about 5 mV to about 10 mV.

Zeta potential measures the surface charge of colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. This measurement is more sensitive and less stress inducing than ISO Method 13099-1:2012 which only uses one angle of detection and required higher voltage for operation. In some embodiments, the zeta potential of the herein described empty lipid nanoparticle compositions lipid is measured using an instrument employing the principle of MP-PALS. Zeta potential can be measured on a Malvern Zetasizer (Nano ZS).

In some embodiments, the lipid nanoparticle core has a neutral charge at a neutral pH.

In some embodiments, greater than about 80% of the cationic agent is on the surface on the nanoparticle. In some embodiments, greater than about 90% of the cationic agent is on the surface on the nanoparticle. In some embodiments, greater than about 95% of the cationic agent is on the surface on the nanoparticle.

In some embodiments, at least about 50% of the polynucleotide or polypeptide payload is encapsulated within the core. In some embodiments, at least about 75% of the polynucleotide or polypeptide payload is encapsulated within the core. In some embodiments, at least about 90% of the polynucleotide or polypeptide payload is encapsulated within the core. In some embodiments, at least about 95% of the polynucleotide or polypeptide payload is encapsulated within the core.

In some embodiments, the nanoparticle has a polydispersity value of less than about 0.4. In some embodiments, the nanoparticle has a polydispersity value of less than about 0.3. In some embodiments, the nanoparticle has a polydispersity value of less than about 0.2.

In some embodiments, the nanoparticle has a mean diameter of about 40 nm to about 150 nm. In some embodiments, the nanoparticle has a mean diameter of about 50 nm to about 100 nm. In some embodiments, the nanoparticle has a mean diameter of about 60 nm to about 120 nm. In some embodiments, the nanoparticle has a mean diameter of about 60 nm to about 100 nm. In some embodiments, the nanoparticle has a mean diameter of about 60 nm to about 80 nm.

In some embodiments, a general polarization of laurdan of the nanoparticle is greater than or equal to about 0.6. In some embodiments, the nanoparticle has a d-spacing of greater than about 6 nm. In some embodiments, the nanoparticle has a d-spacing of greater than about 7 nm.

In some embodiments, at least 50% of the nanoparticles have a surface fluidity value of greater than a threshold polarization level. In some embodiments, at least 75% of the nanoparticles have a surface fluidity value of greater than a threshold polarization level. In some embodiments, at least 90% of the nanoparticles have a surface fluidity value of greater than a threshold polarization level. In some embodiments, at least 95% of the nanoparticles have a surface fluidity value of greater than a threshold polarization level.

In some embodiments, about 10% or greater of cell population has accumulated the nanoparticle when the nanoparticle is contacted with a population of cells. In some embodiments, about 15% or greater of cell population has accumulated the nanoparticle when the nanoparticle is contacted with a population of cells. In some embodiments, about 20% or greater of cell population has accumulated the nanoparticle when the nanoparticle is contacted with a population of cells. In some embodiments, about 5% or greater of cell expresses the polynucleotide or polypeptide when the nanoparticle is contacted with a population of cells. In some embodiments, about 10% or greater of cell expresses the polynucleotide or polypeptide when the nanoparticle is contacted with a population of cells. In some embodiments, the cell population is an epithelial cell population. In some embodiments, the cell population is a respiratory epithelial cell population. In some embodiments, the respiratory epithelial cell population is a lung cell population. In some embodiments, the respiratory epithelial cell population is a nasal cell population. In some embodiments, the respiratory epithelial cell population is an alveolar epithelial cell population. In some embodiments, the respiratory epithelial cell population is a bronchial epithelial cell population. In some embodiments, the respiratory epithelial cell population is an HBE population. In some embodiments, the cell population is a lung cell population. In some embodiments, the cell population is a nasal cell population. In some embodiments, the cell population is an alveolar epithelial cell population. In some embodiments, the cell population is a bronchial epithelial cell population. In some embodiments, the cell population is an HBE population. In some embodiments, the cell population is HeLa population.

### Cationic Agent

The cationic agent can comprise any aqueous soluble molecule or substance that has a net positive charge and can adhere to the surface of a lipid nanoparticle core. Such agent may also be lipid soluble but will also be soluble in aqueous solution. The cationic agent can be charged at physiologic pH. Physiological pH is the pH level normally observed in the human body. Physiological pH can be about 7.30-7.45 or about 7.35-7.45. Physiological pH can be about 7.40. Generally speaking, the cationic agent features a net positive charge at physiologic pH because it contains one or more basic functional groups that are protonated at physiologic pH in aqueous media. For example, the cationic agent can contain one or more amine groups, e.g. primary, secondary, or tertiary amines each having a pKa of 8.0 or greater. The pKa can be greater than about 9.

In some embodiments, the cationic agent can be a cationic lipid which is a water-soluble, amphiphilic molecule in which one portion of the molecule is hydrophobic comprising, for example, a lipid moiety, and where the other portion of the molecule is hydrophilic, containing one or more functional groups which is typically charged at physiologic pH. The hydrophobic portion, comprising the lipid moiety, can serve to anchor the cationic agent to a lipid nanoparticle core. The hydrophilic portion can serve to increase the charge on the surface of a lipid nanoparticle core. For example, the cationic agent can have a solubility of greater than about 1 mg/mL in alcohol. The solubility in alcohol can be greater than about 5 mg/mL. The solubility in alcohol can be greater than about 10 mg/mL. The solubility in alcohol can be greater than about 20 mg/mL in alcohol. The alcohol can be C₁₋₆ alcohol such as ethanol.

The lipid portion of the molecule can be, for example, a structural lipid, fatty acid, or similar hydrocarbyl group.

The structural lipid can be selected from, but is not limited to, a steroid, diterpeniod, triterpenoid, cholestane, ursolic acid, or derivatives thereof.

In some embodiments, the structural lipid is a steroid selected from, but not limited to, cholesterol or a phystosterol. In some embodiments, the structural lipid is an analog of cholesterol. In some embodiments, the structural lipid is a sitosterol, campesterol, or stigmasterol. In some embodiments, the structural lipid is an analog of sitosterol, campesterol, or stigmasterol. In some embodiments, the structural lipid is 3-sitosterol.

The fatty acid comprises 1 to 4 C₆₋₂₀ hydrocarbon chains. The fatty acid can be fully saturated or can contain 1 to 7 double bonds. The fatty acid can contain 1 to 5 heteroatoms either along the main chain or pendent to the main chain.

In some embodiments, the fatty acid comprises two C₁₀₋₁₈ hydrocarbon chains. In some embodiments, the fatty acid comprises two C₁₀₋₁₈ saturated hydrocarbon chains. In some embodiments, the fatty acid comprises two C₁₆ saturated hydrocarbon chain. In some embodiments, the fatty acid comprises two C₁₄ saturated hydrocarbon chain. In some embodiments, the fatty acid comprises two unsaturated C₁₀₋₁₈ hydrocarbon chains. In some embodiments, the fatty acid comprises two C₁₆₋₁₈ hydrocarbon chains, each with one double bond. In some embodiments, the fatty acid comprises three C₈₋₁₈ saturated hydrocarbon chains.

The hydrocarbyl group consists of 1 to 4 C₆₋₂₀ alkyl, alkenyl, or alkynyl chains or 3 to 10 membered cycloalkyl, cycloalkenyl, or cycloalkynyl groups.

In some embodiments, the hydrocarbyl chain is a C₈₋₁₀ alkyl. In some embodiments, the hydrocarbyl chain is C₈₋₁₀ alkenyl.

The hydrophilic portion can comprise 1 to 5 functional groups that would be charged at physiologic pH, 7.3 to 7.4. The hydrophilic group can comprise a basic functional group that would be protonated and positively charged at physiologic pH. At least one of the basic functional groups has a pKa of 8 or greater.

In some embodiments, the hydrophilic portion comprises an amine group. The amine group can comprise one to four primary, secondary, or tertiary amines and mixtures thereof. The primary, secondary, or tertiary amines can be part of larger amine containing functional group selected from, but not limited to, —C(═N—)—N—, —C═C—N—, —C═N—, or —N—C(═N—)—N—. The amine can be contained in a three to eight membered heteroalkyl or heteroaryl ring.

In some embodiments, the amine group comprises one or two terminal primary amines. In some embodiments, the amine group comprises one or two terminal primary amines and one internal secondary amine. In some embodiments, the amine group comprises one or two tertiary amines. In some embodiments, the tertiary amine is (CH₃)₂N—. In some embodiments, amine group comprises one to two terminal (CH₃)₂N—.

The hydrophilic portion can comprise a phosphonium group. The counter ion of the phosphonium ion consists of an anion with a charge of one.

In some embodiments, three of the substituents on the phosphonium are isopropyl groups. In some embodiments, the counter ion is a halo, hydrogen sulfate, nitrite, chlorate, or hydrogen carbonate. In some embodiments, the counter ion is a bromide.

In some embodiments, the cationic agent is a cationic lipid which is a sterol amine. A sterol amine has, for its hydrophobic portion, a sterol, and for its hydrophilic portion, an amine group. The sterol group is selected from, but not limited to, cholesterol, sitosterol, campesterol, stigmasterol or derivatives thereof. The amine group can comprise one to five primary, secondary, tertiary amines, or mixtures thereof. At least one of the amines has a pKa of 8 or greater and is charged at physiological pH. The primary, secondary, or tertiary amines can be part of a larger amine containing functional group selected from, but not limited to —C(═N—)—N—, —C═C—N—, —C═N—, or —N—C(═N—)—N—. The amine can be contained in a three to eight membered heteroalkyl or heteroaryl ring.

In some embodiments, the amine group of the sterol amine comprises one or two terminal primary amines. In some embodiments, the amine group comprises one or two terminal primary amines and one internal secondary amine. In some embodiments, the amine group comprises one or two tertiary amines. In some embodiments, the tertiary amine is (CH₃)₂N—. In some embodiments, amine group comprises one to two terminal (CH₃)₂N—.

Sterol amines useful in the nanoparticles of the invention include molecules having Formula (A1):

A-L-B (A1)

or a salt thereof, wherein:
A is an amine group, L is an optional linker, and B is a sterol.

In some embodiments, the amine group is an alkyl (e.g., C₁₋₁₄ alkyl, C₁₋₁₂ alkyl, C₁₋₁₀ alkyl, etc.), 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl), wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof, wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl). In some embodiments, the linker is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, or —SS—CH₂—CH₂—C(O)N—. In some embodiments, the sterol group is a cholesterol, sitosterol, campesterol, stigmasterol or derivatives thereof.

In some embodiments, the sterol amine has Formula A2a:

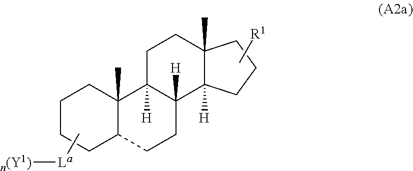

or a salt thereof, wherein:
---- is a single or double bond
R₁ is C₁₋₁₄ alkyl or C₁₋₁₄ alkenyl;
L^{a} is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, —SS—CH₂—CH₂—C(O)N—, or a group of formula (a):

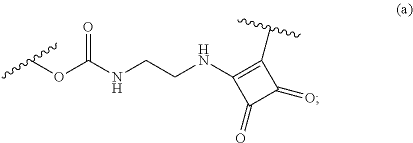 [SMILES: *OC(=O)NCCNc1c(*)c(=O)c1=O]

Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl)
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and n=1 or 2.

In some embodiments, the sterol amine has Formula A2a:

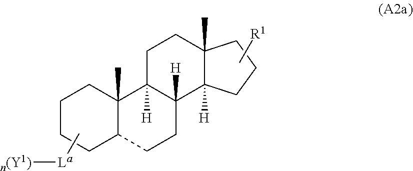

or a salt thereof, wherein:
---- is a single or double bond
R₁ is C₁₋₁₄ alkyl or C₁₋₁₄ alkenyl;
L^{a} is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, —SS—CH₂—CH₂—C(O)N—, or a group of formula (a):

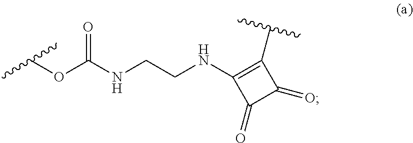 [SMILES: *OC(=O)NCCNc1c(*)c(=O)c1=O]

Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl)
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and n=1 or 2;
with the proviso that the compound of Formula A2a is other than:

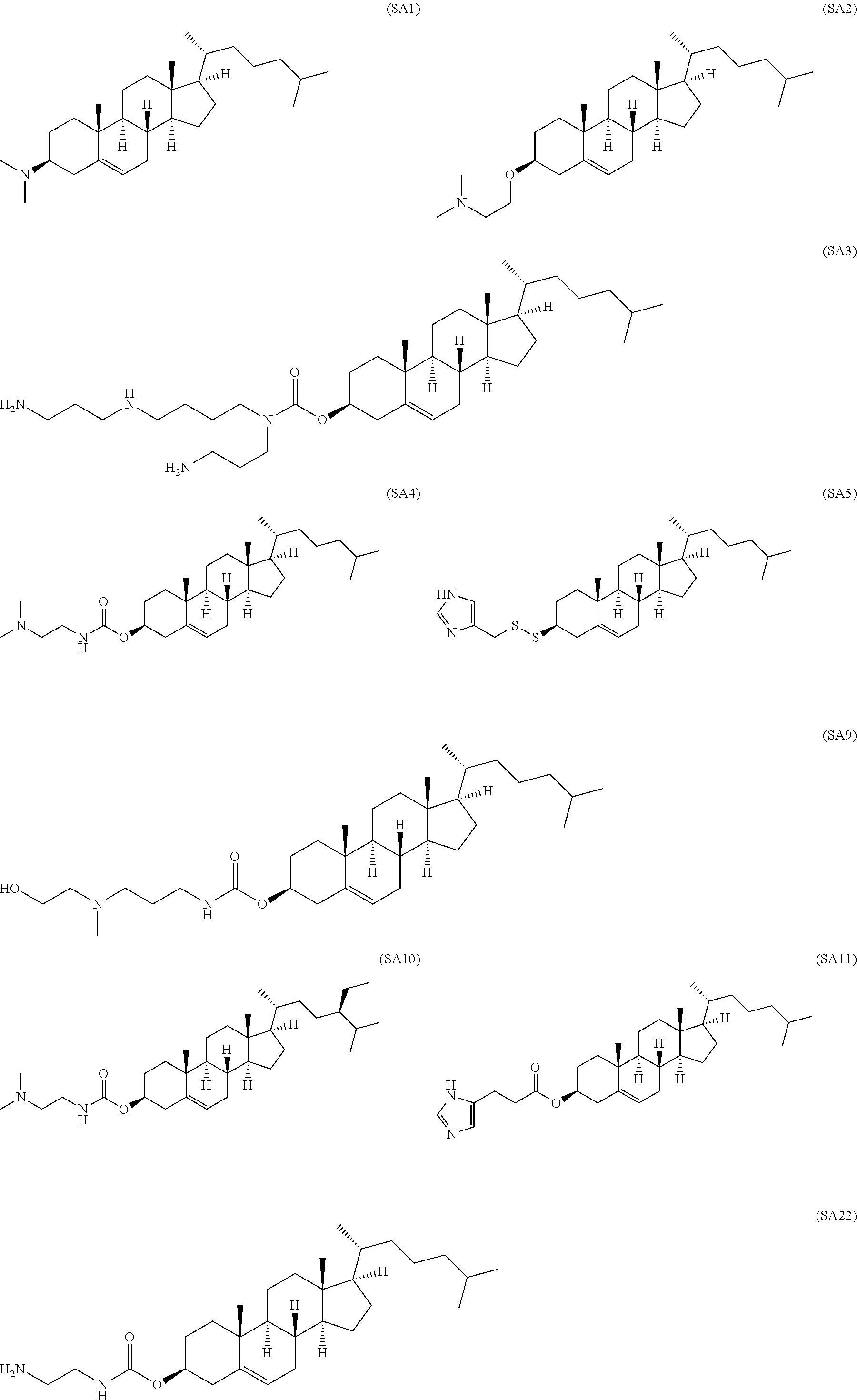

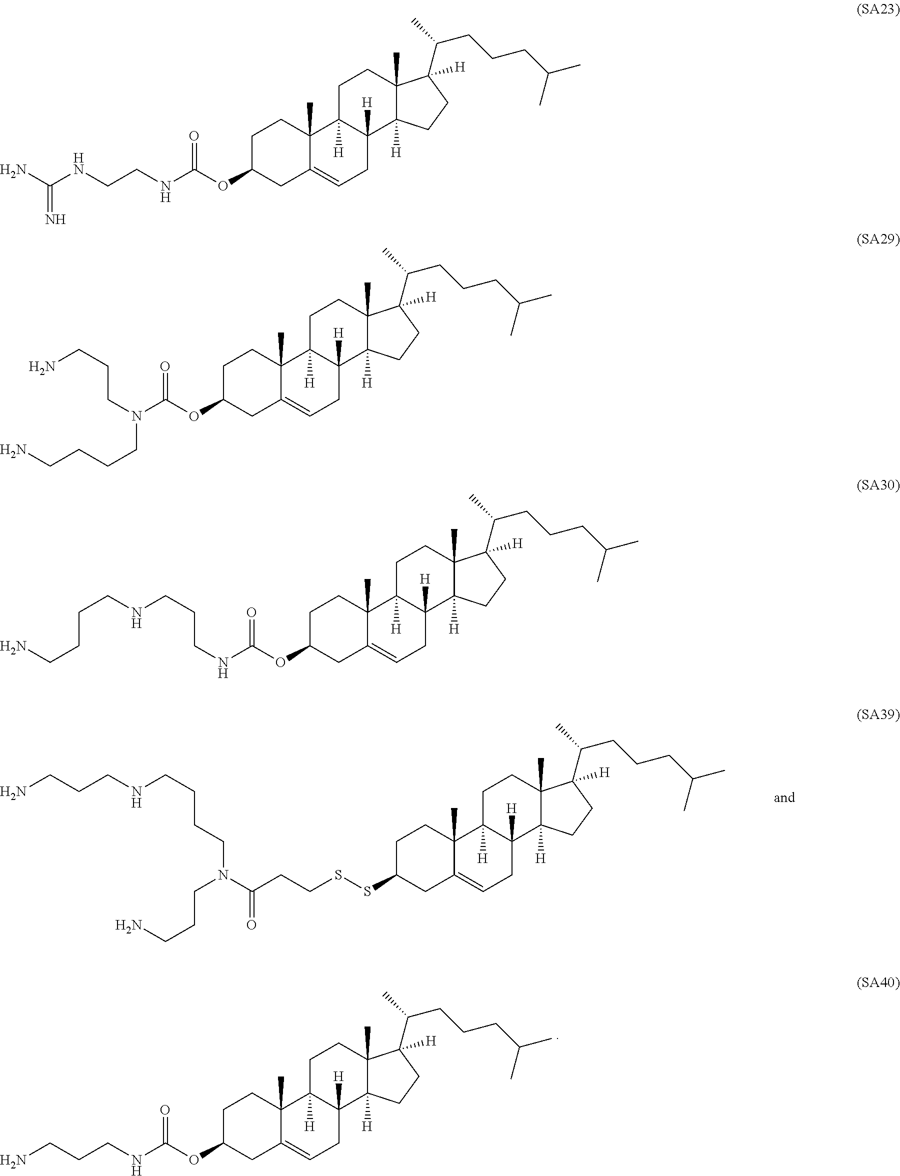

In some embodiments, ---- is a double bond. In some embodiments, ---- is a single bond.

In some embodiments, L^{a} is —OC(═O), —OC(═O)N—, or —OC(═O)—CH₂—CH₂—C(═O)N—.

In some embodiments, n is 1. In some embodiments, n is 2.

In some embodiments, R₁ is C₁₋₁₄ alkyl. In some embodiments, R₁ is C₁₋₁₄ alkenyl. In some embodiments, R₁ is

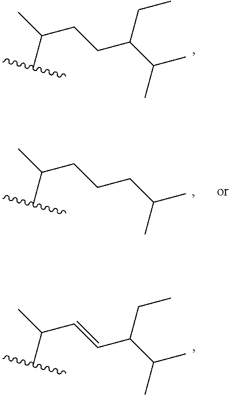

In some embodiments, Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, OH, —C₁₋₆ alkyl-OH, or NH₂.

In some embodiments, the sterol amine has Formula A2:

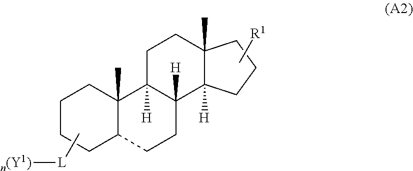

or a salt thereof, wherein:
---- is a single or double bond
R₁ is C₁₋₁₄ alkyl or C₁₋₁₄ alkenyl;
L is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, or —SS—CH₂—CH₂—C(O)N—;
Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and
      n=1 or 2.

In some embodiments, the sterol amine has Formula A3a:

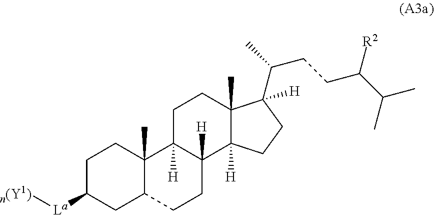

or a salt thereof, wherein:
---- is a single or double bond;
R₂ is H or C₁₋₆ alkyl;
L^{a} is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, —SS—CH₂—CH₂—C(O)N—, or a group of formula (a):

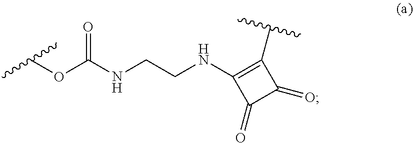 [SMILES: *OC(=O)NCCNc1c(*)c(=O)c1=O]

Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and n=1 or 2.

In some embodiments, the sterol amine has Formula A3a:

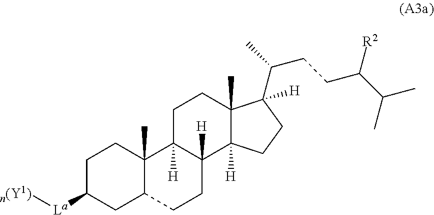

or a salt thereof, wherein:
---- is a single or double bond;
R₂ is H or C₁₋₆ alkyl;
L^{a} is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, —SS—CH₂—CH₂—C(O)N—, or a group of formula (a):

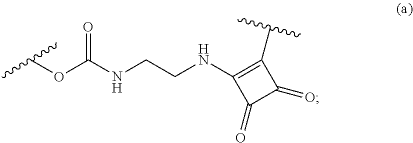 [SMILES: *OC(=O)NCCNc1c(*)c(=O)c1=O]

Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and n=1 or 2;
with the proviso that the compound of Formula A2a is other than:

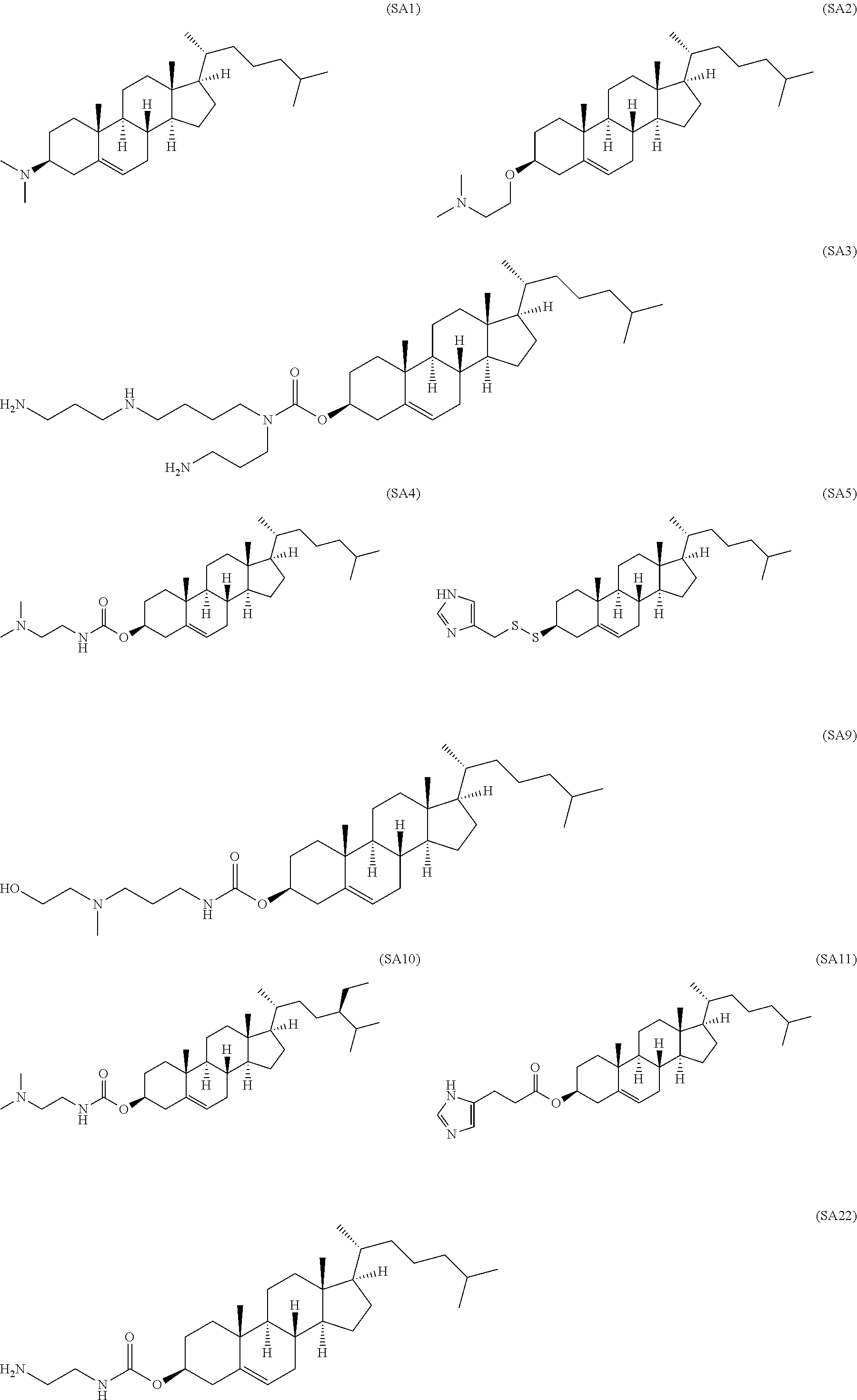

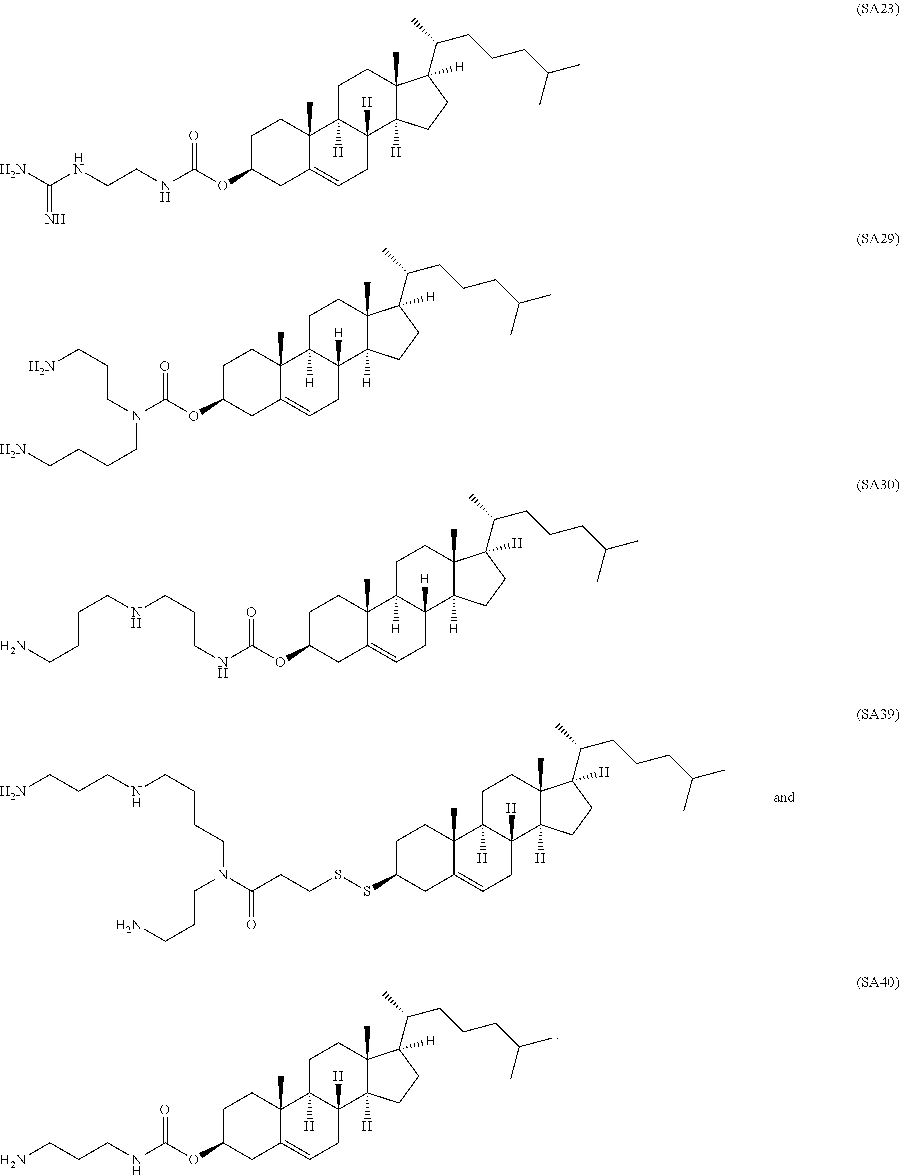

In some embodiments, ---- is a double bond. In some embodiments, ---- is a single bond.

In some embodiments, L^{a} is —OC(═O), —OC(═O)N—, or —OC(═O)—CH₂—CH₂—C(═O)N—.

In some embodiments, n is 1. In some embodiments, n is 2.

In some embodiments, R₂ is H. In some embodiment, R₂ is ethyl.

In some embodiments, Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, OH, —C₁₋₆ alkyl-OH, or NH₂.

In some embodiments, the sterol amine has Formula A3:

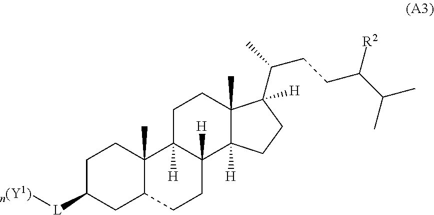

or a salt thereof, wherein:
---- is a single or double bond;
R₂ is H or C₁₋₆ alkyl;
L is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, or —SS—CH₂—CH₂—C(O)N—;
Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and n=1 or 2.

In some embodiments, Y¹ is selected from:

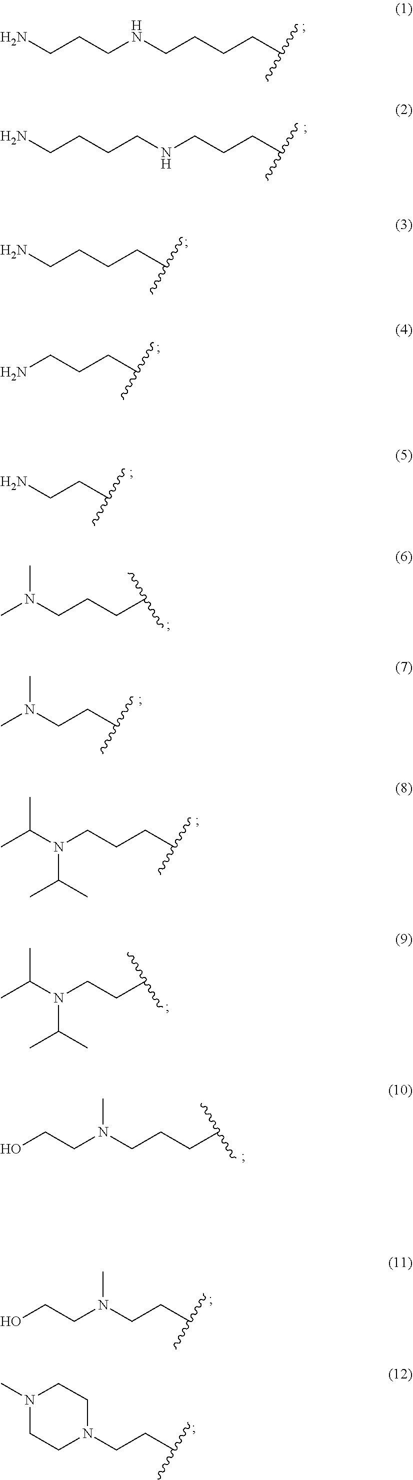

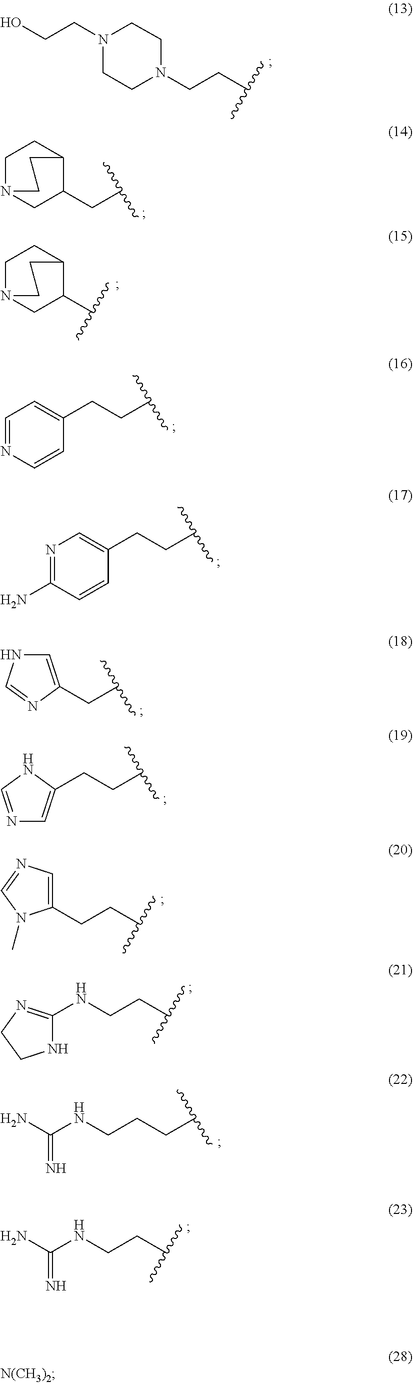

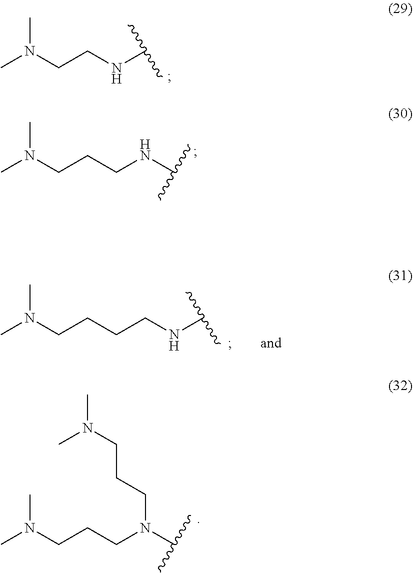

In some embodiments Y¹ is selected from:

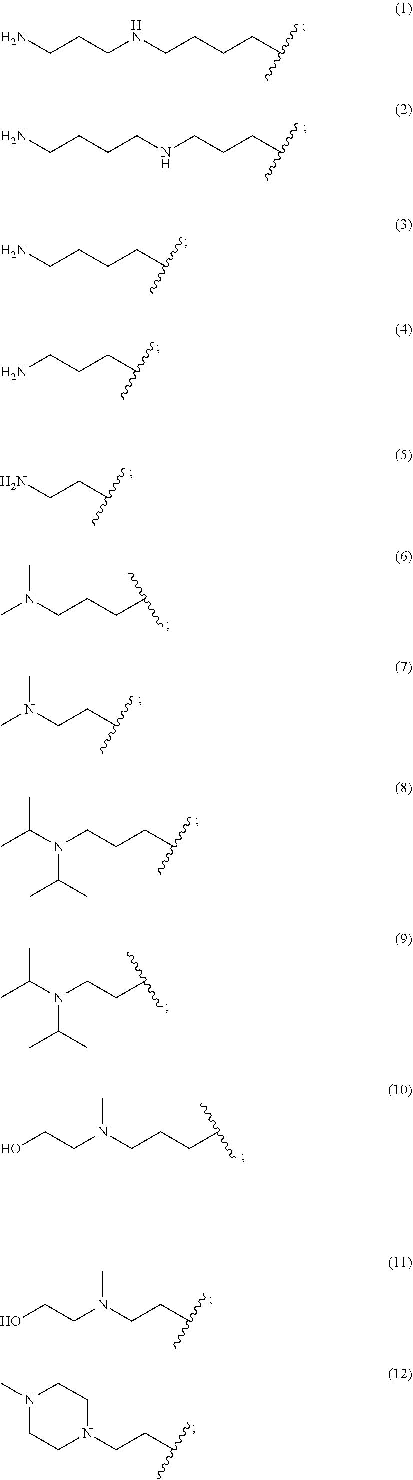

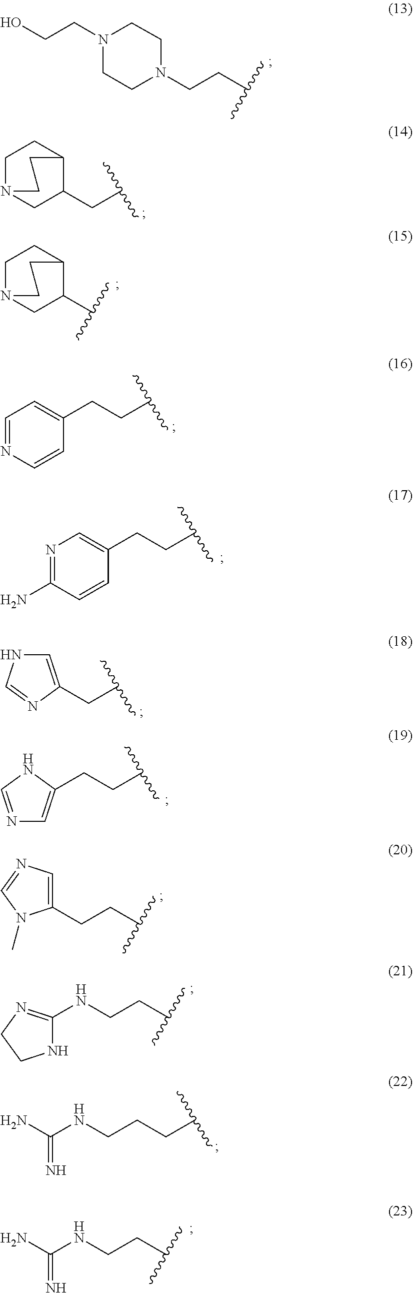

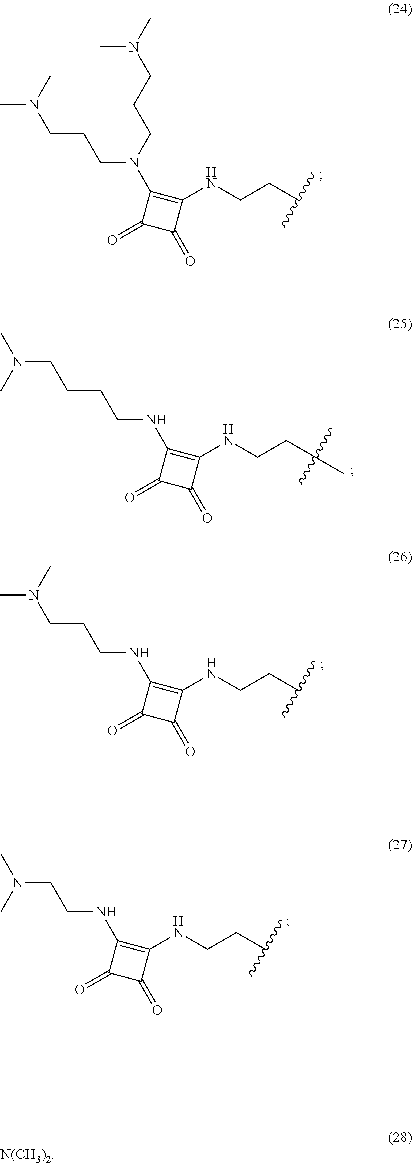 [SMILES: *CCNc1c(N(CCCN(C)C)CCCN(C)C)c(=O)c1=O ; *CCNc1c(NCCCCN(C)C)c(=O)c1=O ; *CCNc1c(NCCCN(C)C)c(=O)c1=O ; *CCNc1c(NCCN(C)C)c(=O)c1=O]

In some embodiments, the sterol amine has Formula A4:

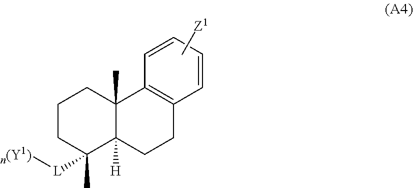

or a salt thereof, wherein:
Z¹ is OH or C₃₋₆ alkyl;
L is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, or —SS—CH₂—CH₂—C(O)N—;
Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
   wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, OH, O(C₁₋₆ alkyl), C₁₋₆ alkyl-OH, NH₂, NH(C₁₋₆ alkyl), N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, NH(3 to 8 membered heterocycloalkyl), and NH(5 to 6 membered heteroaryl); and n=1 or 2.

In some embodiments, Z¹ is OH. In some embodiments, Z¹ is C₃₋₆ alkyl.

In some embodiments, L is —C(═O)N—, —CH₂—NH—C(═O)—, or —C(═O)O—.

In some embodiments, Y¹ is C₁₋₁₀ alkyl comprising one to five primary, secondary, or tertiary amines or combination thereof. In some embodiments, Y¹ is

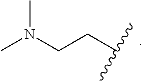 [SMILES: *CCN(C)C]

In some embodiments, n is 1. In some embodiments, n is 2.

In some embodiments, the sterol amine has Formula A5:

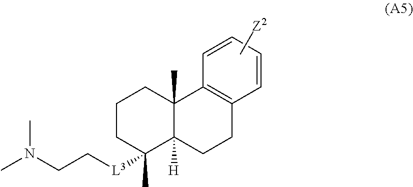

or a salt thereof, wherein:
Z² is OH or isopropyl;
L³ is —CH₂—NH—C(O)—, —C(O)NH—, or —C(O)O—.

In some embodiments, the sterol amine is selected from:

TABLE 1

| Sterol amine | |
|---|---|
| no. | Structure |
| SA1 | 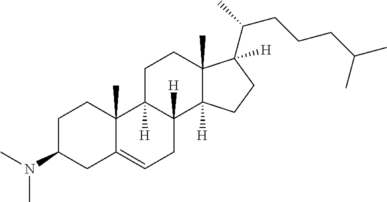 |
| SA2 | 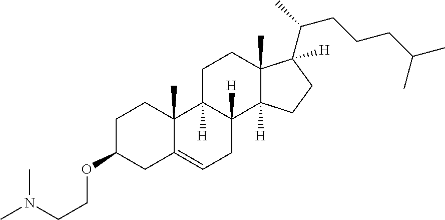 |
| SA3 | 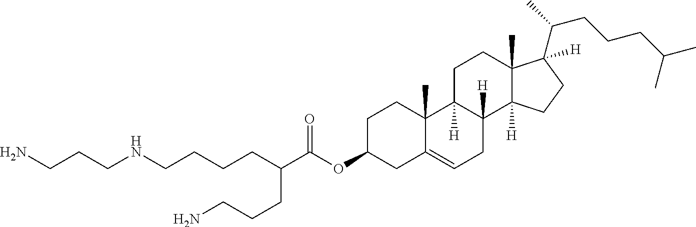 |
| SA4 | 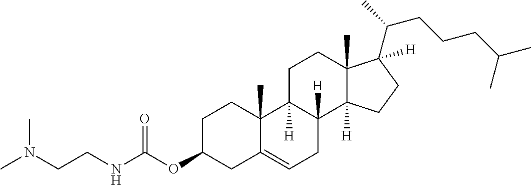 |
| SA5 | 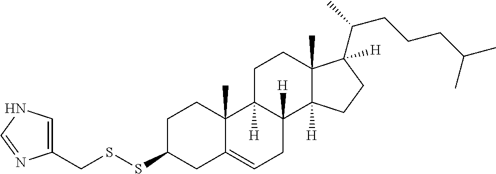 |
| SA6 | 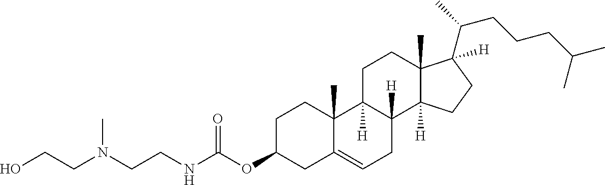 |
| SA7 | 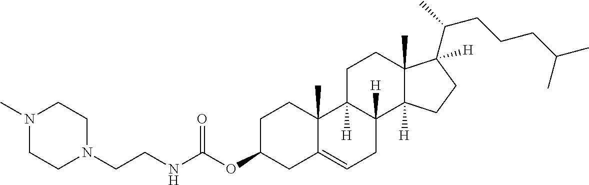 |
| SA8 | 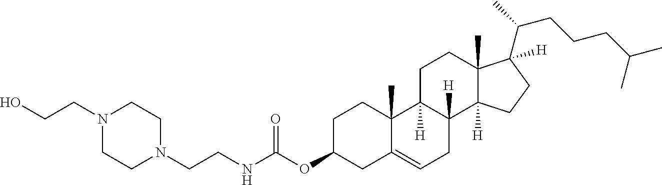 |
| SA9 | 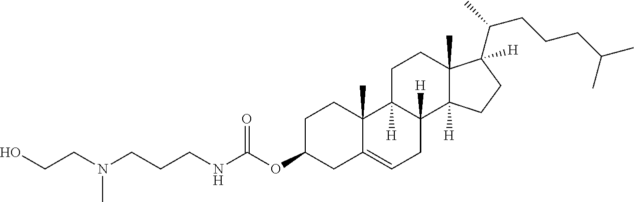 |
| SA10 | 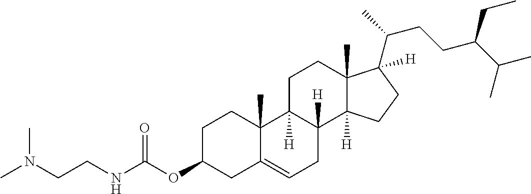 |
| SA11 | 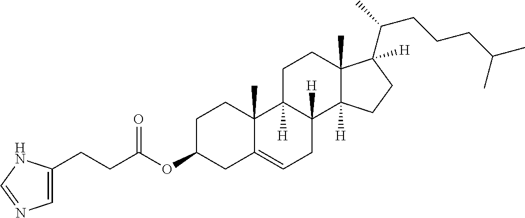 |
| SA12 | 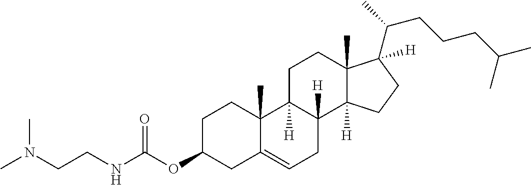 |
| SA13 | 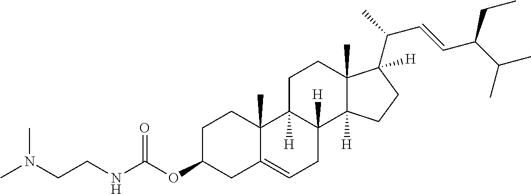 |
| SA14 | 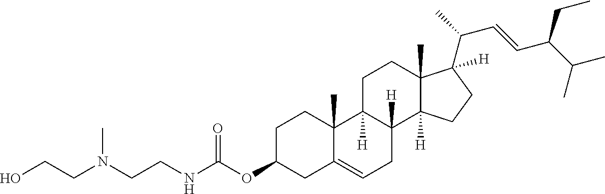 |
| SA15 | 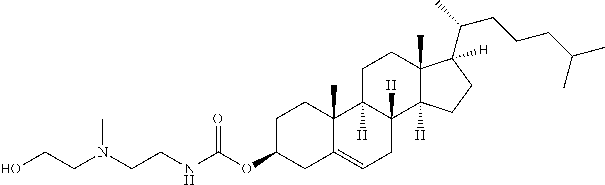 |
| SA16 | 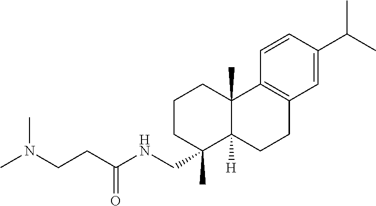 [SMILES: CC(C)c1ccc2c(c1)CC[C@H]1[C@](C)(CNC(=O)CCN(C)C)CCC[C@]21C] |
| SA17 | 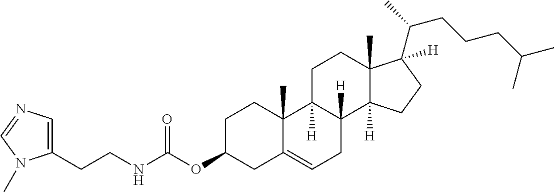 |
| SA18 | 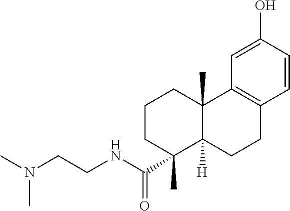 [SMILES: CN(C)CCNC(=O)[C@]1(C)CCC[C@]2(C)c3cc(O)ccc3CC[C@@H]12] |
| SA19 | 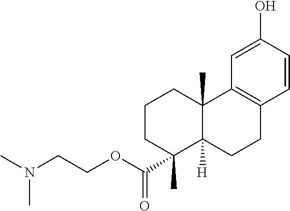 [SMILES: CN(C)CCOC(=O)[C@]1(C)CCC[C@]2(C)c3cc(O)ccc3CC[C@@H]12] |
| SA20 | 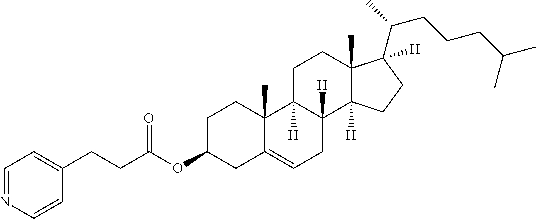 |
| SA21 | 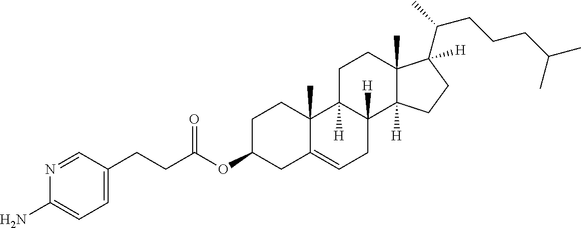 |
| SA22 | 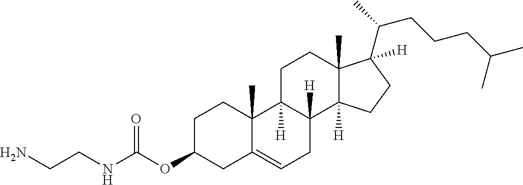 |
| SA23 | 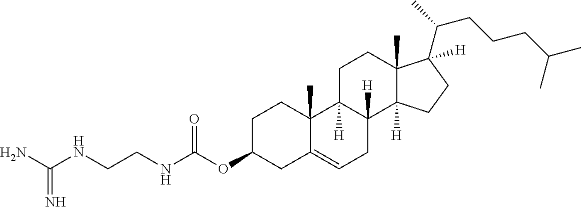 |
| SA24 | 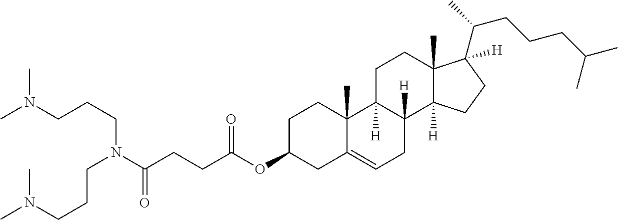 |
| SA25 | 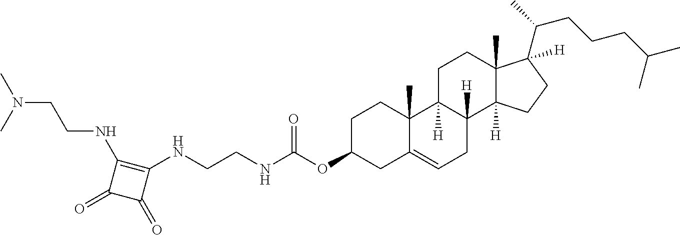 |
| SA26 | 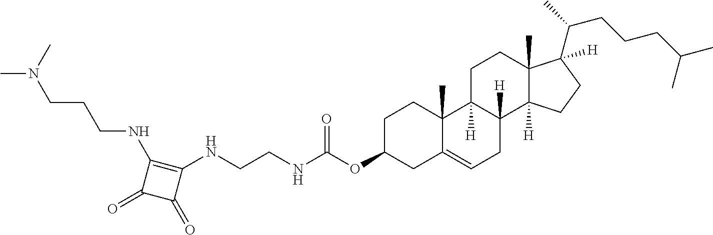 |
| SA27 | 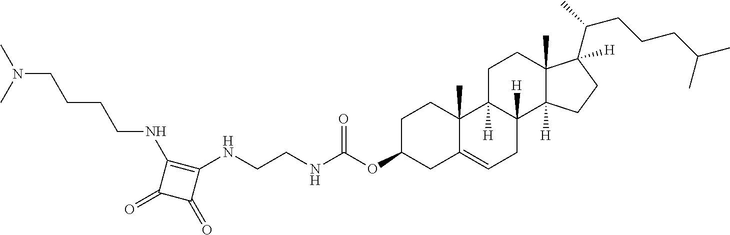 |
| SA28 | 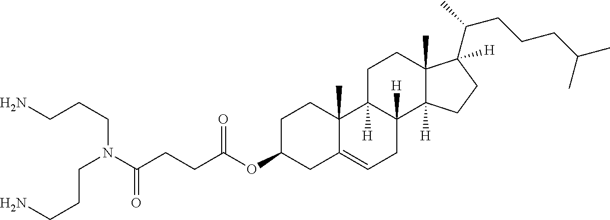 |
| SA29 | 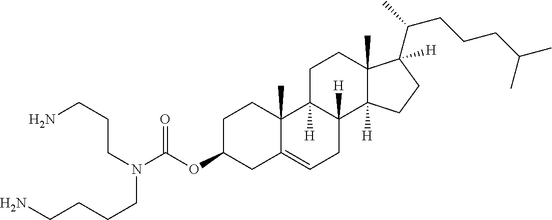 |
| SA30 | 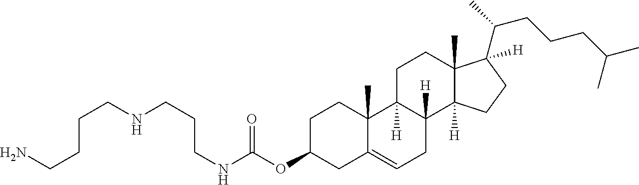 |
| SA31 | 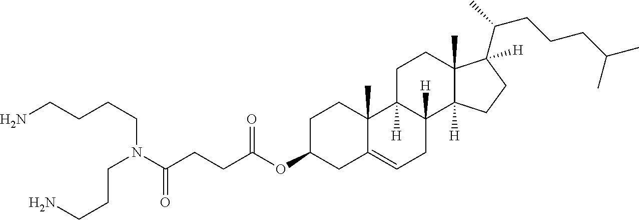 |
| SA32 | 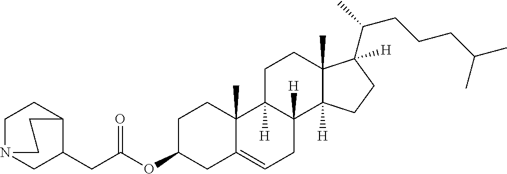 |
| SA33 | 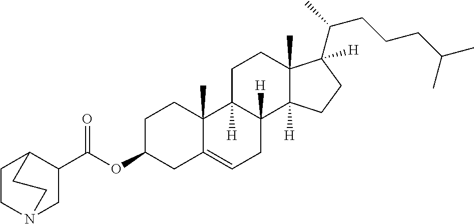 |
| SA34 | 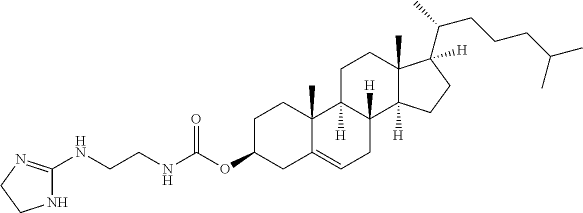 |
| SA35 | 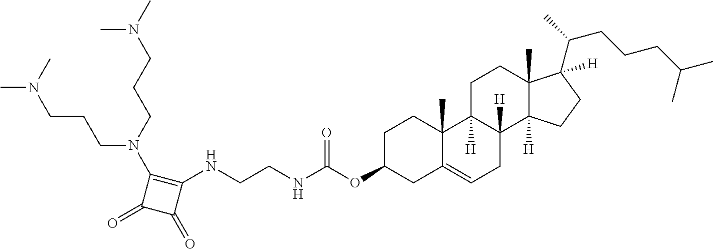 |
| SA36 | 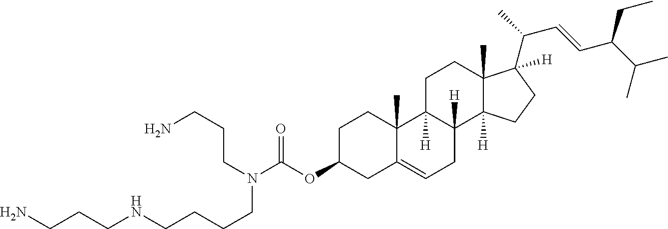 |
| SA37 | 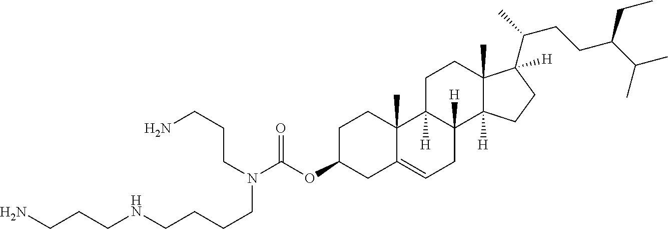 |
| SA38 | 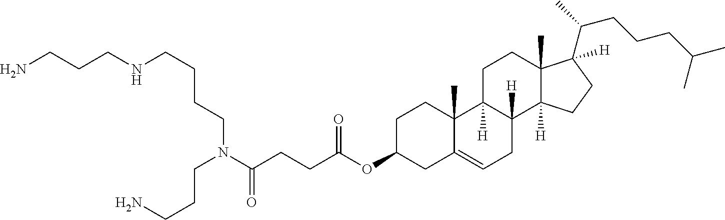 |
| SA39 | 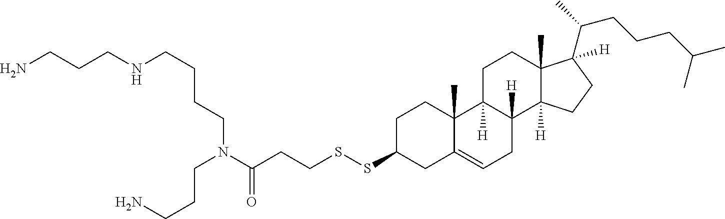 |
| SA40 | 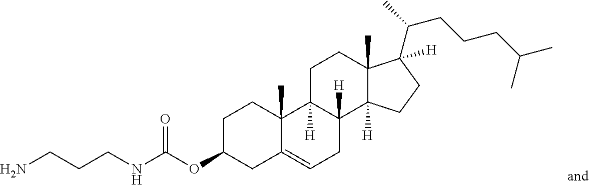 |
| SA41 | 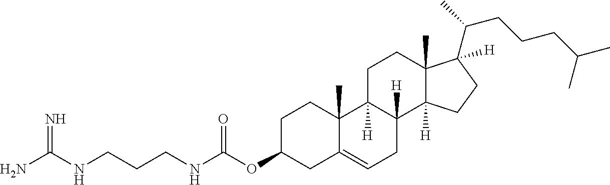 |

or a salt thereof.

In some embodiments, the sterol amine is selected from:

TABLE 2

| Sterol amine no. | Structure |
|---|---|
| SA1 | 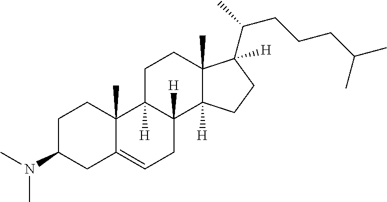 |
| SA2 | 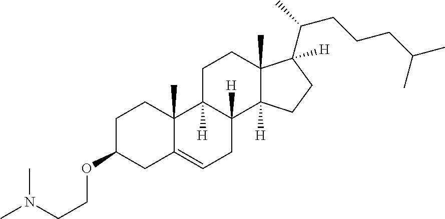 |
| SA3 | 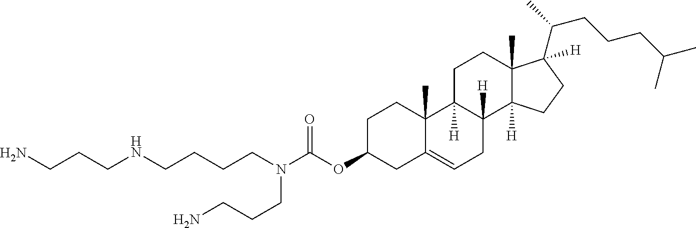 |
| SA4 | 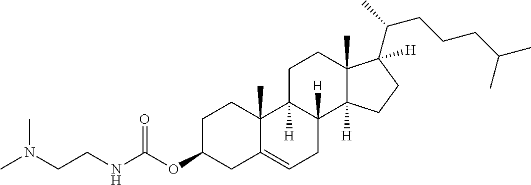 |
| SA5 | 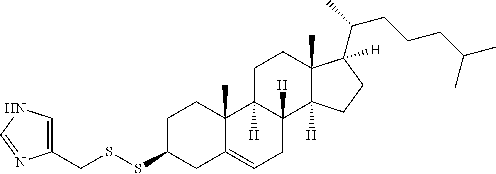 |
| SA6 | 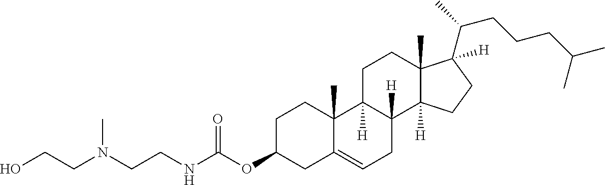 |
| SA7 | 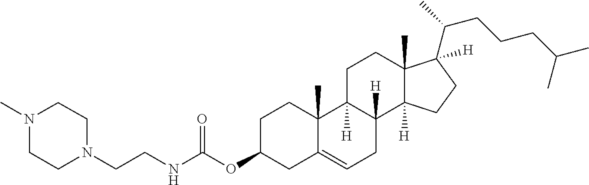 |
| SA8 | 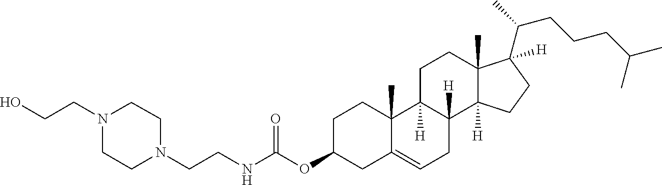 |
| SA9 | 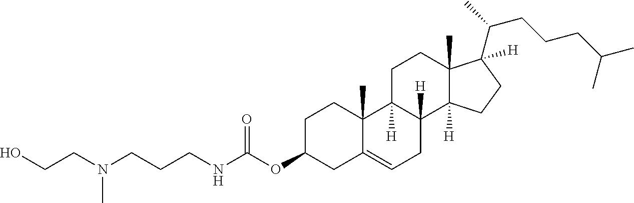 |
| SA10 | 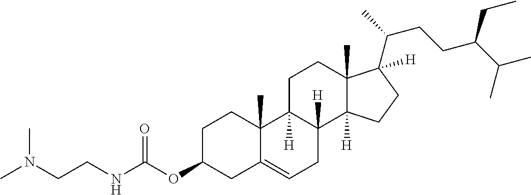 |
| SA11 | 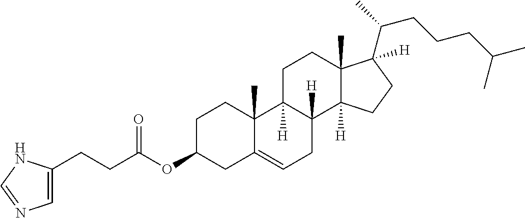 |
| SA12 | 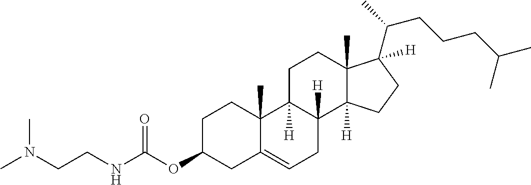 |
| SA13 | 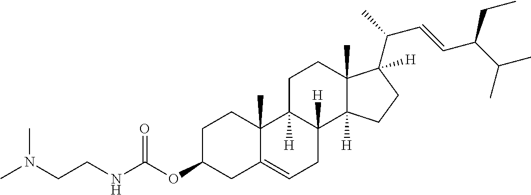 |
| SA14 | 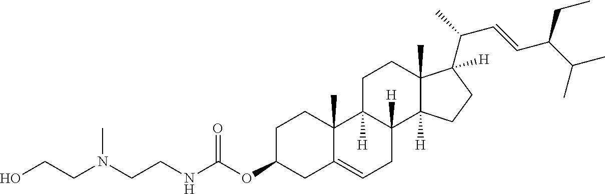 |
| SA15 | 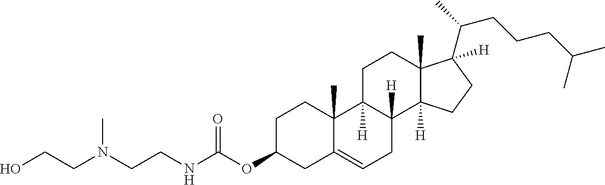 |
| SA16 | 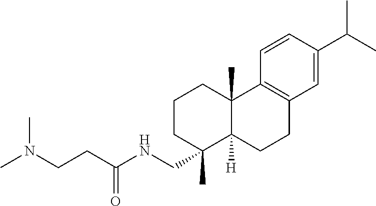 [SMILES: CC(C)c1ccc2c(c1)CC[C@H]1[C@](C)(CNC(=O)CCN(C)C)CCC[C@]21C] |
| SA17 | 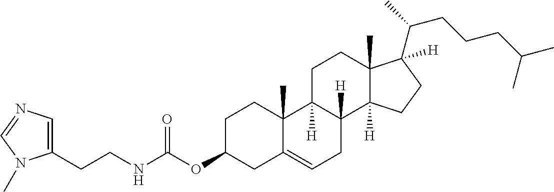 |
| SA18 | 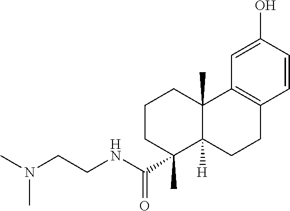 [SMILES: CN(C)CCNC(=O)[C@]1(C)CCC[C@]2(C)c3cc(O)ccc3CC[C@@H]12] |
| SA19 | 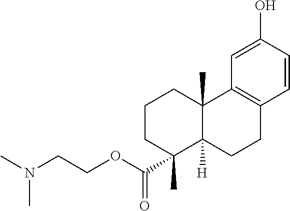 [SMILES: CN(C)CCOC(=O)[C@]1(C)CCC[C@]2(C)c3cc(O)ccc3CC[C@@H]12] |
| SA20 | 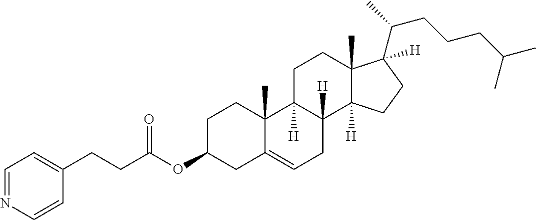 |
| SA21 | 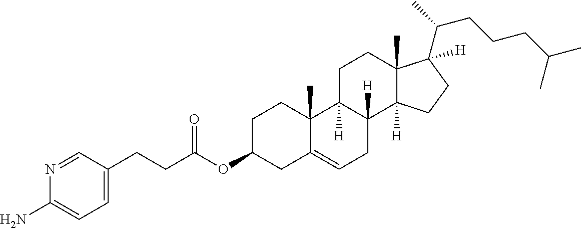 |
| SA22 | 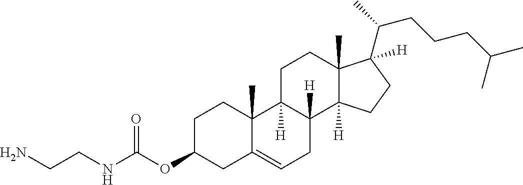 |
| SA23 | 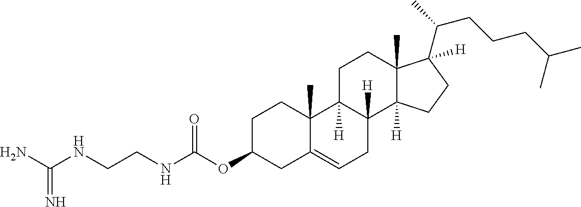 |
| SA24 | 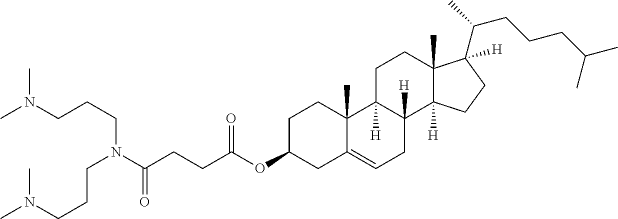 |
| SA25 | 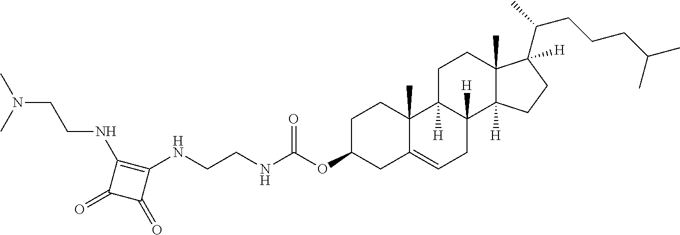 |
| SA26 | 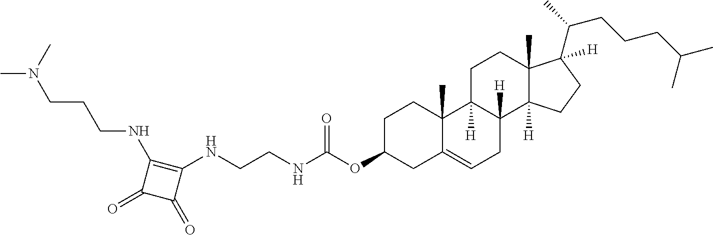 |
| SA27 | 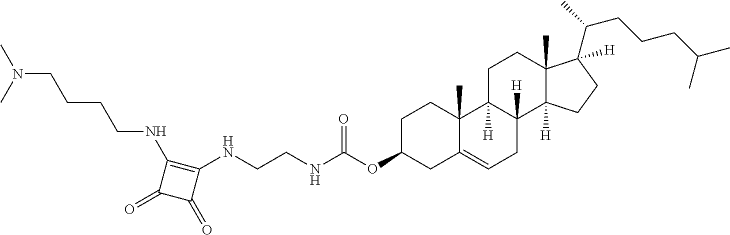 |
| SA28 | 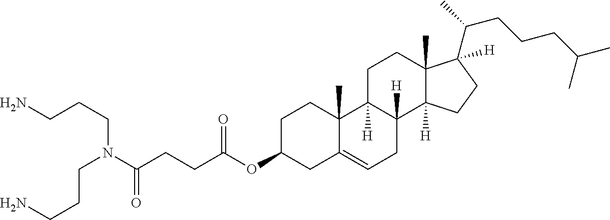 |
| SA29 | 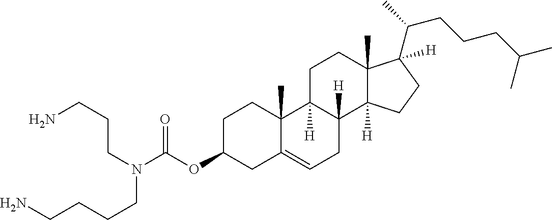 |
| SA30 | 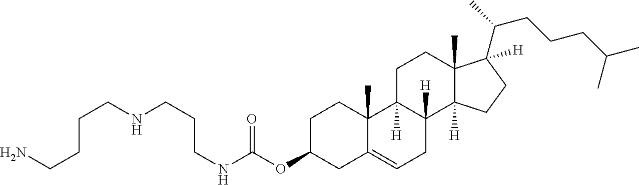 |
| SA31 | 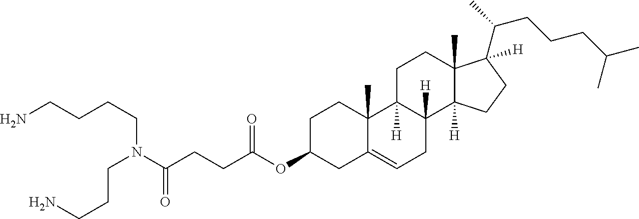 |
| SA32 | 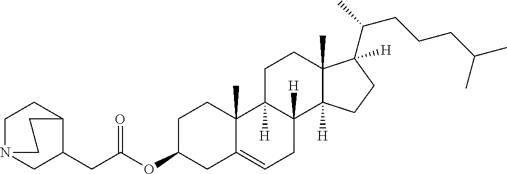 |
| SA33 | 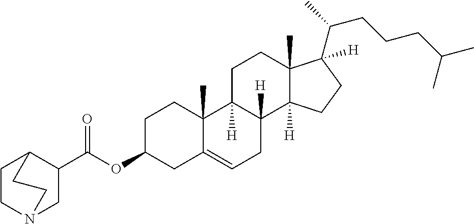 |
| SA34 | 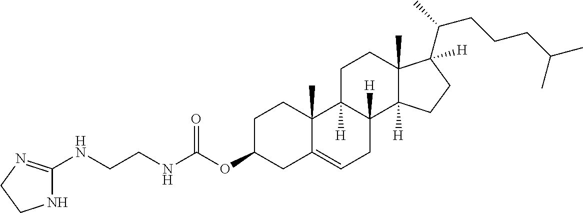 |
| SA35 | 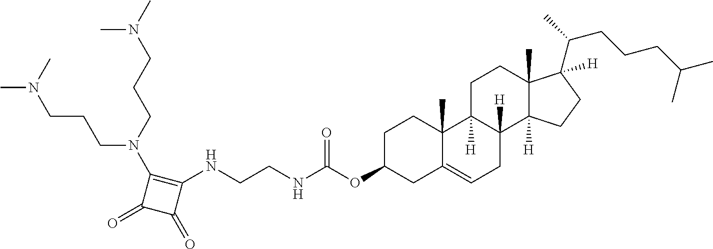 |
| SA36 | 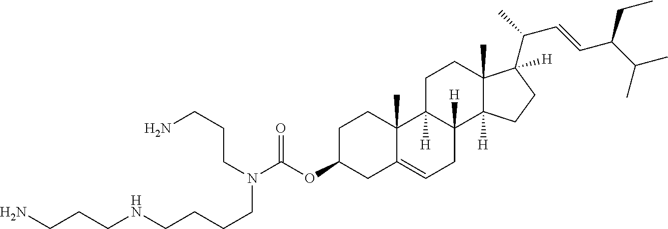 |
| SA37 | 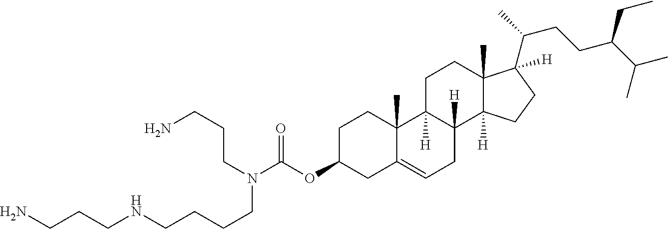 |
| SA38 | 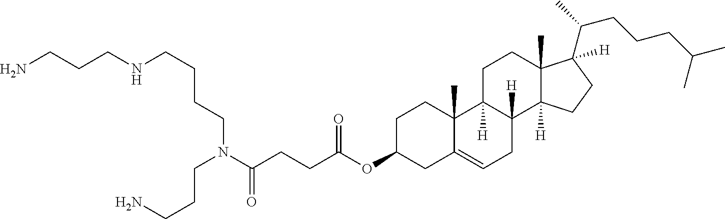 |
| SA39 | 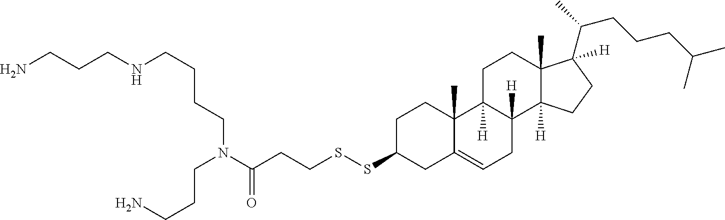 |
| SA40 | 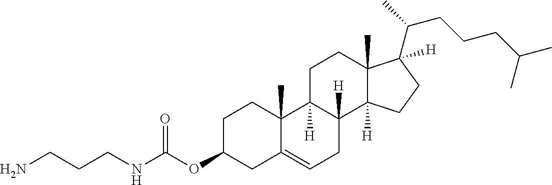 |
| SA41 | 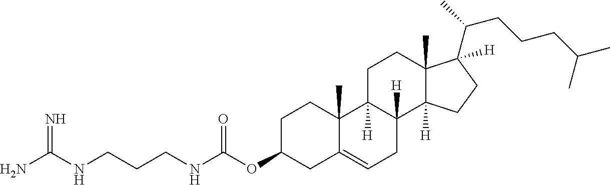 |
| SA42 | 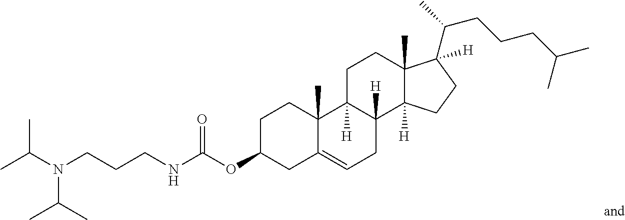 |
| SA43 | 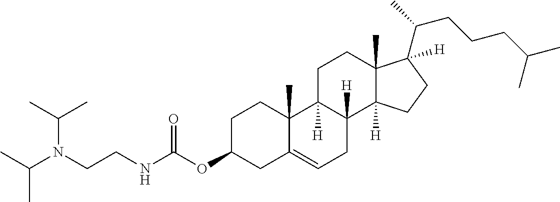 |

In some embodiments, the sterol amine is selected from:

TABLE 4

| Sterol amine no. | Structure |
|---|---|
| SA6 | 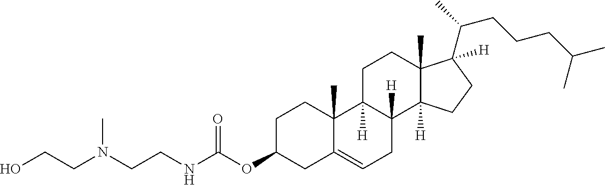 |
| SA7 | 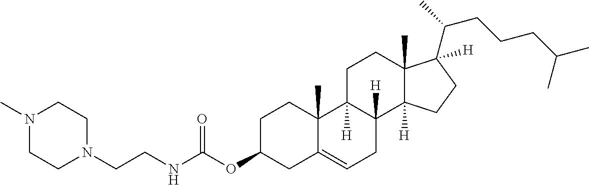 |
| SA8 | 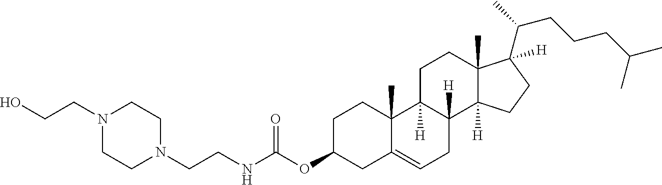 |
| SA12 | 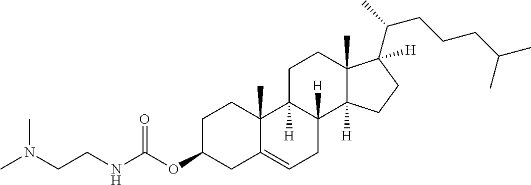 |
| SA13 | 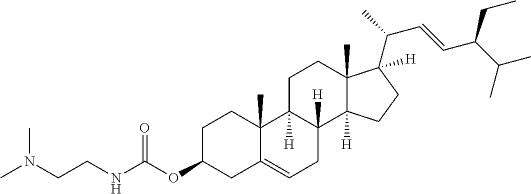 |
| SA14 | 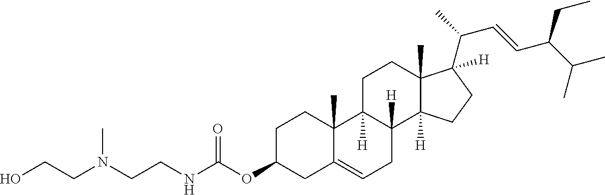 |
| SA15 | 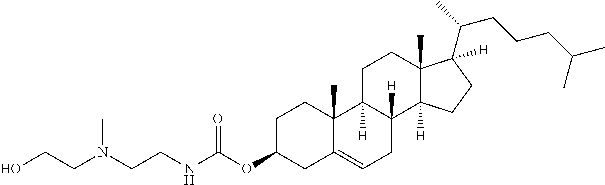 |
| SA16 | 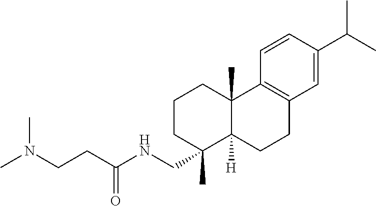 [SMILES: CC(C)c1ccc2c(c1)CC[C@H]1[C@](C)(CNC(=O)CCN(C)C)CCC[C@]21C] |
| SA17 | 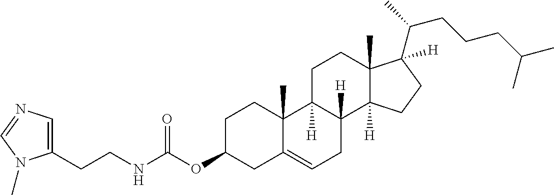 |
| SA18 | 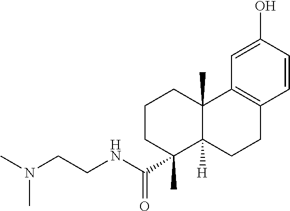 [SMILES: CN(C)CCNC(=O)[C@]1(C)CCC[C@]2(C)c3cc(O)ccc3CC[C@@H]12] |
| SA19 | 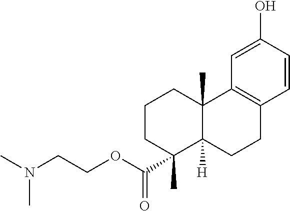 [SMILES: CN(C)CCOC(=O)[C@]1(C)CCC[C@]2(C)c3cc(O)ccc3CC[C@@H]12] |
| SA20 | 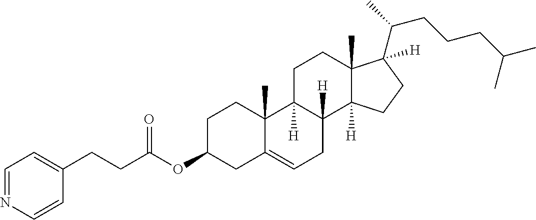 |
| SA21 | 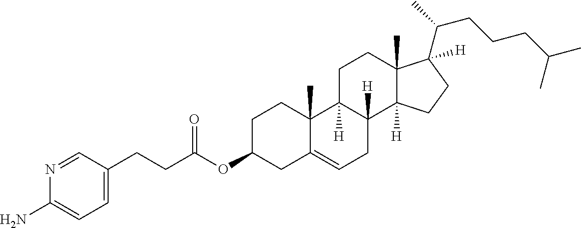 |
| SA24 | 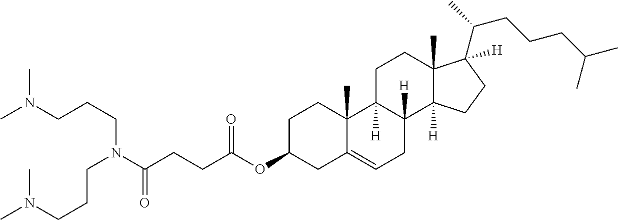 |
| SA25 | 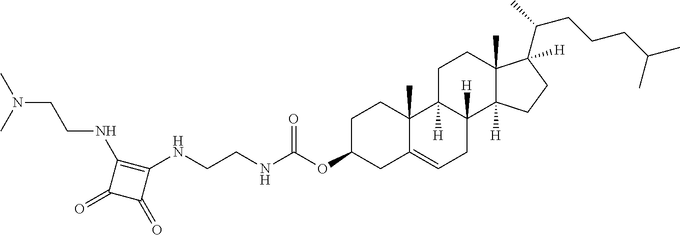 |
| SA26 | 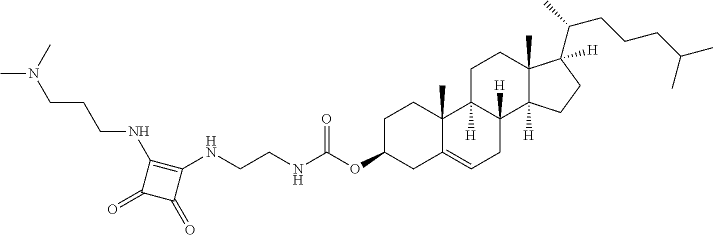 |
| SA27 | 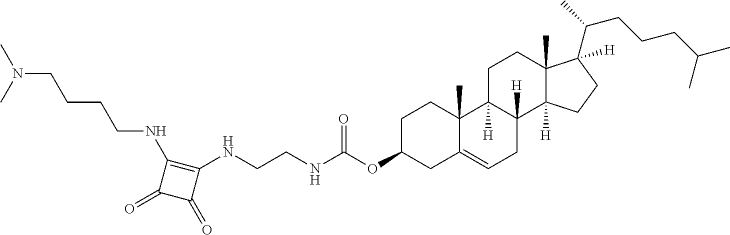 |
| SA28 | 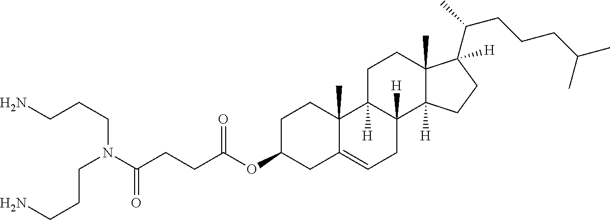 |
| SA31 | 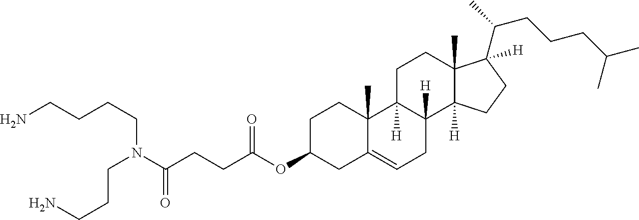 |
| SA32 | 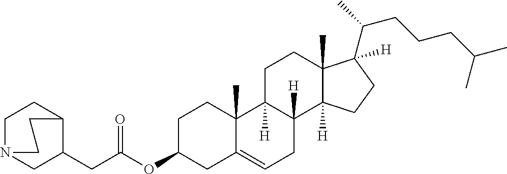 |
| SA33 | 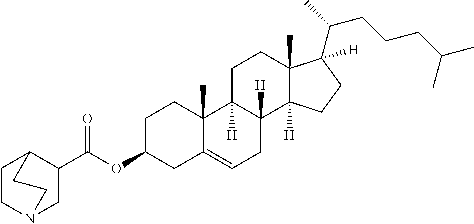 |
| SA34 | 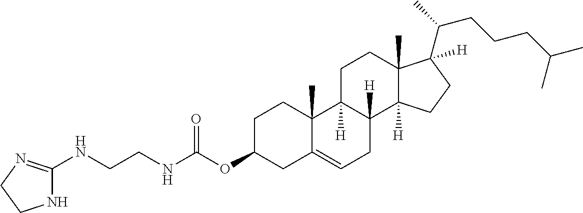 |
| SA35 | 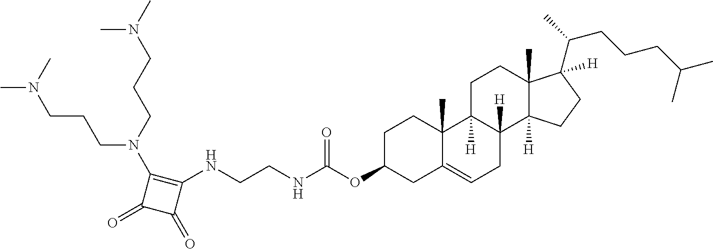 |
| SA36 | 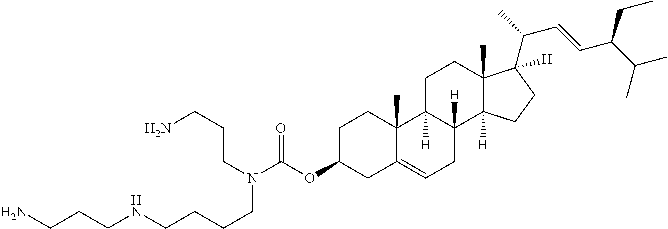 |
| SA37 | 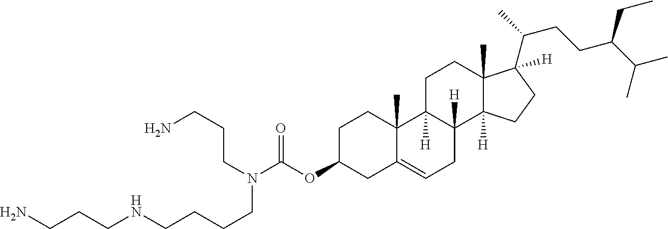 |
| SA38 | 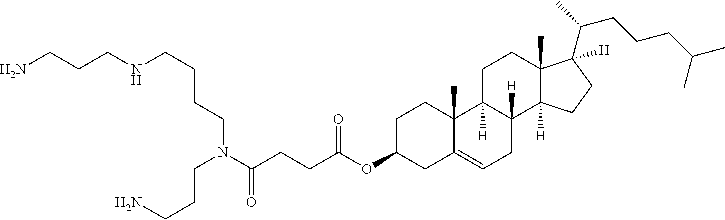 |
| SA41 | 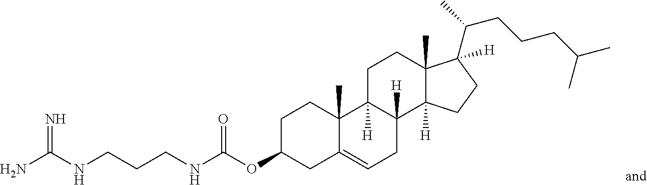 |
| SA42 | 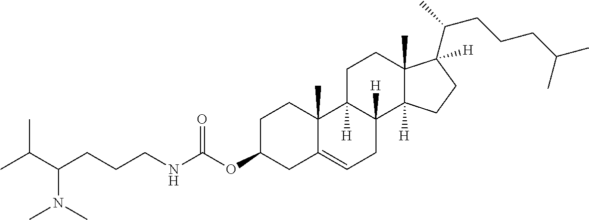 |

or salt thereof.

In some embodiments, the sterol amine is selected from:

TABLE 5

| Sterol amine no. | Structure |
|---|---|
| SA16 | 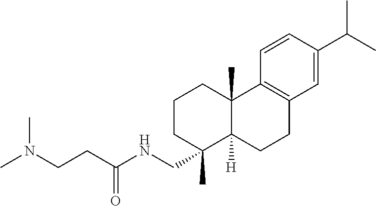 [SMILES: CC(C)c1ccc2c(c1)CC[C@H]1[C@](C)(CNC(=O)CCN(C)C)CCC[C@]21C] |
| SA18 | 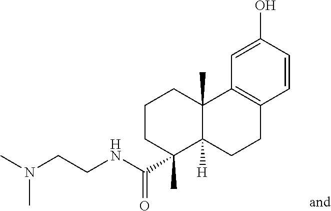 [SMILES: CN(C)CCNC(=O)[C@]1(C)CCC[C@]2(C)c3cc(O)ccc3CC[C@@H]12] |
| SA19 | 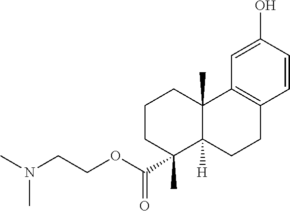 [SMILES: CN(C)CCOC(=O)[C@]1(C)CCC[C@]2(C)c3cc(O)ccc3CC[C@@H]12] |

or salt thereof.

In some embodiments, the sterol amine is SA3:

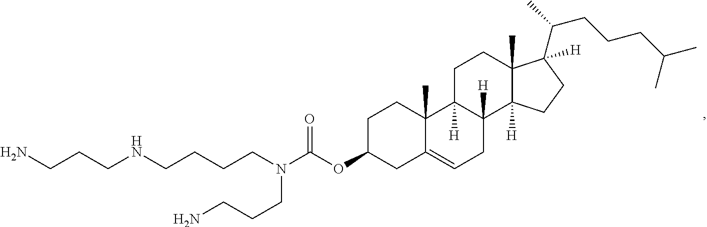

or a salt thereof, which is also referred to as GL-67. SA3 or GL-67 can be prepared according to known processes in the art or purchased from a commercial vendor such as Avanti® Polar Lipids, Inc. (SKU 890893).

In some embodiments, the cationic lipid is a modified amino acid, such as a modified arginine, in which an amino acid residue having an amine-containing side chain is appended to a hydrophobic group such as a sterol (e.g., cholesterol or derivative thereof), fatty acid, or similar hydrocarbyl group. At least one amine of the modified amino acid portion has a pKa of 8.0 or greater. At least one amine of the modified amino acid portion is positively charged at physiological pH. The amino acid residue can include but is not limited to arginine, histidine, lysine, tryptophan, ornithine, and 5-hydroxylysine. The amino acid is bonded to the hydrophobic group through a linker.

In some embodiments, the modified amino acid is a modified arginine.

In some embodiments, the cationic agent is a non-lipid cationic agent. Examples of non-lipid cationic agent include e.g., benzalkonium chloride, cetylpyridium chloride, L-lysine monohydrate, or tromethamine.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

### Ionizable Lipid

As used herein, the term “ionizable lipid” has its ordinary meaning in the art and may refer to a lipid comprising one or more charged moieties. In some embodiments, an ionizable lipid may be positively charged or negatively charged. For instance, an ionizable lipid may be positively charged at lower pHs, in which case it could be referred to as “cationic lipid.” In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid. In one embodiment, the ionizable amino lipid may have a positively charged hydrophilic head and a hydrophobic tail that are connected via a linker structure.

In some embodiments, the nanoparticle described herein comprises about 30 mol % to about 60 mol % of ionizable lipid. In some embodiments, the nanoparticle comprises about 40 mol % to about 50 mol % of ionizable lipid.

A lipid nanoparticle composition of the invention may include one or more ionizable (e.g., ionizable amino) lipids (e.g., lipids that may have a positive or partial positive charge at physiological pH). Ionizable lipids may be selected from the non-limiting group consisting of 3-(didodecylamino)-N1,N1,4-tridodecyl-1-piperazineethanamine (KL10), N1-[2-(didodecylamino)ethyl] N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8 [(30)-cholest-5-en-3-yloxy]octyl}oxy) N,N dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S) 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable lipid may also be a lipid including a cyclic amine group.

Ionizable lipids can also be the compounds disclosed in International Publication No. WO 2017/075531 A1, hereby incorporated by reference in its entirety. For example, the ionizable amino lipids include, but not limited to:

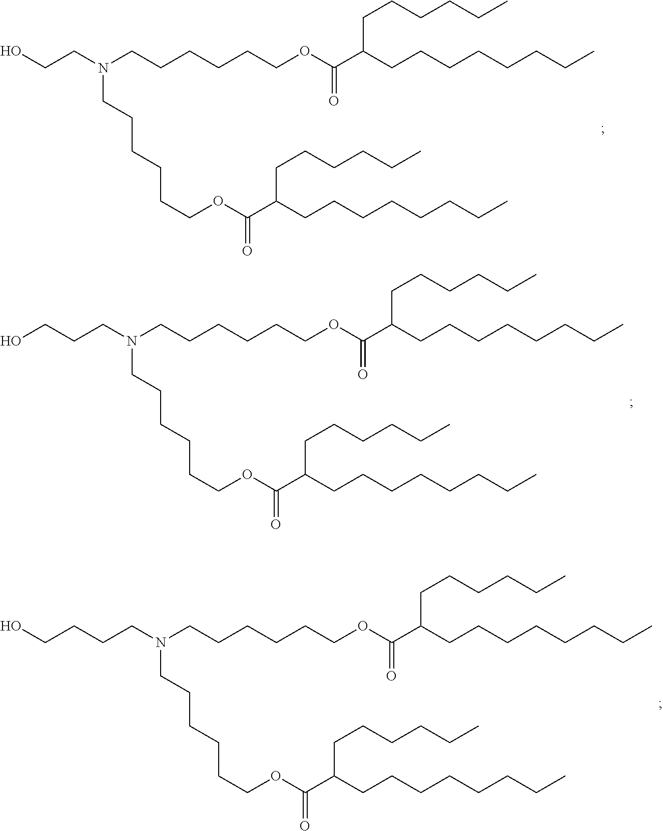 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids can also be the compounds disclosed in International Publication No. WO 2015/199952 A1, hereby incorporated by reference in its entirety. For example, the ionizable amino lipids include, but not limited to:

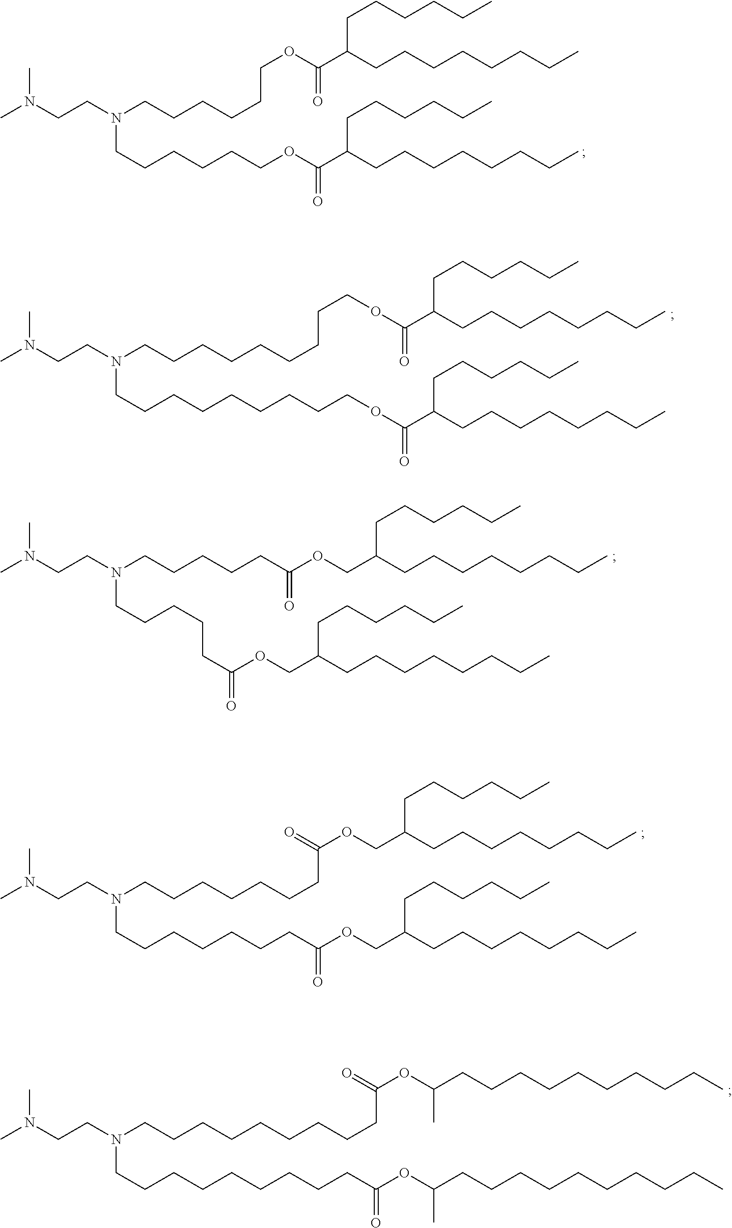

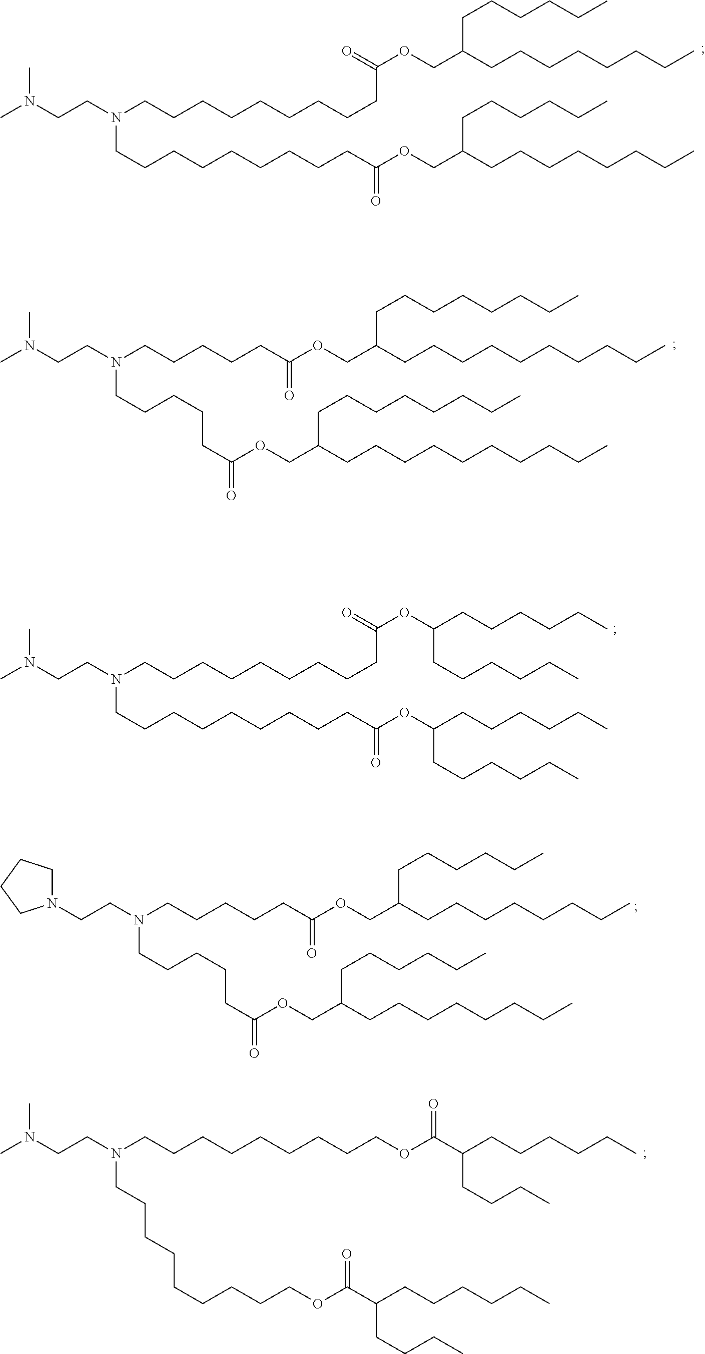

and any combination thereof.

In one embodiment, the ionizable lipid may be selected from, but not limited to, an ionizable lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and S20130225836; the contents of each of which are herein incorporated by reference in their entirety.

In another embodiment, the ionizable lipid may be selected from, but not limited to, formula A described in International Publication Nos. WO2013116126 or US20130225836; the contents of each of which is herein incorporated by reference in their entirety. In yet another embodiment, the ionizable lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety.

As a non-limiting example, a cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-1 6, 19-dien-8-amine, (13Z,16Z)-N,N-dimethyldocosa-13,16-dien-5-amine, (12Z,15Z)-N,N dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl] pyrrolidine, (20Z)-N,N-dimethylheptacos-20-en-1 0-amine, (15Z)-N,N-dimethyl eptacos-15-en-1 0-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-1 0-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2 undecylcyclopropyl]tetradecan-5-amine, N,N-dimethyl-3-{7-[(1S,2R)-2-octylcyclopropyl]heptyl} dodecan-1-amine, 1-[(1R,2S)-2-hepty lcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

Additional examples of ionizable lipids include the following:

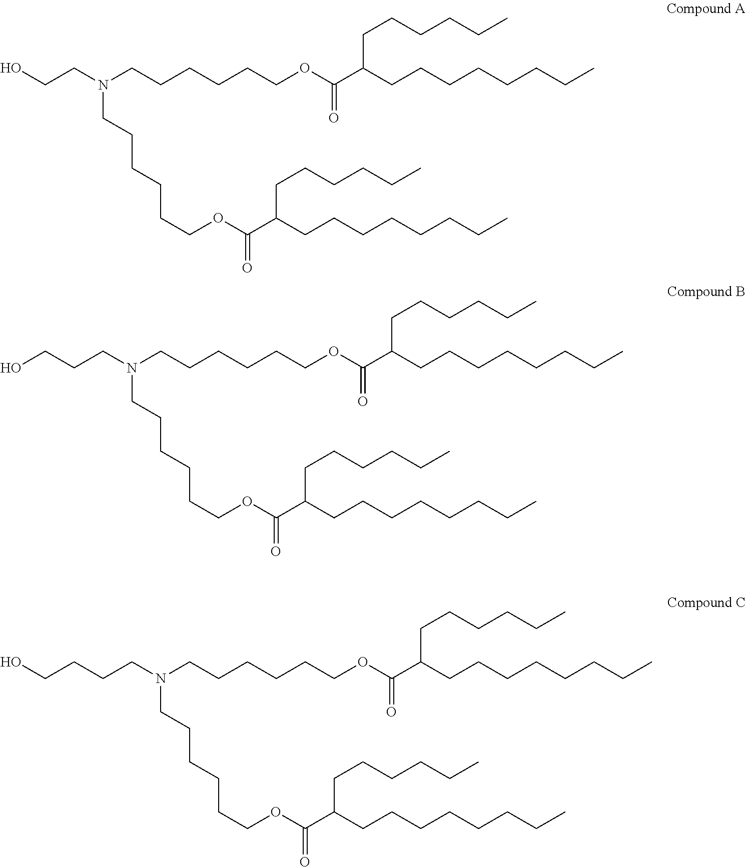 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety. In another embodiment, the lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In some embodiments, the ionizable lipid may be a compound of Formula (I):

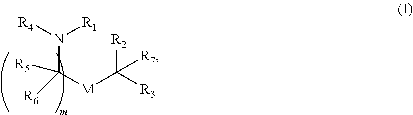

or a salt or isomer thereof, wherein:
R₁ is selected from the group consisting of H, C₅₋₃₀ alkyl, C₅₋₃₀ alkenyl, —R*YR″, —YR″, —(CH₂)ₙ(NR₄)R″M′R′, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle, wherein the carbocycle is optionally substituted with C₆ cycloalkyl or C₅ alkyl;
R₄ is selected from the group consisting of a C₃₋₆ carbocycle, —(CH₂)ₙQ, —(CH₂)ₙCHQR,
   —CHQR, —CQ(R)₂, —CH(CH₂Q)₂, and unsubstituted C₁₋₆ alkyl, wherein the C₃₋₆ carbocycle is optionally substituted with —OH or —OMe;
each Q is independently selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂ R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR;
or Q is selected from:

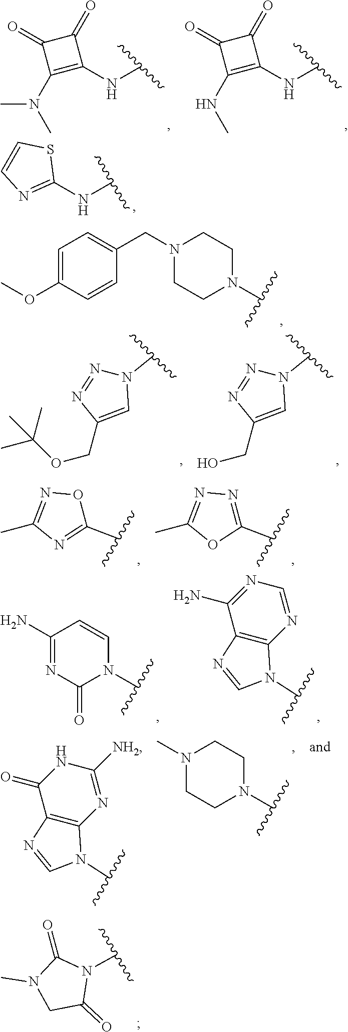

each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H, wherein C₁₋₃ alkyl is optionally substituted with —OH, —C(O)OH, —OMe, —O-benzyl,
each R′ is independently selected from the group consisting of C₁₋₁₈ alkyl, C₂₋₁₈ alkenyl, —R*YR″, —YR″, and H, wherein C₁₋₁₈ alkyl is optionally substituted with -OMe;
each R″ is independently selected from the group consisting of H, C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, the ionizable lipid may be a compound of Formula (I):

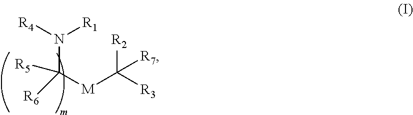

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
   —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂,
   —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
   —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
   —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈,
   —O(CH₂)ₙOR, —N(R)C(═NR₉)N(R)₂, —N(R)C(═CHR₉)N(R)₂, —OC(O)N(R)₂, —N(R)C(O)OR,
   —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
   —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR,
   —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈,
   —O(CH₂)ₙOR, —N(R)C(═NR₉)N(R)₂, —N(R)C(═CHR₉)N(R)₂, —OC(O)N(R)₂, —N(R)C(O)OR,
   —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, N02, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
   —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
   —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈,
   —O(CH₂)ₙOR, —N(R)C(═NR₉)N(R)₂, —N(R)C(═CHR₉)N(R)₂, —OC(O)N(R)₂, —N(R)C(O)OR,
   —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

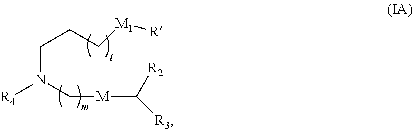

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is

OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

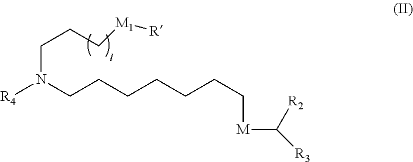

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

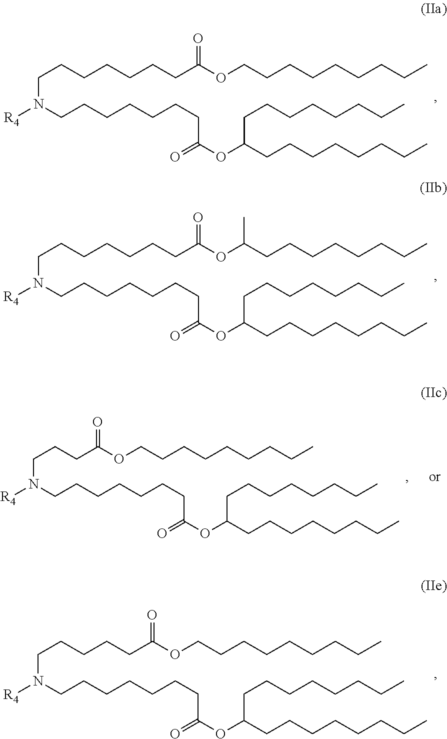

or a salt or isomer thereof, wherein R₄ is as described herein.

In some embodiments, a subset of compounds of Formula (I) includes those of Formula (IId):

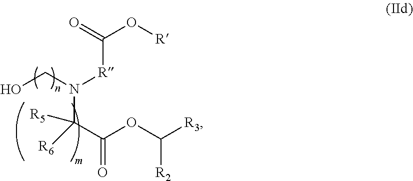

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, the compound of Formula (I) is selected from the group consisting of:

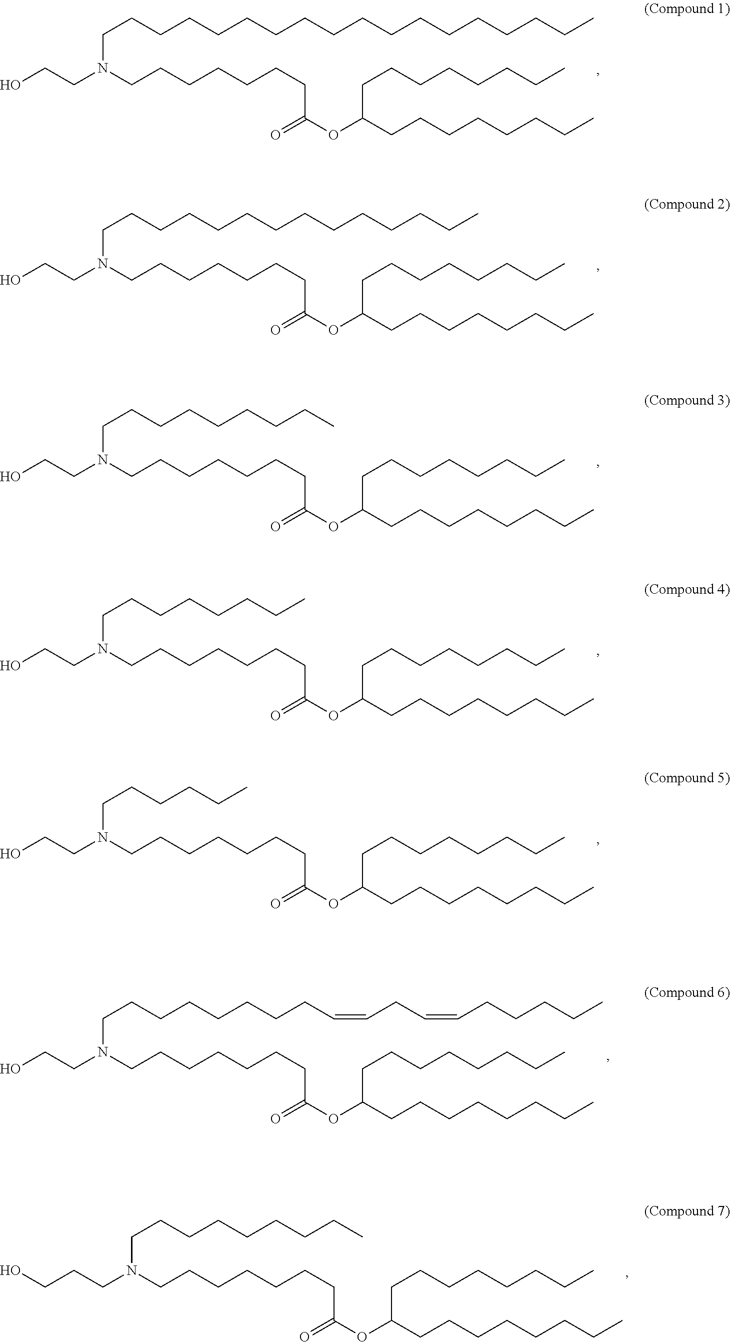

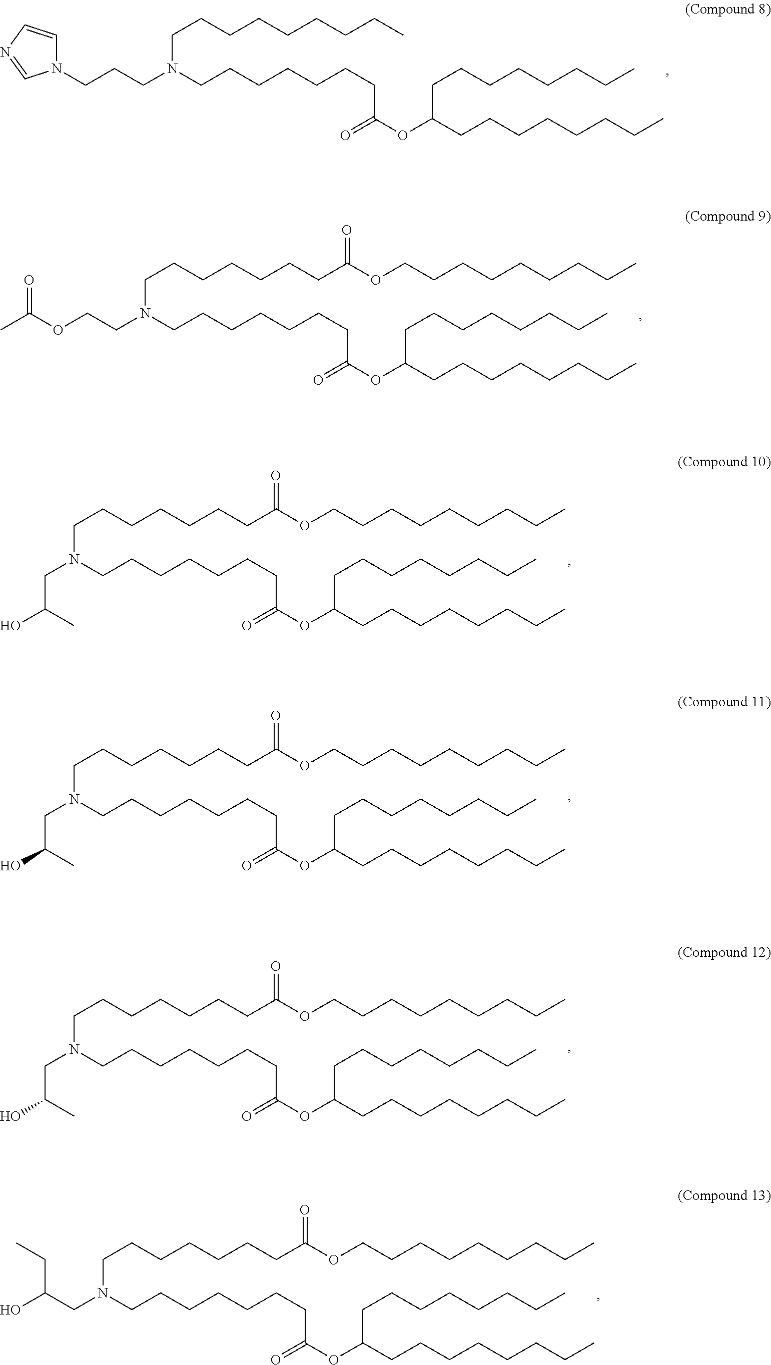

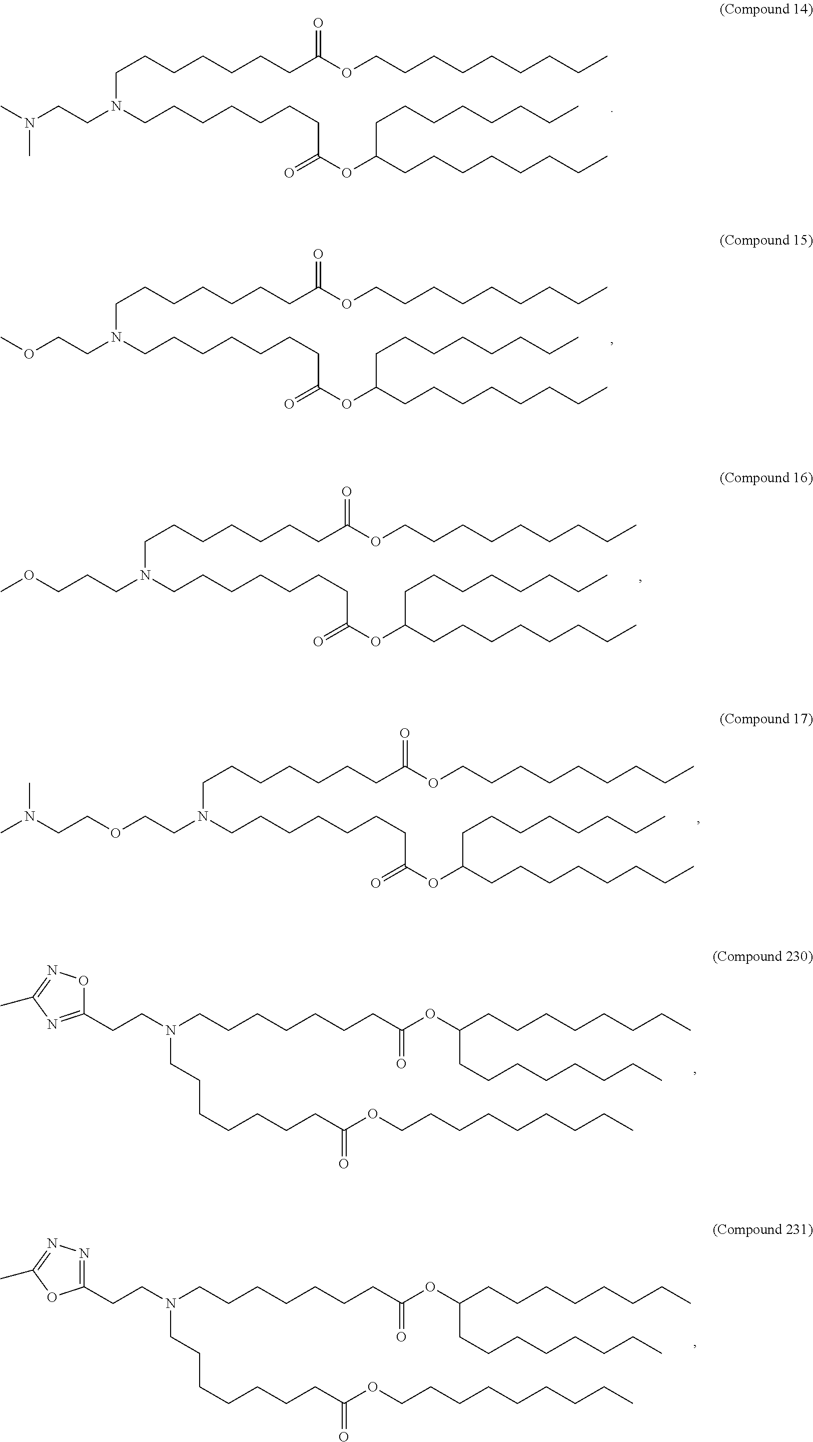

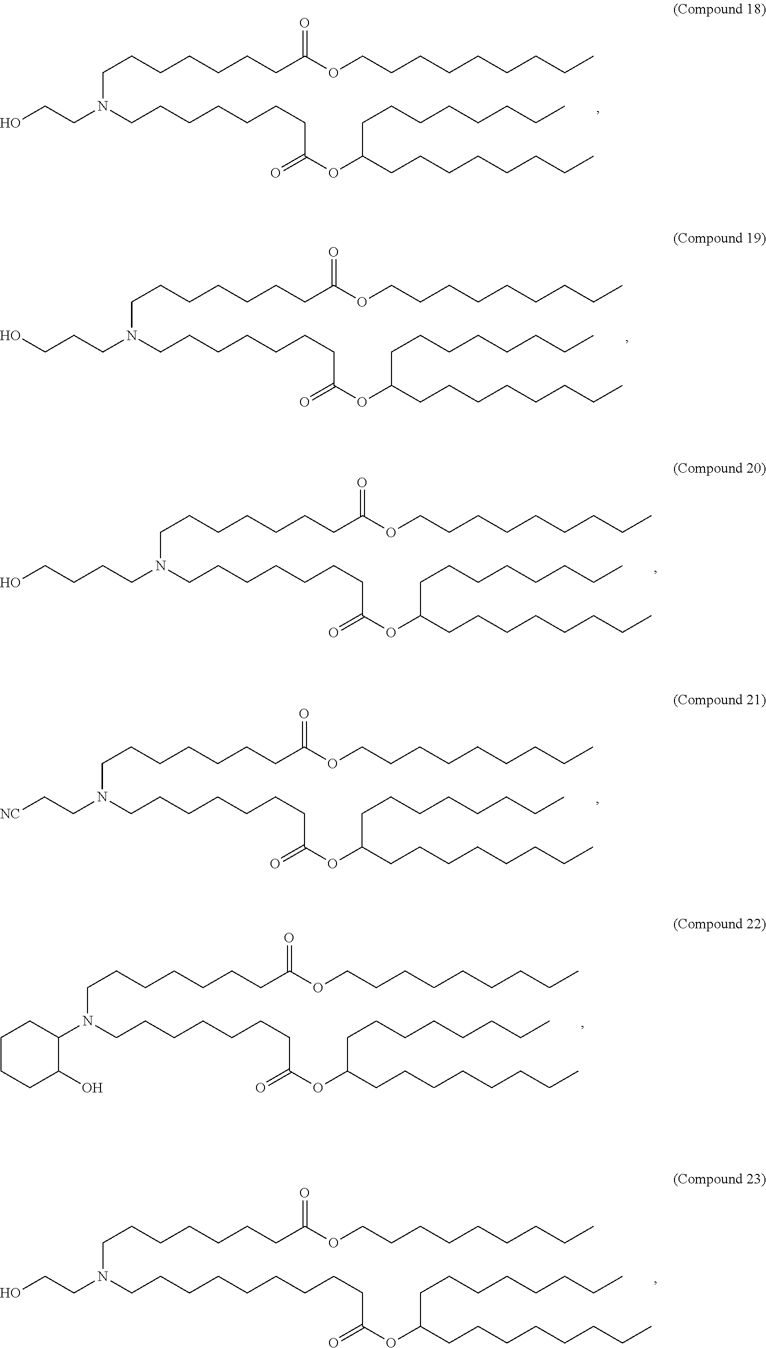 [SMILES: CCCCCCCCCOC(=O)CCCCCCCN(CCO)CCCCCCCC(=O)OC(CCCCCCCC)CCCCCCCC ; CCCCCCCCCOC(=O)CCCCCCCN(CCCO)CCCCCCCC(=O)OC(CCCCCCCC)CCCCCCCC ; CCCCCCCCCOC(=O)CCCCCCCN(CCCCO)CCCCCCCC(=O)OC(CCCCCCCC)CCCCCCCC ; CCCCCCCCCOC(=O)CCCCCCCN(CCC#N)CCCCCCCC(=O)OC(CCCCCCCC)CCCCCCCC ; CCCCCCCCCOC(=O)CCCCCCCN(CCCCCCCC(=O)OC(CCCCCCCC)CCCCCCCC)C1CCCCC1O ; CCCCCCCCCOC(=O)CCCCCCCN(CCO)CCCCCCCCCC(=O)OC(CCCCCCCC)CCCCCCCC]

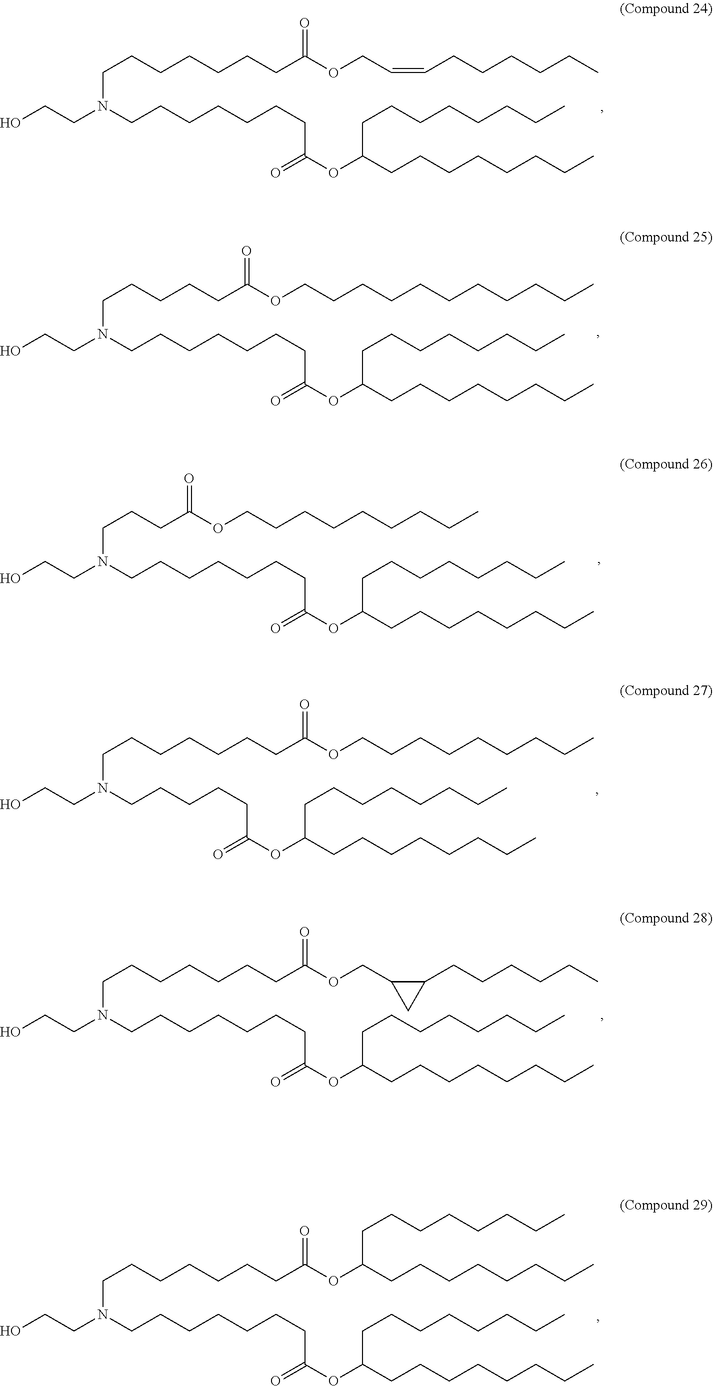 [SMILES: CCCCCC/C=C\COC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCCOC(=O)CCCN(CCO)CCCCCCCC(=O)OC(CCCCCCCC)CCCCCCCC ; CCCCCCCCCOC(=O)CCCCCCCN(CCO)CCCCCC(=O)OC(CCCCCCCC)CCCCCCCC ; CCCCCCCCC(CCCCCCCC)OC(=O)CCCCCCCN(CCO)CCCCCCCC(=O)OCC1CC1CCCCCC ; CCCCCCCCC(CCCCCCCC)OC(=O)CCCCCCCN(CCO)CCCCCCCC(=O)OC(CCCCCCCC)CCCCCCCC]

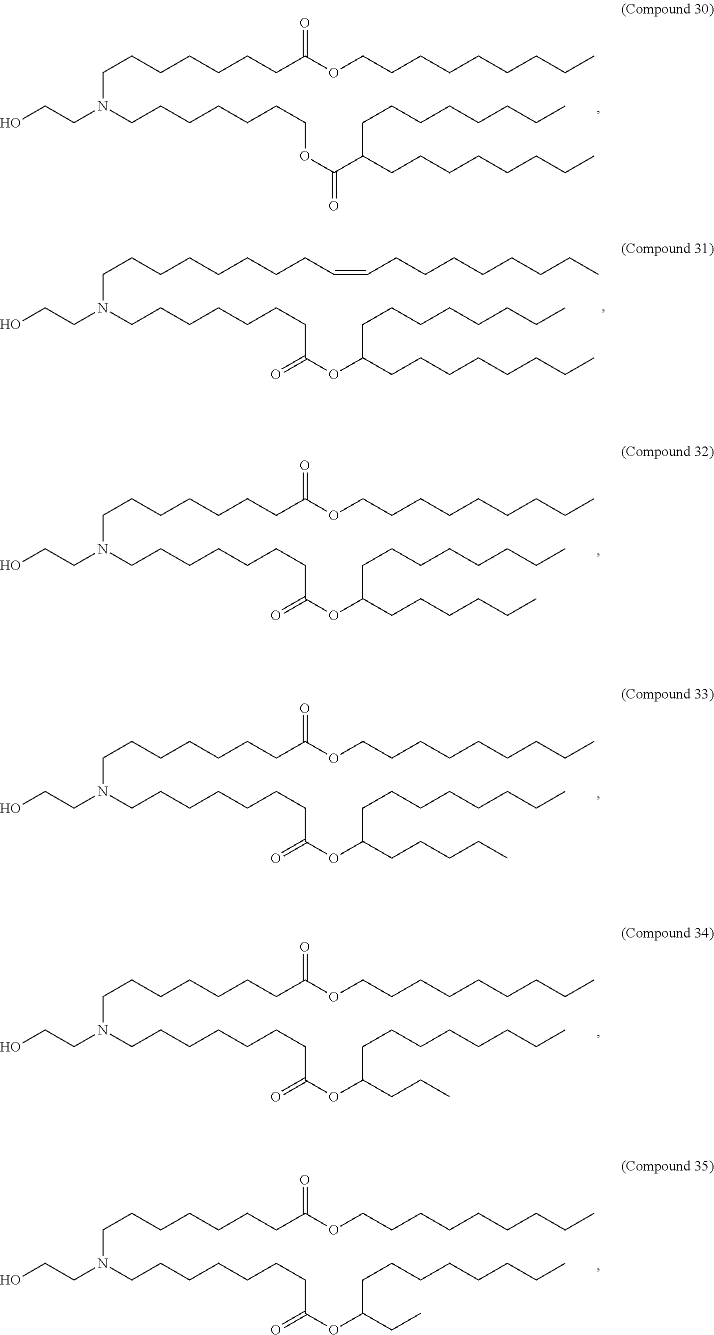 [SMILES: CCCCCCCCCOC(=O)CCCCCCCN(CCO)CCCCCCCOC(=O)C(CCCCCCCC)CCCCCCCC ; CCCCCCCC/C=C\CCCCCCCCN(CCO)CCCCCCCC(=O)OC(CCCCCCCC)CCCCCCCC ; CCCCCCCCCOC(=O)CCCCCCCN(CCO)CCCCCCCC(=O)OC(CCCCCC)CCCCCCCC ; CCCCCCCCCOC(=O)CCCCCCCN(CCO)CCCCCCCC(=O)OC(CCCCC)CCCCCCCC ; CCCCCCCCCOC(=O)CCCCCCCN(CCO)CCCCCCCC(=O)OC(CCC)CCCCCCCC ; CCCCCCCCCOC(=O)CCCCCCCN(CCO)CCCCCCCC(=O)OC(CC)CCCCCCCC]

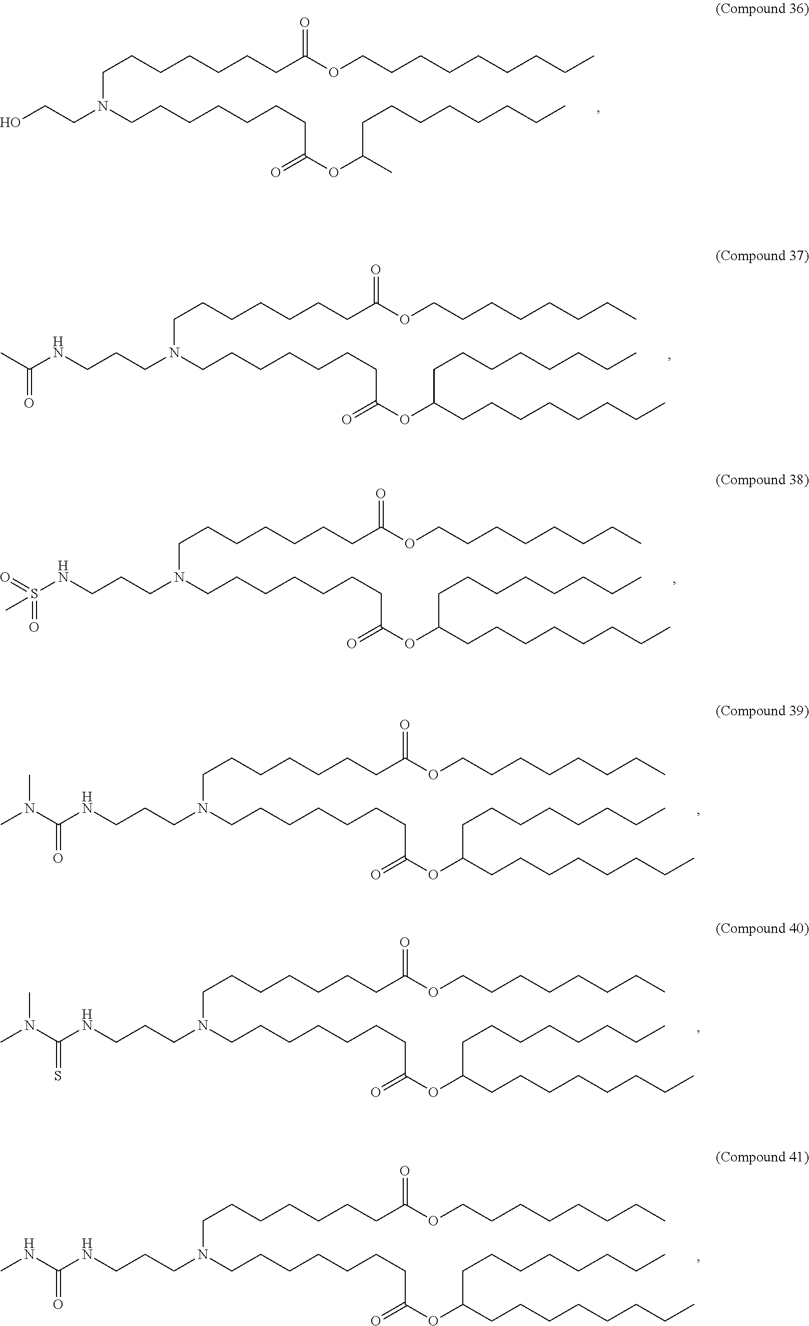

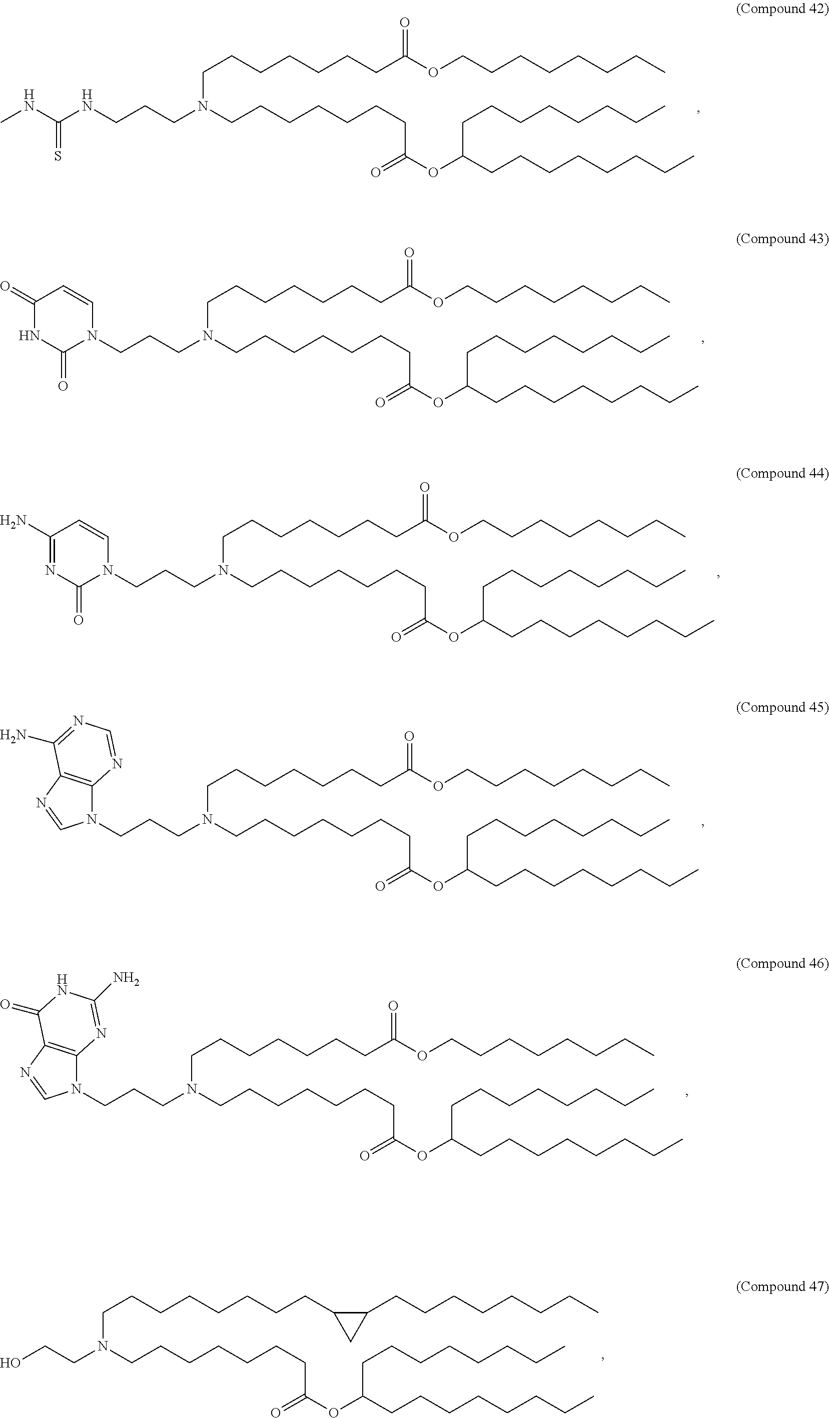 [SMILES: CCCCCCCCOC(=O)CCCCCCCN(CCCCCCCC(=O)OC(CCCCCCCC)CCCCCCCC)CCCNC(=S)NC ; CCCCCCCCOC(=O)CCCCCCCN(CCCCCCCC(=O)OC(CCCCCCCC)CCCCCCCC)CCCn1ccc(=O)[nH]c1=O ; CCCCCCCCOC(=O)CCCCCCCN(CCCCCCCC(=O)OC(CCCCCCCC)CCCCCCCC)CCCn1ccc(N)nc1=O ; CCCCCCCCOC(=O)CCCCCCCN(CCCCCCCC(=O)OC(CCCCCCCC)CCCCCCCC)CCCn1cnc2c(N)ncnc21 ; CCCCCCCCOC(=O)CCCCCCCN(CCCCCCCC(=O)OC(CCCCCCCC)CCCCCCCC)CCCn1cnc2c(=O)[nH]c(N)nc21 ; CCCCCCCCC(CCCCCCCC)OC(=O)CCCCCCCN(CCO)CCCCCCCCC1CC1CCCCCCCC]

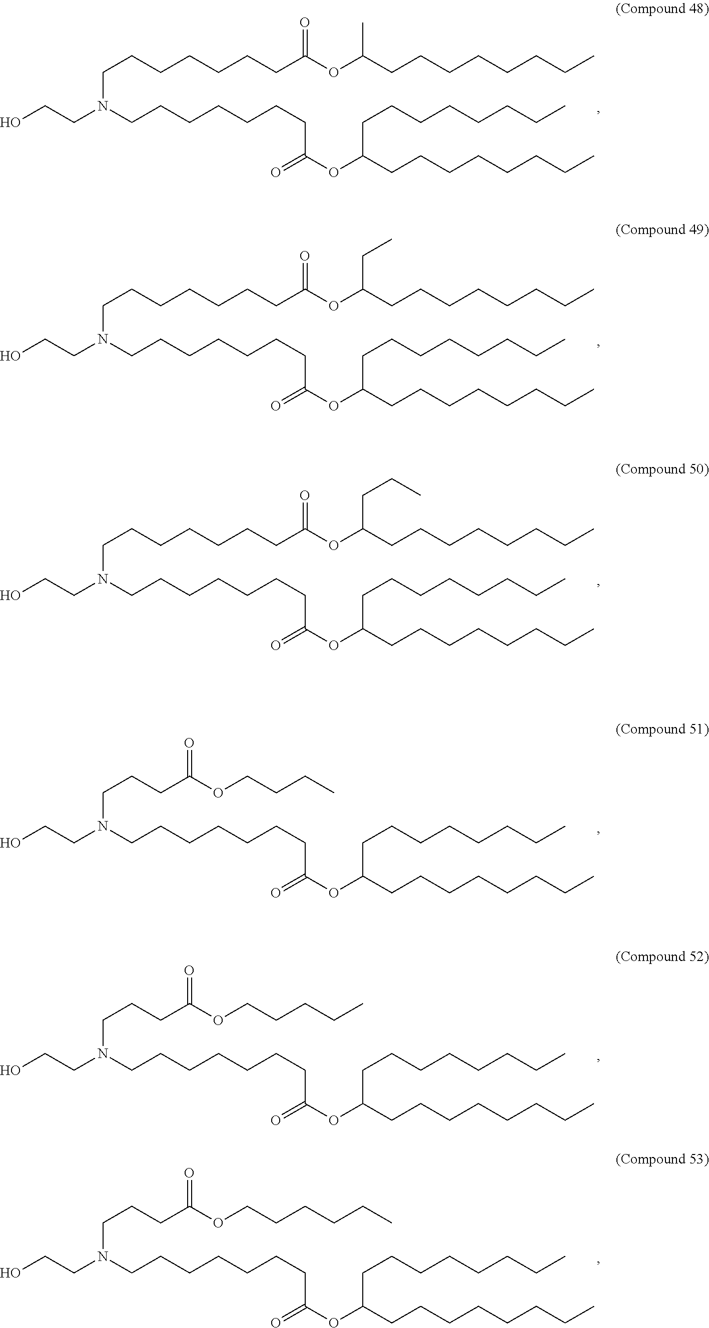 [SMILES: CCCCCCCCC(C)OC(=O)CCCCCCCN(CCO)CCCCCCCC(=O)OC(CCCCCCCC)CCCCCCCC ; CCCCCCCCC(CC)OC(=O)CCCCCCCN(CCO)CCCCCCCC(=O)OC(CCCCCCCC)CCCCCCCC ; CCCCCCCCC(CCC)OC(=O)CCCCCCCN(CCO)CCCCCCCC(=O)OC(CCCCCCCC)CCCCCCCC ; CCCCCCCCC(CCCCCCCC)OC(=O)CCCCCCCN(CCO)CCCC(=O)OCCCC ; CCCCCCCCC(CCCCCCCC)OC(=O)CCCCCCCN(CCO)CCCC(=O)OCCCCC ; CCCCCCCCC(CCCCCCCC)OC(=O)CCCCCCCN(CCO)CCCC(=O)OCCCCCC]

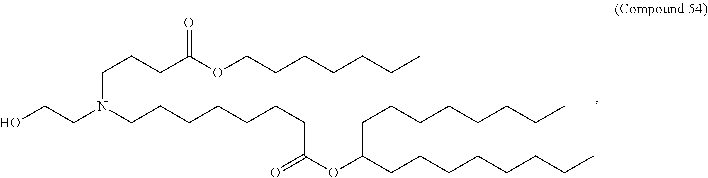 [SMILES: CCCCCCCCC(CCCCCCCC)OC(=O)CCCCCCCN(CCO)CCCC(=O)OCCCCCCC]

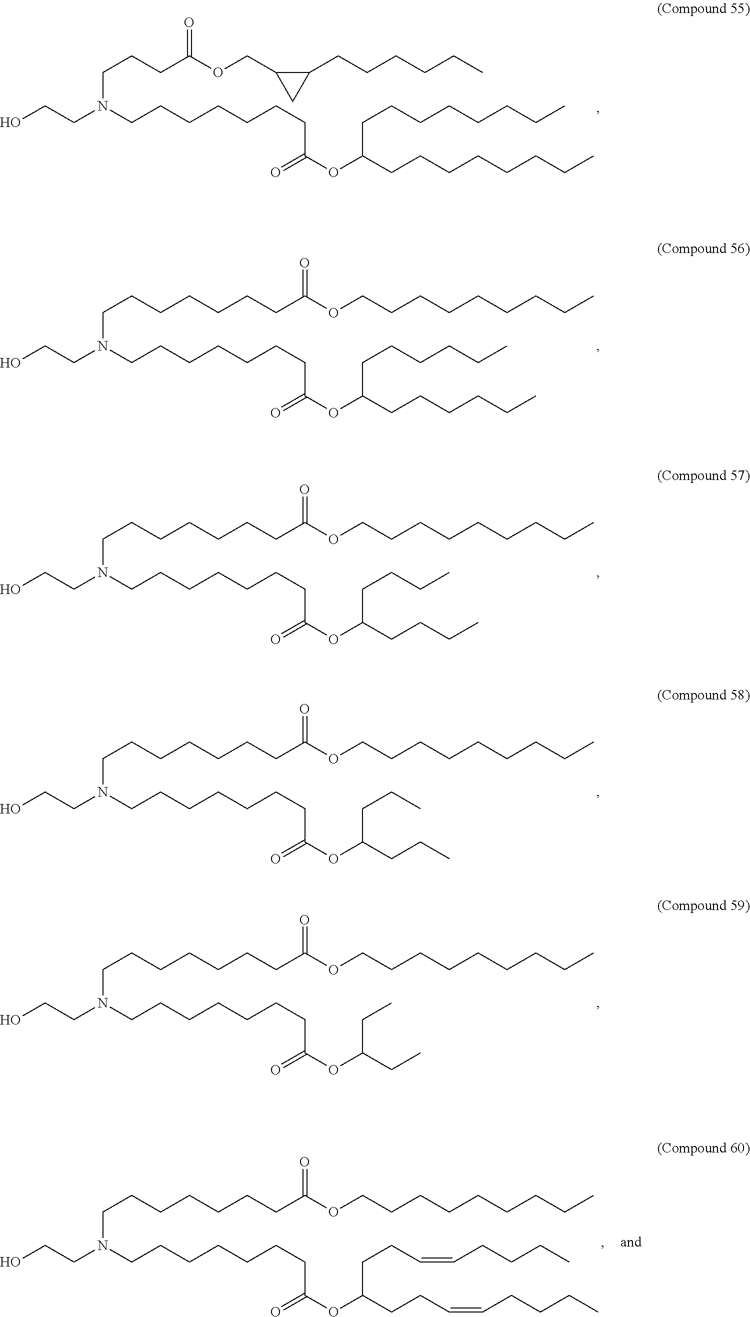 [SMILES: CCCCCCCCC(CCCCCCCC)OC(=O)CCCCCCCN(CCO)CCCC(=O)OCC1CC1CCCCCC ; CCCCCCCCCOC(=O)CCCCCCCN(CCO)CCCCCCCC(=O)OC(CCCCCC)CCCCCC ; CCCCCCCCCOC(=O)CCCCCCCN(CCO)CCCCCCCC(=O)OC(CCCC)CCCC ; CCCCCCCCCOC(=O)CCCCCCCN(CCO)CCCCCCCC(=O)OC(CCC)CCC ; CCCCCCCCCOC(=O)CCCCCCCN(CCO)CCCCCCCC(=O)OC(CC)CC ; CCCC/C=C\CCC(CC/C=C\CCCC)OC(=O)CCCCCCCN(CCO)CCCCCCCC(=O)OCCCCCCCCC]

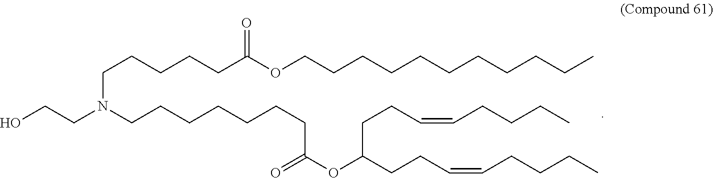 [SMILES: CCCC/C=C\CCC(CC/C=C\CCCC)OC(=O)CCCCCCCN(CCO)CCCCCC(=O)OCCCCCCCCCCC]

In further embodiments, the compound of Formula (I) is selected from the group consisting of:

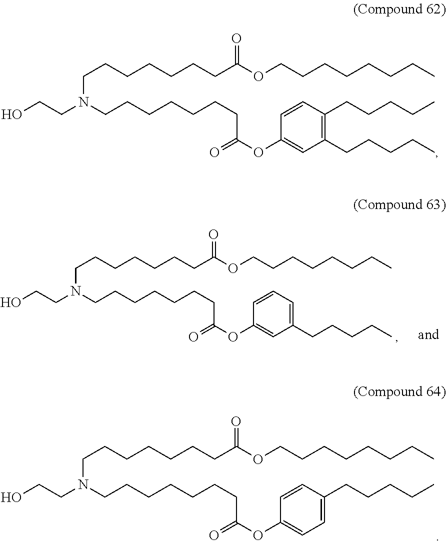 [SMILES: CCCCCCCCOC(=O)CCCCCCCN(CCO)CCCCCCCC(=O)Oc1ccc(CCCCC)c(CCCCC)c1 ; CCCCCCCCOC(=O)CCCCCCCN(CCO)CCCCCCCC(=O)Oc1cccc(CCCCC)c1 ; CCCCCCCCOC(=O)CCCCCCCN(CCO)CCCCCCCC(=O)Oc1ccc(CCCCC)cc1]

In some embodiments, the compound of Formula (I) is selected from the group consisting of:

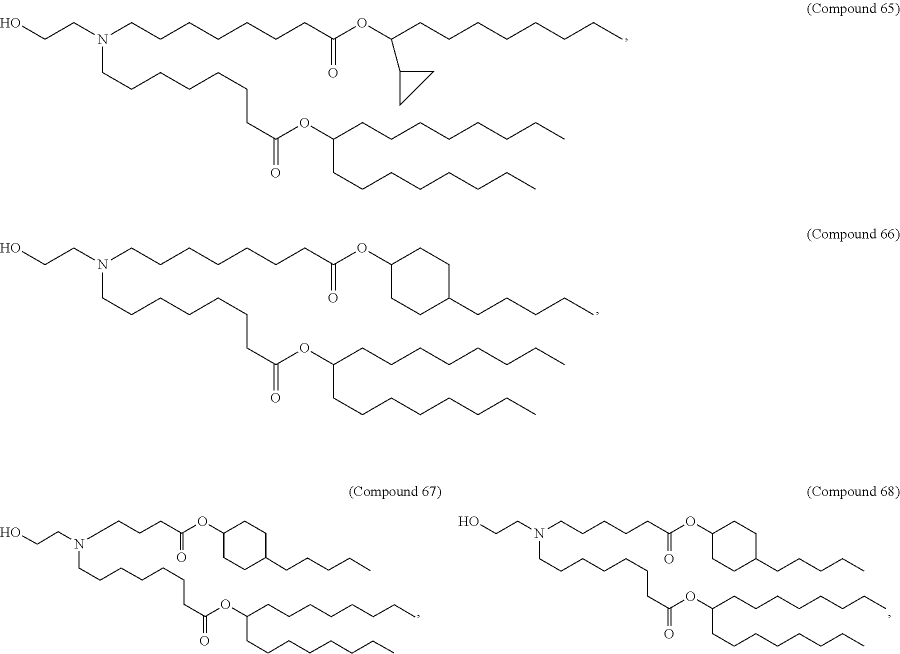 [SMILES: CCCCCCCCC(CCCCCCCC)OC(=O)CCCCCCCN(CCO)CCCCCCCC(=O)OC(CCCCCCCC)C1CC1 ; CCCCCCCCC(CCCCCCCC)OC(=O)CCCCCCCN(CCO)CCCCCCCC(=O)OC1CCC(CCCCC)CC1 ; CCCCCCCCC(CCCCCCCC)OC(=O)CCCCCCCN(CCO)CCCC(=O)OC1CCC(CCCCC)CC1 ; CCCCCCCCC(CCCCCCCC)OC(=O)CCCCCCCN(CCO)CCCCCC(=O)OC1CCC(CCCCC)CC1]

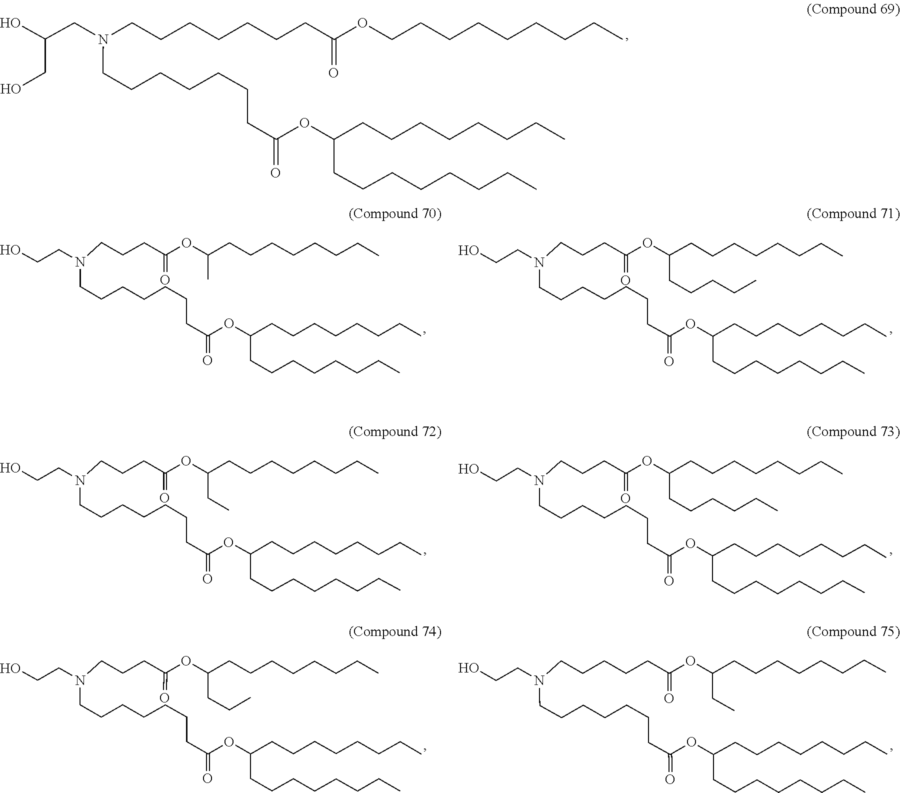

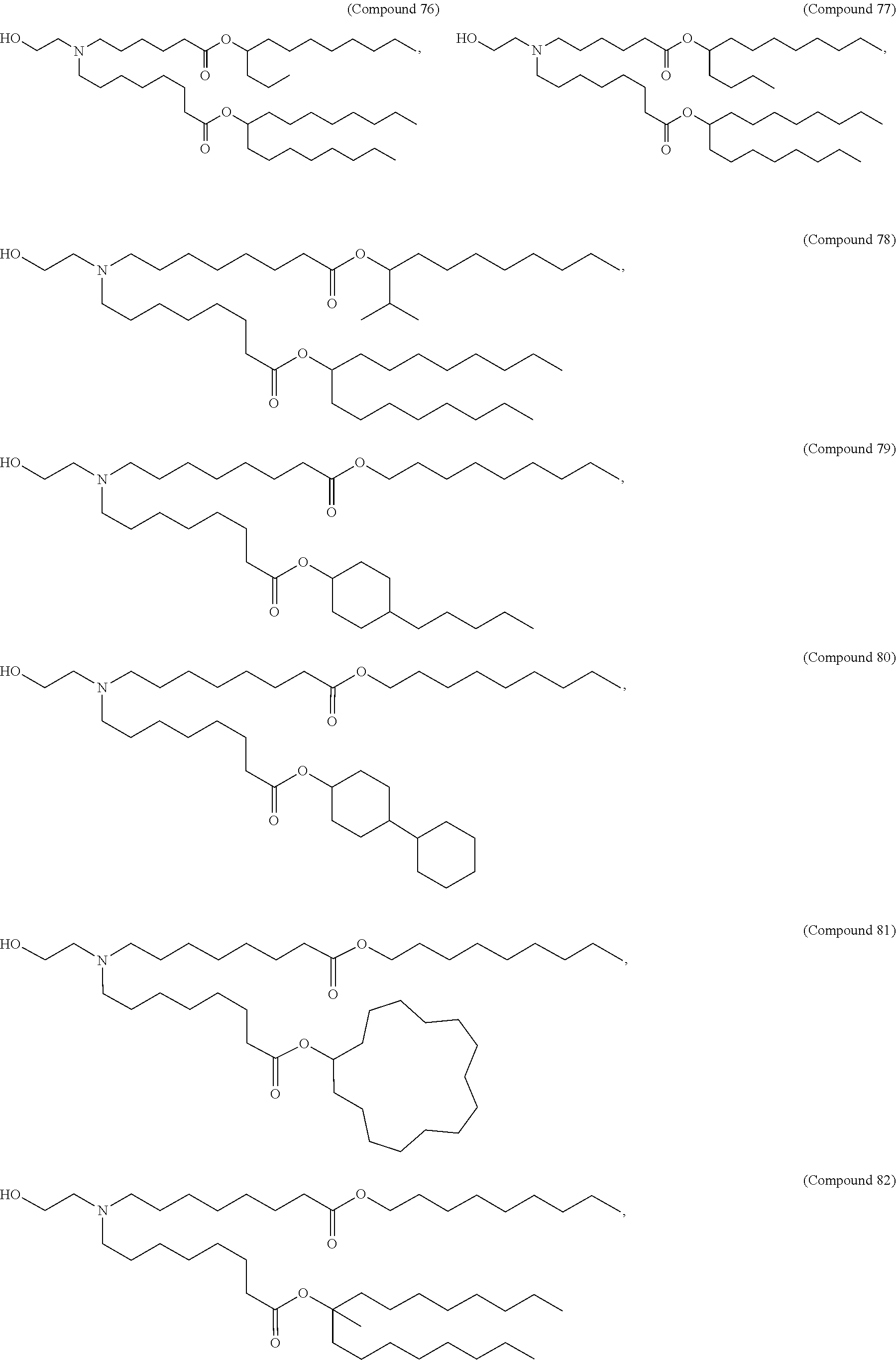

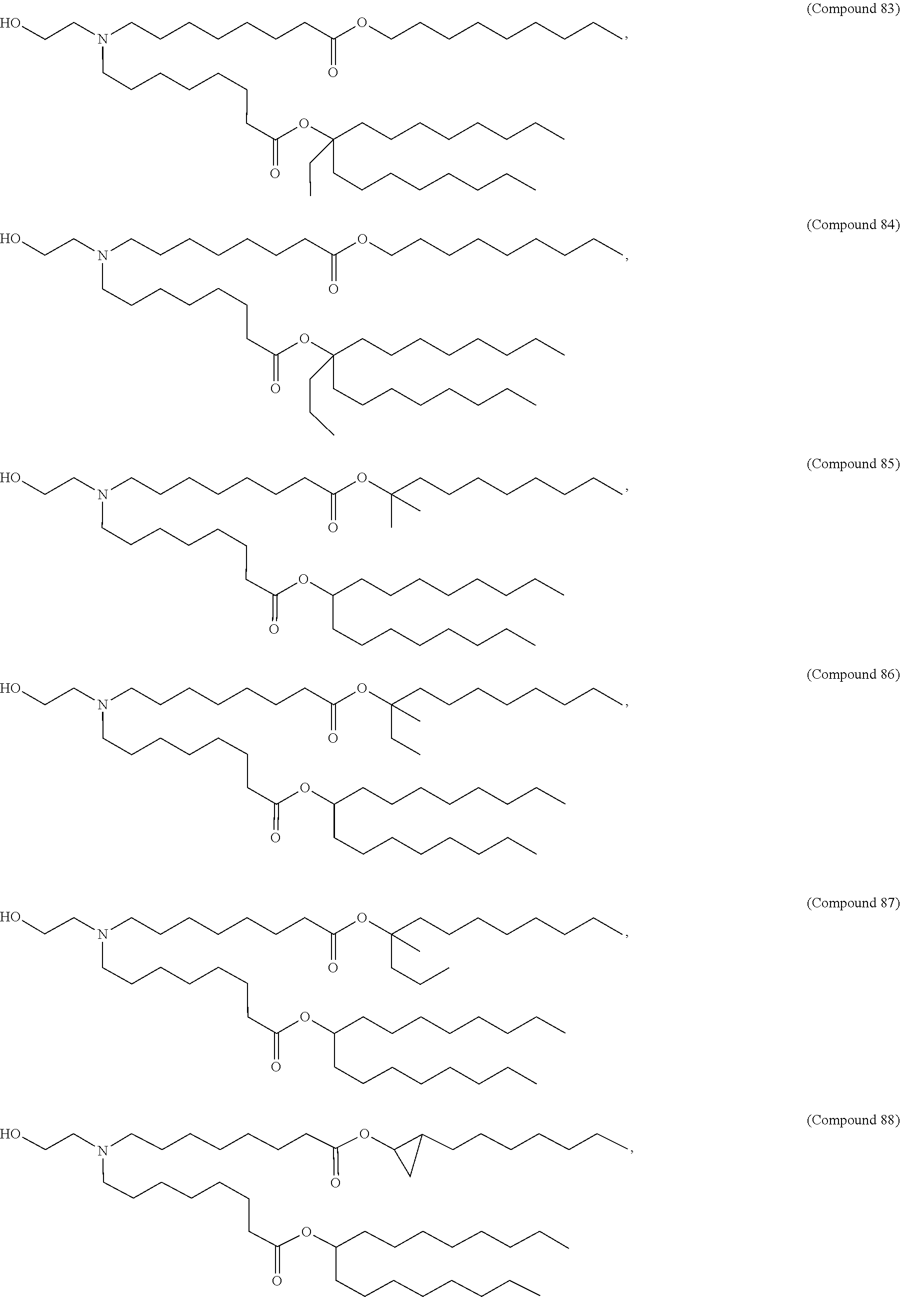

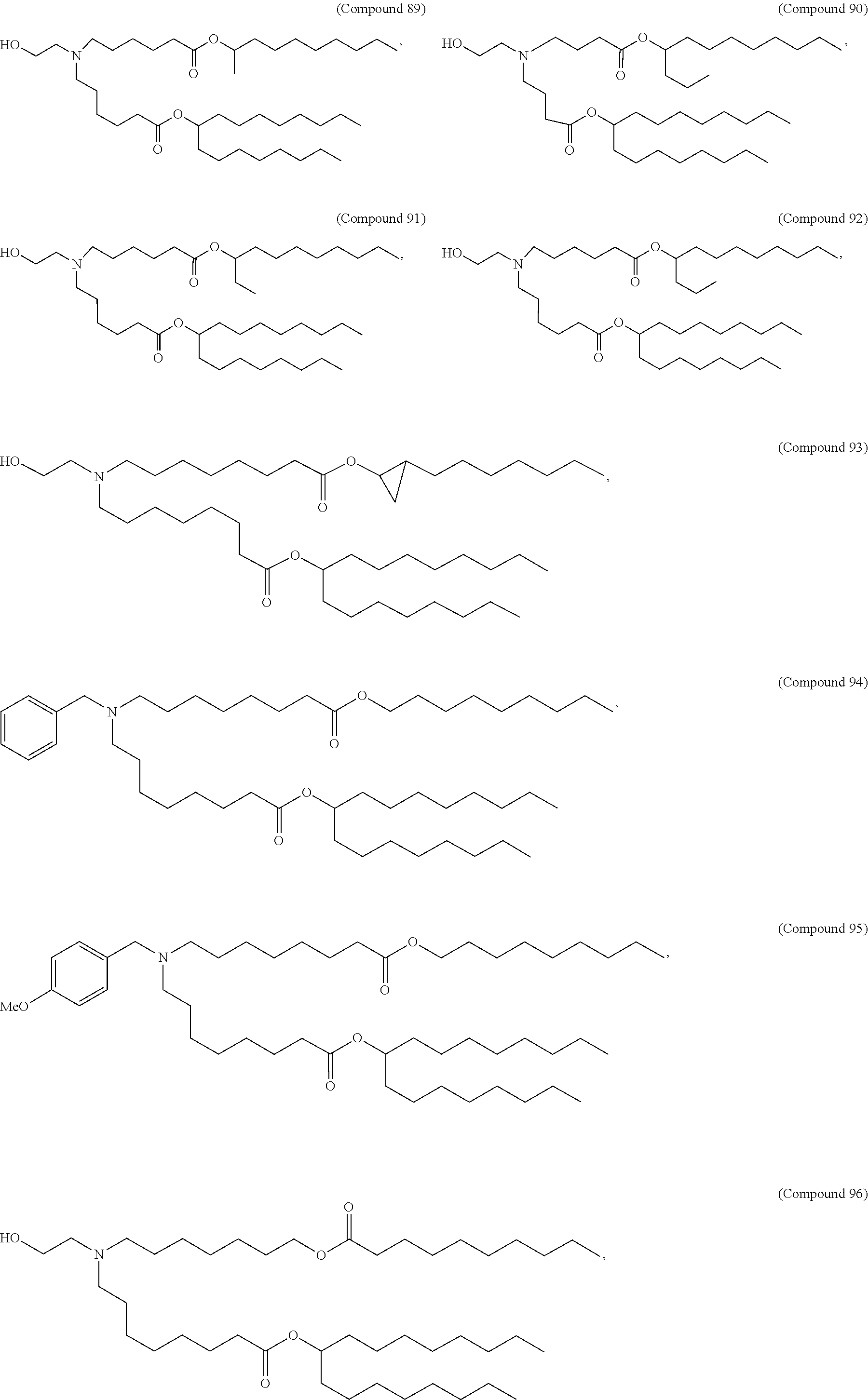

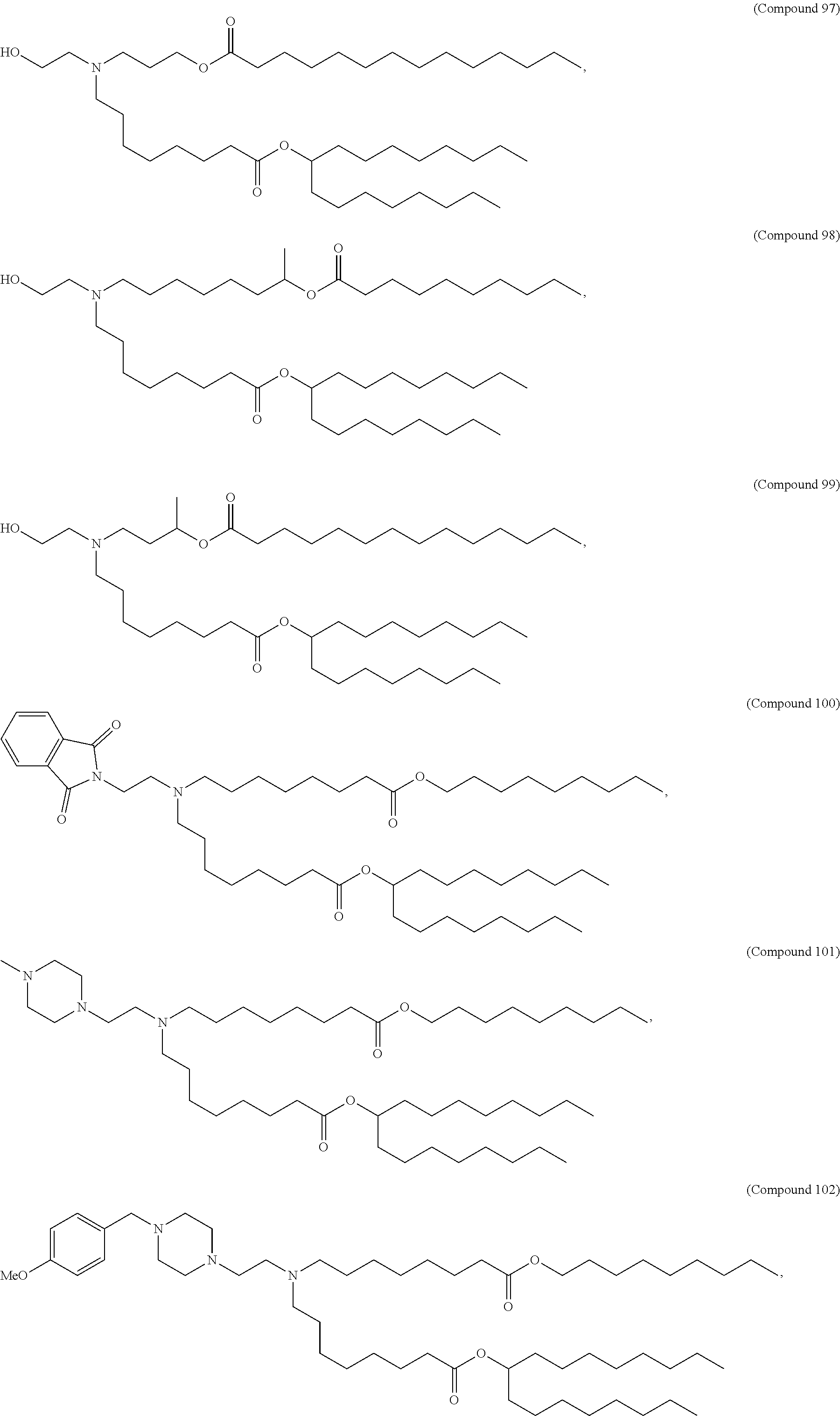

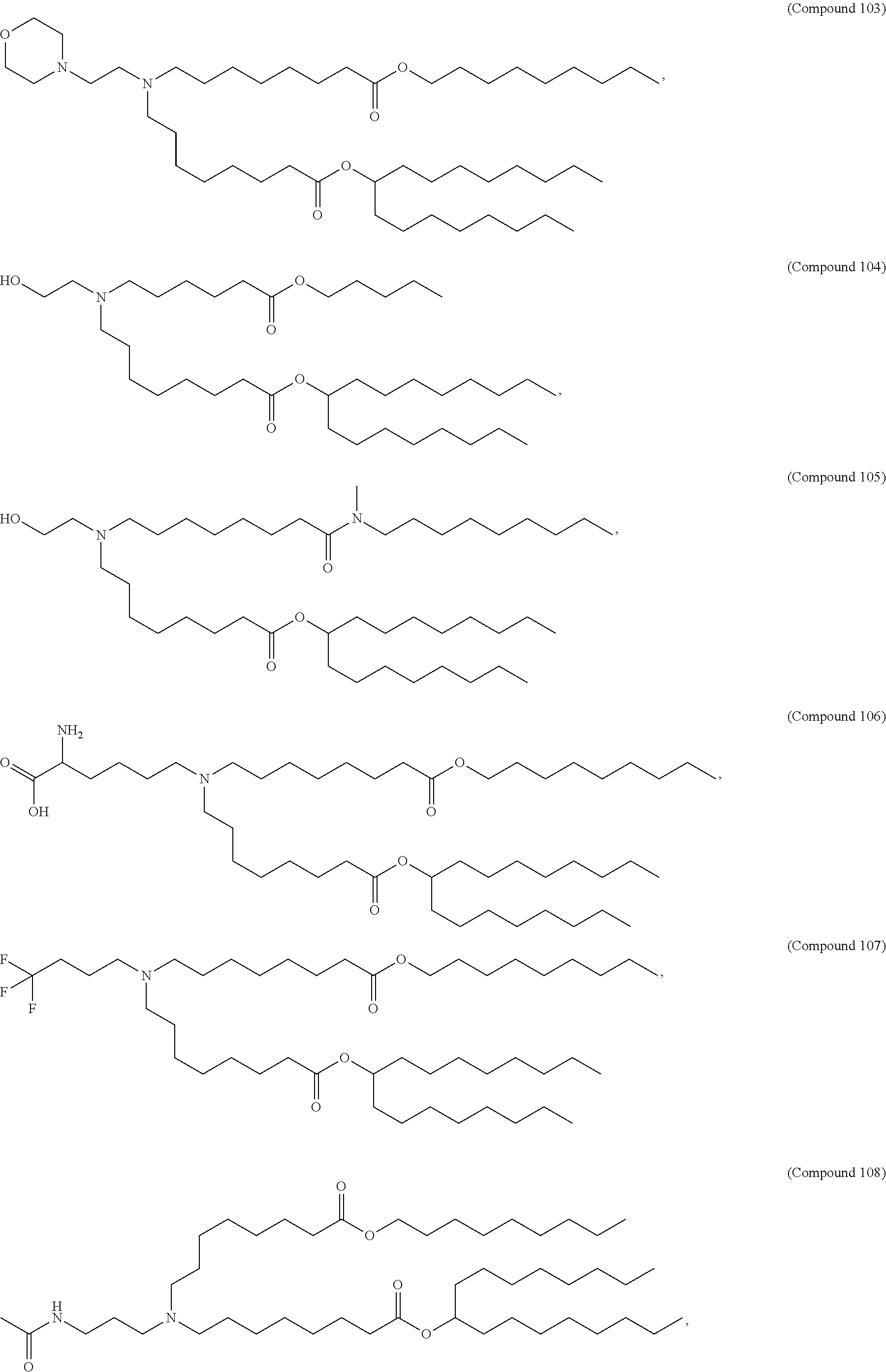

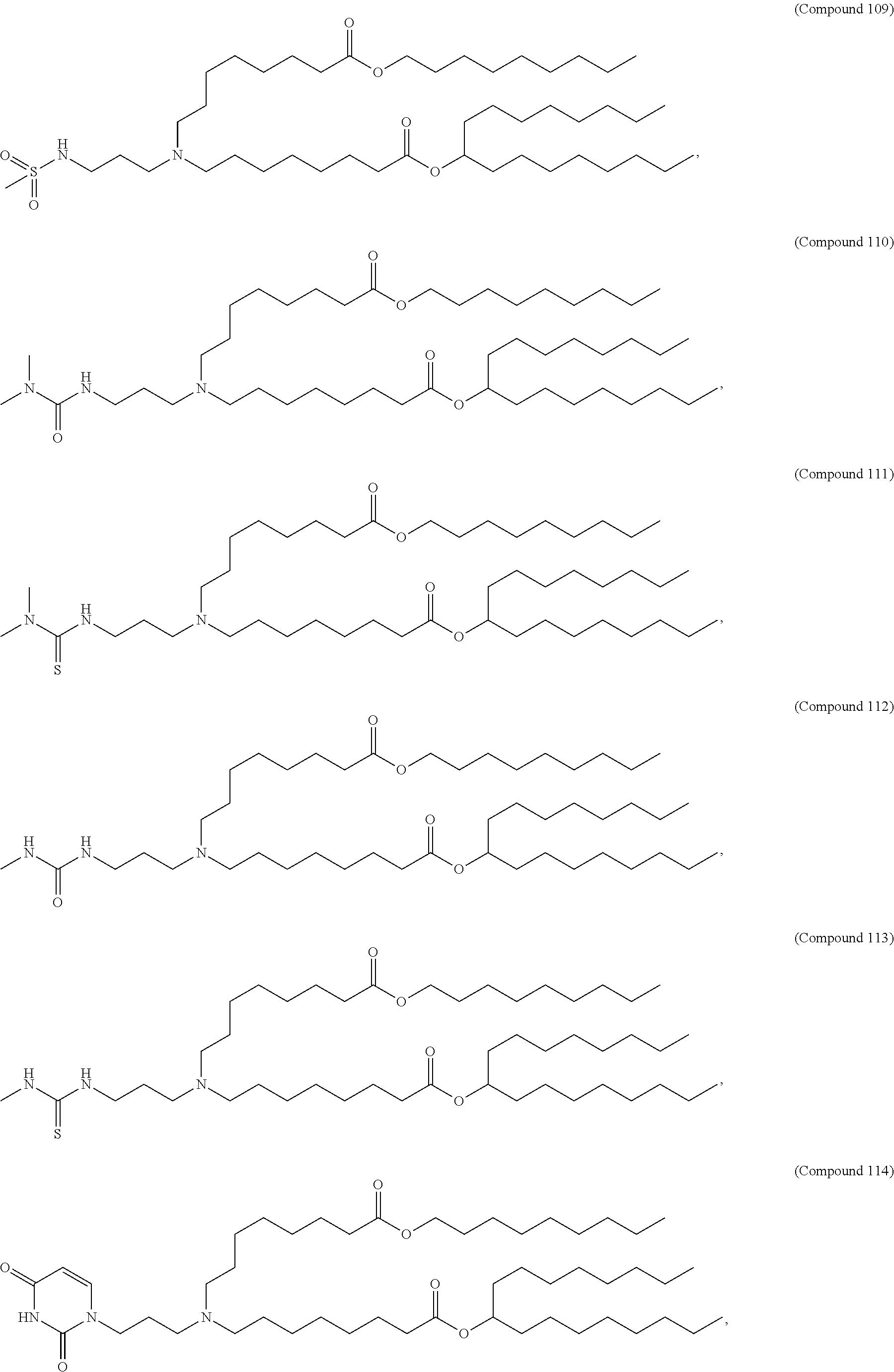

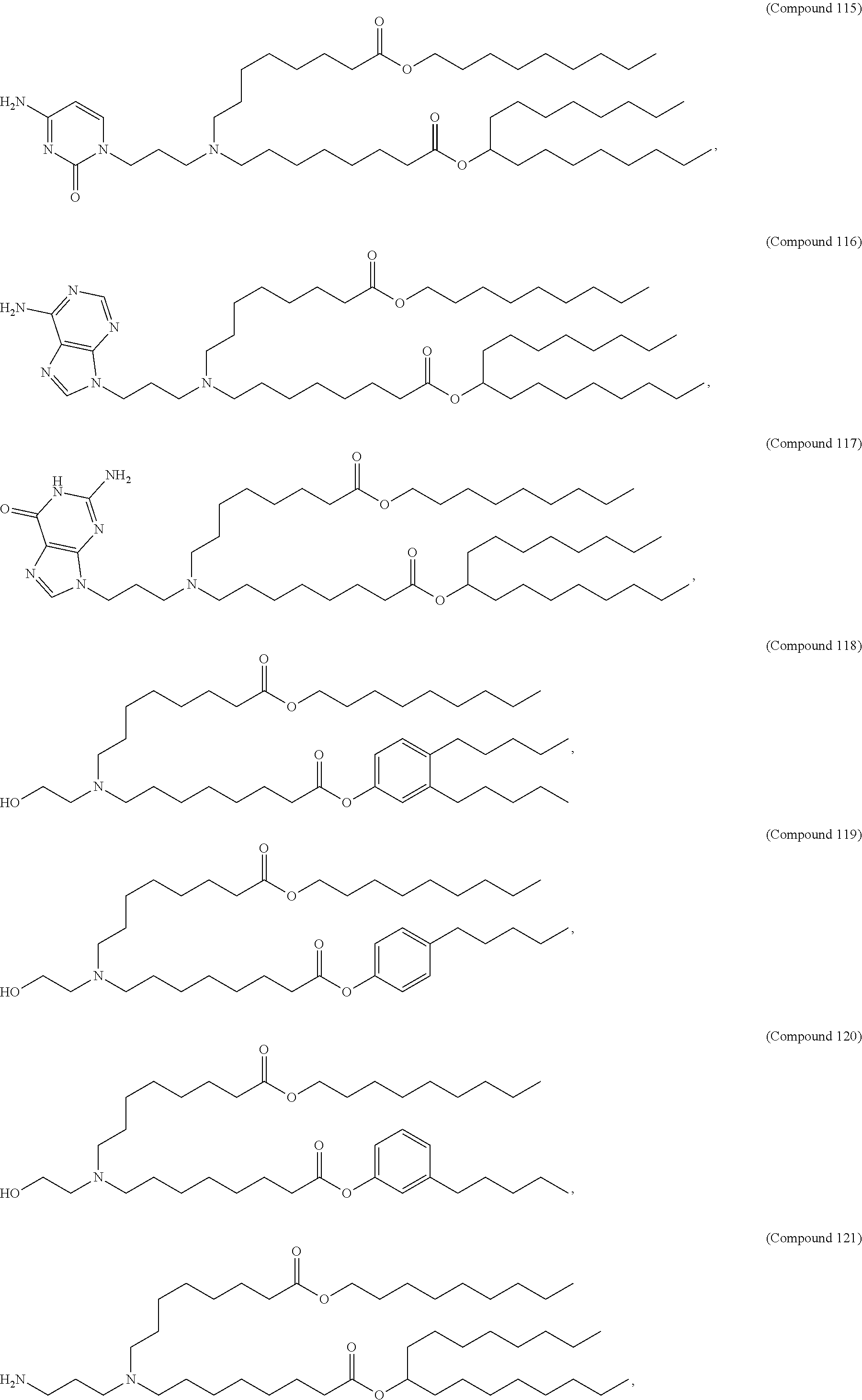

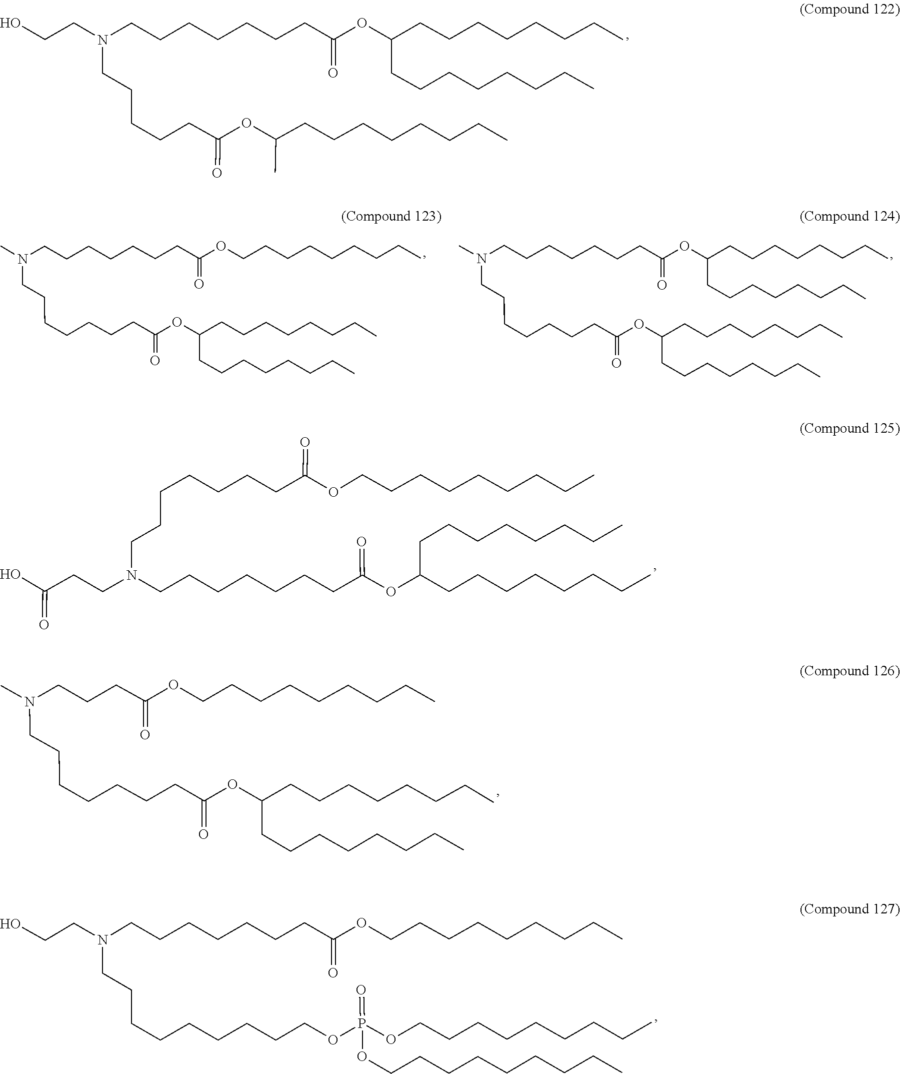

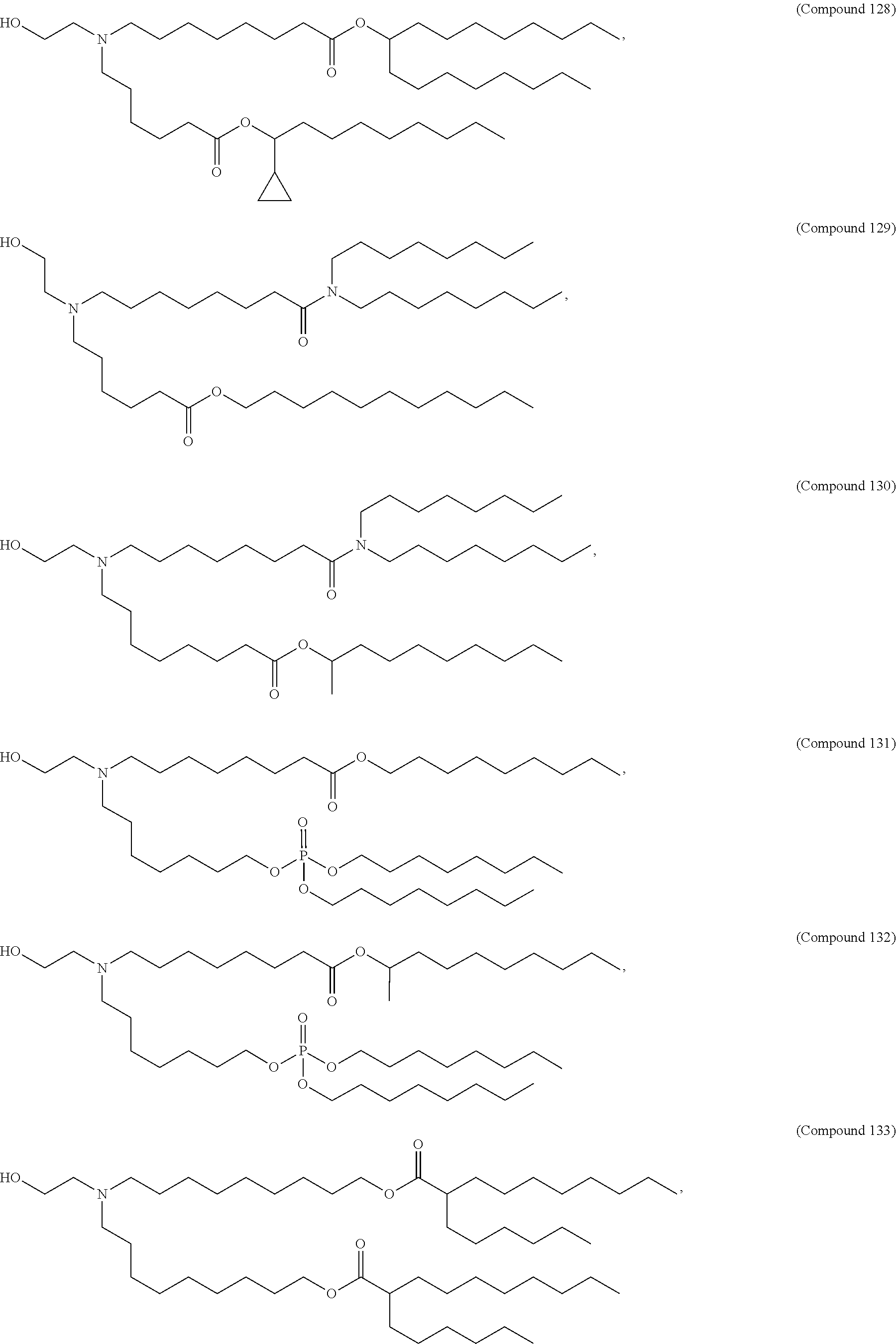 [SMILES: CCCCCCCCC(CCCCCCCC)OC(=O)CCCCCCCN(CCO)CCCCCC(=O)OC(CCCCCCCC)C1CC1 ; CCCCCCCCCCCOC(=O)CCCCCN(CCO)CCCCCCCC(=O)N(CCCCCCCC)CCCCCCCC ; CCCCCCCCC(C)OC(=O)CCCCCCCN(CCO)CCCCCCCC(=O)N(CCCCCCCC)CCCCCCCC ; CCCCCCCCCOC(=O)CCCCCCCN(CCO)CCCCCCCOP(=O)(OCCCCCCCC)OCCCCCCCC ; CCCCCCCCOP(=O)(OCCCCCCCC)OCCCCCCCN(CCO)CCCCCCCC(=O)OC(C)CCCCCCCC ; CCCCCCCCC(CCCCCC)C(=O)OCCCCCCCCCN(CCO)CCCCCCCCCOC(=O)C(CCCCCC)CCCCCCCC]

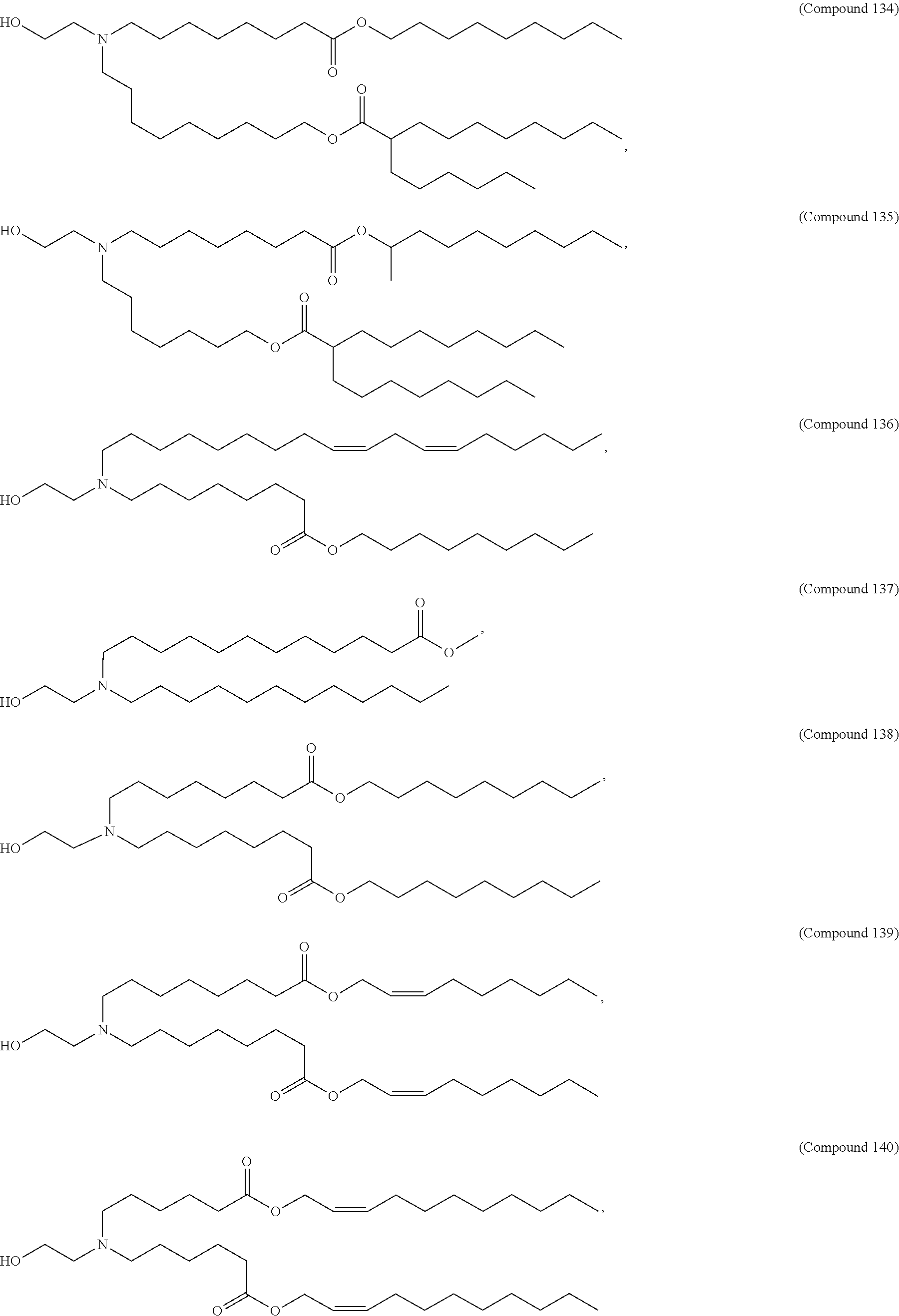

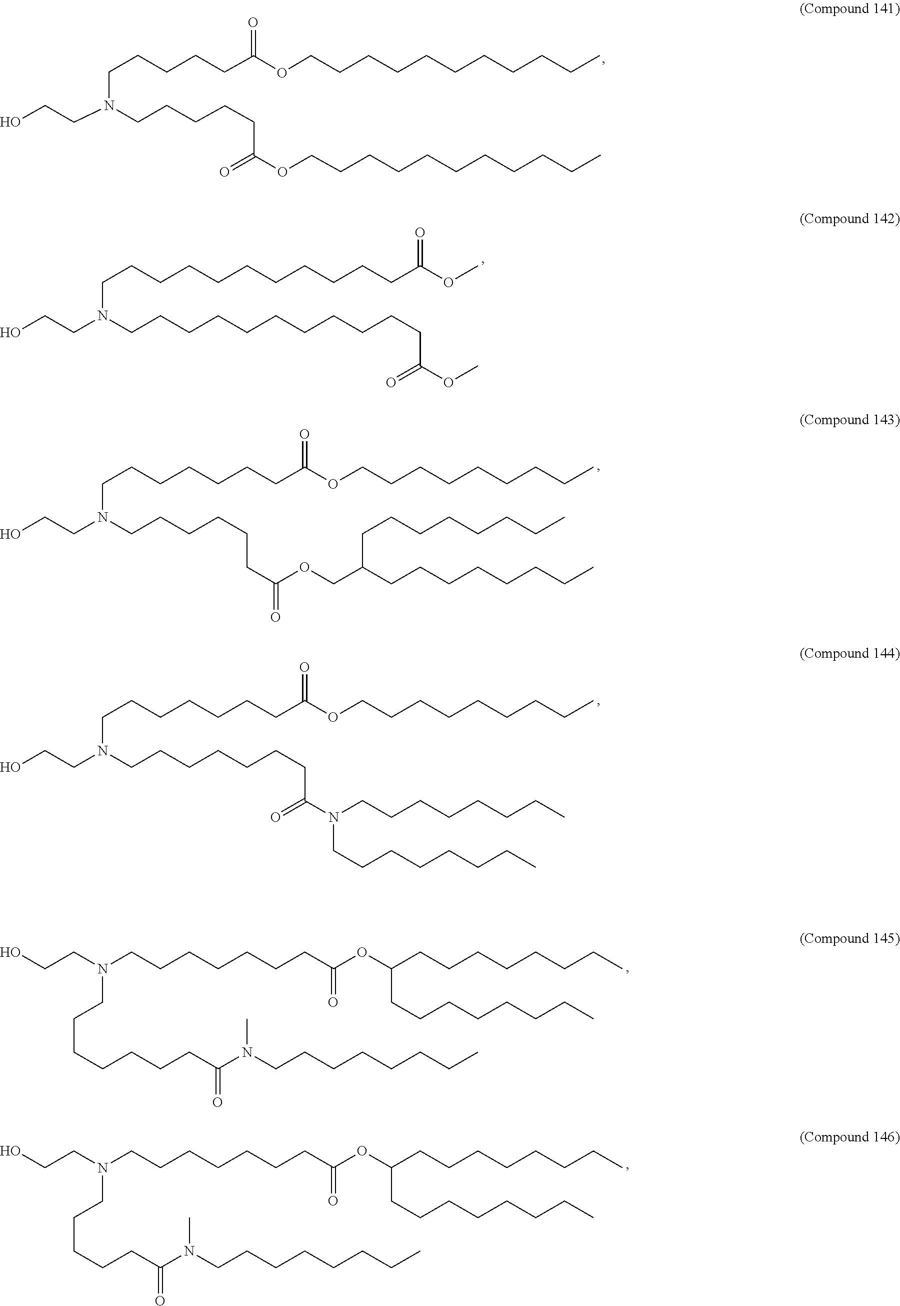 [SMILES: CCCCCCCCCCCOC(=O)CCCCCN(CCO)CCCCCC(=O)OCCCCCCCCCCC ; COC(=O)CCCCCCCCCCCN(CCO)CCCCCCCCCCCC(=O)OC ; CCCCCCCCCOC(=O)CCCCCCCN(CCO)CCCCCCC(=O)OCC(CCCCCCCC)CCCCCCCC ; CCCCCCCCCOC(=O)CCCCCCCN(CCO)CCCCCCCC(=O)N(CCCCCCCC)CCCCCCCC ; CCCCCCCCC(CCCCCCCC)OC(=O)CCCCCCCN(CCO)CCCCCCCC(=O)N(C)CCCCCCCC ; CCCCCCCCC(CCCCCCCC)OC(=O)CCCCCCCN(CCO)CCCCCC(=O)N(C)CCCCCCCC]

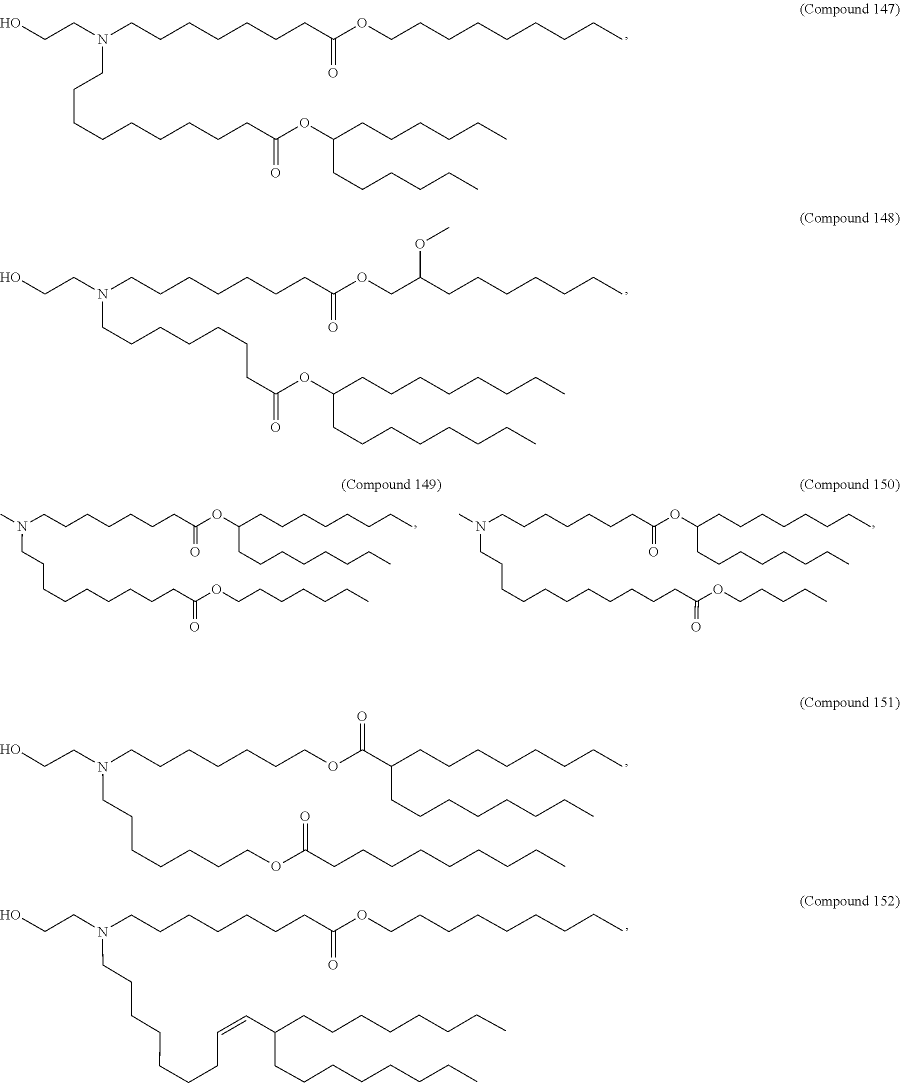

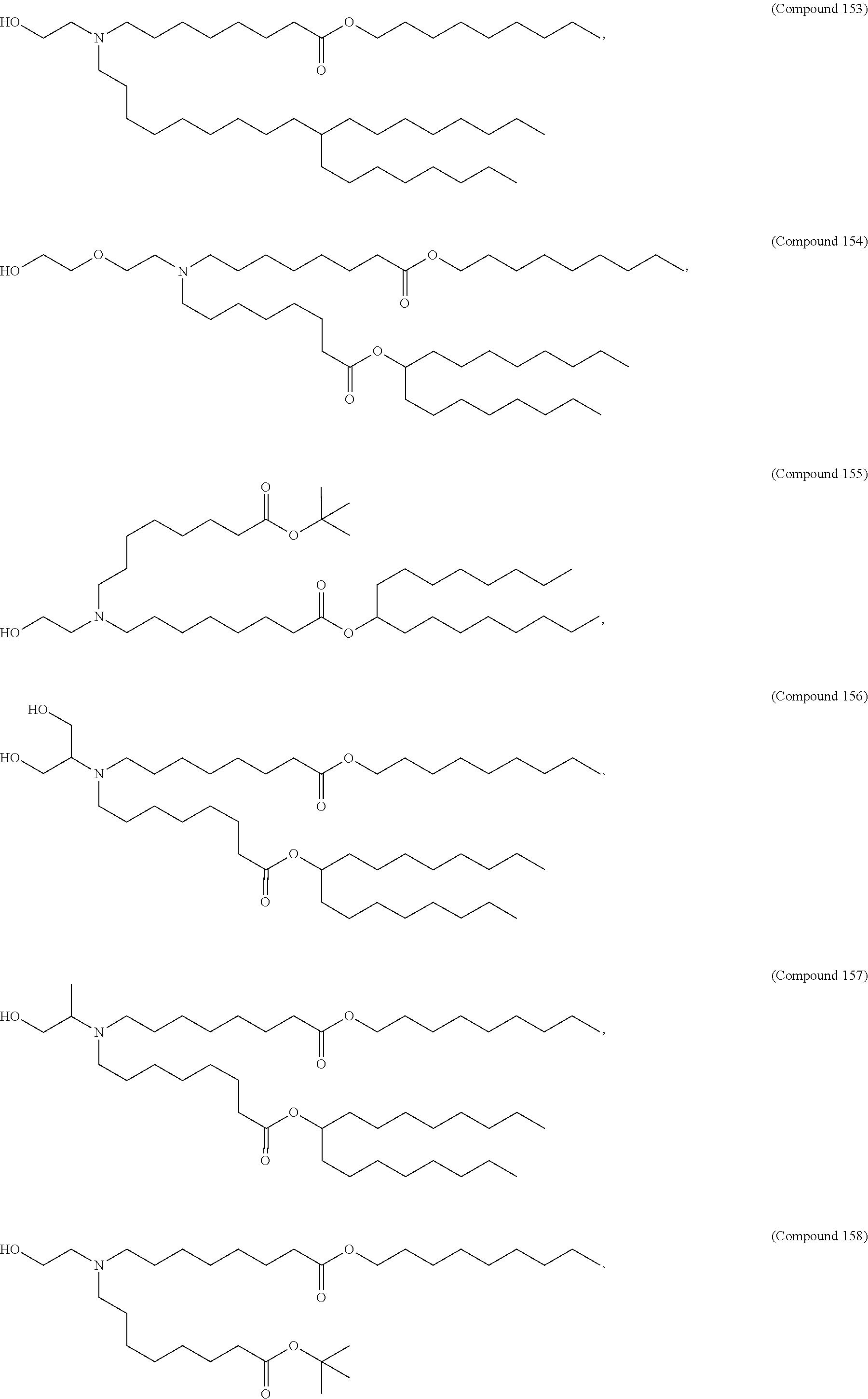 [SMILES: CCCCCCCCCOC(=O)CCCCCCCN(CCO)CCCCCCCCCC(CCCCCCCC)CCCCCCCC ; CCCCCCCCCOC(=O)CCCCCCCN(CCCCCCCC(=O)OC(CCCCCCCC)CCCCCCCC)CCOCCO ; CCCCCCCCC(CCCCCCCC)OC(=O)CCCCCCCN(CCO)CCCCCCCC(=O)OC(C)(C)C ; CCCCCCCCCOC(=O)CCCCCCCN(CCCCCCCC(=O)OC(CCCCCCCC)CCCCCCCC)C(CO)CO ; CCCCCCCCCOC(=O)CCCCCCCN(CCCCCCCC(=O)OC(CCCCCCCC)CCCCCCCC)C(C)CO ; CCCCCCCCCOC(=O)CCCCCCCN(CCO)CCCCCCCC(=O)OC(C)(C)C]

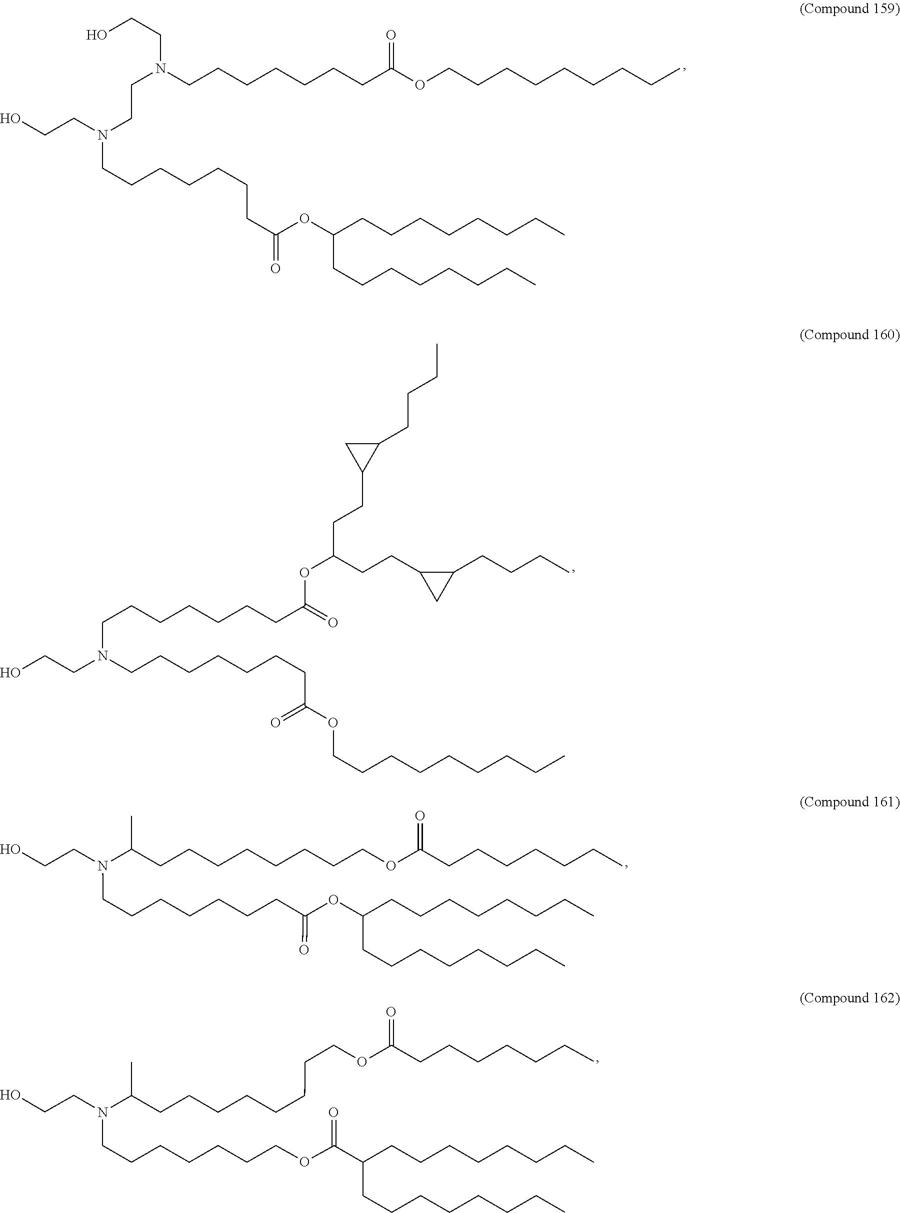

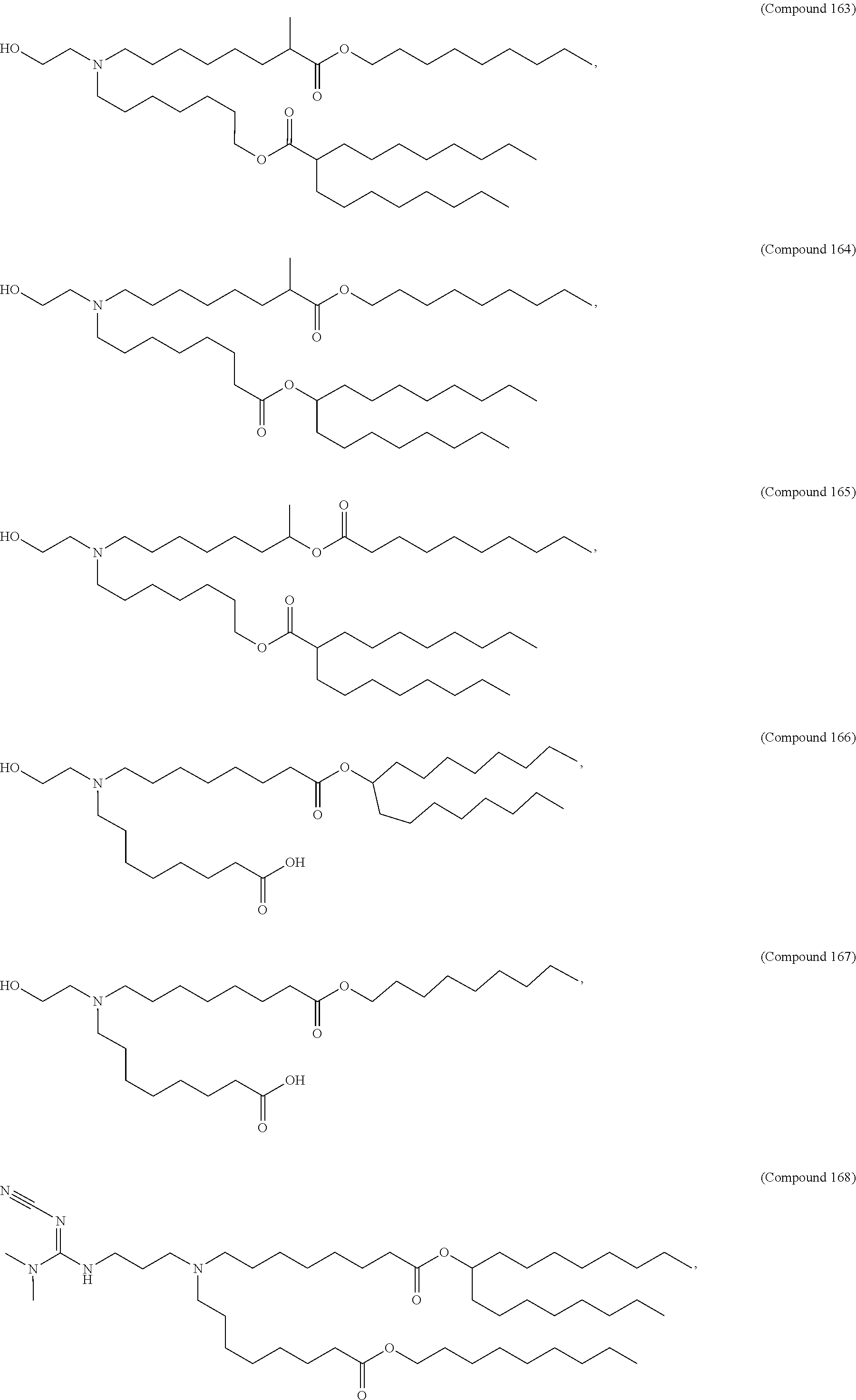 [SMILES: CCCCCCCCCOC(=O)C(C)CCCCCCN(CCO)CCCCCCCOC(=O)C(CCCCCCCC)CCCCCCCC ; CCCCCCCCCOC(=O)C(C)CCCCCCN(CCO)CCCCCCCC(=O)OC(CCCCCCCC)CCCCCCCC ; CCCCCCCCCC(=O)OC(C)CCCCCCN(CCO)CCCCCCCOC(=O)C(CCCCCCCC)CCCCCCCC ; CCCCCCCCC(CCCCCCCC)OC(=O)CCCCCCCN(CCO)CCCCCCCC(=O)O ; CCCCCCCCCOC(=O)CCCCCCCN(CCO)CCCCCCCC(=O)O ; CCCCCCCCCOC(=O)CCCCCCCN(CCCCCCCC(=O)OC(CCCCCCCC)CCCCCCCC)CCCN/C(=N/C#N)N(C)C]

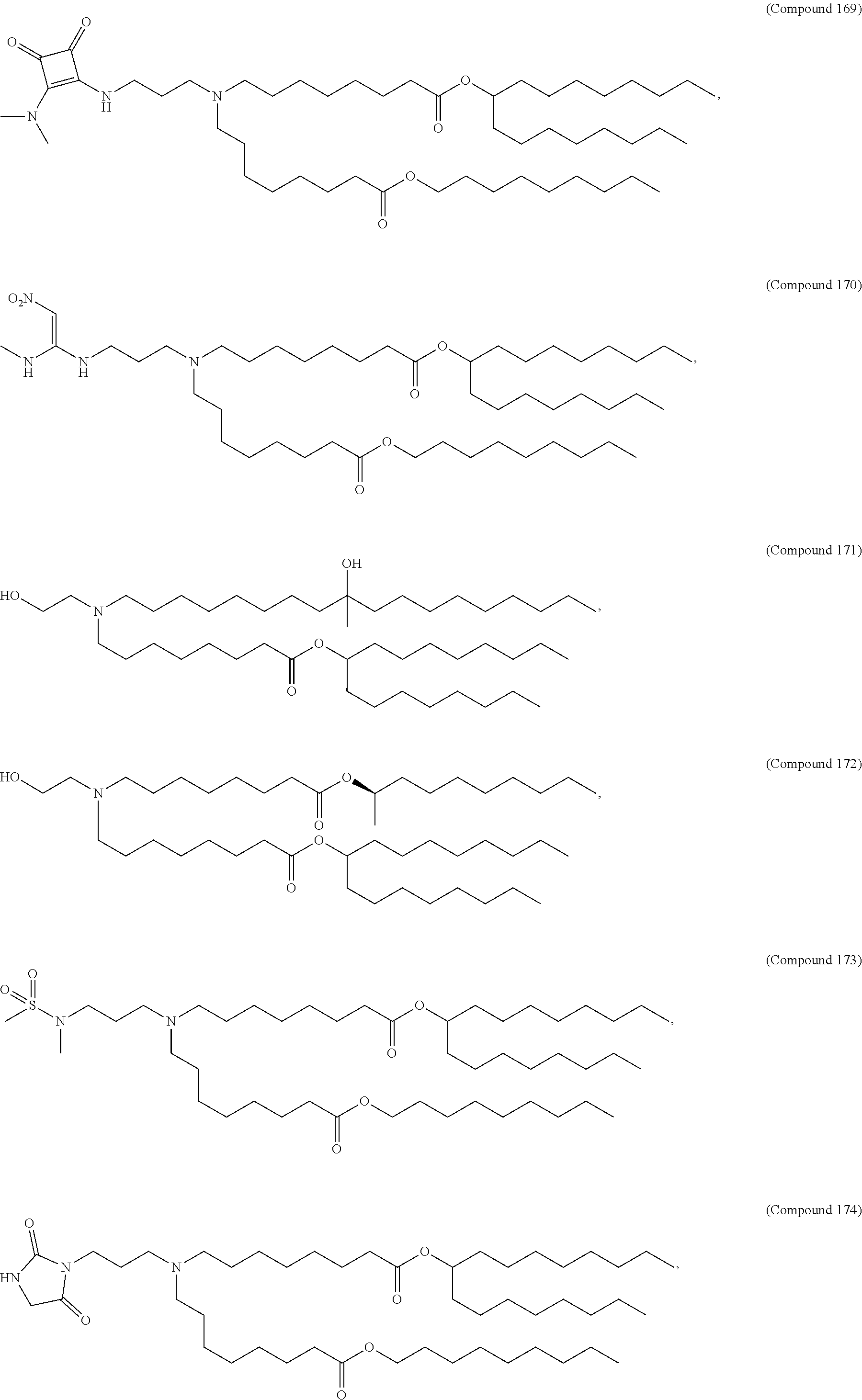

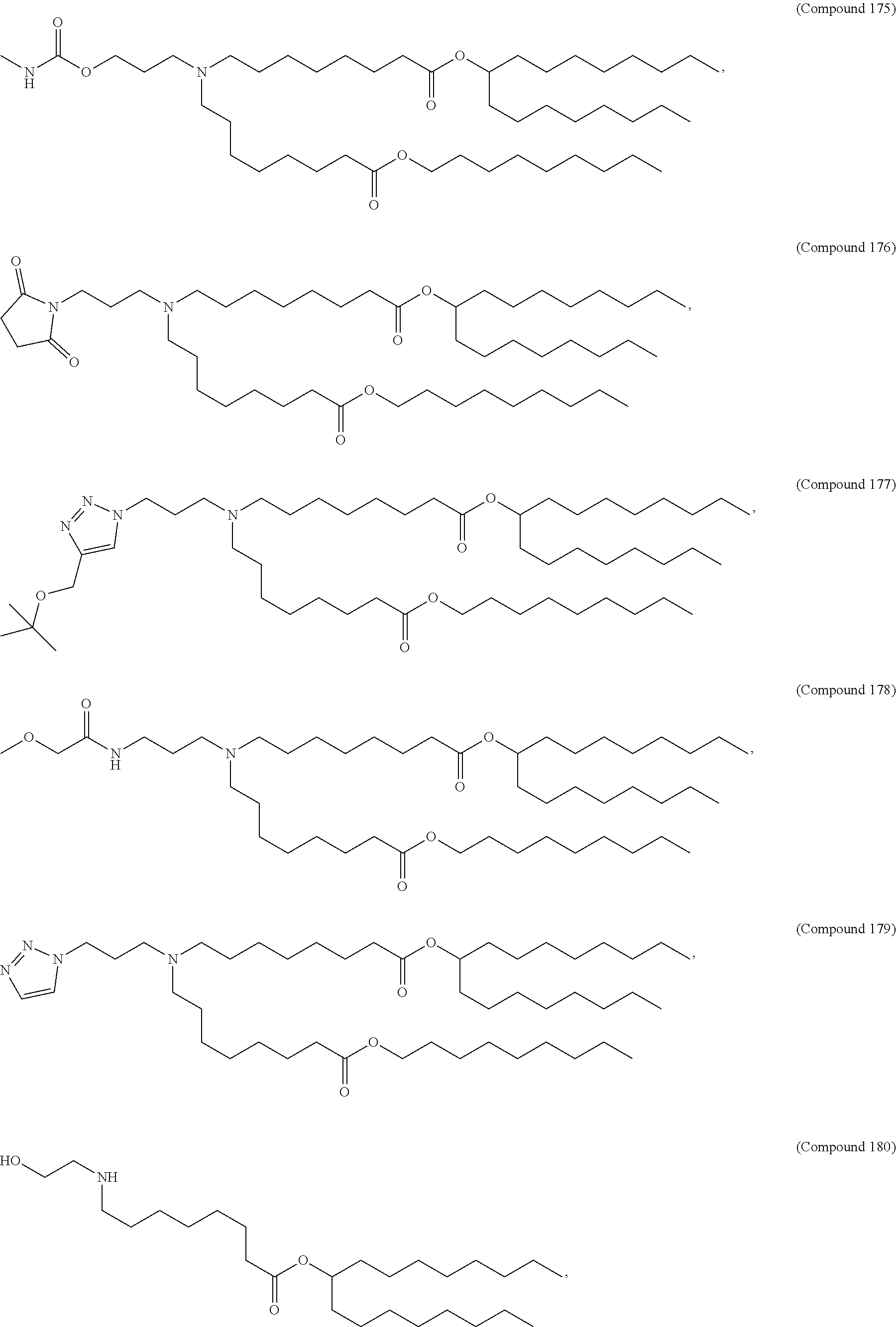

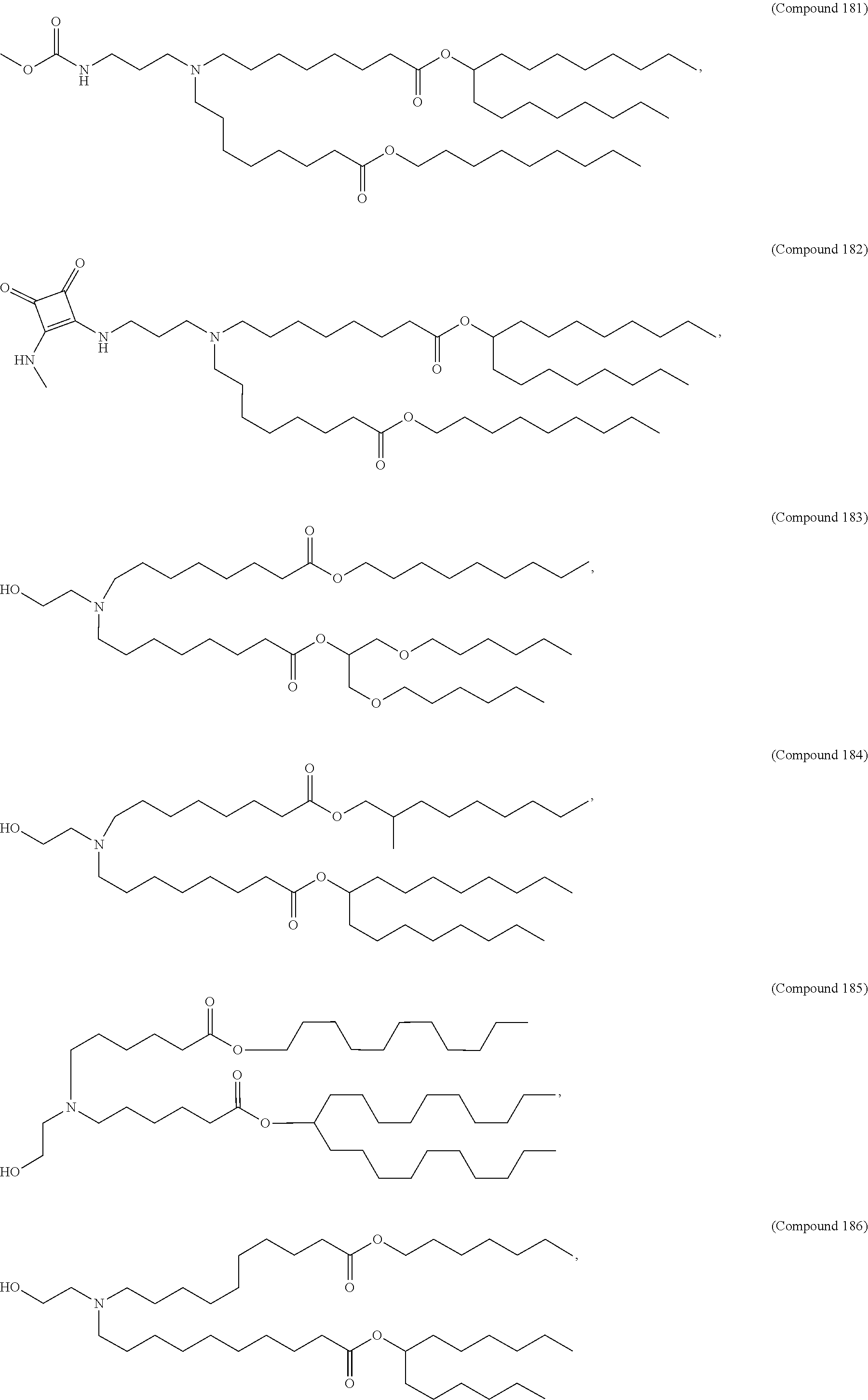 [SMILES: CCCCCCCCCOC(=O)CCCCCCCN(CCCCCCCC(=O)OC(CCCCCCCC)CCCCCCCC)CCCNC(=O)OC ; CCCCCCCCCOC(=O)CCCCCCCN(CCCCCCCC(=O)OC(CCCCCCCC)CCCCCCCC)CCCNc1c(NC)c(=O)c1=O ; CCCCCCCCCOC(=O)CCCCCCCN(CCO)CCCCCCCC(=O)OC(COCCCCCC)COCCCCCC ; CCCCCCCCC(CCCCCCCC)OC(=O)CCCCCCCN(CCO)CCCCCCCC(=O)OCC(C)CCCCCCC ; CCCCCCCCCCCOC(=O)CCCCCN(CCO)CCCCCC(=O)OC(CCCCCCCCCC)CCCCCCCCCC ; CCCCCCCOC(=O)CCCCCCCCCN(CCO)CCCCCCCCCC(=O)OC(CCCCCC)CCCCCC]

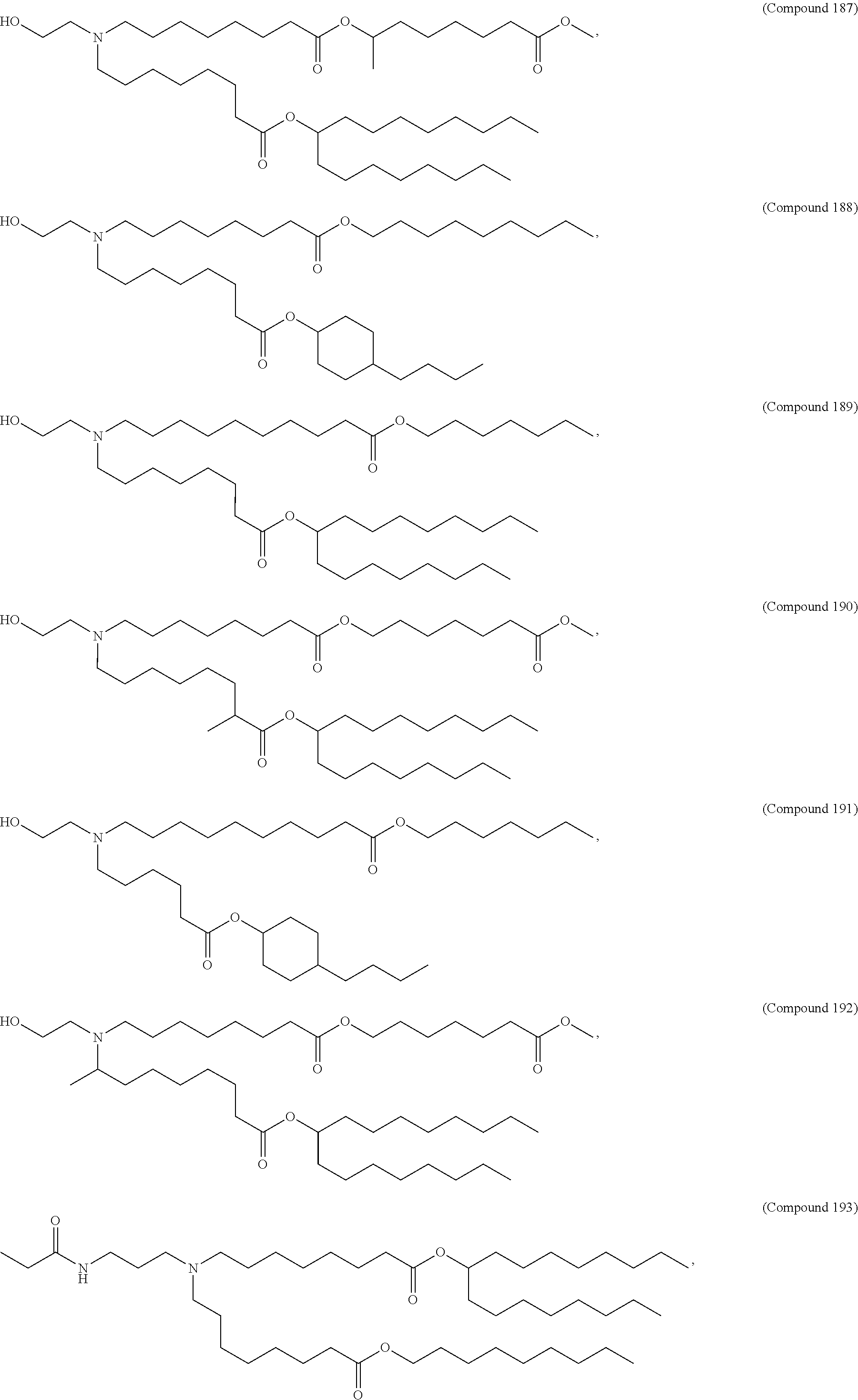

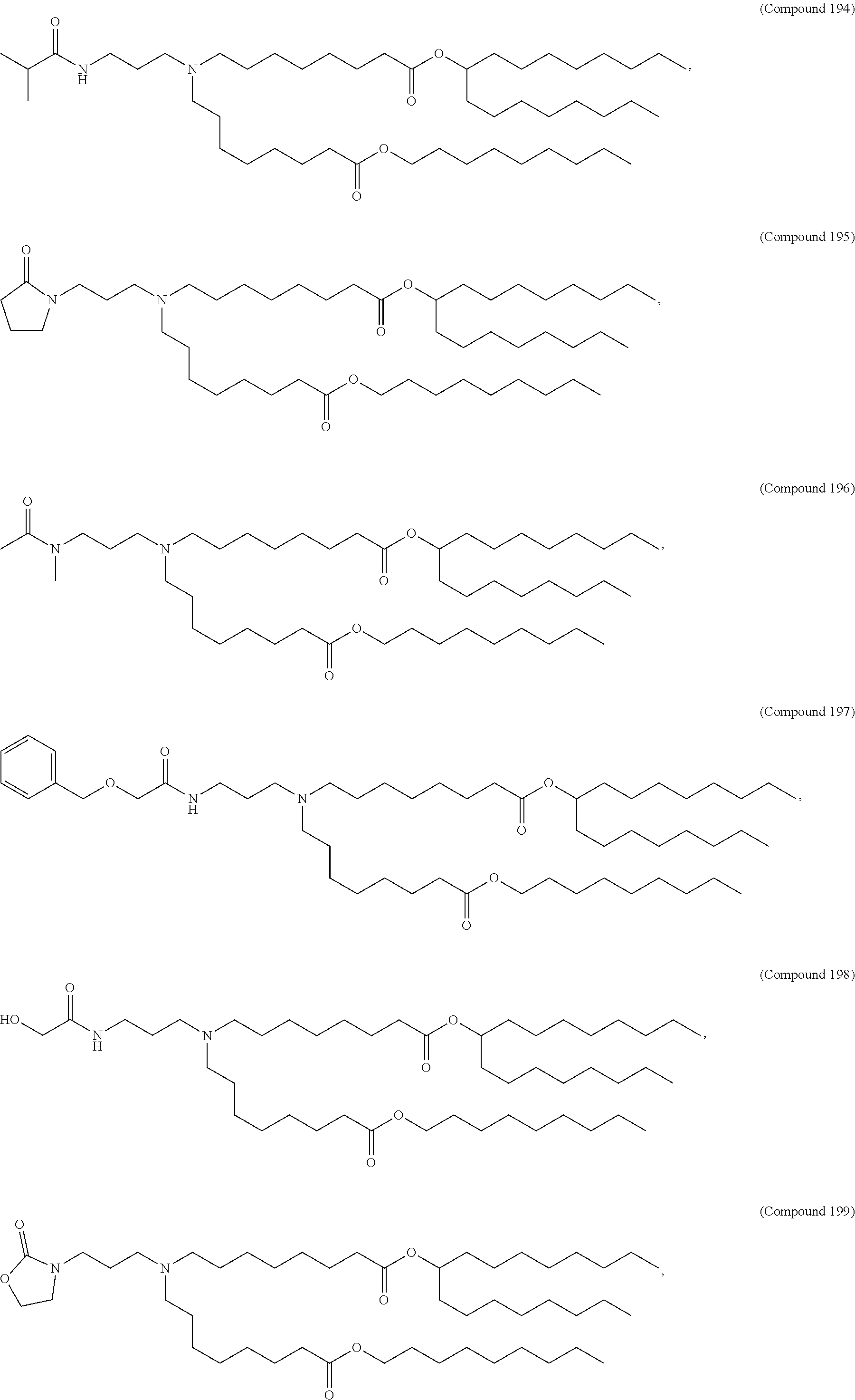

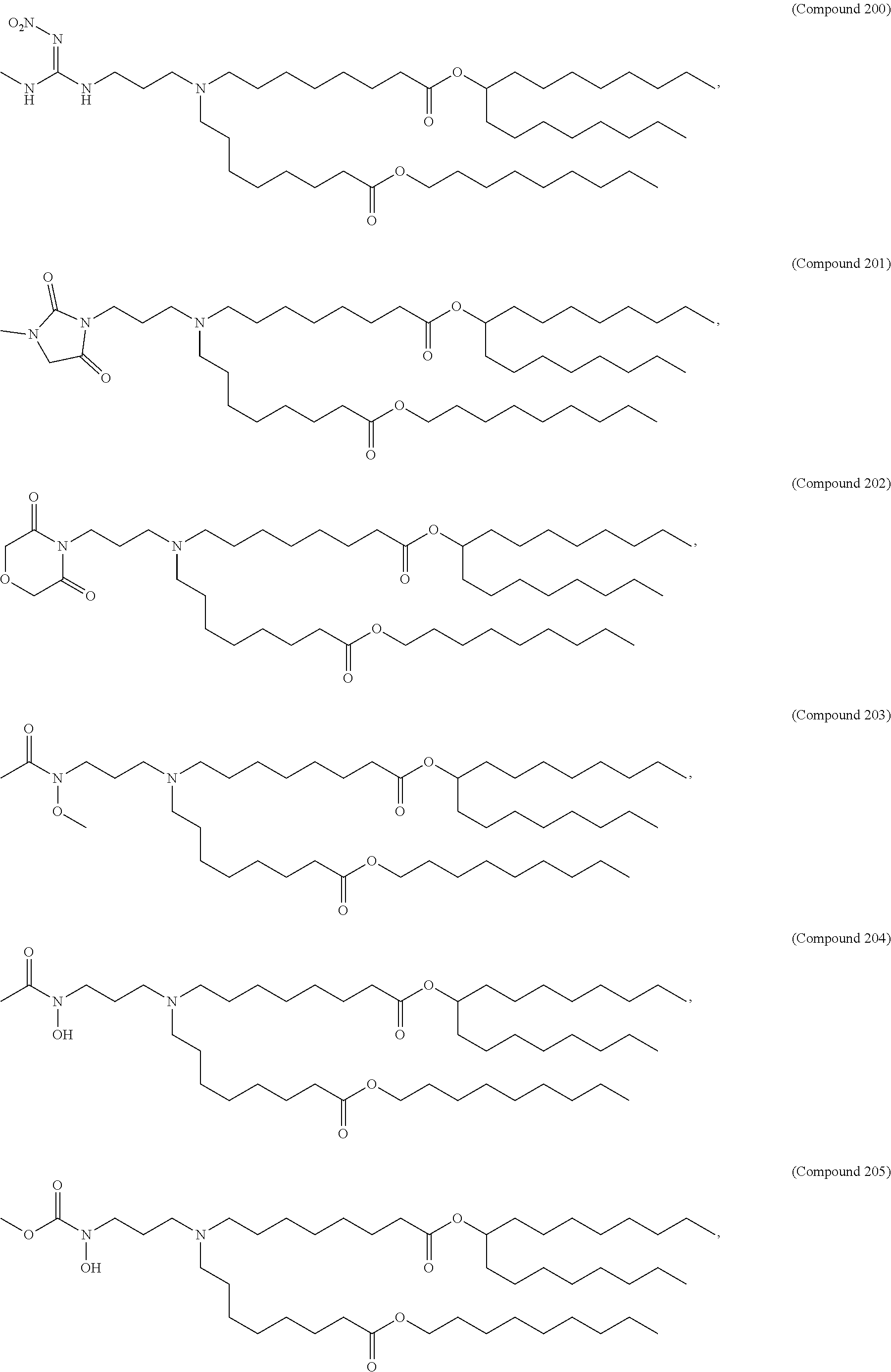

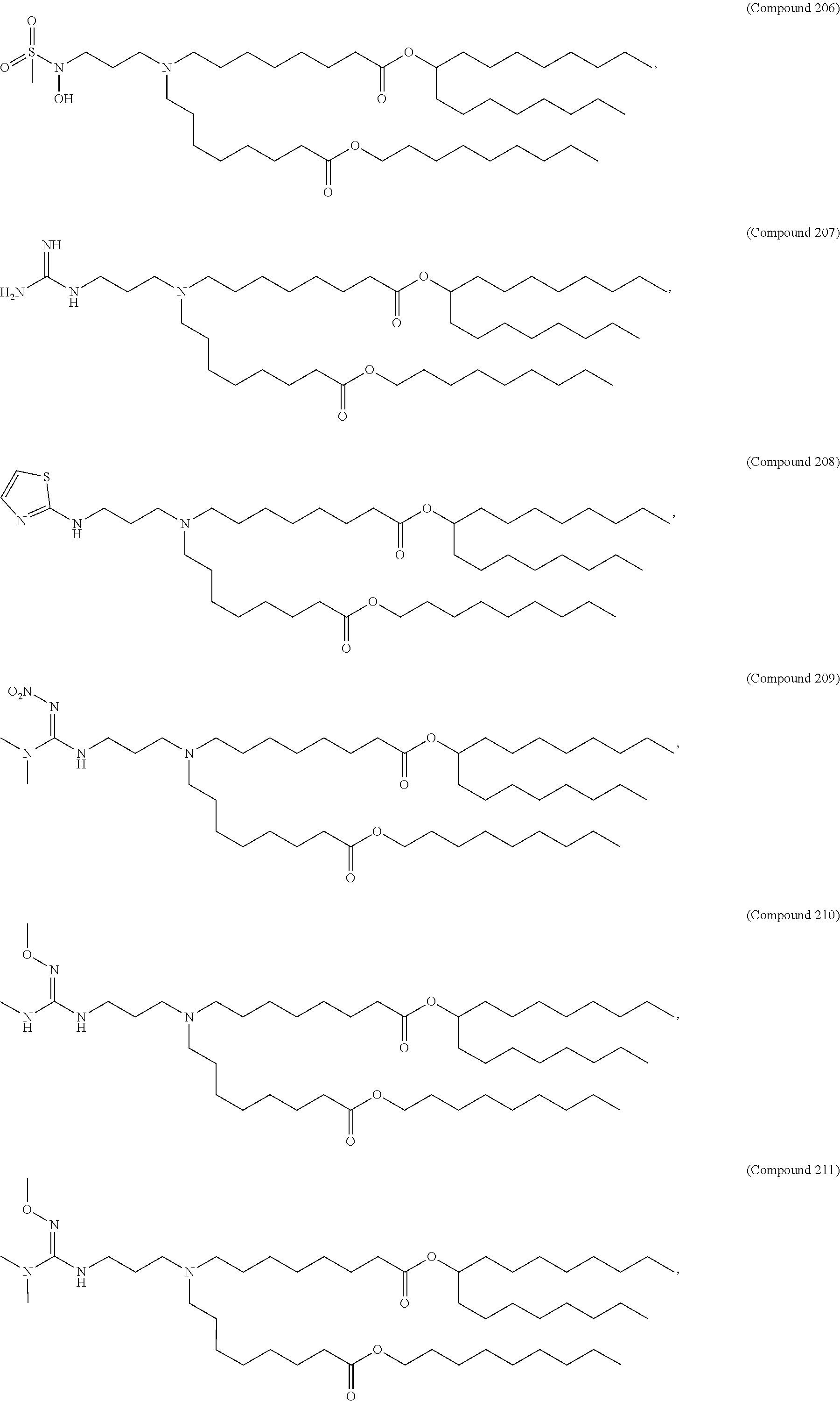

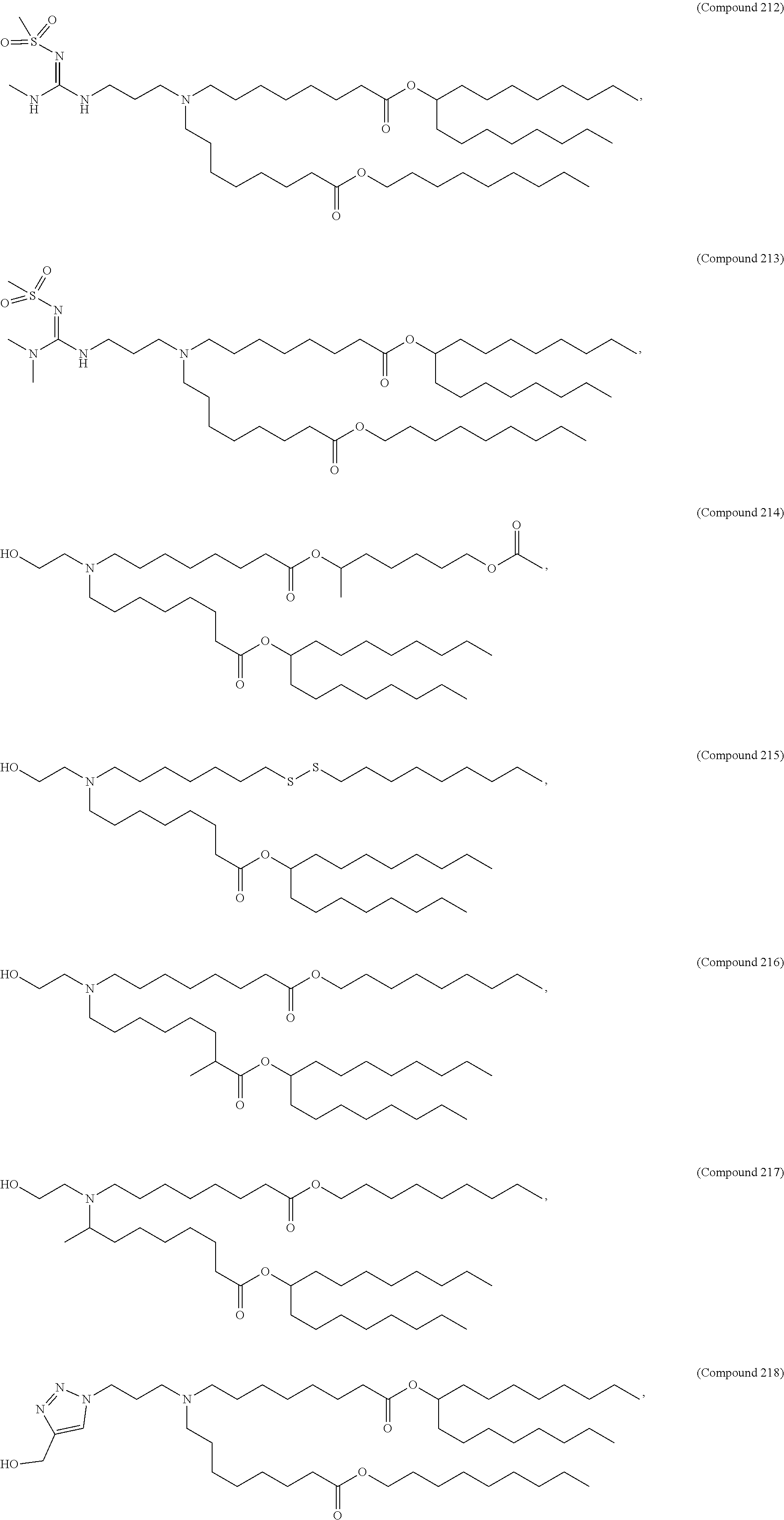

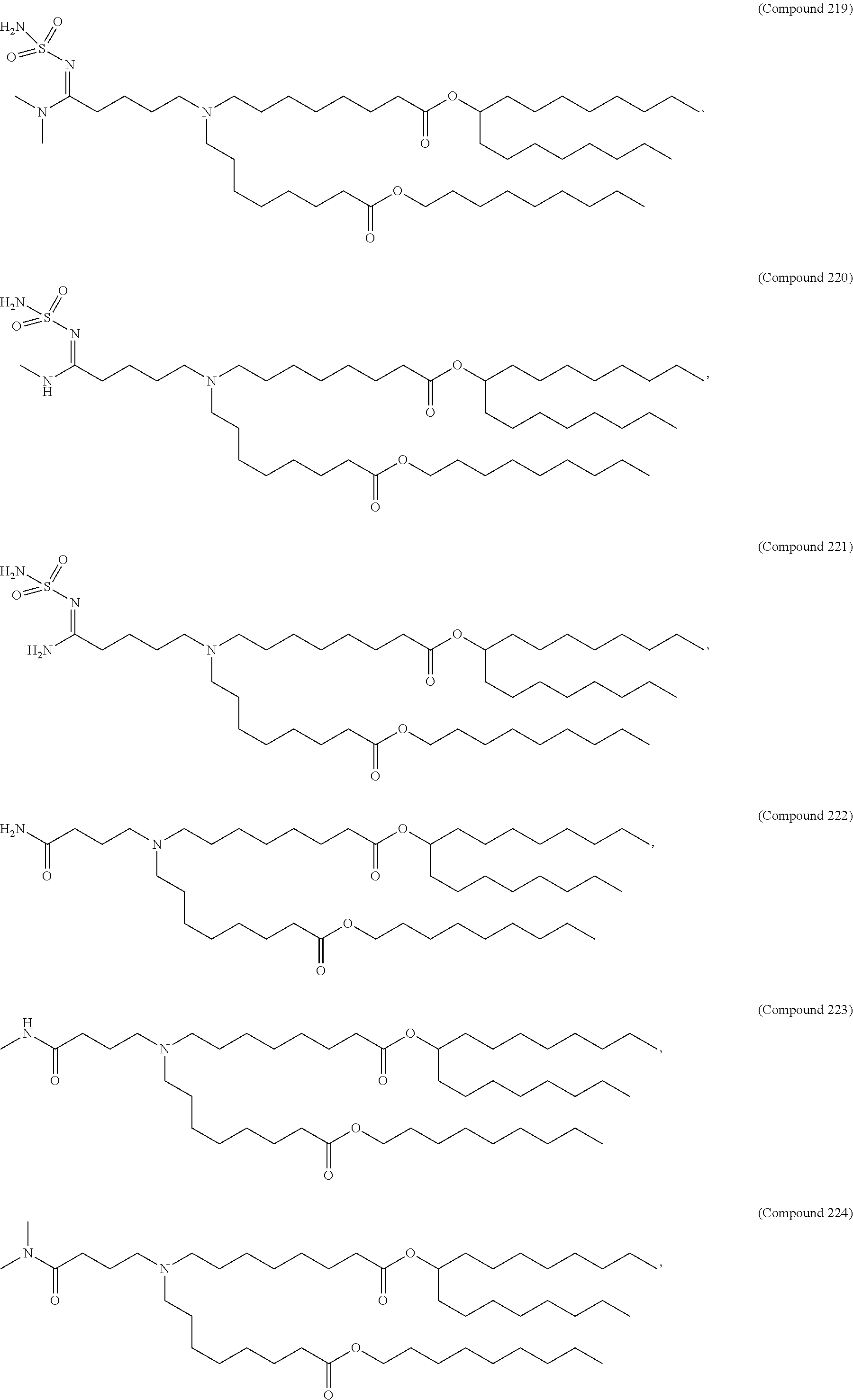

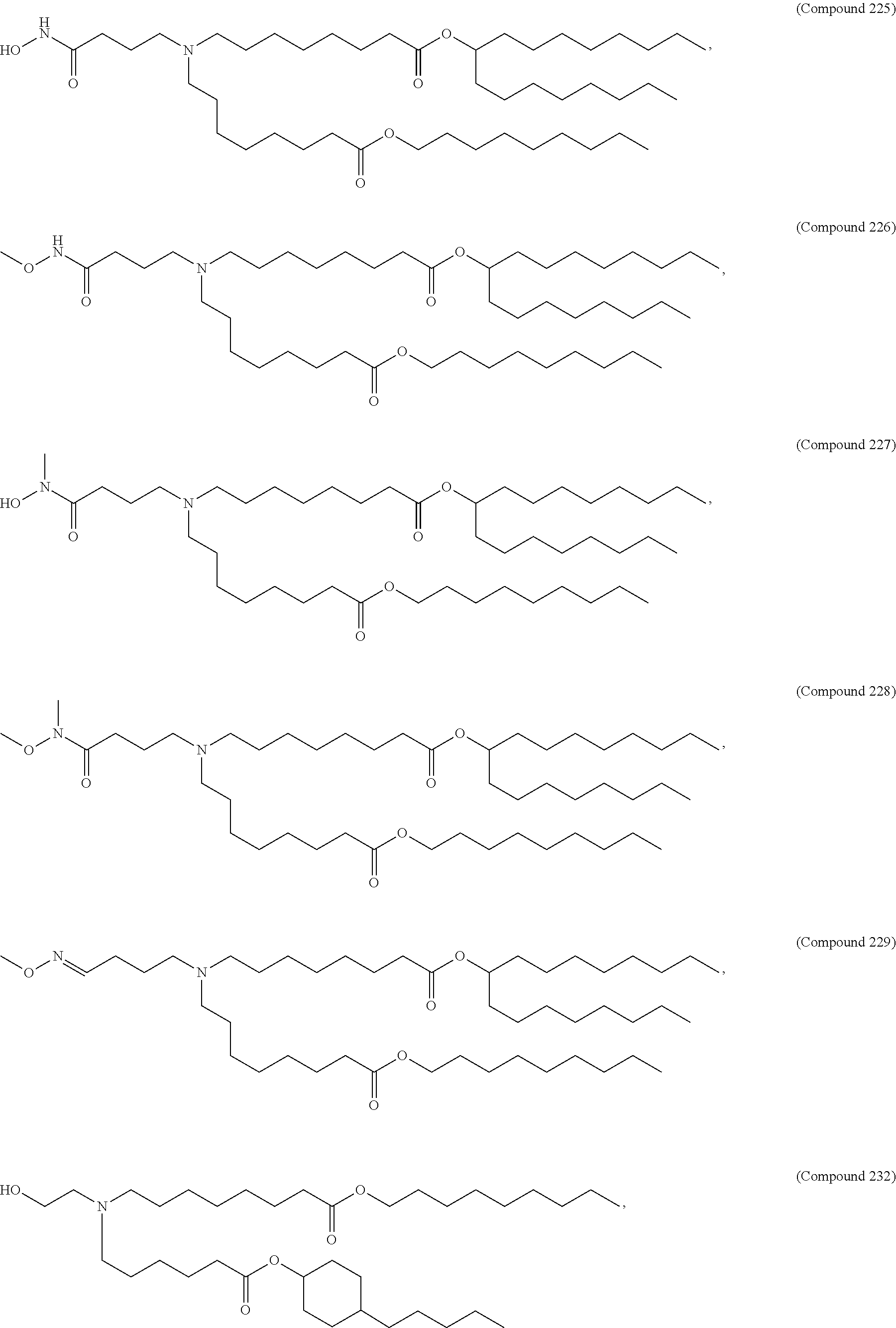

salts and isomers thereof.

In some embodiments, the ionizable lipid is compound 429:

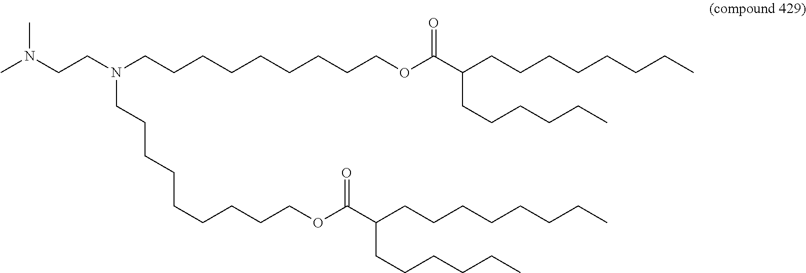 [SMILES: CCCCCCCCC(CCCCCC)C(=O)OCCCCCCCCCN(CCCCCCCCCOC(=O)C(CCCCCC)CCCCCCCC)CCN(C)C]

salt thereof.

In some embodiments, the ionizable lipid is compound 18:

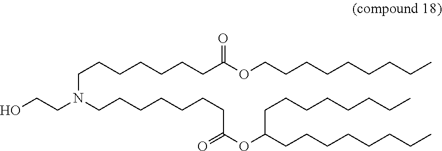 [SMILES: CCCCCCCCCOC(=O)CCCCCCCN(CCO)CCCCCCCC(=O)OC(CCCCCCCC)CCCCCCCC]

or a salt thereof.

In some embodiments, a lipid nanoparticle composition includes a lipid component comprising a compound as described herein (e.g., a compound according to Formula (I), (IA), (II), (IIa), (IIb), (IIc), (IId) or (IIe)).

In some embodiments LNPs may be comprised of ionizable lipids including a central piperazine moiety. Such LNPs advantageously may be composed of an ionizable lipid, a phospholipid and a PEG lipid and may optionally include a structural lipid or may lack a structural lipid. In some embodiments the phospholipid is a DSPC or DOP.

The ionizable lipids including a central piperazine moiety described herein may be advantageously used in lipid nanoparticle compositions for the delivery of therapeutic and/or prophylactic agents to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent has an increased therapeutic index as compared to a corresponding formulation which comprises a reference lipid (e.g., MC3, KC2, or DLinDMA) and the same therapeutic or prophylactic agent.

Lipids may be compounds of Formula (III),

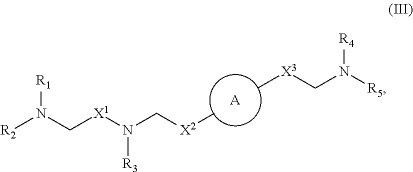

or salts or isomers thereof, wherein
ring A is

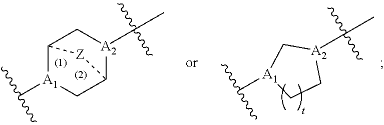

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —S C(S)—, —CH(OH)—, —P(O)(OR′)O—, —S(O)₂—, an aryl group, and a heteroaryl group;
X¹, X², and X³ are independently selected from the group consisting of a bond, —CH₂—,
   —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl,
wherein when ring A is

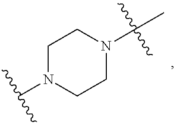 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (IIIa1)-(IIIa6):

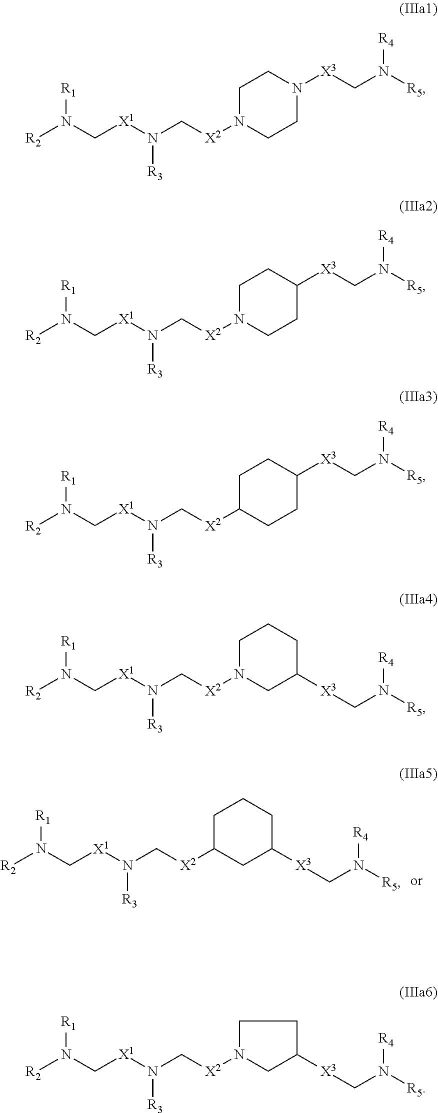

The compounds of Formula (III) or any of (IIIa1)-(IIIa6) include one or more of the following features when applicable.

In some embodiments, ring A is

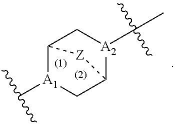

In some embodiments, ring A is

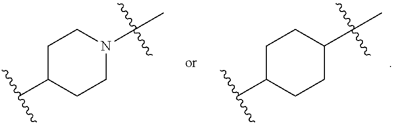 [SMILES: *C1CCN(*)CC1 ; *C1CCC(*)CC1]

In some embodiments, ring A is

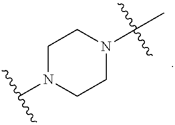 [SMILES: *N1CCN(*)CC1]

In some embodiments, ring A is

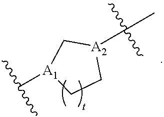

In some embodiments, ring A is

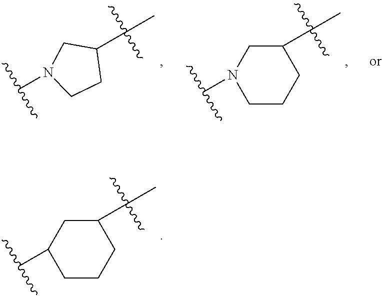 [SMILES: *C1CCN(*)C1 ; *C1CCCN(*)C1 ; *C1CCCC(*)C1]

In some embodiments, ring A is

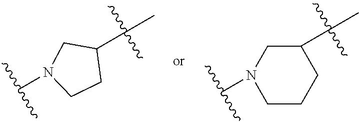 [SMILES: *C1CCN(*)C1 ; *C1CCCN(*)C1]

wherein ring, in which the N atom is connected with X².

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, at least one of X¹, X², and X³ is not —CH₂—. For example, in certain embodiments, X¹ is not —CH₂—. In some embodiments, at least one of X¹, X², and X³ is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, X³ is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—. In other embodiments, X³ is —CH₂—.

In some embodiments, X³ is a bond or —(CH₂)₂—.

In some embodiments, R₁ and R₂ are the same. In certain embodiments, R₁, R₂, and R₃ are the same. In some embodiments, R₄ and R₅ are the same. In certain embodiments, R₁, R₂, R₃, R₄, and R₅ are the same.

In some embodiments, at least one of R₁, R₂, R₃, R₄, and R₅ is —R″MR′. In some embodiments, at most one of R₁, R₂, R₃, R₄, and R₅ is —R″MR′. For example, at least one of R₁, R₂, and R₃ may be —R″MR′, and/or at least one of R₄ and R₅ is —R″MR′. In certain embodiments, at least one M is —C(O)O—. In some embodiments, each M is —C(O)O—. In some embodiments, at least one M is —OC(O)—. In some embodiments, each M is —OC(O)—. In some embodiments, at least one M is —OC(O)O—. In some embodiments, each M is —OC(O)O—. In some embodiments, at least one R″ is C₃ alkyl. In certain embodiments, each R″ is C₃ alkyl. In some embodiments, at least one R″ is C₅ alkyl. In certain embodiments, each R″ is C₅ alkyl. In some embodiments, at least one R″ is C₆ alkyl. In certain embodiments, each R″ is C₆ alkyl. In some embodiments, at least one R″ is C₇ alkyl. In certain embodiments, each R″ is C₇ alkyl. In some embodiments, at least one R′ is C₅ alkyl. In certain embodiments, each R′ is C₅ alkyl. In other embodiments, at least one R′ is C₁ alkyl. In certain embodiments, each R′ is C₁ alkyl. In some embodiments, at least one R′ is C₂ alkyl. In certain embodiments, each R′ is C₂ alkyl.

In some embodiments, at least one of R₁, R₂, R₃, R₄, and R₅ is C₁₂ alkyl. In certain embodiments, each of R₁, R₂, R₃, R₄, and R₅ are C₁₂ alkyl.

In certain embodiments, the compound is selected from the group consisting of:

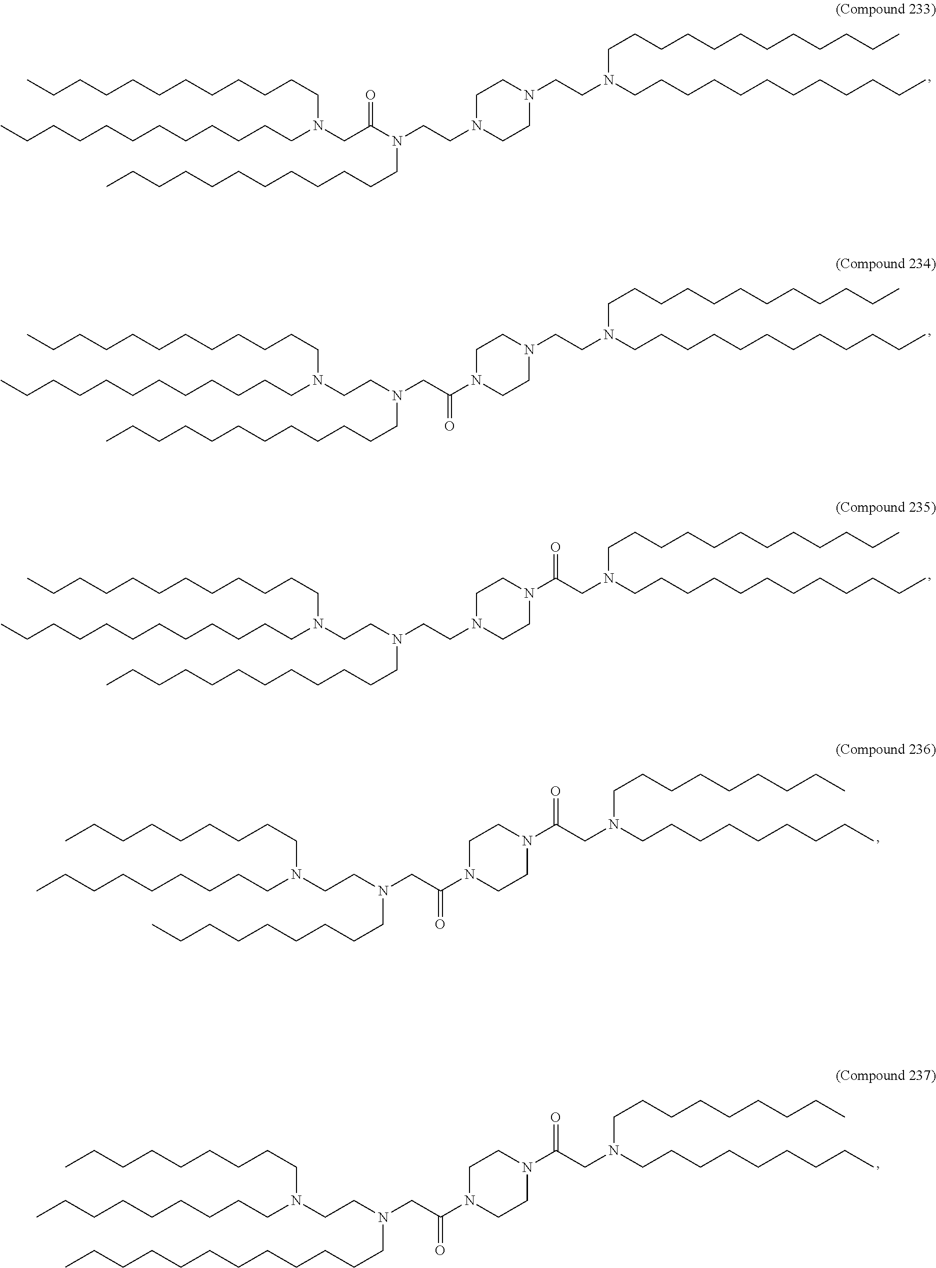

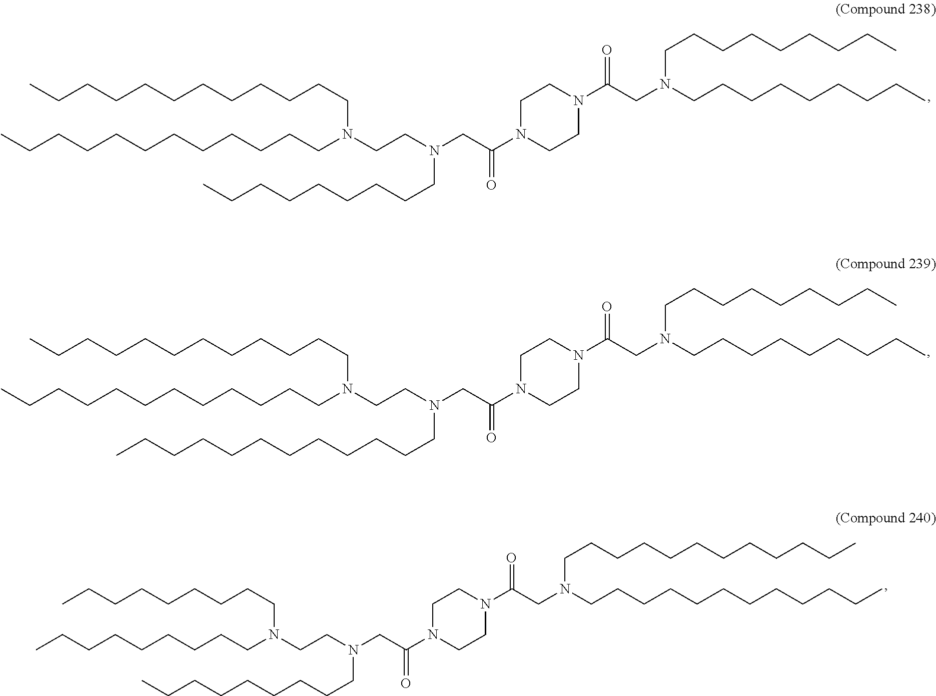

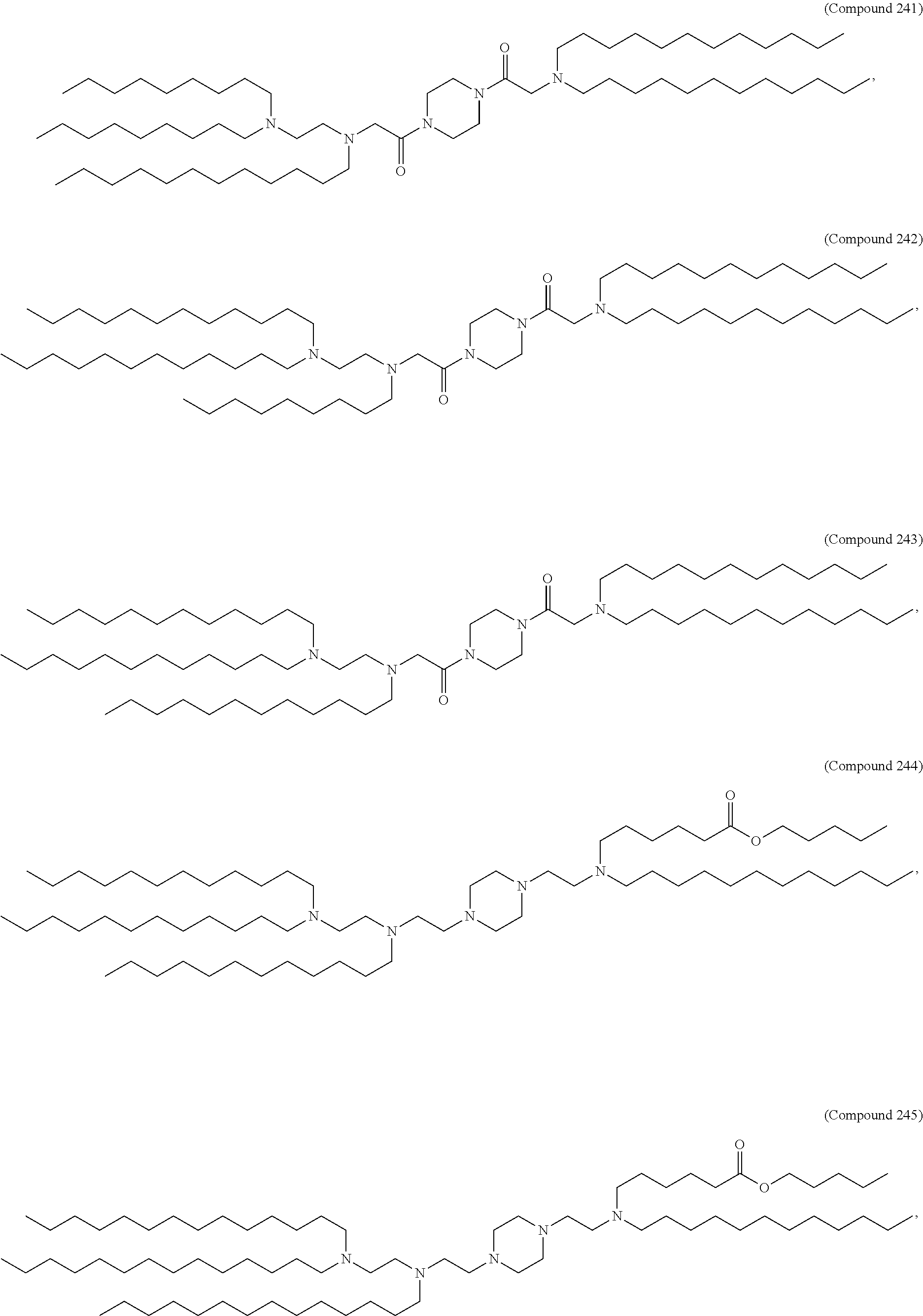

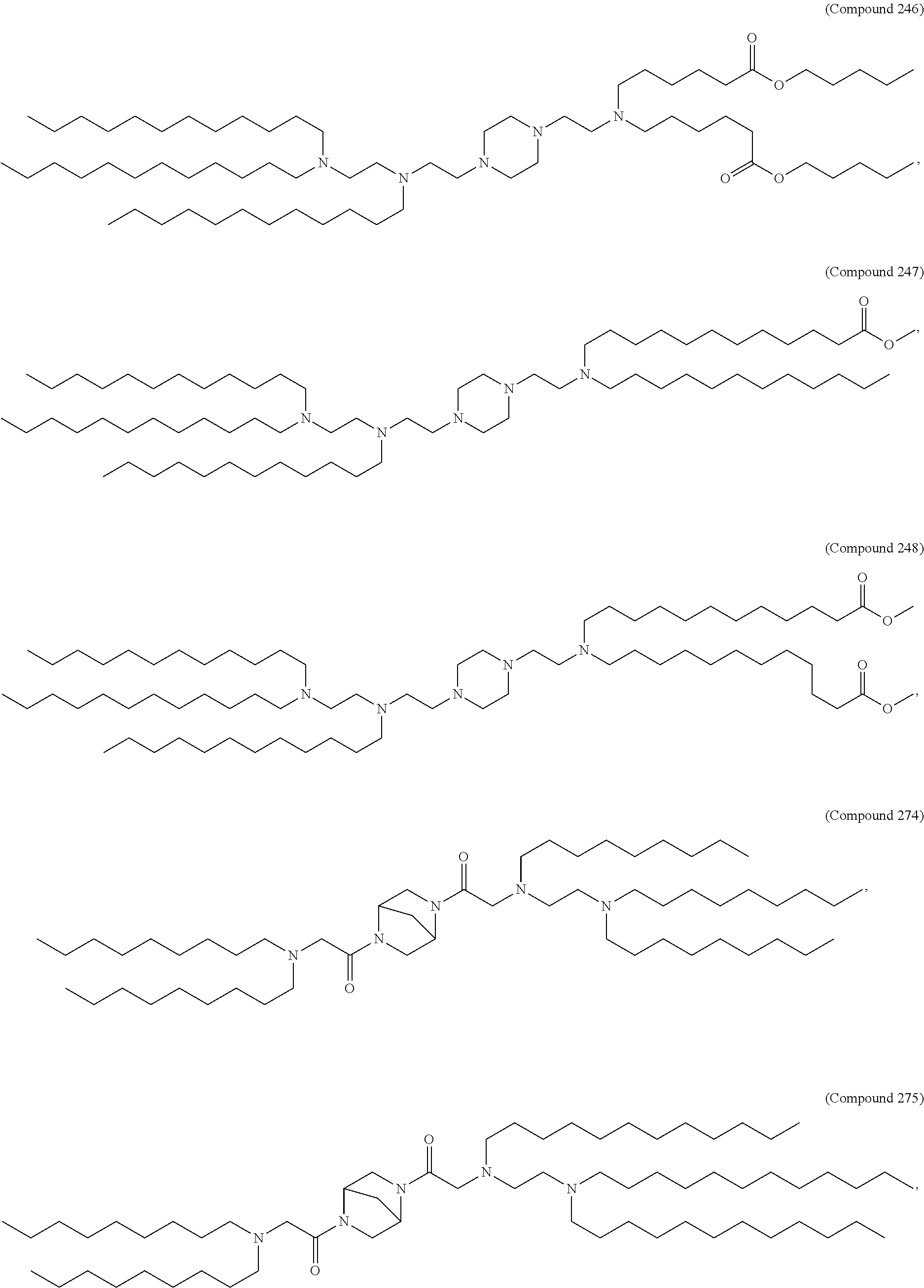

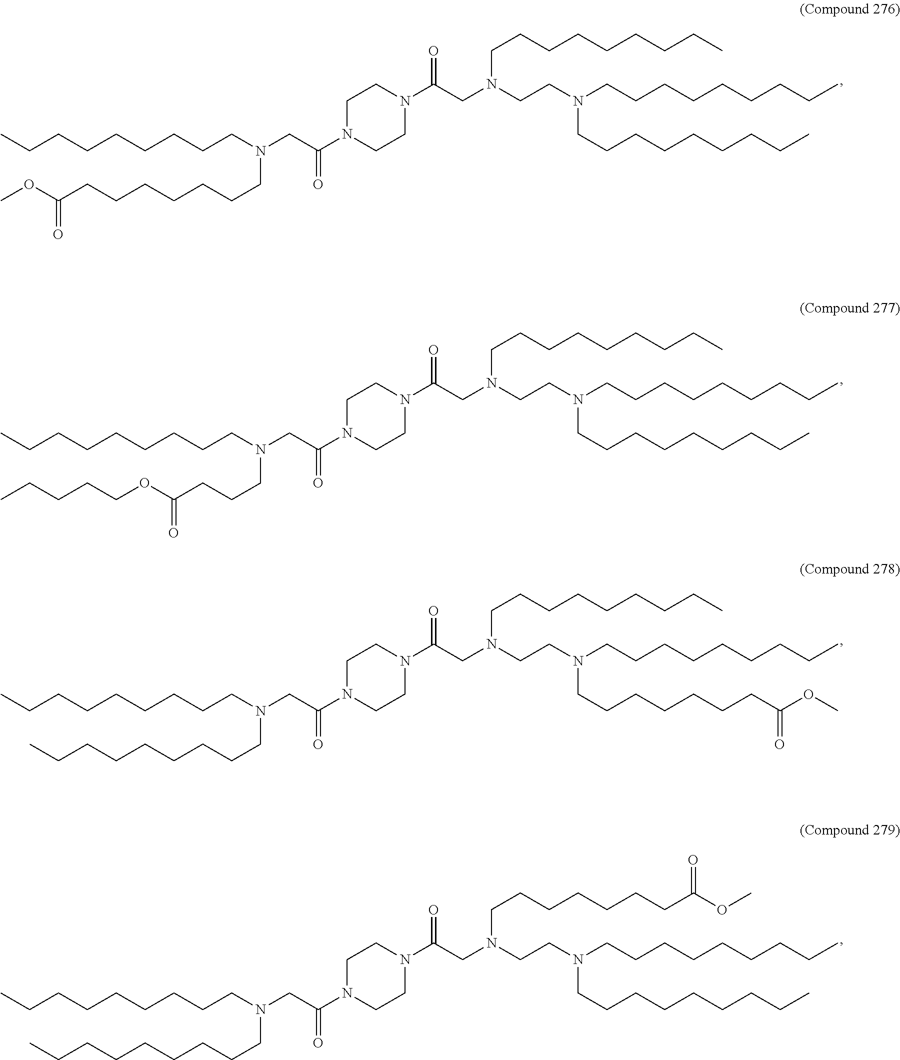

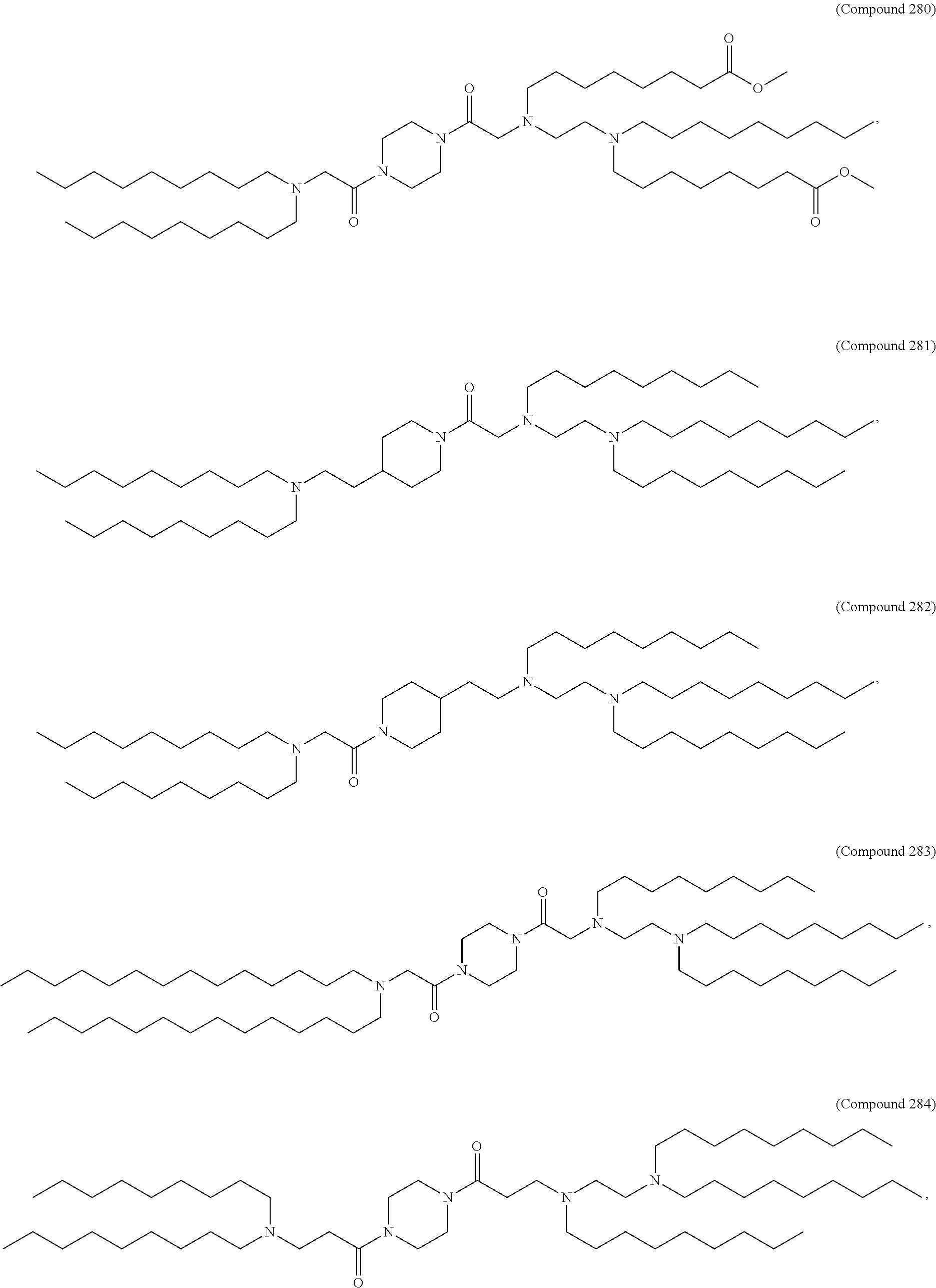

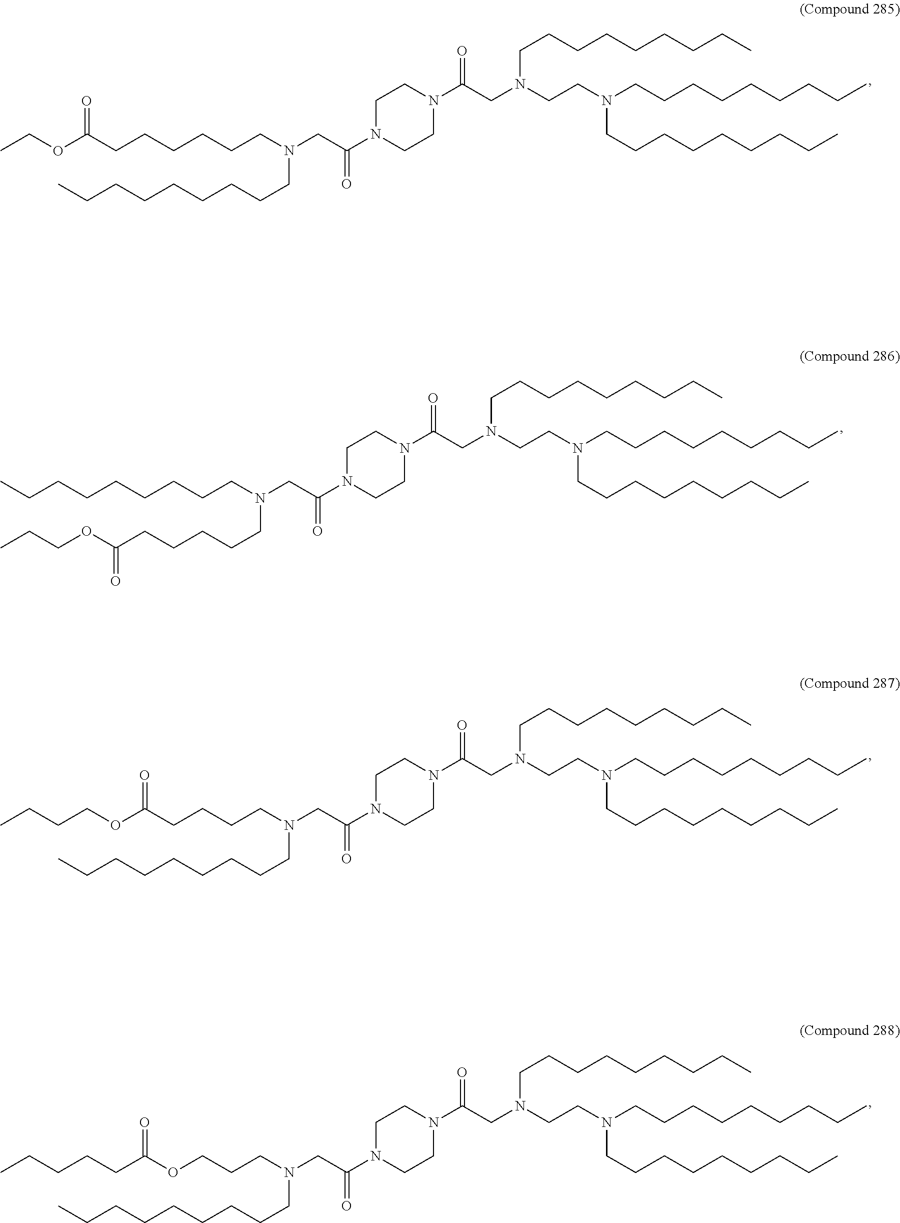

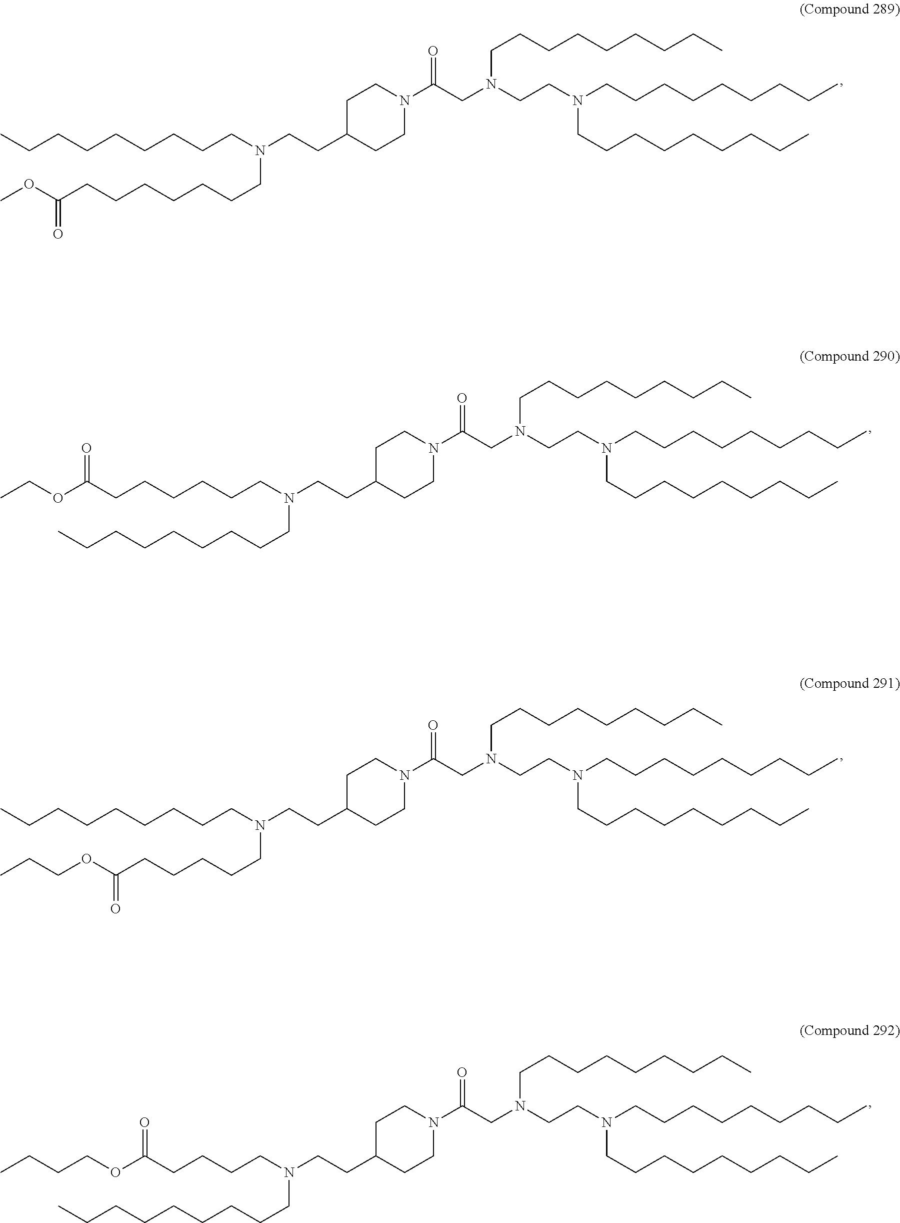

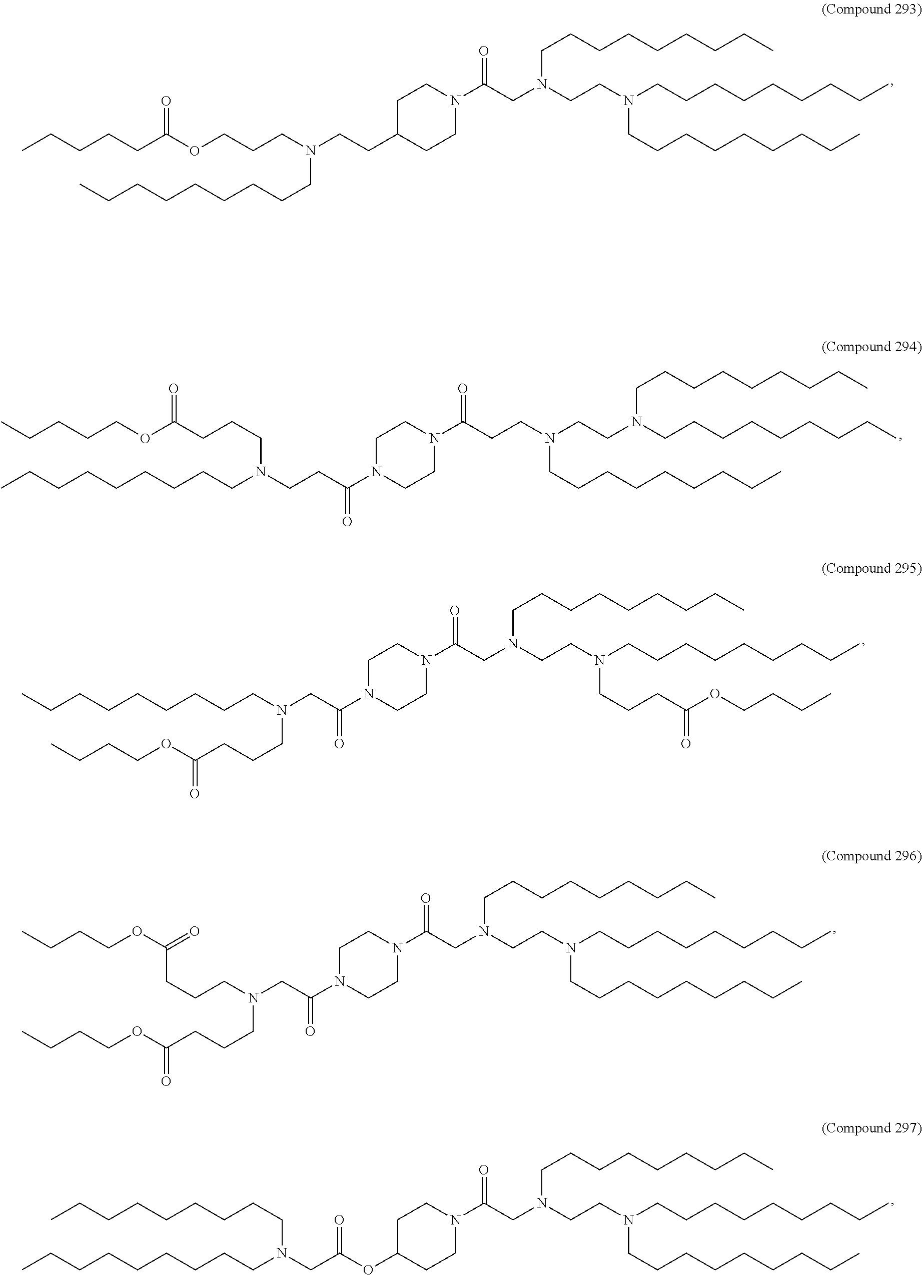

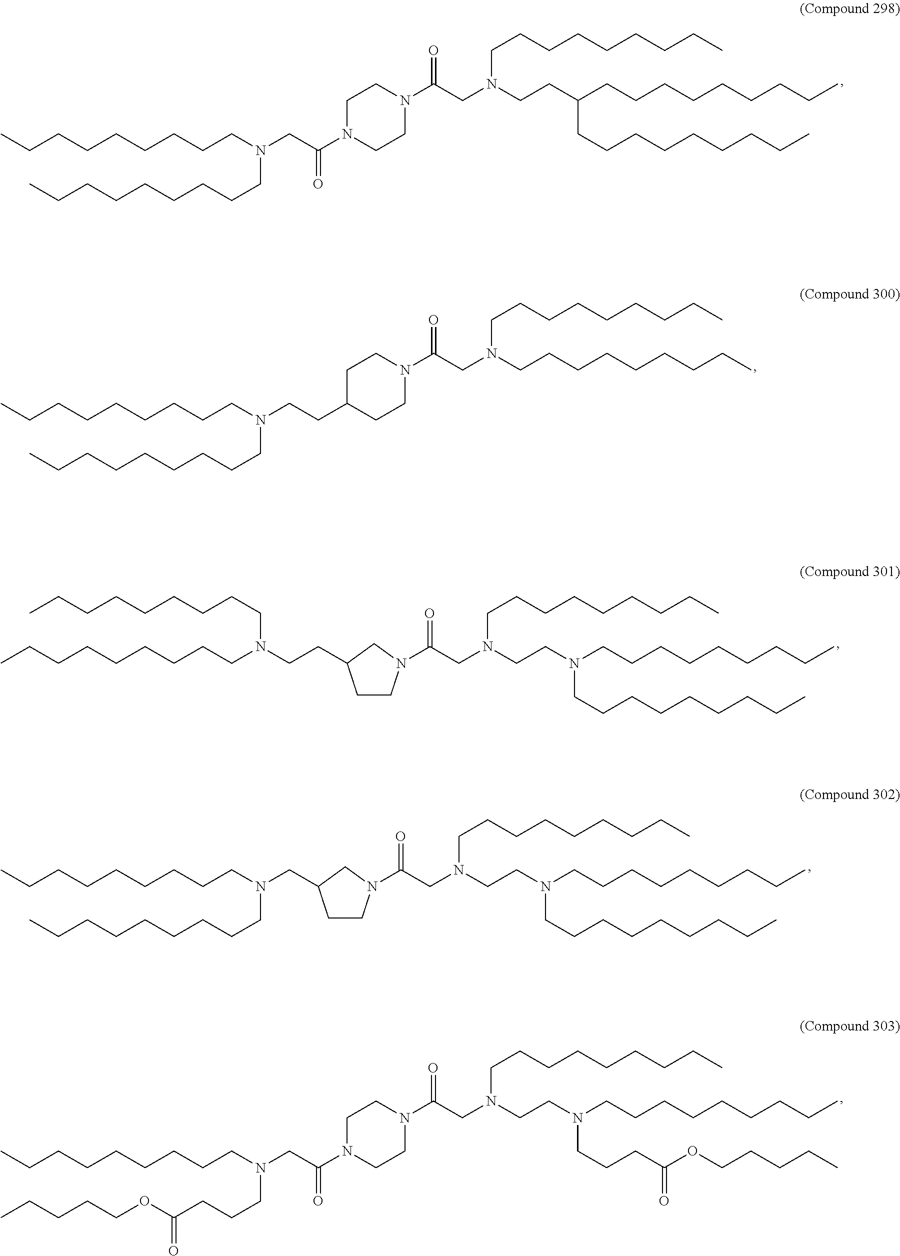

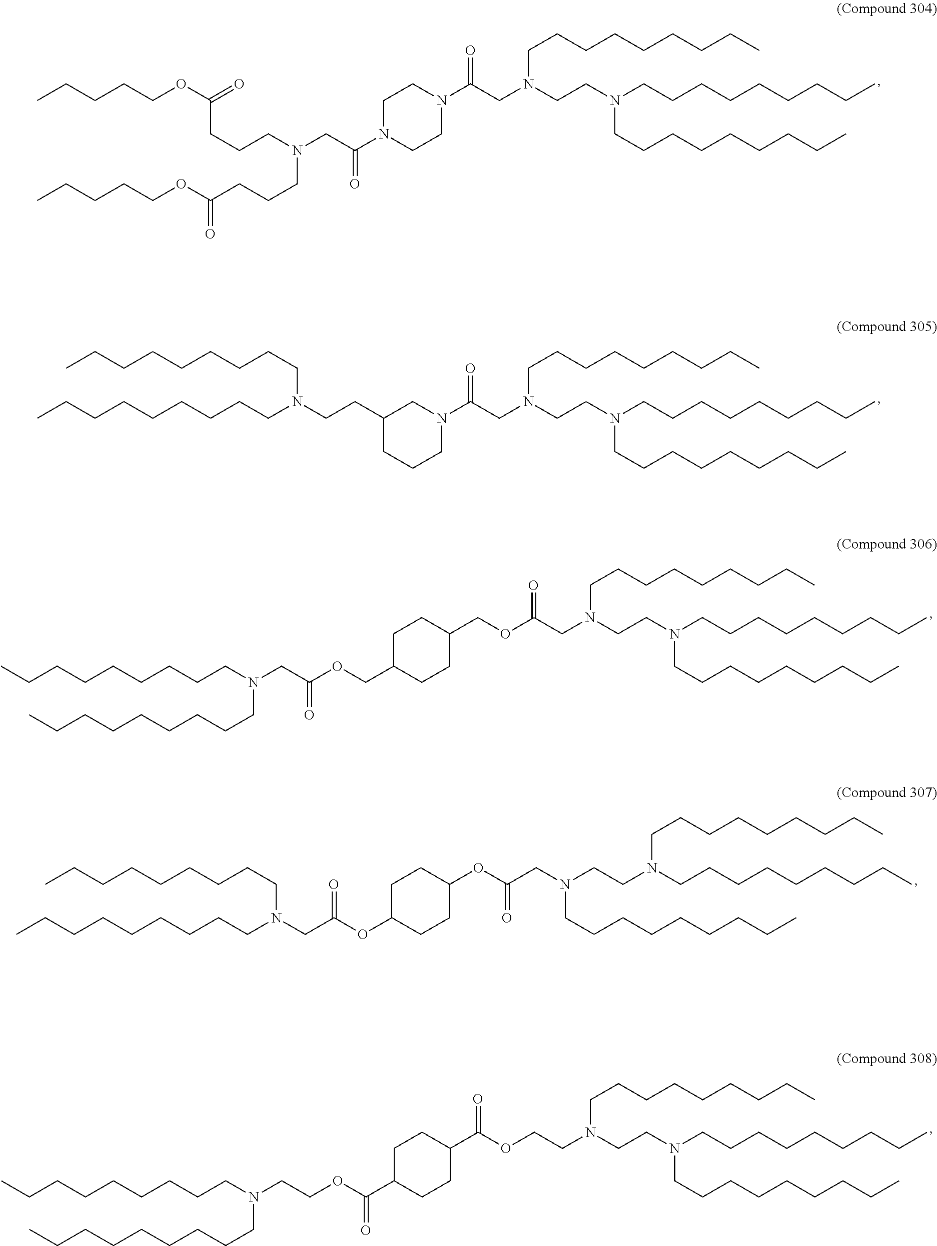

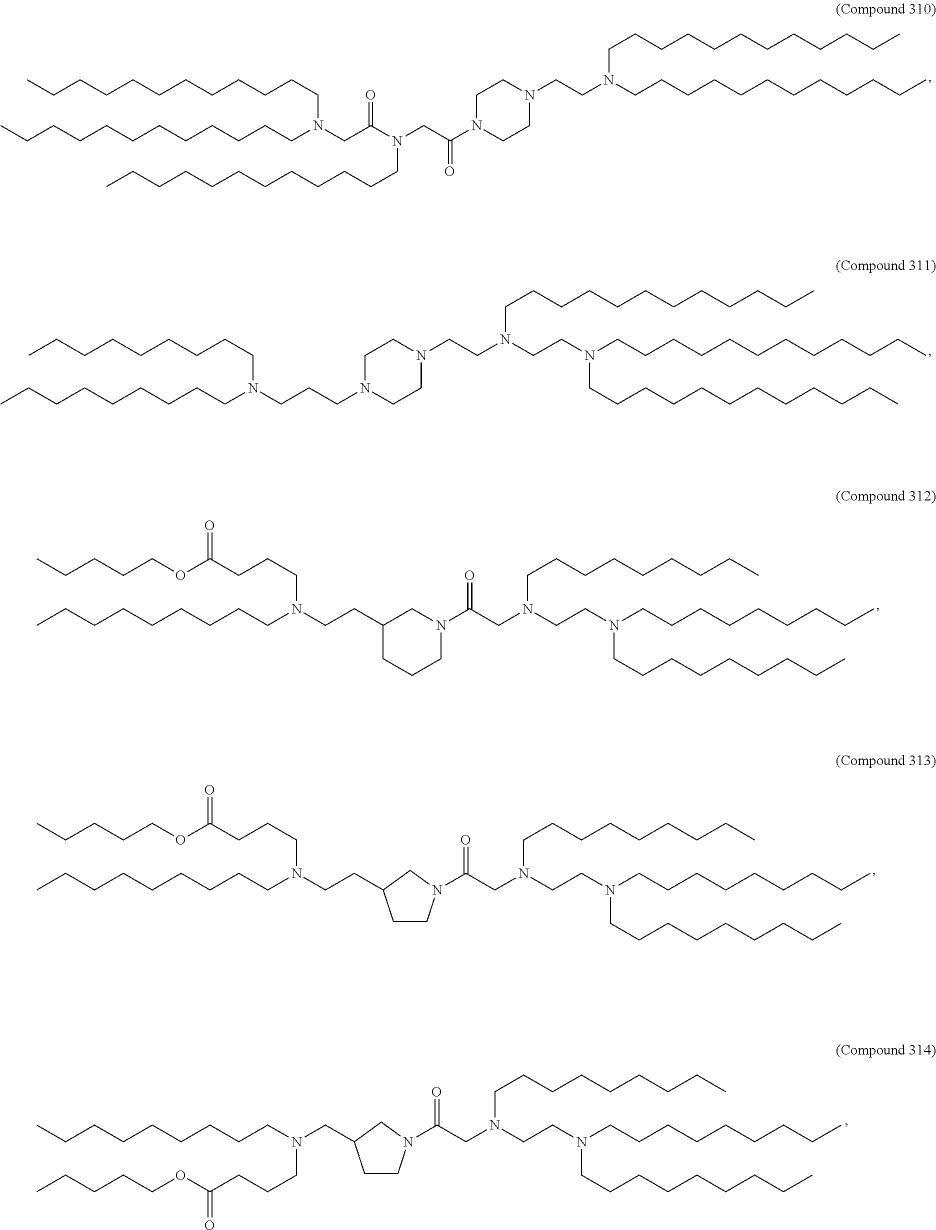

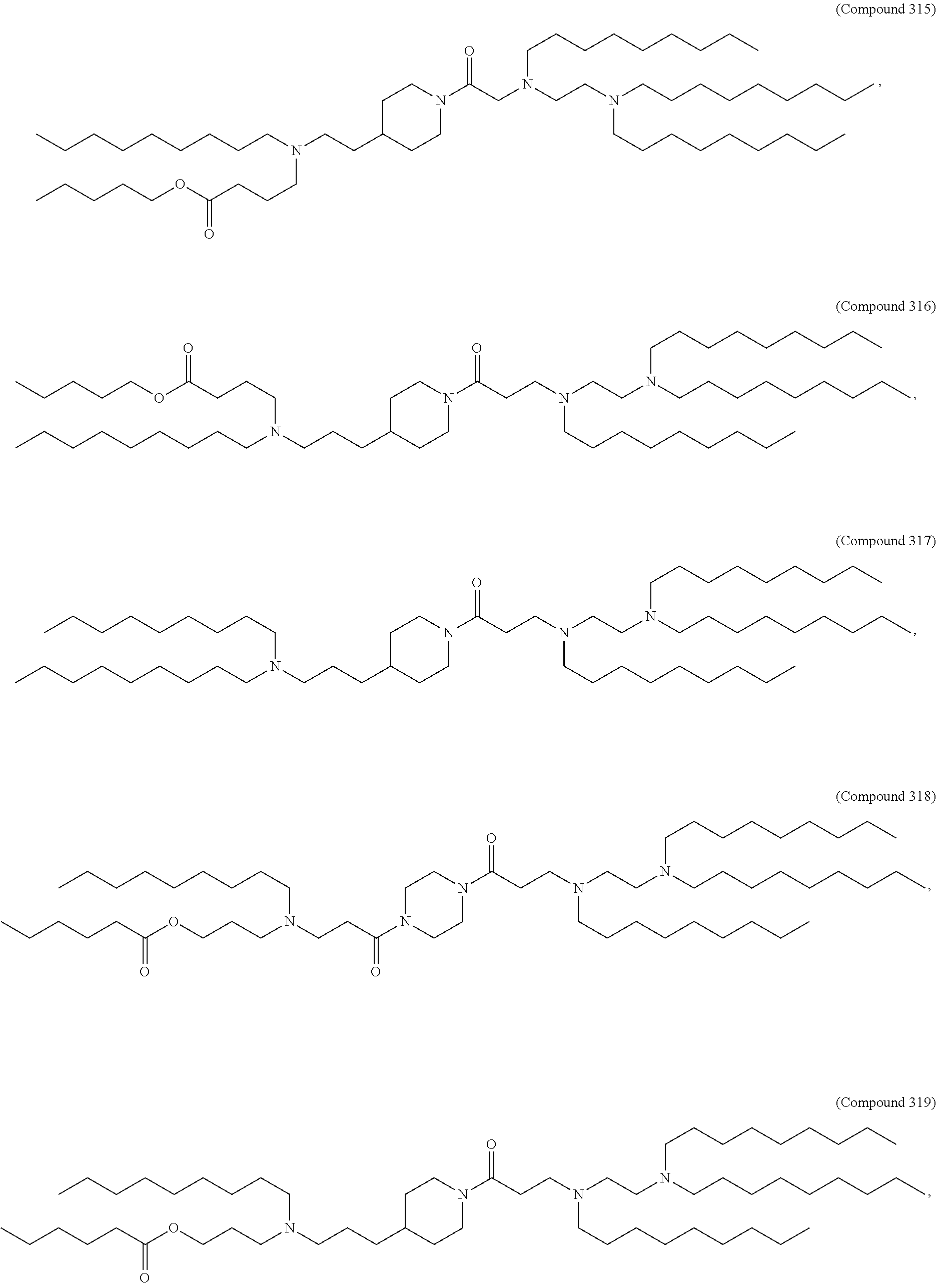

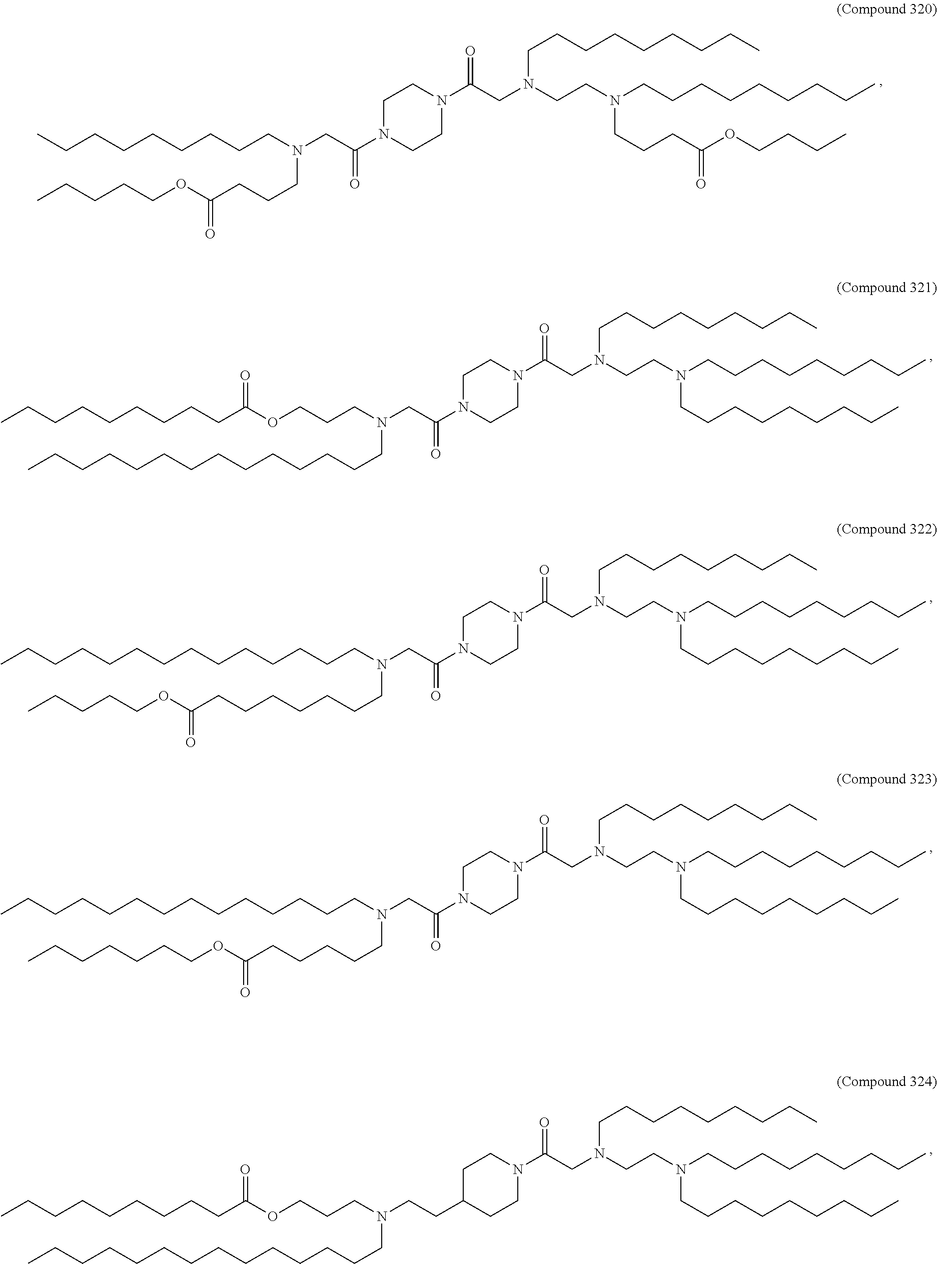

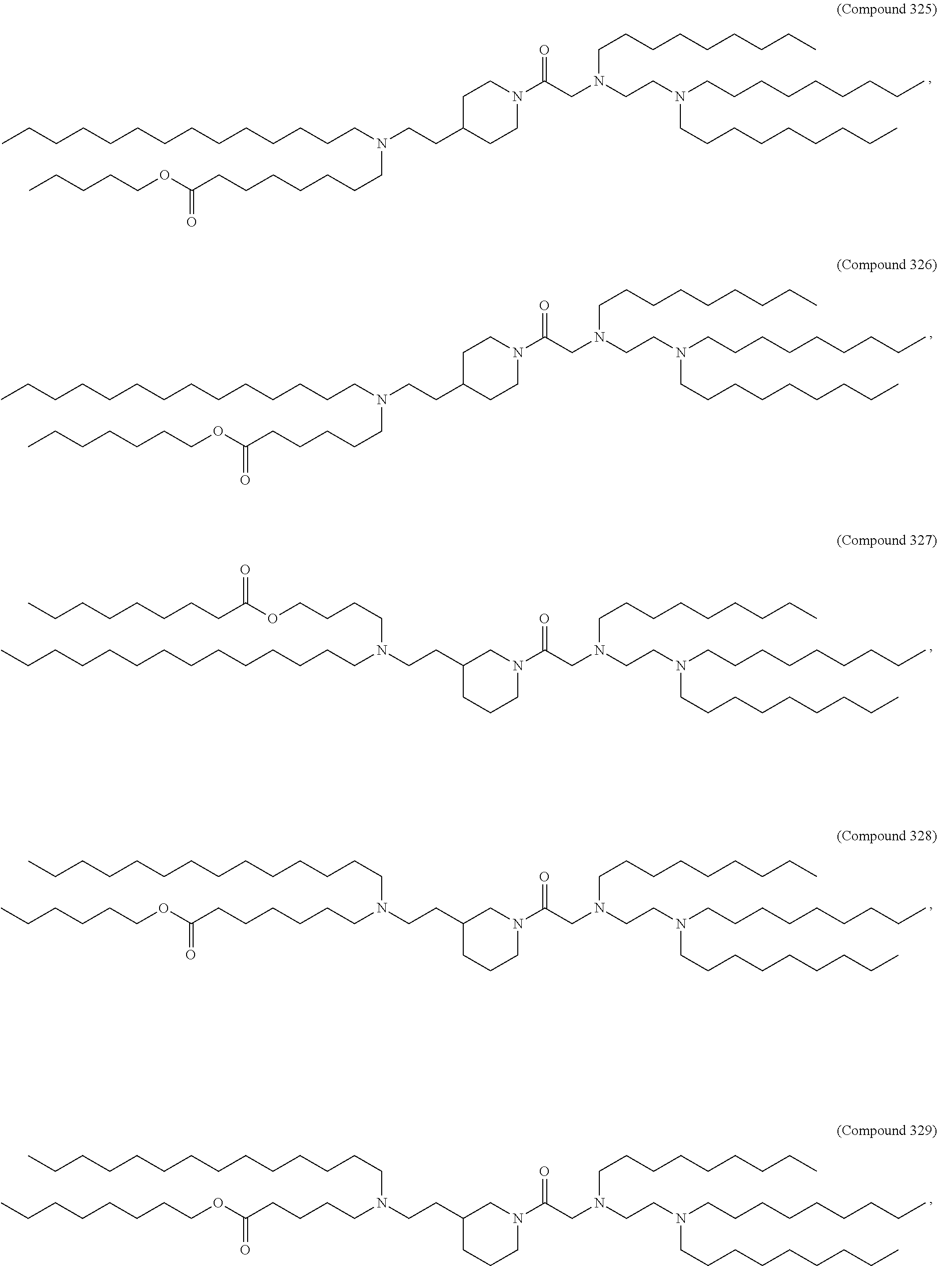

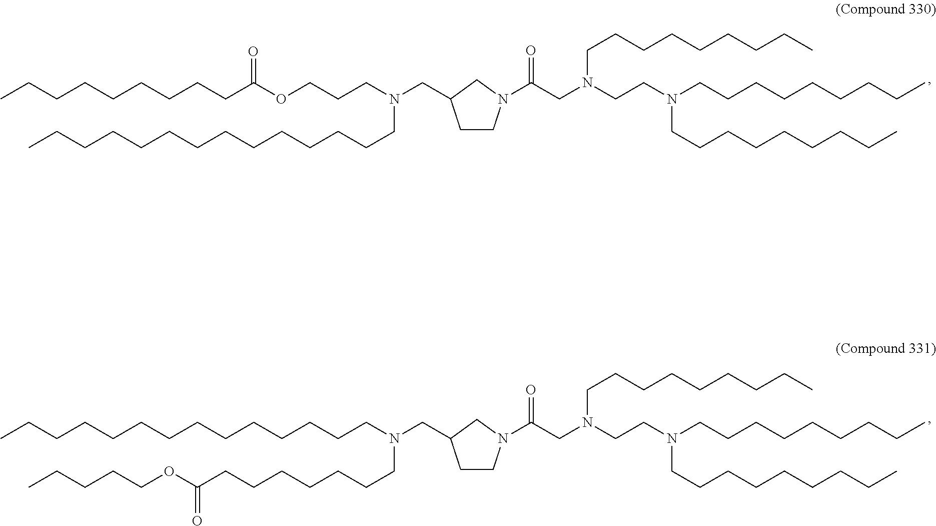

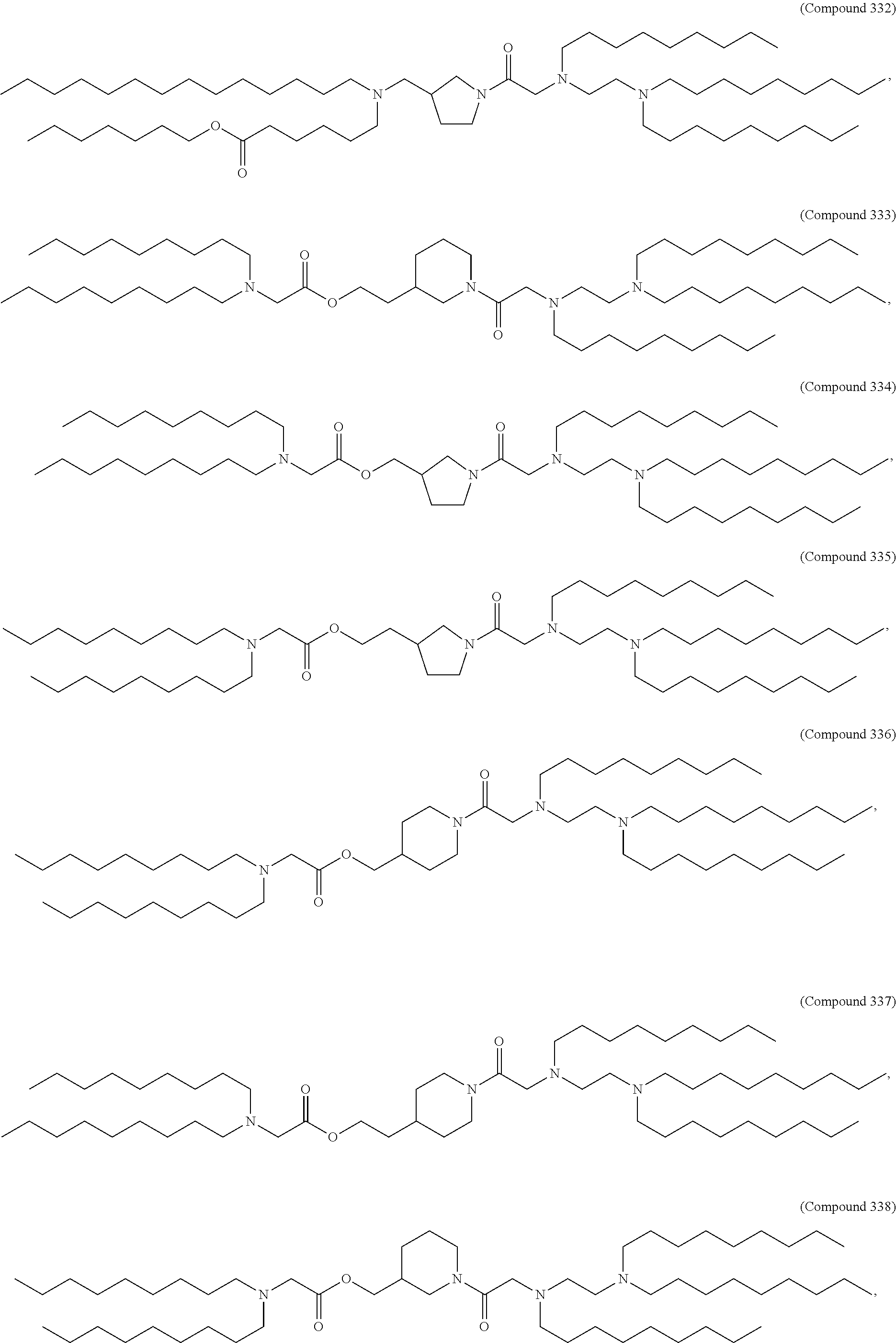

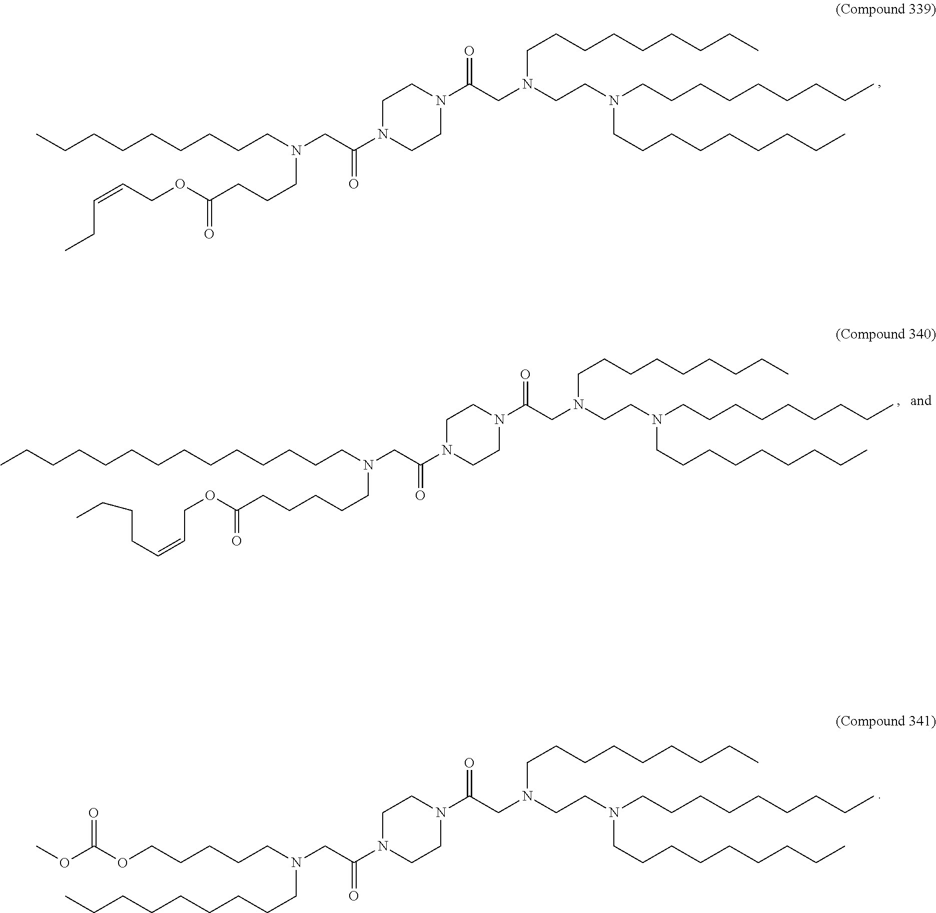

In other embodiments, a lipid has the Formula (IV)

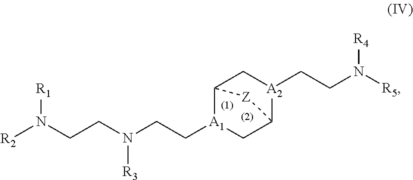

or a salt or isomer thereof, wherein
A₁ and A₂ are each independently selected from CH or N and at least one of A₁ and A₂ is N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₆₋₂₀ alkyl and C₆₋₂₀ alkenyl;
wherein when ring A is

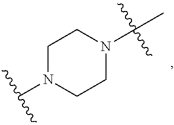 [SMILES: *N1CCN(*)CC1]

then
i) R₁, R₂, R₃, R₄, and R₅ are the same, wherein R₁ is not C₁₂ alkyl, C₁₈ alkyl, or C₁₈ alkenyl;
ii) only one of R₁, R₂, R₃, R₄, and R₅ is selected from C₆₋₂₀ alkenyl;
iii) at least one of R₁, R₂, R₃, R₄, and R₅ have a different number of carbon atoms than at least one other of R₁, R₂, R₃, R₄, and R₅;
iv) R₁, R₂, and R₃ are selected from C₆₋₂₀ alkenyl, and R₄ and R₅ are selected from C₆₋₂₀ alkyl; or
v) R₁, R₂, and R₃ are selected from C₆₋₂₀ alkyl, and R₄ and R₅ are selected from C₆₋₂₀ alkenyl.

In some embodiments, the compound is of Formula (IVa):

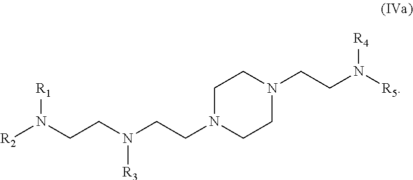

The compounds of Formula (IV) or (IVa) include one or more of the following features when applicable.

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, R₁, R₂, R₃, R₄, and R₅ are the same, and are not C₁₂ alkyl, C₁₈ alkyl, or C₁₈ alkenyl. In some embodiments, R₁, R₂, R₃, R₄, and R₅ are the same and are C₉ alkyl or C₁₄ alkyl.

In some embodiments, only one of R₁, R₂, R₃, R₄, and R₅ is selected from C₆₋₂₀ alkenyl. In certain such embodiments, R₁, R₂, R₃, R₄, and R₅ have the same number of carbon atoms. In some embodiments, R₄ is selected from C₅₋₂₀ alkenyl. For example, R₄ may be C₁₂ alkenyl or C₁₈ alkenyl.

In some embodiments, at least one of R₁, R₂, R₃, R₄, and R₅ have a different number of carbon atoms than at least one other of R₁, R₂, R₃, R₄, and R₅.

In certain embodiments, R₁, R₂, and R₃ are selected from C₆₋₂₀ alkenyl, and R₄ and R₅ are selected from C₆₋₂₀ alkyl. In other embodiments, R₁, R₂, and R₃ are selected from C₆₋₂₀ alkyl, and R₄ and R₅ are selected from C₆₋₂₀ alkenyl. In some embodiments, R₁, R₂, and R₃ have the same number of carbon atoms, and/or R₄ and R₅ have the same number of carbon atoms. For example, R₁, R₂, and R₃, or R₄ and R₅, may have 6, 8, 9, 12, 14, or 18 carbon atoms. In some embodiments, R₁, R₂, and R₃, or R₄ and R₅, are C₁₈ alkenyl (e.g., linoleyl). In some embodiments, R₁, R₂, and R₃, or R₄ and R₅, are alkyl groups including 6, 8, 9, 12, or 14 carbon atoms.

In some embodiments, R₁ has a different number of carbon atoms than R₂, R₃, R₄, and R₅. In other embodiments, R₃ has a different number of carbon atoms than R₁, R₂, R₄, and R₅. In further embodiments, R₄ has a different number of carbon atoms than R₁, R₂, R₃, and R₅.

In some embodiments, the compound is selected from the group consisting of:

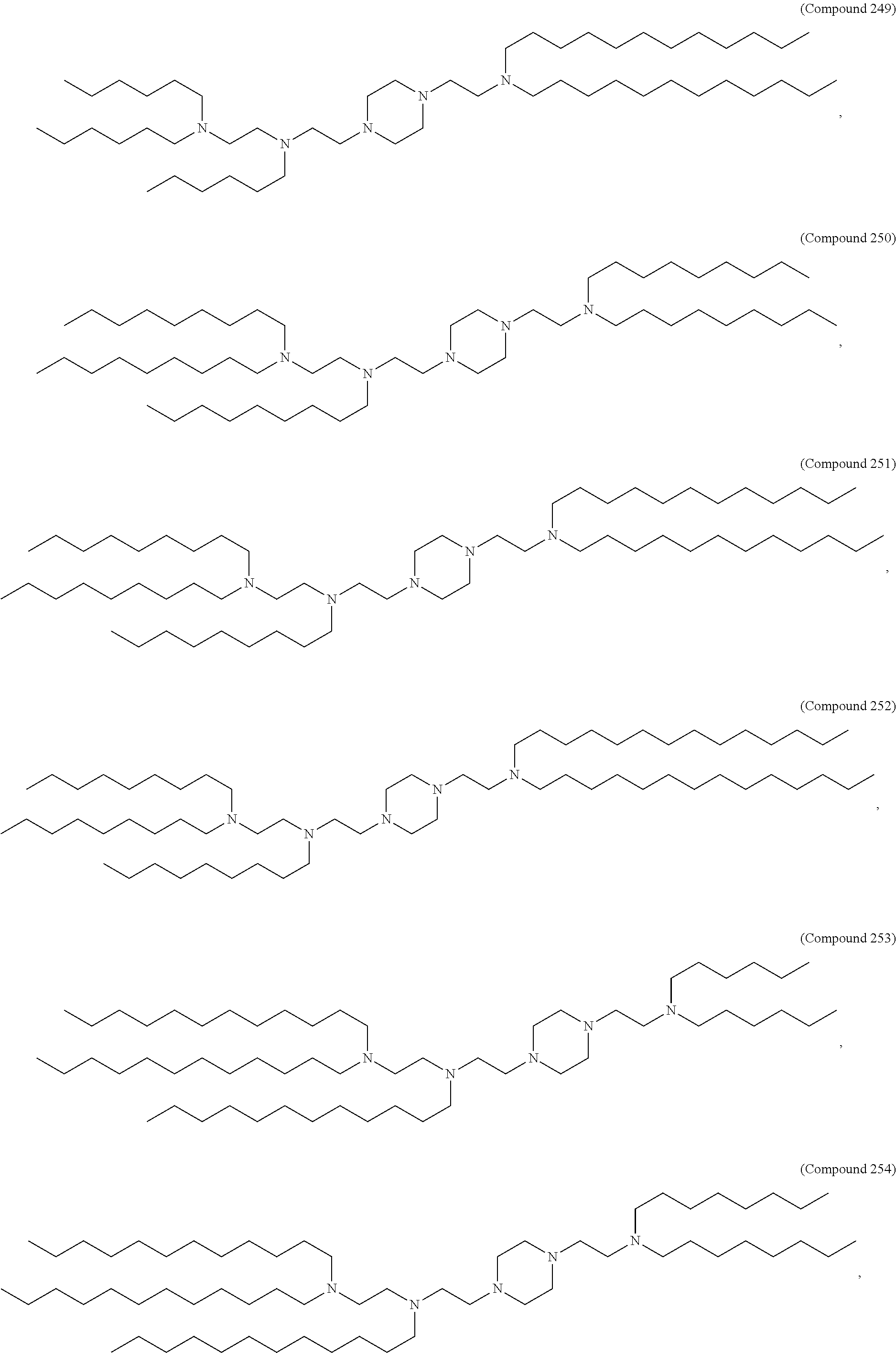

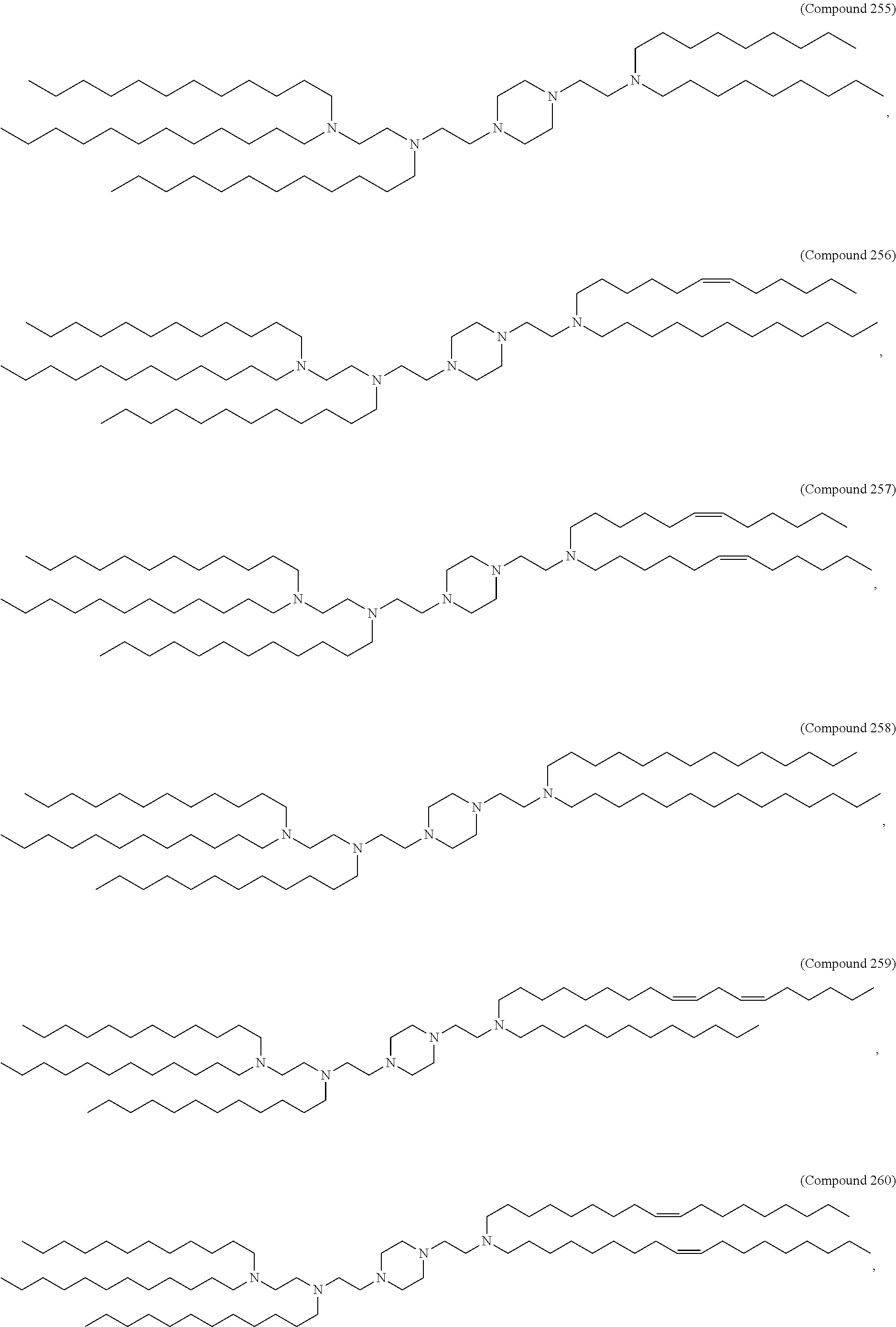

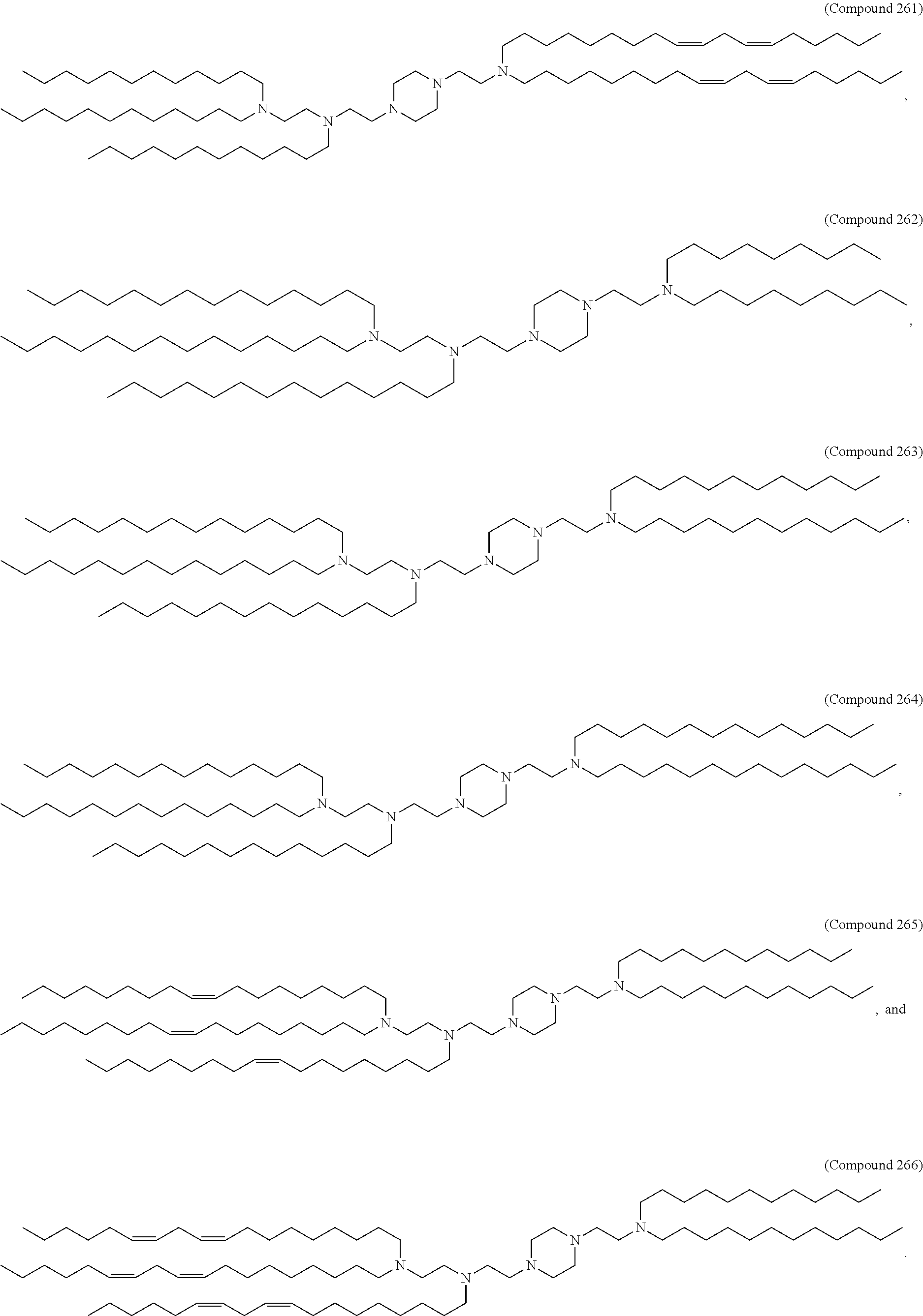

In other embodiments, the compound has the Formula (V)

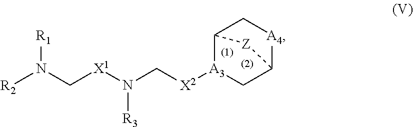

or a salt or isomer thereof, in which
A₃ is CH or N;
A₄ is CH₂ or NH; and at least one of A₃ and A₄ is N or NH;
Z is CH₂ or absent wherein when Z is CH₂, the dashed lines (1) and (2) each represent a single bond; and when Z is absent, the dashed lines (1) and (2) are both absent;
R₁, R₂, and R₃ are independently selected from the group consisting of C₅₋₂₀ alkyl, C₅₋₂₀ alkenyl, —R″MR′, —R*YR″, —YR″, and —R*OR″;
each M is independently selected
   from —C(O)O—, —OC(O)—, —C(O)N(R′)—, —N(R′)C(O)—, —C(O)—, —C(S)—, —C(S)S—, —SC(S)—, —C H(OH)—, —P(O)(OR′)O—, —S(O)₂—, an aryl group, and a heteroaryl group; X¹ and X² are independently selected from the group consisting of —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl.

In some embodiments, the compound is of Formula (Va):

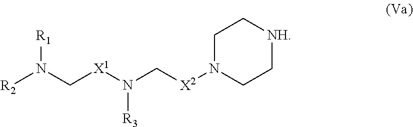

The compounds of Formula (V) or (Va) include one or more of the following features when applicable.

In some embodiments, Z is CH₂.

In some embodiments, Z is absent.

In some embodiments, at least one of A₃ and A₄ is N or NH.

In some embodiments, A₃ is N and A₄ is NH.

In some embodiments, A₃ is N and A₄ is CH₂.

In some embodiments, A₃ is CH and A₄ is NH.

In some embodiments, at least one of X¹ and X² is not —CH₂—. For example, in certain embodiments, X¹ is not —CH₂—. In some embodiments, at least one of X¹ and X² is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, R₁, R₂, and R₃ are independently selected from the group consisting of C₅₋₂₀ alkyl and C₅₋₂₀ alkenyl. In some embodiments, R₁, R₂, and R₃ are the same. In certain embodiments, R₁, R₂, and R₃ are C₆, C₉, C₁₂, or C₁₄ alkyl. In other embodiments, R₁, R₂, and R₃ are C₁₈ alkenyl. For example, R₁, R₂, and R₃ may be linoleyl.

In some embodiments, the compound is selected from the group consisting of:

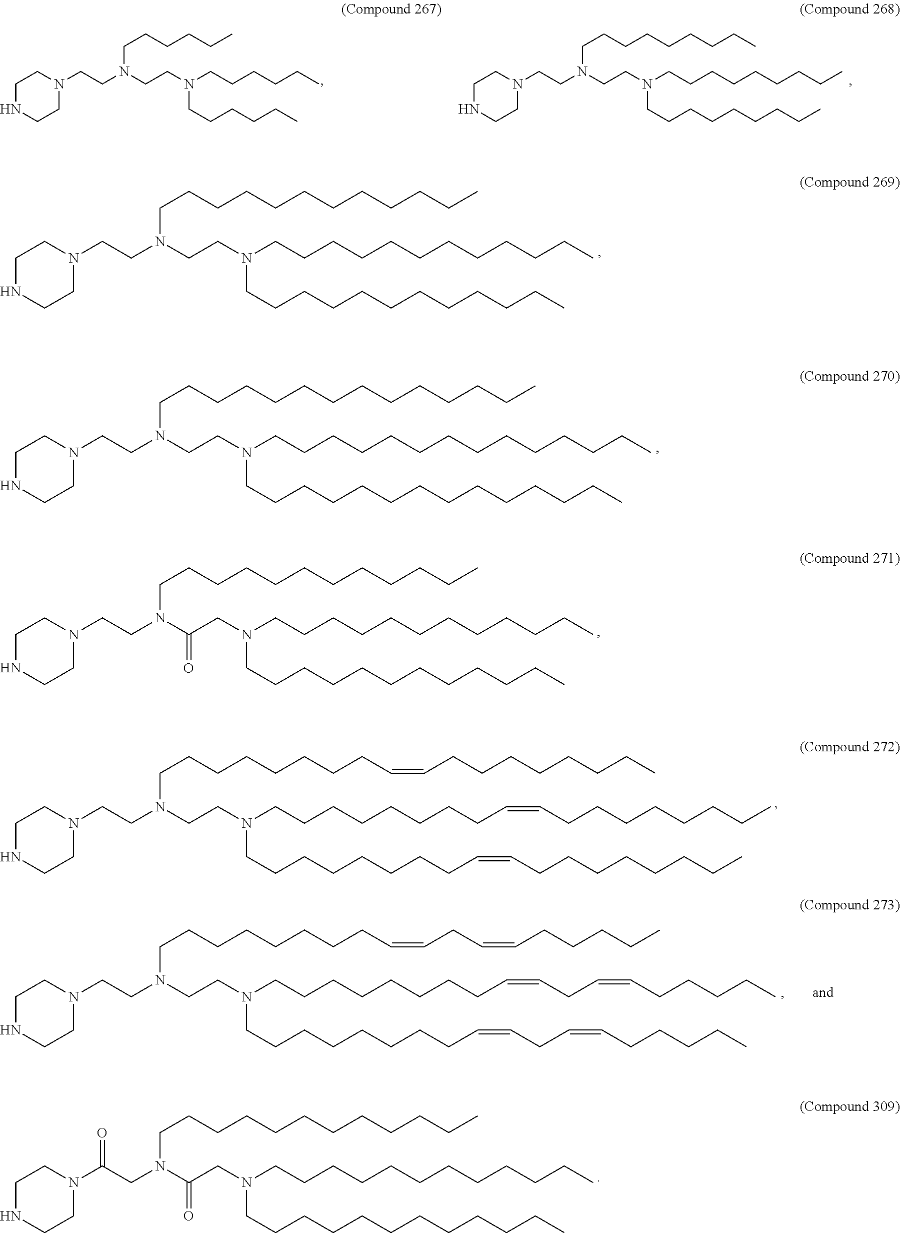

In another aspect, the disclosure provides a compound according to Formula (VI):

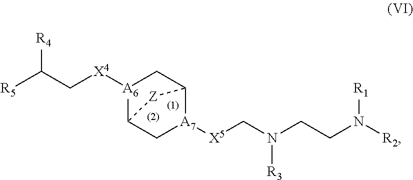

or a salt or isomer thereof, in which
A₆ and A₇ are each independently selected from CH or N, wherein at least one of A₆ and A₇ is N;
Z is CH₂ or absent wherein when Z is CH₂, the dashed lines (1) and (2) each represent a single bond; and when Z is absent, the dashed lines (1) and (2) are both absent;
X⁴ and X⁵ are independently selected from the group consisting of —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
R₁, R₂, R₃, R₄, and R₅ each are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and each R″ is independently selected from the group consisting of C₃₋₁₂ alkyl and C₃₋₁₂ alkenyl.

In some embodiments, R₁, R₂, R₃, R₄, and R₅ each are independently selected from the group consisting of C₆₋₂₀ alkyl and C₆₋₂₀ alkenyl.

In some embodiments, R₁ and R₂ are the same. In certain embodiments, R₁, R₂, and R₃ are the same. In some embodiments, R₄ and R₅ are the same. In certain embodiments, R₁, R₂, R₃, R₄, and R₅ are the same.

In some embodiments, at least one of R₁, R₂, R₃, R₄, and R₅ is C₉₋₁₂ alkyl. In certain embodiments, each of R₁, R₂, R₃, R₄, and R₅ independently is C₉, C₁₂ or C₁₄ alkyl. In certain embodiments, each of R₁, R₂, R₃, R₄, and R₅ is C₉ alkyl.

In some embodiments, A₆ is N and A₇ is N. In some embodiments, A₆ is CH and A₇ is N.

In some embodiments, X4 is —CH₂— and X₅ is —C(O)—. In some embodiments, X₄ and X₅ are —C(O)—.

In some embodiments, when A₆ is N and A₇ is N, at least one of X₄ and X₅ is not —CH₂—, e.g., at least one of X₄ and X₅ is —C(O)—. In some embodiments, when A₆ is N and A₇ is N, at least one of R₁, R₂, R₃, R₄, and R₅ is —R″MR′.

In some embodiments, at least one of R₁, R₂, R₃, R₄, and R₅ is not —R″MR′.

In some embodiments, the compound is

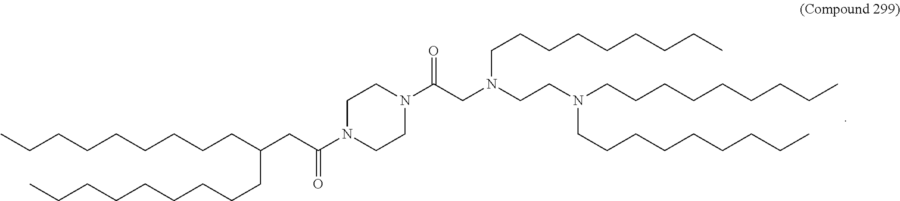 [SMILES: CCCCCCCCCC(CCCCCCCCC)CC(=O)N1CCN(C(=O)CN(CCCCCCCCC)CCN(CCCCCCCCC)CCCCCCCCC)CC1]

In an embodiment, the compound has the following formula:

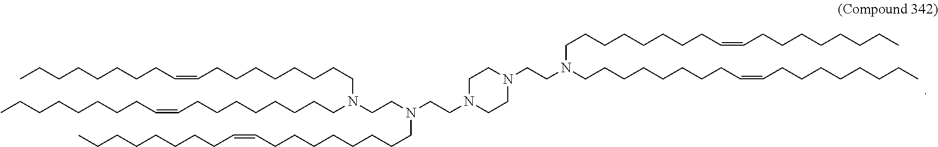 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCC/C=C\CCCCCCCC)CCCCCCCC/C=C\CCCCCCCC)CC1]

### PEG and PEG-Modified Lipids

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid. In some embodiments, a PEG lipid is DMG-PEG 2k or Compound 428.

In some embodiments, the PEG-modified lipids are a modified form of PEG DMG. PEG-DMG has the following structure:

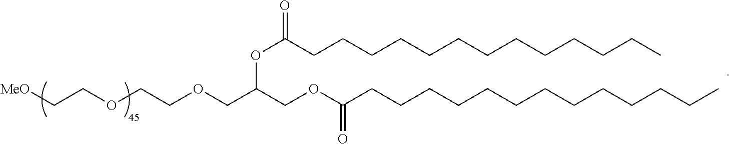

In some embodiments, the nanoparticle described herein comprises about 1 mol % to about 5 mol % of PEG-lipid. In some embodiments, the nanoparticle comprises about 1 mol % to about 2.5 mol % of PEG-lipid.

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (VII). Provided herein are compounds of Formula (VII):

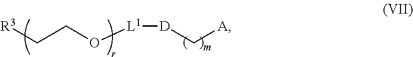

or salts thereof, wherein:
R₃ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
D is a moiety obtained by click chemistry or a moiety cleavable under physiological conditions;
m is 0, 1, 2, 3, 4, 5, 6, 7, 8, 9, or 10;
A is of the formula:

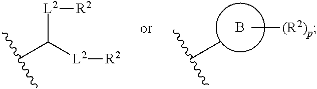

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O—, —C(O)O—, —OC(O)—, —OC(O)O—, —OC(O)N(R^{N})—, —NR^{N}C(O)O—, or —NR^{N}C(O)N(R^{N})—;
each instance of R₂ is independently optionally substituted C₁₋₃₀ alkyl, optionally substituted C₁₋₃₀ alkenyl, or optionally substituted C₁₋₃₀ alkynyl; optionally wherein one or more methylene units of R₂ are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (VII) is a PEG-OH lipid (i.e., R³ is —OR^{O}, and R^{O} is hydrogen). In certain embodiments, the compound of Formula (VII) is of Formula (VII-OH):

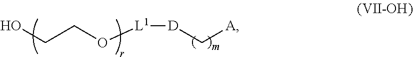

or a salt thereof.

In certain embodiments, D is a moiety obtained by click chemistry (e.g., triazole). In certain embodiments, the compound of Formula (VII) is of Formula (VII-a-1) or (VII-a-2):

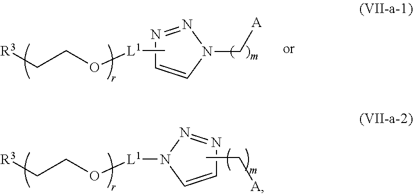

or a salt thereof.

In certain embodiments, the compound of Formula (VII) is of one of the following formulae:

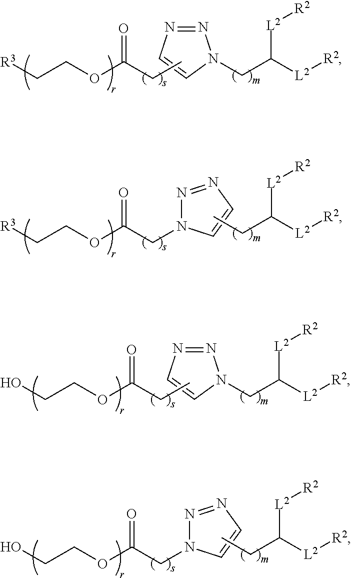

or a salt thereof, wherein

s is 0, 1, 2, 3, 4, 5, 6, 7, 8, 9, or 10.

In certain embodiments, the compound of Formula (VII) is of one of the following formulae:

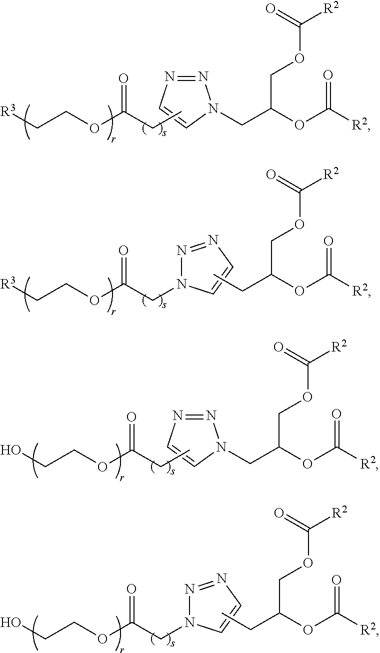

or a salt thereof.

In certain embodiments, a compound of Formula (VII) is of one of the following formulae:

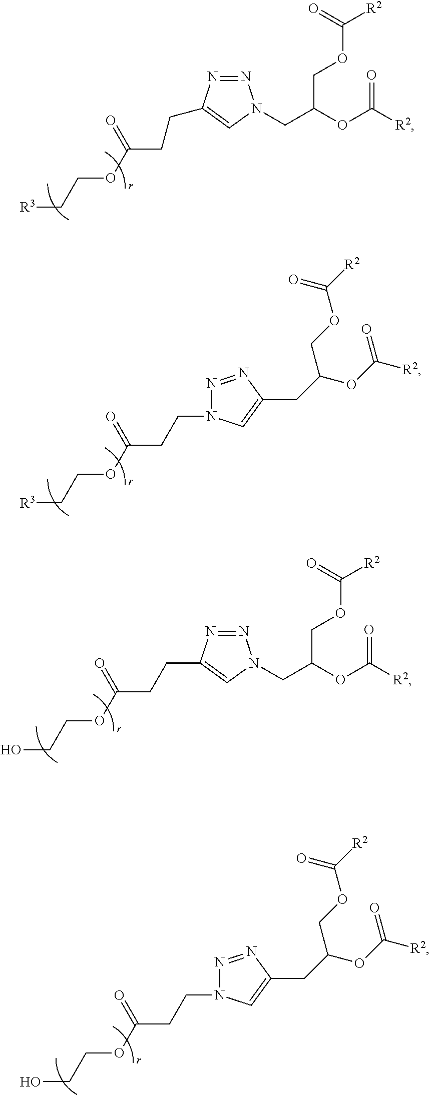

or a salt thereof.

In certain embodiments, a compound of Formula (VII) is of one of the following formulae, wherein r is 1-100:

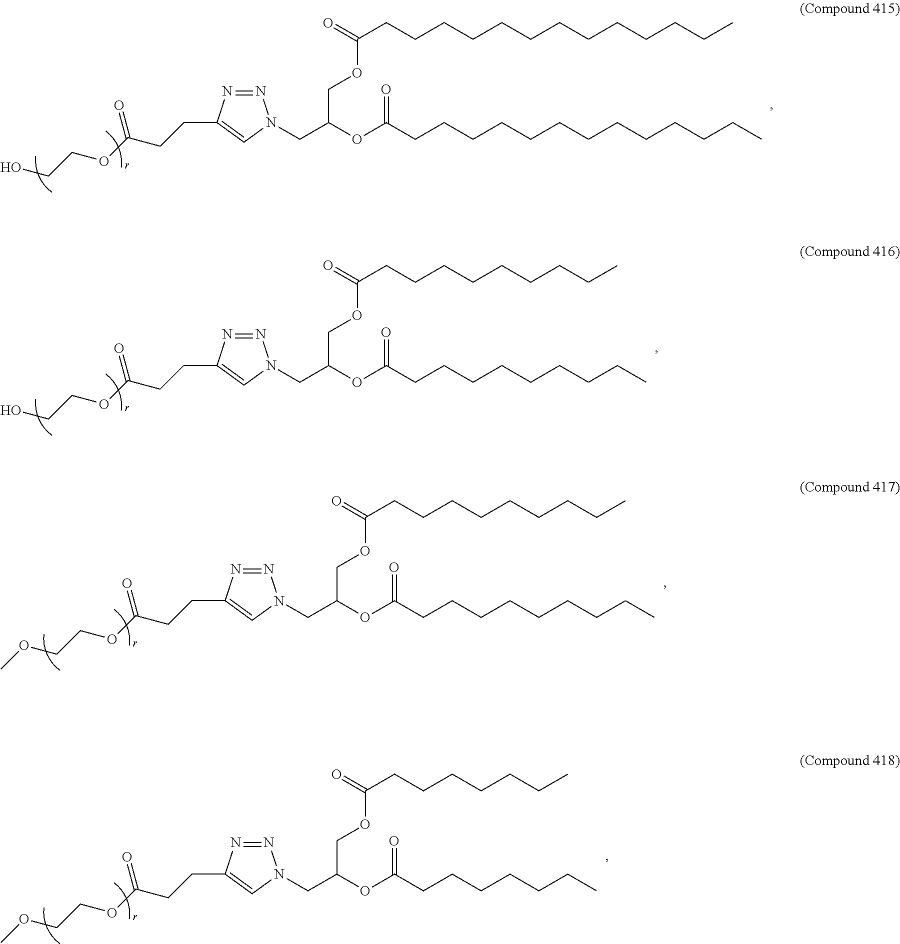

or a salt thereof.

In certain embodiments, D is a moiety cleavable under physiological conditions (e.g., ester, amide, carbonate, carbamate, urea). In certain embodiments, a compound of Formula (VII) is of Formula (VII-b-1) or (VII-b-2):

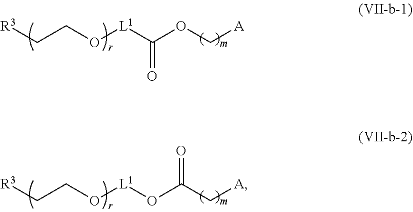

or a salt thereof.

In certain embodiments, a compound of Formula (VII) is of Formula (VII-b-1-OH) or (VII-b-2-OH):

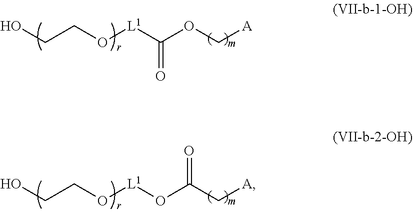

or a salt thereof.

In certain embodiments, the compound of Formula (VII) is of one of the following formulae:

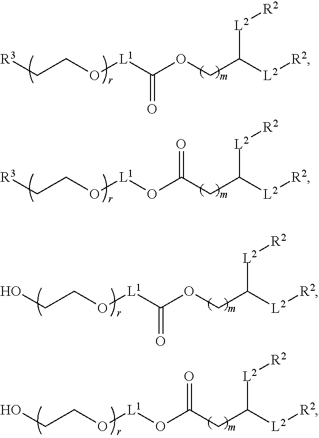

or a salt thereof.

In certain embodiments, a compound of Formula (VII) is of one of the following formulae:

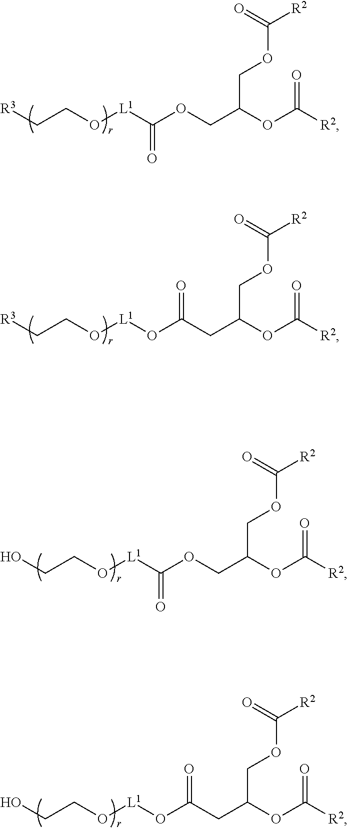

or a salt thereof.

In certain embodiments, a compound of Formula (VII) is of one of the following formulae:

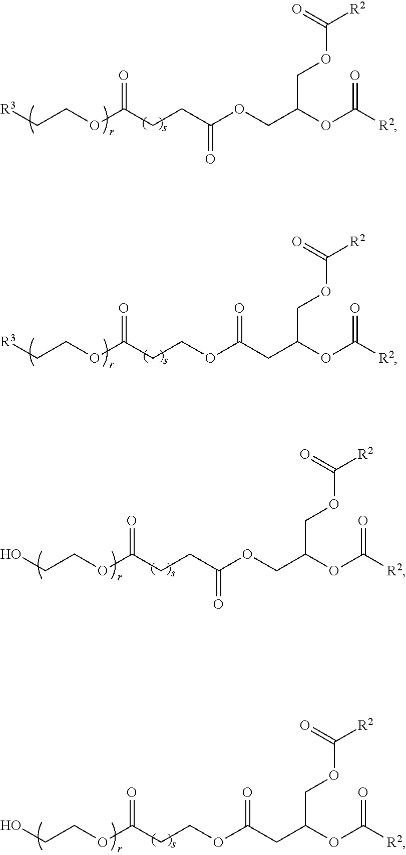

or a salt thereof.

In certain embodiments, a compound of Formula (VII) is of one of the following formulae:

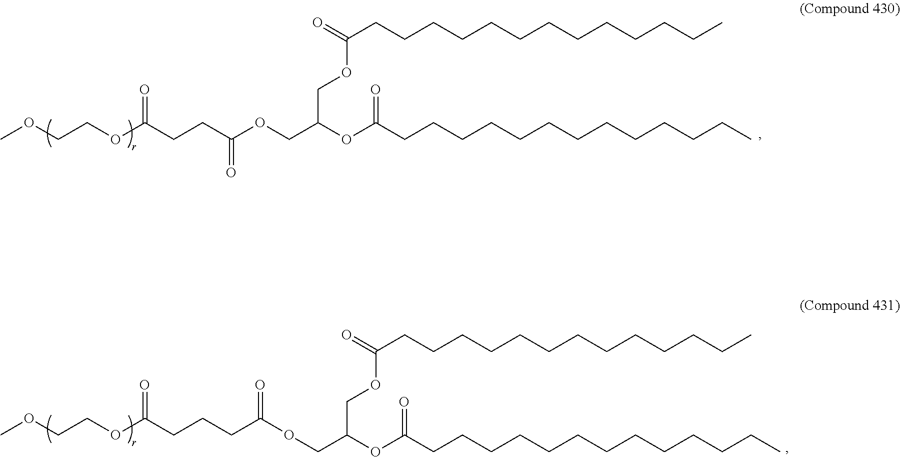

or salts thereof.

In certain embodiments, a PEG lipid useful in the present invention is a PEGylated fatty acid. In certain embodiments, a PEG lipid useful in the present invention is a compound of Formula (VIII). Provided herein are compounds of Formula (VIII):

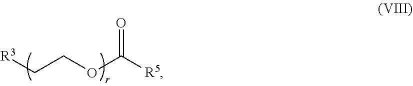

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and
each instance of R^{N} is independently hydrogen, optionally substituted alkyl, or a nitrogen protecting group.

In certain embodiments, the compound of Formula (VIII) is of Formula

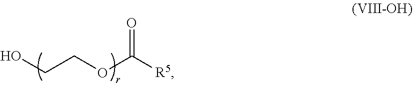

or a salt thereof.

In certain embodiments, a compound of Formula (VIII) is of one of the following formulae:

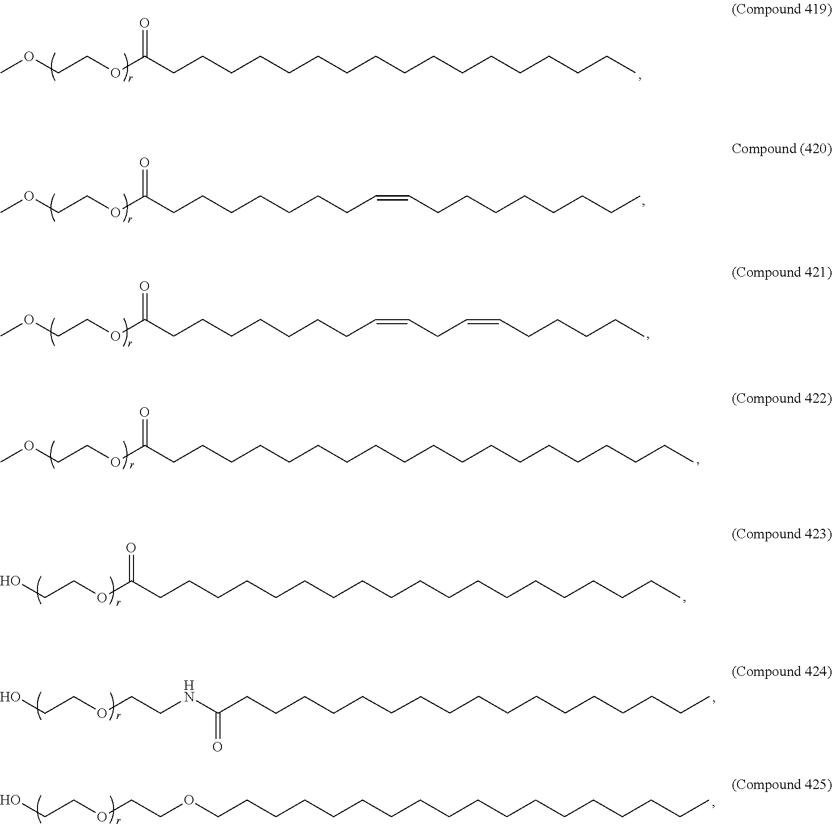

or a salt thereof. In some embodiments, r is 45.

In certain embodiments, a compound of Formula (VIII) is of one of the following formulae:

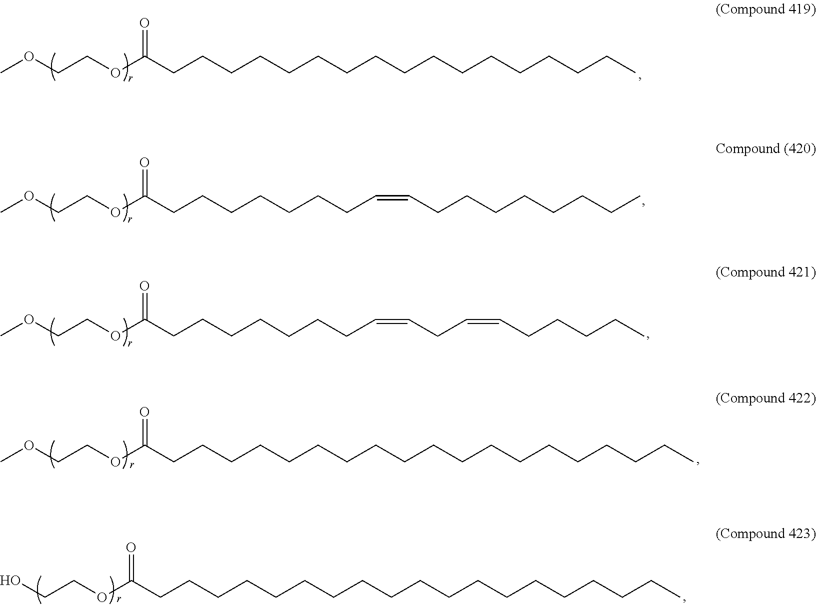

or a salt thereof. In some embodiments, r is 45.

In yet other embodiments the compound of Formula (VIII) is:

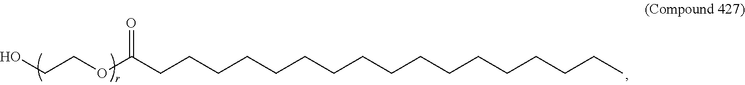

or a salt thereof.

In some embodiments, the compound of Formula (VIII) is

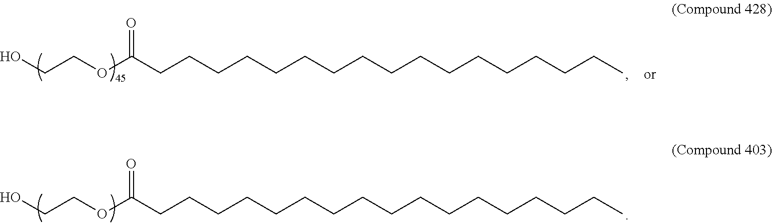

In certain embodiments, the PEG lipid is one of the following formula:

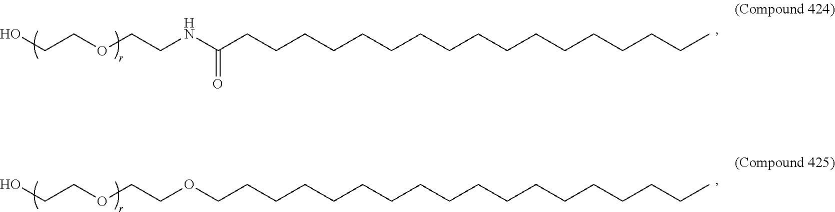

or a salt thereof. In some embodiments, r is 45.

### Phospholipids

Phospholipids, as defined herein, are any lipids that comprise a phosphate group. Phospholipids are a subset of non-cationic lipids. The lipid component of a lipid nanoparticle composition may include one or more phospholipids, such as one or more (poly)unsaturated lipids. Phospholipids may assemble into one or more lipid bilayers. In general, phospholipids may include a phospholipid moiety and one or more fatty acid moieties. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

In some embodiments, the nanoparticle described herein comprises about 5 mol % to about 15 mol % of phospholipid. In some embodiments, the nanoparticle comprises about 8 mol % to about 13 mol % of phospholipid. In some embodiments, the nanoparticle comprises about 10 mol % to about 12 mol % of phospholipid.

Phospholipids useful or potentially useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-diphytanoyl-sn-glycero-3-phosphocholine (4ME 16:0 PC), 1,2-diphytanoyl-sn-glycero-3-phospho-(1′-rac-glycerol) (sodium salt) (4ME 16:0 PG), 1,2-diphytanoyl-sn-glycero-3-phospho-L-serine (sodium salt) (4ME 16:0 PS), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, and 1,2-dioleoyl-sn-glycero-3-phospho-rac-(1-glycerol) sodium salt (DOPG), and sphingomyelin. Each possibility represents a separate embodiment of the present invention.

In some embodiments, a lipid nanoparticle composition includes DSPC. In certain embodiments, a lipid nanoparticle composition includes DOPE. In some embodiments, a lipid nanoparticle composition includes both DSPC and DOPE. In some embodiments, the lipid nanoparticle includes:
1,2-diphytanoyl-sn-glycero-3-phosphoethanolamine (4ME 16:0 PE)

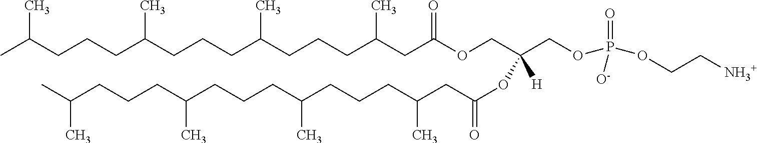 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

1,2-diphytanoyl-sn-glycero-3-phosphocholine (4ME 16:0 PC)

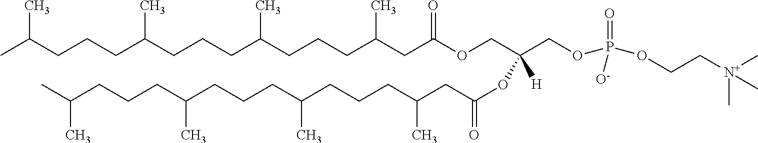 [SMILES: CC(C)CCCC(C)CCCC(C)CCCC(C)CC(=O)OC[C@H](COP(=O)([O-])OCC[N+](C)(C)C)OC(=O)CC(C)CCCC(C)CCCC(C)CCCC(C)C]

1,2-diphytanoyl-sn-glycero-3-phospho-(1′-rac-glycerol) (sodium salt) (4ME 16:0 PG), or

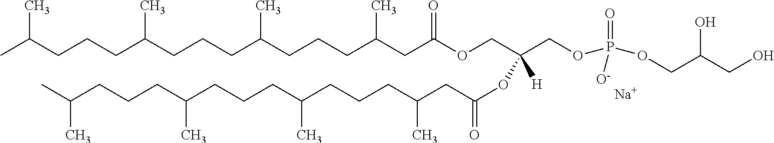 [SMILES: CC(C)CCCC(C)CCCC(C)CCCC(C)CC(=O)OC[C@H](COP(=O)([O-])OCC(O)CO)OC(=O)CC(C)CCCC(C)CCCC(C)CCCC(C)C ; [Na+]]

1,2-diphytanoyl-sn-glycero-3-phospho-L-serine (sodium salt) (4ME 16:0 PS)

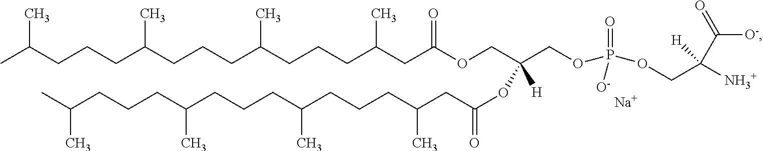

or a mixture thereof.

Examples of phospholipids include, but are not limited to, the following:

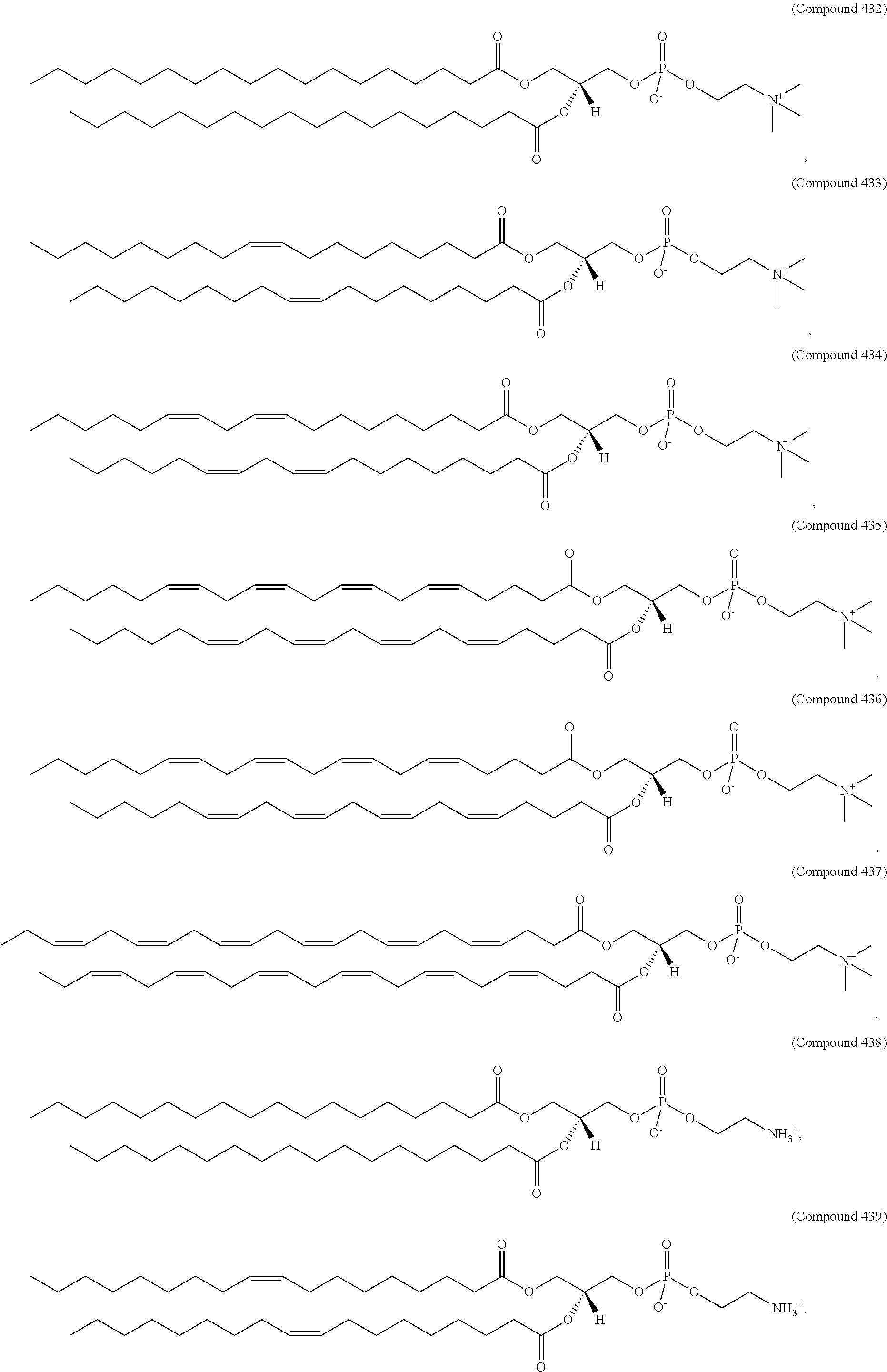

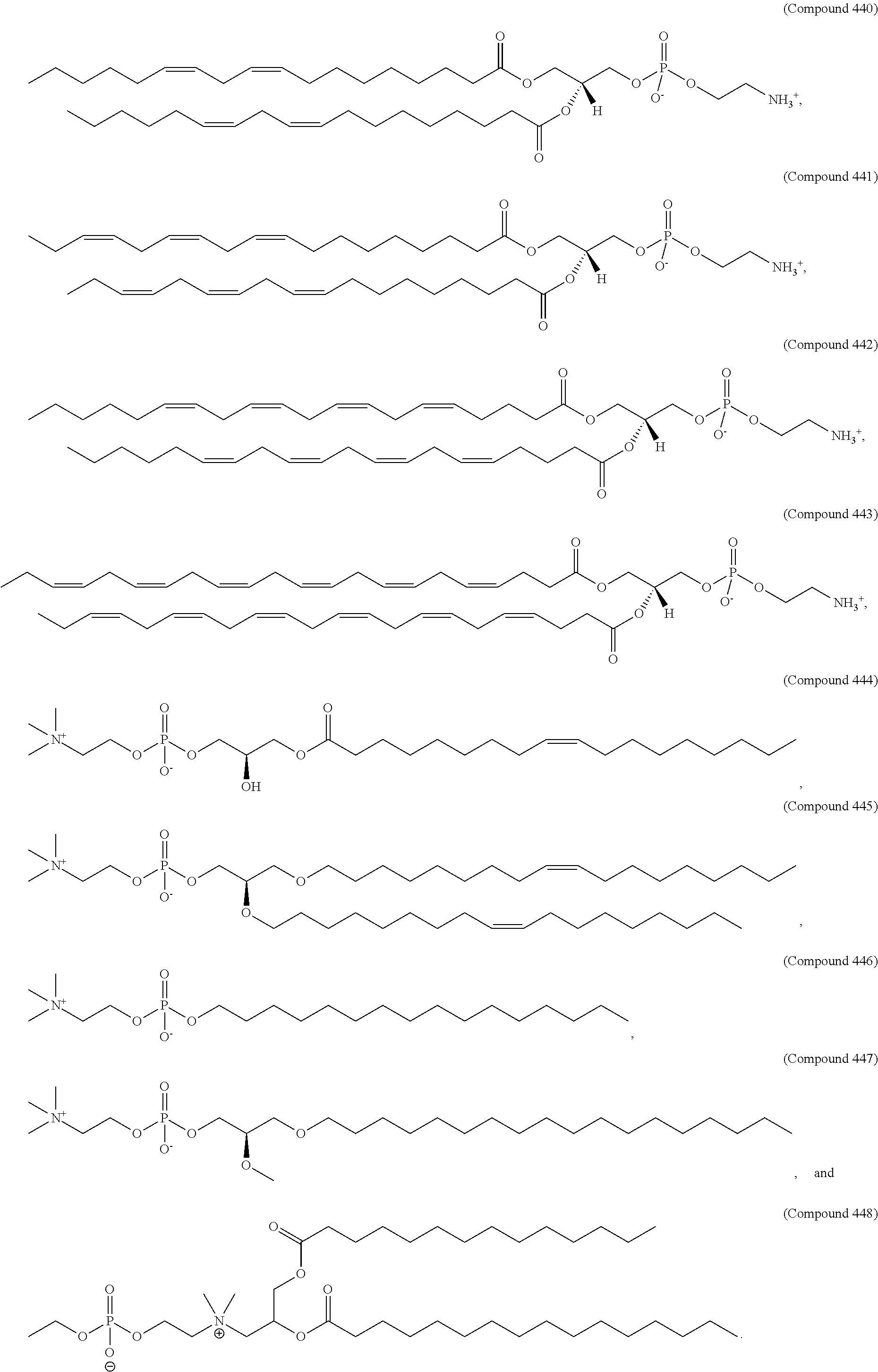

In certain embodiments, a phospholipid useful or potentially useful in the present invention is an analog or variant of DSPC.

In certain embodiments, a phospholipid useful or potentially useful in the present invention is a compound of Formula (IX):

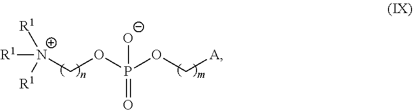

or a salt thereof, wherein:
each R¹ is independently H or optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

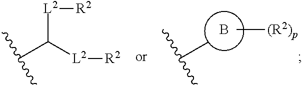

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

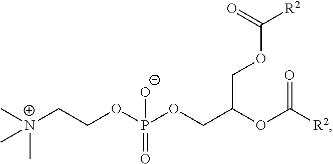

wherein each instance of R₂ is independently unsubstituted alkyl, unsubstituted alkenyl, or unsubstituted alkynyl.

In certain embodiments, a phospholipid useful or potentially useful in the present invention is a compound of Formula (IX):

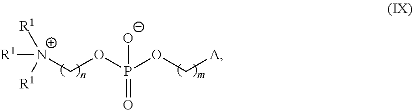

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

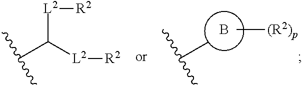

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R₂ are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

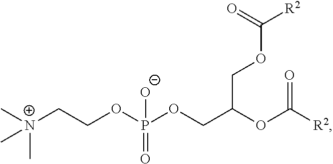

wherein each instance of R₂ is independently unsubstituted alkyl, unsubstituted alkenyl, or unsubstituted alkynyl.

### Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

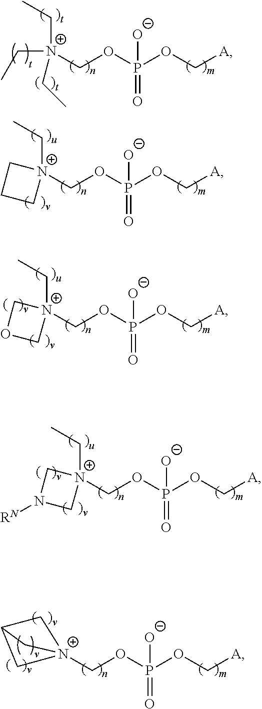

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (IX) is of one of the following formulae:

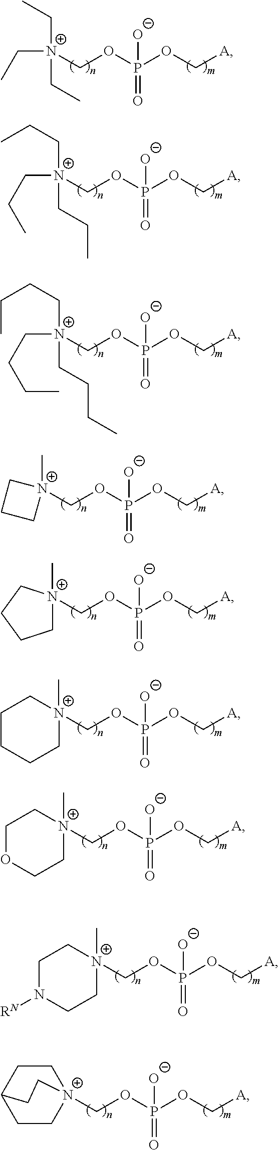

or a salt thereof.

In certain embodiments, a compound of Formula (IX) is one of the following:

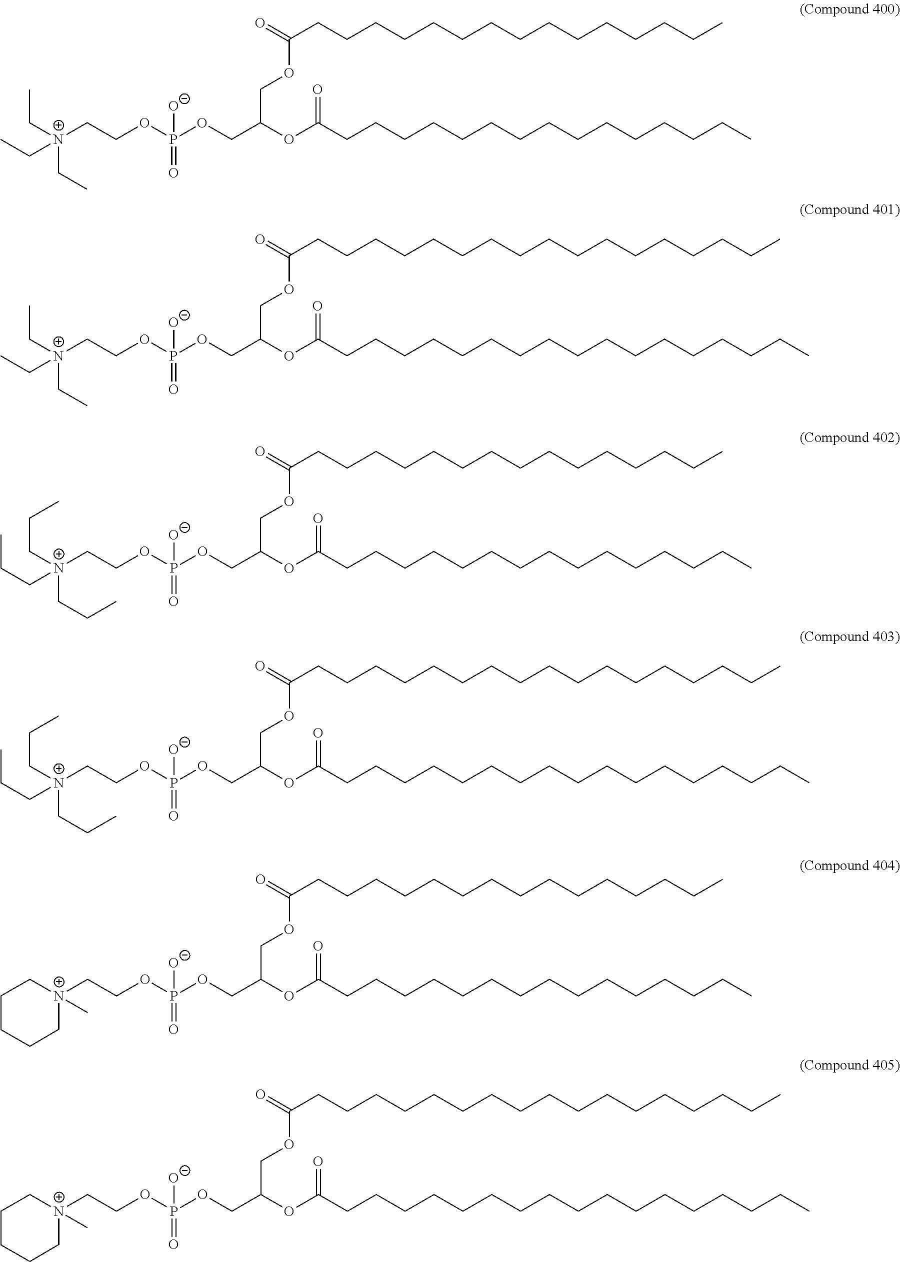

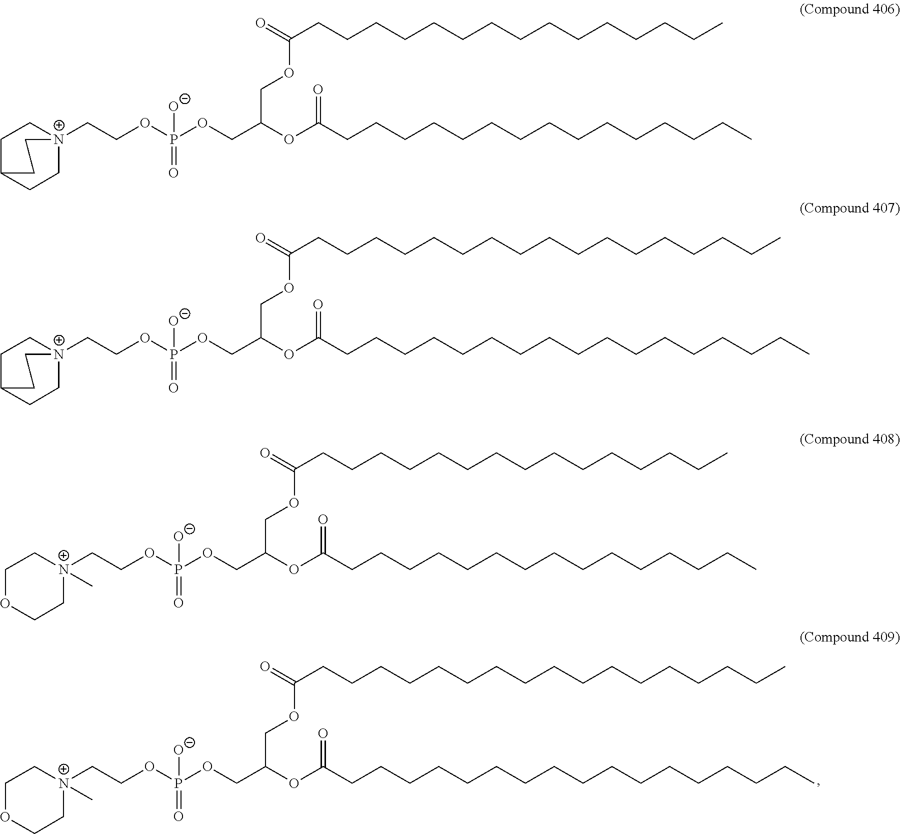 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In certain embodiments, a compound of Formula (IX) is of Formula (IX-a):

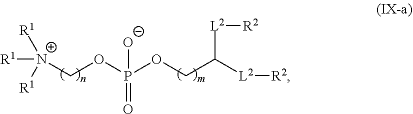

or a salt thereof.

In certain embodiments, phospholipids useful or potentially useful in the present invention comprise a modified core. In certain embodiments, a phospholipid with a modified core described herein is DSPC, or analog thereof, with a modified core structure. For example, in certain embodiments of Formula (IX-a), group A is not of the following formula:

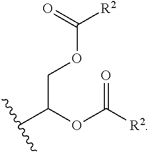

In certain embodiments, the compound of Formula (IX-a) is of one of the following formulae:

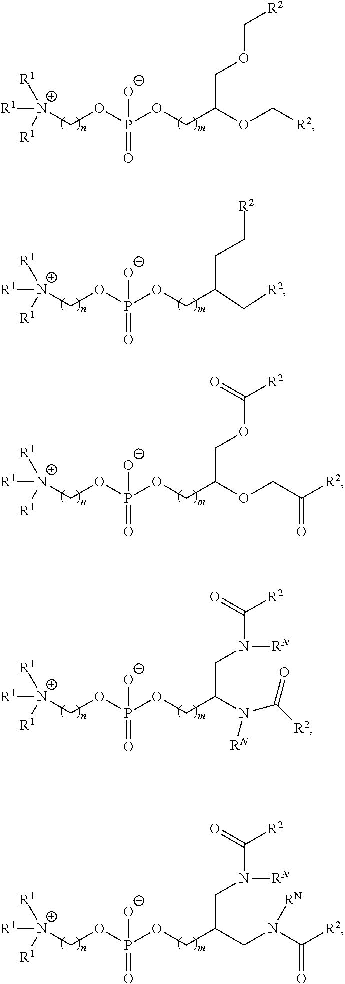

or a salt thereof.

In certain embodiments, a compound of Formula (IX) is one of the following:

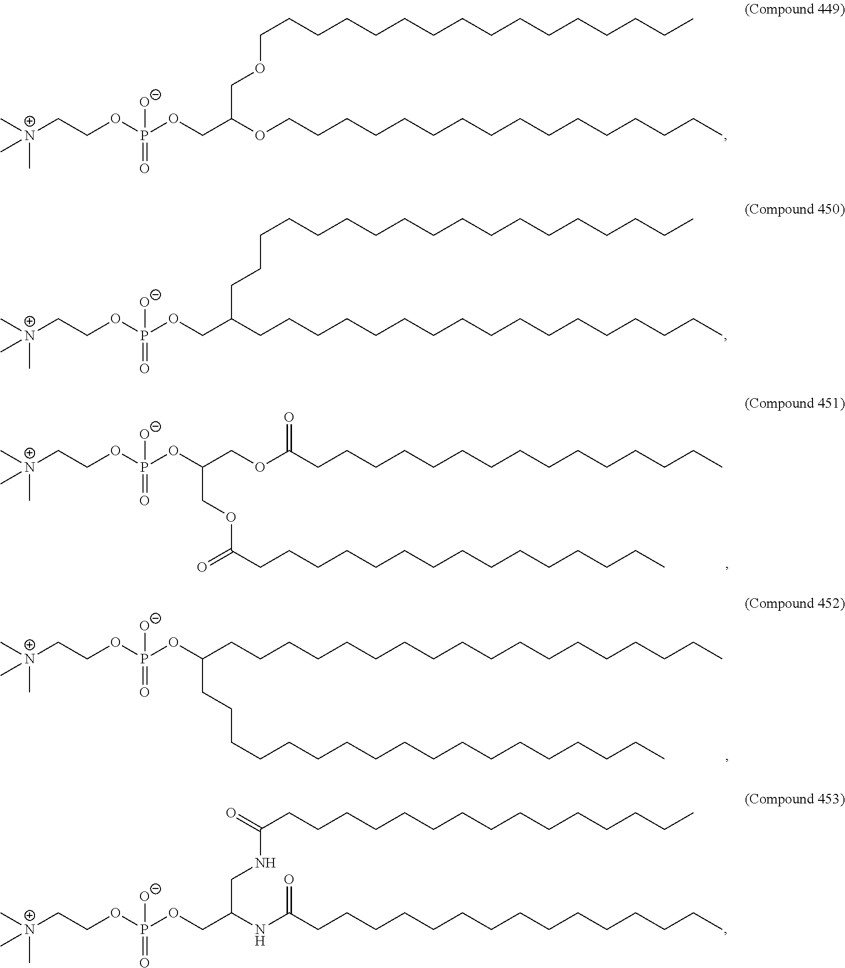

or salts thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IX) is of Formula (IX-b):

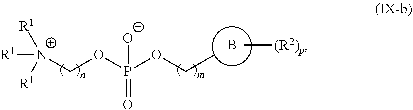

or a salt thereof.

In certain embodiments, the compound of Formula (IX-b) is of Formula (IX-b-1):

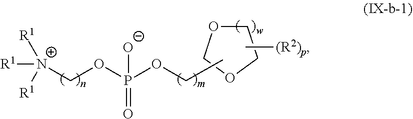

or a salt thereof, wherein:

w is 0, 1, 2, or 3.

In certain embodiments, the compound of Formula (IX-b) is of Formula (IX-b-2):

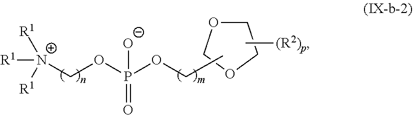

or a salt thereof.

In certain embodiments, the compound of Formula (IX-b) is of Formula (IX-b-3):

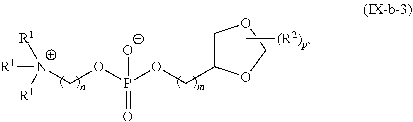

or a salt thereof.

In certain embodiments, the compound of Formula (IX-b) is of Formula (IX-b-4):

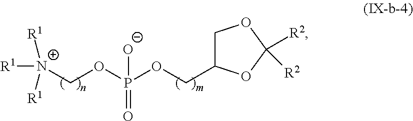

or a salt thereof.

In certain embodiments, the compound of Formula (IX-b) is one of the following:

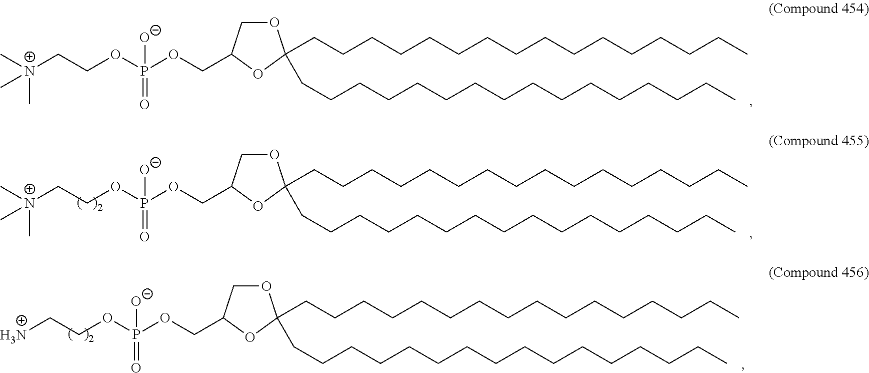

or salts thereof.

### Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IX) is of Formula (IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (IX) is of Formula (IX-c).

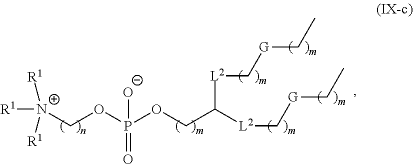

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (IX-c) is of Formula (IX-c-1):

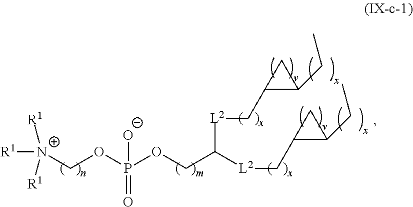

or salt thereof, wherein:

each instance of v is independently 1, 2, or 3.

In certain embodiments, the compound of Formula (IX-c) is of Formula (IX-c-2):

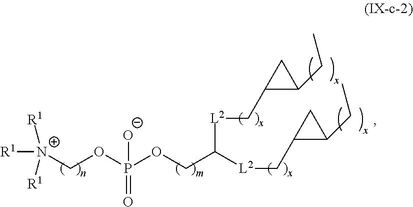

or a salt thereof.

In certain embodiments, the compound of Formula (IX-c) is of the following formula:

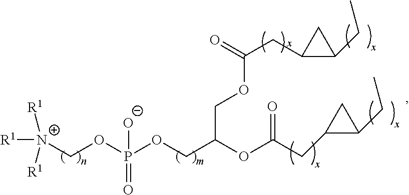

or a salt thereof.

In certain embodiments, the compound of Formula (IX-c) is the following:

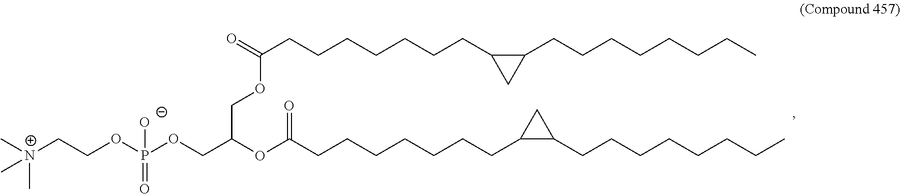 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (IX-c) is of Formula (IX-c-3):

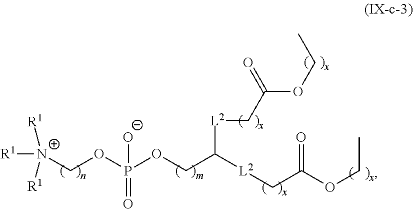

or a salt thereof.

In certain embodiments, the compound of Formula (IX-c) is of the following formulae:

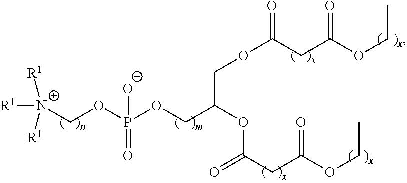

or a salt thereof.

In certain embodiments, the compound of Formula (IX-c) is the following:

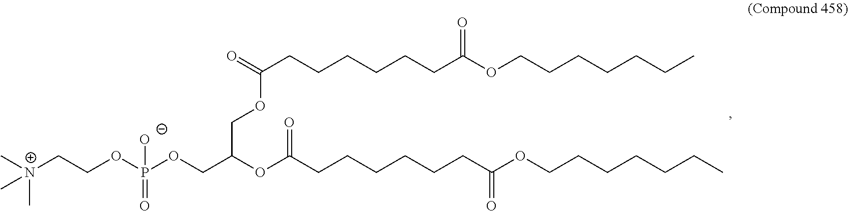 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IX), wherein n is 1, 3, 4, 5, 6, 7, 8, 9, or 10. For example, in certain embodiments, a compound of Formula (IX) is of one of the following formulae:

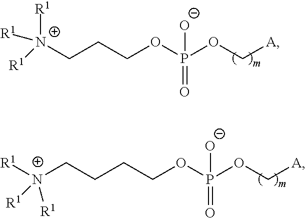

or a salt thereof.

In certain embodiments, a compound of Formula (IX) is one of the following:

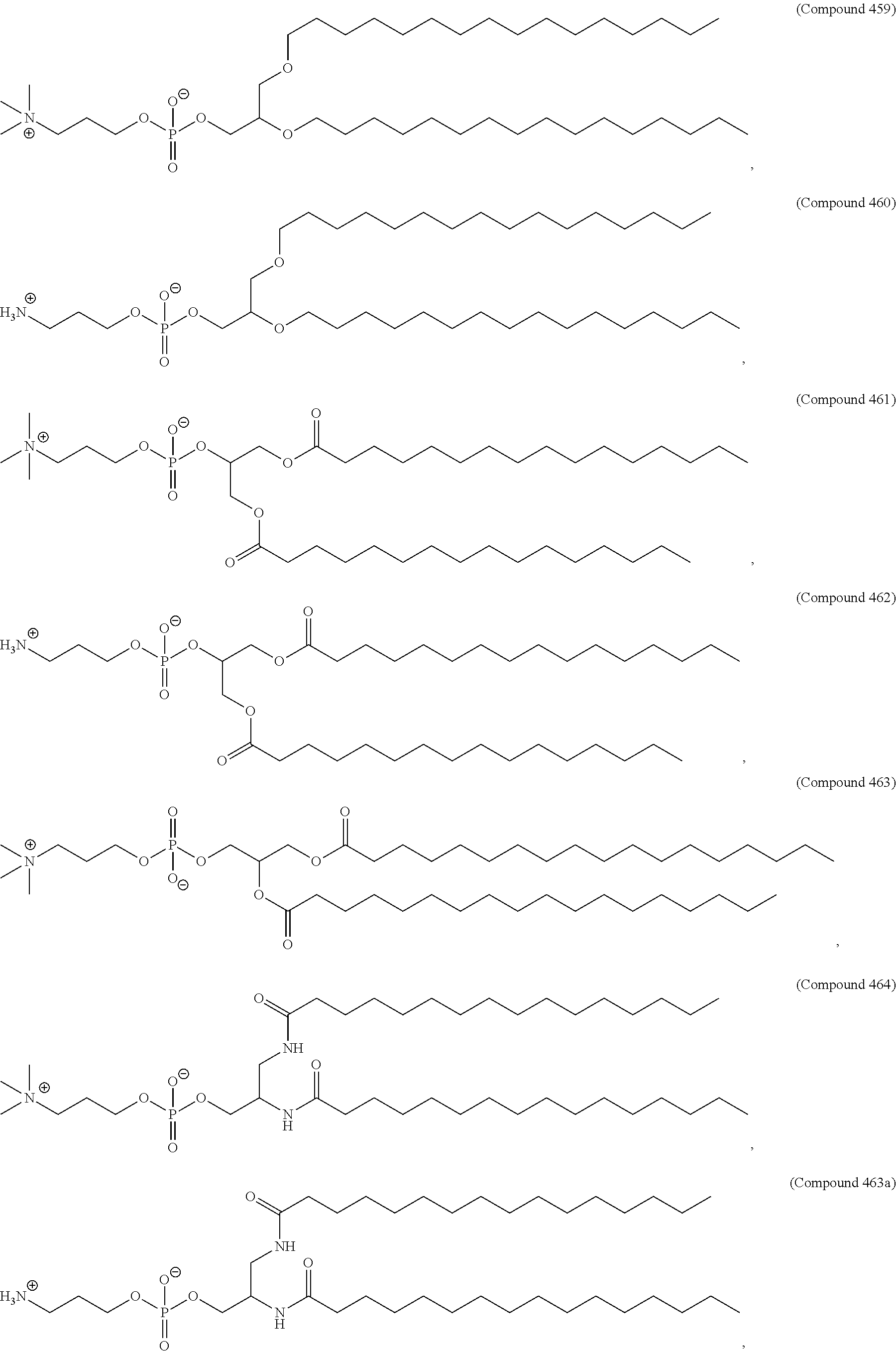

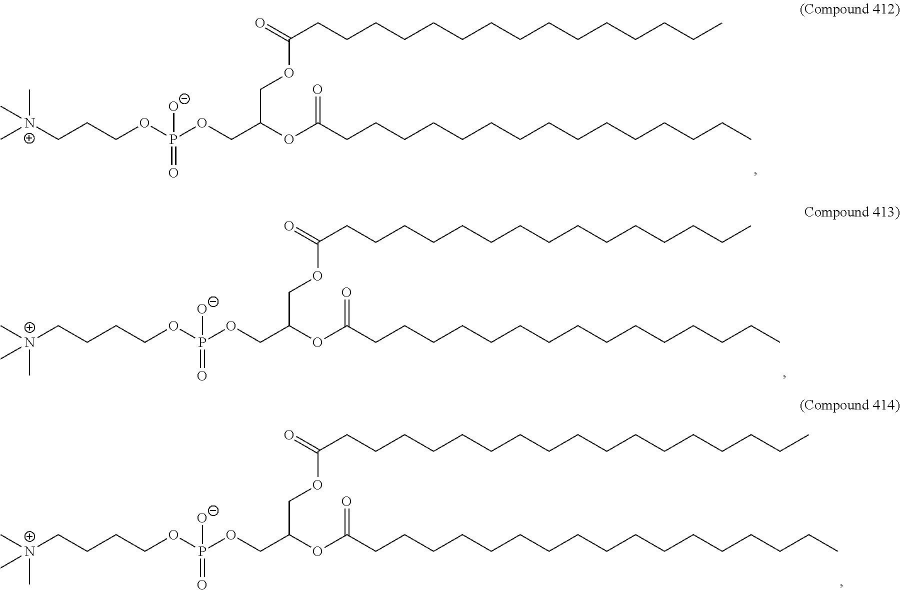

or salts thereof

### Alternative Lipids

In certain embodiments, an alternative lipid is used in place of a phospholipid of the invention. Non-limiting examples of such alternative lipids include the following:

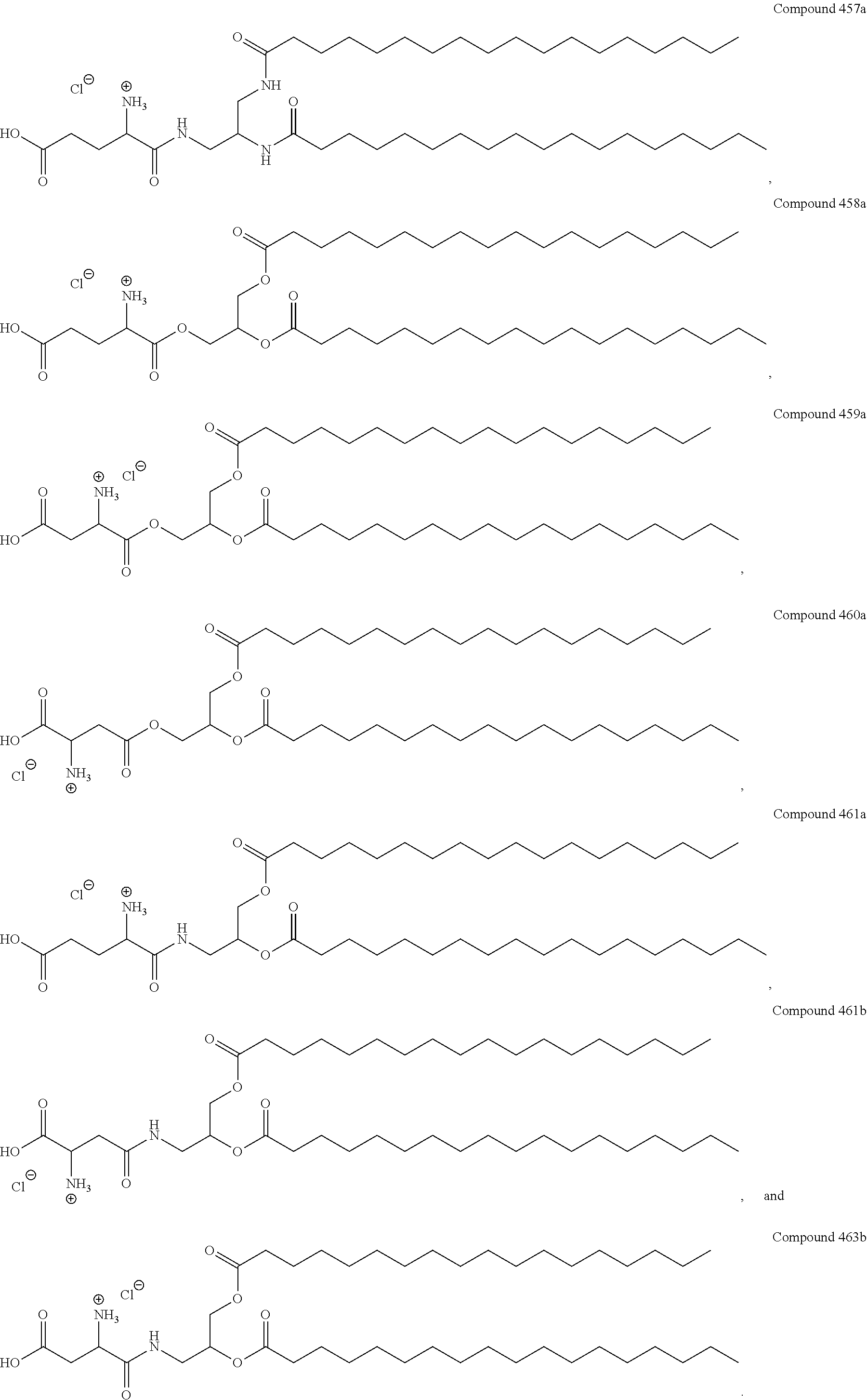

### Structural Lipids

The lipid component of a lipid nanoparticle composition may include one or more structural lipids. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

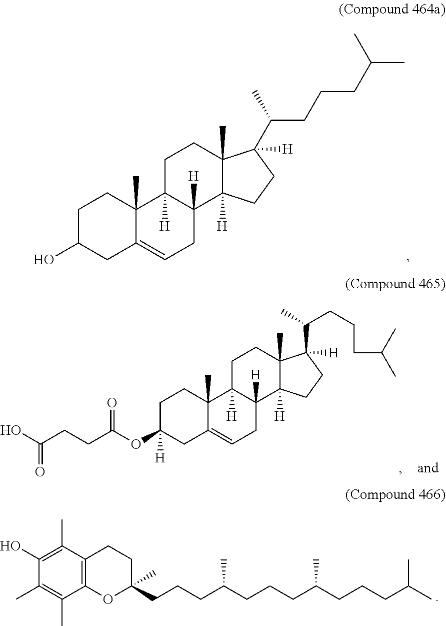

In some embodiments, the nanoparticle described herein can comprise about 20 mol % to about 60 mol % structural lipid. In some embodiments, the nanoparticle comprises about 30 mol % to about 50 mol % of structural lipid. In some embodiments, the nanoparticle comprises about 35 mol % of structural lipid. In some embodiments, the nanoparticle comprises about 40 mol % structural lipid. In some embodiments, the structural lipid is cholesterol or a compound having the following structure:

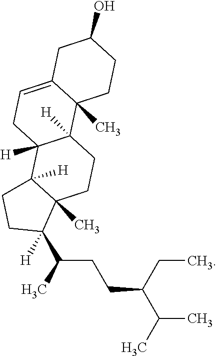

### Payload Molecules

The compositions of the disclosure can be used to deliver a wide variety of different agents to an airway cell. An airway cell can be a cell lining the respiratory tract, e.g., in the mouth, nose, throat, or lungs. The therapeutic agent is capable of mediating (e.g., directly mediating or via a bystander effect) a therapeutic effect in such an airway cell. Typically the therapeutic agent delivered by the composition is a nucleic acid, although non-nucleic acid agents, such as small molecules, chemotherapy drugs, peptides, polypeptides and other biological molecules are also encompassed by the disclosure. Nucleic acids that can be delivered include DNA-based molecules (i.e., comprising deoxyribonucleotides) and RNA-based molecules (i.e., comprising ribonucleotides). Furthermore, the nucleic acid can be a naturally occurring form of the molecule or a chemically-modified form of the molecule (e.g., comprising one or more modified nucleotides).

### Agents for Enhancing Protein Expression

In one embodiment, the therapeutic agent is an agent that enhances (i.e., increases, stimulates, upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors).

In one embodiment, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., that encodes and can express a transcript. For example, the DNA therapeutic agent can encode a protein of interest, to thereby increase expression of the protein of interest in an airway upon delivery by an LNP. In some embodiments, the DNA molecule can be naturally-derived, e.g., isolated from a natural source. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, the payload comprises a genetic modulator, i.e., at least one component of a system which modifies a nucleic acid sequence in a DNA molecule, e.g., by altering a nucleobase, e.g., introducing an insertion, a deletion, a mutation (e.g., a missense mutation, a silent mutation or a nonsense mutation), a duplication, or an inversion, or any combination thereof. In some embodiments, the genetic modulator comprises a DNA base editor, CRISPR/Cas gene editing system, a zinc finger nuclease (ZFN) system, a Transcription activator-like effector nuclease (TALEN) system, a meganuclease system, or a transposase system, or any combination thereof.

In some embodiments, the genetic modulator comprises a template DNA. In some embodiments, the genetic modulator does not comprise a template DNA. In some embodiments, the genetic modulator comprises a template RNA. In some embodiments, the genetic modulator does not comprise a template RNA.

In some embodiments, the genetic modulator is a CRISPR/Cas gene editing system. In some embodiments, the CRISPR/Cas gene editing system comprises a guide RNA (gRNA) molecule comprising a targeting sequence specific to a sequence of a target gene and a peptide having nuclease activity, e.g., endonuclease activity, e.g., a Cas protein or a fragment (e.g., biologically active fragment) or a variant thereof, e.g., a Cas9 protein, a fragment (e.g., biologically active fragment) or a variant thereof, a Cas3 protein, a fragment (e.g., biologically active fragment) or a variant thereof, a Cas12a protein, a fragment (e.g., biologically active fragment) or a variant thereof, a Cas 12e protein, a fragment (e.g., biologically active fragment) or a variant thereof, a Cas 13 protein, a fragment (e.g., biologically active fragment) or a variant thereof, or a Cas14 protein, a fragment (e.g., biologically active fragment) or a variant thereof.

In some embodiments, the CRISPR/Cas gene editing system comprises a gRNA molecule comprising a targeting sequence specific to a sequence of a target gene, and a nucleic acid encoding a peptide having nuclease activity, e.g., endonuclease activity, e.g., a Cas protein or a fragment (e.g., biologically active fragment) or variant thereof, e.g., a Cas9 protein, a fragment (e.g., biologically active fragment) or a variant thereof; a Cas3 protein, a fragment (e.g., biologically active fragment) or a variant thereof; a Cas12a protein, a fragment (e.g., biologically active fragment) or a variant thereof; a Cas12e protein, a fragment (e.g., biologically active fragment) or a variant thereof; a Cas13 protein, a fragment (e.g., biologically active fragment) or a variant thereof; or a Cas14 protein, a fragment (e.g., biologically active fragment) or a variant thereof.

In some embodiments, the CRISPR/Cas gene editing system comprises a nucleic acid encoding a gRNA molecule comprising a targeting sequence specific to a sequence of a target gene, and a Cas9 protein, a fragment (e.g., biologically active fragment) or a variant thereof.

In some embodiments, the CRISPR/Cas gene editing system comprises a nucleic acid encoding a gRNA molecule comprising a targeting sequence specific to a sequence of a target gene, and a nucleic acid encoding a Cas9 protein, a fragment (e.g., biologically active fragment) or a variant thereof.

In some embodiments, the CRISPR/Cas gene editing system further comprises a template DNA. In some embodiments, the CRISPR/Cas gene editing system further comprises a template RNA. In some embodiments, the CRISPR/Cas gene editing system further comprises a Reverse transcriptase.

In some embodiments of any of the methods, compositions, or cells disclosed herein, the genetic modulator is a zinc finger nuclease (ZFN) system. In some embodiments, the ZFN system comprises a peptide having: a Zinc finger DNA binding domain, a fragment (e.g., biologically active fragment) or a variant thereof; and/or nuclease activity, e.g., endonuclease activity. In some embodiments, the ZFN system comprises a peptide having a Zn finger DNA binding domain. In some embodiments, the Zn finger binding domain comprises 1, 2, 3, 4, 5, 6, 7, 8 or more Zinc fingers. In some embodiments, the ZFN system comprises a peptide having nuclease activity e.g., endonuclease activity. In some embodiments, the peptide having nuclease activity is a type-II restriction 1-like endonuclease, e.g., a FokI endonuclease. In some embodiments, the ZFN system comprises a nucleic acid encoding a peptide having: a Zinc finger DNA binding domain, a fragment (e.g., biologically active fragment) or a variant thereof; and/or nuclease activity, e.g., endonuclease activity.

In some embodiments, the ZFN system comprises a nucleic acid encoding a peptide having a Zn finger DNA binding domain. In some embodiments, the Zn finger binding domain comprises 1, 2, 3, 4, 5, 6, 7, 8 or more Zinc fingers. In some embodiments, the ZFN system comprises a nucleic acid encoding a peptide having nuclease activity e.g., endonuclease activity. In some embodiments, the peptide having nuclease activity is a type-II restriction 1-like endonuclease, e.g., a FokI endonuclease.

In some embodiments, the system further comprises a template, e.g., template DNA.

In some embodiments of any of the methods, compositions, or cells disclosed herein, the genetic modulator is a Transcription activator-like effector nuclease (TALEN) system. In some embodiments, the system comprises a peptide having: a Transcription activator-like (TAL) effector DNA binding domain, a fragment (e.g., biologically active fragment) or a variant thereof; and/or nuclease activity, e.g., endonuclease activity. In some embodiments, the system comprises a peptide having a TAL effector DNA binding domain, a fragment (e.g., biologically active fragment) or a variant thereof. In some embodiments, the system comprises a peptide having nuclease activity, e.g., endonuclease activity. In some embodiments, the peptide having nuclease activity is a type-II restriction 1-like endonuclease, e.g., a FokI endonuclease.

In some embodiments, the system comprises a nucleic acid encoding a peptide having: a Transcription activator-like (TAL) effector DNA binding domain, a fragment (e.g., biologically active fragment) or a variant thereof; and/or nuclease activity, e.g., endonuclease activity. In some embodiments, the system comprises a nucleic acid encoding a peptide having a Transcription activator-like (TAL) effector DNA binding domain, a fragment (e.g., biologically active fragment) or a variant thereof. In some embodiments, the system comprises a nucleic acid encoding a peptide having nuclease activity, e.g., endonuclease activity. In some embodiments, the peptide having nuclease activity is a type-II restriction 1-like endonuclease, e.g., a FokI endonuclease.

In some embodiments, the system further comprises a template, e.g., a template DNA.

In some embodiments of any of the methods, compositions, or cells disclosed herein, the genetic modulator is a meganuclease system. In some embodiments, the meganuclease system comprises a peptide having a DNA binding domain and nuclease activity, e.g., a homing endonuclease. In some embodiments, the homing endonuclease comprises a LAGLIDADG endonuclease, GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.

In some embodiments, the meganuclease system comprises a nucleic acid encoding a peptide having a DNA binding domain and nuclease activity, e.g., a homing endonuclease. In some embodiments, the homing endonuclease comprises a LAGLIDADG endonuclease, GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.

In some embodiments, the system further comprises a template, e.g., a template DNA.

In some embodiments of any of the methods, compositions, or cells disclosed herein, the genetic modulator is a transposase system. In some embodiments, the transposase system comprises a nucleic acid sequence encoding a peptide having reverse transcriptase and/or nuclease activity, e.g., a retrotransposon, e.g., an LTR retrotransposon or a non-LTR retrotransposon. In some embodiments, the transposase system comprises a template, e.g., an RNA template.

In one embodiment, the therapeutic agent is an RNA therapeutic agent. The RNA molecule can be a single-stranded RNA, a double-stranded RNA (dsRNA) or a molecule that is a partially double-stranded RNA, i.e., has a portion that is double-stranded and a portion that is single-stranded. The RNA molecule can be a circular RNA molecule or a linear RNA molecule.

An RNA therapeutic agent can be an RNA therapeutic agent that is capable of transferring a gene into a cell, e.g., encodes a protein of interest, to thereby increase expression of the protein of interest in an airway cell. In some embodiments, the RNA molecule can be naturally-derived, e.g., isolated from a natural source. In other embodiments, the RNA molecule is a synthetic molecule, e.g., a synthetic RNA molecule produced in vitro.

Non-limiting examples of RNA therapeutic agents include messenger RNAs (mRNAs) (e.g., encoding a protein of interest), modified mRNAs (mmRNAs), mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA), locked nucleic acids (LNAs) and that encode components of CRISPR/Cas9 technology, each of which is described further in subsections below. In some embodiments, the RNA modulator comprises an RNA base editor system. In some embodiments, the RNA base editor system comprises: a deaminase, e.g., an RNA-specific adenosine deaminase (ADAR); a Cas protein, a fragment (e.g., biologically active fragment) or a variant thereof, and/or a guide RNA. In some embodiments, the RNA base editor system further comprises a template, e.g., a DNA or RNA template.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m27,02′GppppG, m7Gpppm7G, m73′dGpppG, m27,03′GpppG, m27,03′GppppG, and m27,02′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2,N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine.

Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Therapeutic Agents for Reducing Protein Expression

In one embodiment, the therapeutic agent is a therapeutic agent that reduces (i.e., decreases, inhibits, downregulates) protein expression. In one embodiment, the therapeutic agent reduces protein expression in the target airway cell Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

### Peptide/Polypeptide Therapeutic Agents

In one embodiment, the therapeutic agent is a peptide therapeutic agent. In one embodiment the therapeutic agent is a polypeptide therapeutic agent.

In some embodiments, the therapeutic payload or prophylactic payload comprises an mRNA encoding: a secreted protein; a membrane-bound protein; or an intercellular protein, or peptides, polypeptides or biologically active fragments thereof.

In some embodiments, the therapeutic payload or prophylactic payload comprises an mRNA encoding a secreted protein, a peptide, a polypeptide or a biologically active fragment thereof. In some embodiments, the therapeutic payload or prophylactic payload comprises an mRNA encoding a membrane-bound protein, a peptide, a polypeptide or a biologically active fragment thereof. In some embodiments, the therapeutic payload or prophylactic payload comprises an mRNA encoding an intracellular protein, a peptide, a polypeptide or a biologically active fragment thereof. In some embodiments, the therapeutic payload or prophylactic payload comprises a protein, polypeptide, or peptide.

In some embodiments, the peptide or polypeptide is naturally-derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In one embodiment, the peptide or polypeptide therapeutic agent of the composition is a naturally occurring peptide or polypeptide. In one embodiment, the peptide or polypeptide therapeutic agent of the composition is a modified version of a naturally occurring peptide or polypeptide (e.g., contains less than 3, less than 5, less than 10, less than 15, less than 20, or less than 25 amino substitutions, deletions, or additions compared to its wild type, naturally occurring peptide or polypeptide counterpart).

### LNPs Comprising Cationic Agents

The LNPs of the invention comprise a LNP core and a cationic agent disposed primarily on the outer surface of the core. Such LNPs have a greater than neutral zeta potential at physiologic pH.

Core lipid nanoparticles typically comprise one or more of the following components: lipids (which may include ionizable amino lipids, phospholipids, helper lipids which may be neutral lipids, zwitterionic lipid, anionic lipids, and the like), structural lipids such as cholesterol or cholesterol analogs, fatty acids, polymers, stabilizers, salts, buffers, solvent, and the like.

Certain of the LNP cores provided herein comprise an ionizable lipid, such as an ionizable lipid, e.g., an ionizable amino lipid, a phospholipid, a structural lipid, and optionally a stabilizer (e.g., a molecule comprising polyethylene glycol) which may or may not be provided conjugated to another lipid.

The structural lipid may be but is not limited to a sterol such as for example cholesterol. The structural lipid can be β-sitosterol.

The helper lipid is a non-cationic lipid. The helper lipid may comprise at least one fatty acid chain of at least 8C and at least one polar headgroup moiety.

When a molecule comprising polyethylene glycol (i.e. PEG) is used, it may be used as a stabilizer. In some embodiments, the molecule comprising polyethylene glycol may be polyethylene glycol conjugated to a lipid and thus may be provided as PEG-c-DOMG or PEG-DMG, for example. Certain of the LNPs provided herein comprise no or low levels of PEGylated lipids, including no or low levels of alkyl-PEGylated lipids, and may be referred to herein as being free of PEG or PEGylated lipid. Thus, some LNPs comprise less than 0.5 mol % PEGylated lipid. In some instances, PEG may be an alkyl-PEG such as methoxy-PEG. Still other LNPs comprise non-alkyl-PEG such as hydroxy-PEG, and/or non-alkyl-PEGylated lipids such as hydroxy-PEGylated lipids. Certain LNPs provided herein comprise high levels of PEGylated lipids. Some LNPS comprise 0.5 mol % PEGylated lipid. Some LNPs comprise more than 0.5 mol % PEGylated lipid. In some embodiments, the LNPs comprise 1.5 mol % PEGylated lipid. In some embodiments, the LNPs comprise 3.0 mol % PEGylated lipid. In some embodiments, the LNPs comprise 0.1 mol % to 3.0 mol % PEGylated lipid, 0.5 mol % to 2.0 mol % PEGylated lipid, or 1.0 mol % to 1.5 mol % PEGylated lipid.

In some embodiments, a core nanoparticle composition can have the formulation of Compound 18:Phospholipid:Chol: N-lauroyl-D-erythro-sphinganylphosphorylcholine with a mole ratio of 50:10:38.5:1.5. In some embodiments, a nanoparticle core composition can have the formulation of Compound 18:DSPC:Chol:Compound 428 with a mole ratio of 50:10:38.5:1.5.

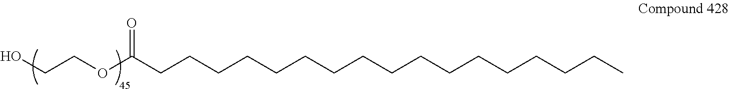

Nanoparticles of the present disclosure comprise at least one compound according to Formula (I). For example, the nanoparticle composition can include one or more of Compounds 1-147. Nanoparticles can also include a variety of other components. For example, the nanoparticle composition can include one or more other lipids in addition to a lipid according to Formula (I) or (II), for example (i) at least one phospholipid, (ii) at least one structural lipid, (iii) at least one PEG-lipid, or (iv) any combination thereof.

In some embodiments, the nanoparticle composition comprises a compound of Formula (I), (e.g., Compounds 18, 25, 26 or 48). In some embodiments, the nanoparticle composition comprises a compound of Formula (I) (e.g., Compounds 18, 25, 26 or 48) and a phospholipid (e.g., DSPC, DOPE, or MSPC). In some embodiments, the nanoparticle composition comprises a compound of Formula (I) (e.g., Compounds 18, 25, 26 or 48) and a phospholipid (e.g., DSPC, DPPC, DOPE, or MSPC).

The present disclosure also provides process of preparing a nanoparticle comprising contacting a lipid nanoparticle with a cationic agent, wherein the lipid nanoparticle comprises:
(a) a lipid nanoparticle core comprising:
   (i) an ionizable lipid,
   (ii) a phospholipid,
   (iii) a structural lipid, and
   (iv) a PEG-lipid, and
(b) a polynucleotide or polypeptide payload encapsulated within the core for delivery into a cell.

In some embodiments, the contacting of the lipid nanoparticle with a cationic agent comprises dissolving the cationic agent in a non-ionic excipient. In some embodiments, the non-ionic excipient is selected from macrogol 15 hydroxystearate (HS 15), 1,2-dimyristoyl-rac-glycero-3-methoxypolyethylene glycol-2000 (DMG-PEG2K), Compound 428, polyoxyethylene sorbitan monooleate [TWEEN®80], and d-α-Tocopherol polyethylene glycol succinate (TPGS). In some embodiments, the non-ionic excipient is macrogol 15 hydroxystearate (HS 15). In some embodiments, the contacting of the lipid nanoparticle with a cationic agent comprises the cationic agent dissolved in a buffer solution. In some embodiments, the buffer solution is a phosphate buffered saline (PBS). In some embodiments, the buffer solution is a Tris-based buffer.

Provided are nanoparticles prepared by the process as described herein, e.g., by contacting the lipid nanoparticle with a cationic agent. In some embodiments, the cationic agent can be a sterol amine such as GL-67. In some embodiments, the lipid nanoparticle core of the lipid nanoparticle optionally comprises a PEG-lipid. In some embodiments, the lipid nanoparticle core forming the lipid nanoparticle which is contacted with the cationic agent is substantially free of PEG-lipid. In some embodiments, the PEG-lipid is added to the lipid nanoparticle together with the cationic agent, prior to the contacting with the cationic agent, or after the contacting with the cationic agent.

In one embodiment, an LNP of the invention can be made using traditional mixing technology in which the nucleic acid payload is mixed with core LNP components to create the core LNP plus payload. Once this loaded core LNP is prepared, the cationic agent is contacted with the loaded core LNP.

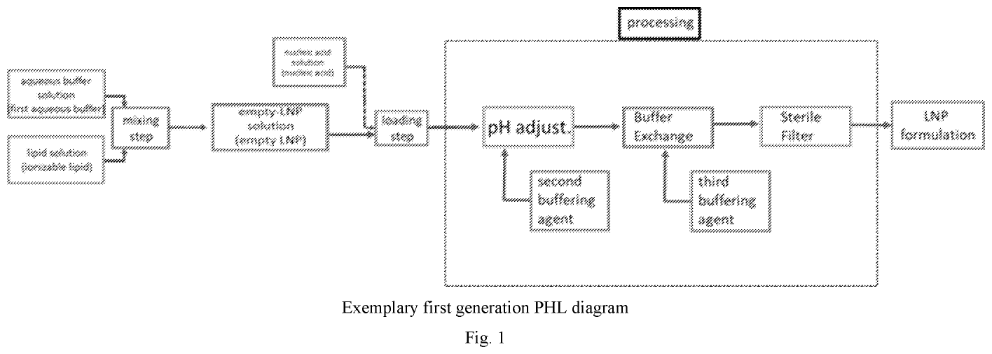

In another embodiment, an LNP of the invention can be made using empty LNPs as the starting point. For example, as shown in FIG. 1, empty LNPs are made prior to loading in the nucleic acid payload. Once the nucleic acid payload is contacted with the LNP, the cationic agent can be added to form an LNP of the invention.

For example, in one embodiment, in the post-hoc loading (PHL) method, empty LNPs are formulated first in a nanoprecipitation step, and buffer exchanged into a low pH buffer (i.e. pH 5). Next, these empty LNPs are introduced to mRNA (also acidified at low pH) through a mixing event. After the mixing step, a pH adjustment method is used to neutralize the pH. Finally, a PEG lipid, e.g., DMG-PEG-2k is added to stabilize the particle. These particles are then concentrated to the target concentration and filtered. A cationic agent, e.g., GL67 is added.

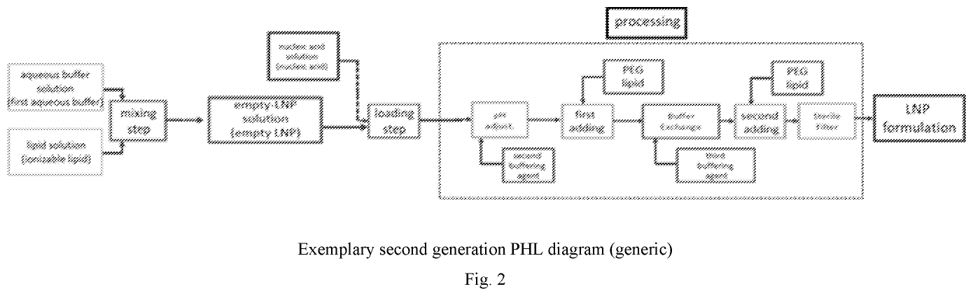

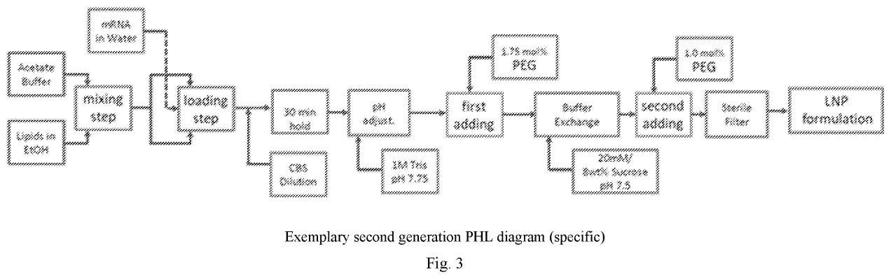

A variation of the empty LNP starting point is illustrated in FIG. 2. FIG. 2 shows that the lipids of the LNP, excluding the PEG lipids, are used to form an empty LNP. The nucleic acid solution is then contacted with the empty LNPs, forming loaded LNPs. The PEG lipids are added at one or two points during further processing of the loaded LNPs and the cationic agent can be added at any point during that further processing, illustrated by the dotted box in FIG. 2. FIG. 3 is a more specific version of the process in FIG. 2 and, again, the cationic agent can be added at any point during the further processing of the loaded LNP.

In some embodiments, an LNP of the invention can be prepared using nanoprecipitation, which is the unit operation in which the LNPs are self-assembled from their individual lipid components by way of kinetic mixing and subsequent maturation and continuous dilution. This unit operation includes three individual steps, which are: mixing of the aqueous and organic inputs, maturation of the LNPs, and dilution after a controlled residence time. Due to the continuous nature of these steps, they are considered one unit operation. The unit operation includes the continuous inline combination of three liquid streams with one inline maturation step: mixing of the aqueous buffer with lipid stock solution, maturation via controlled residence time, and dilution of the nanoparticles. The nanoprecipitation itself occurs in the scale-appropriate mixer, which is designed to allow continuous, high-energy, combination of the aqueous solution with the lipid stock solution dissolved in ethanol. The aqueous solution and the lipid stock solution both flow simultaneously into the mixing hardware continuously throughout this operation. The ethanol content, which keeps the lipids dissolved, is abruptly reduced and the lipids all precipitate with each other. The particles are thus self-assembled in the mixing chamber.

One of the objectives of unit operation is to exchange the solution into a fully aqueous buffer, free of ethanol, and to reach a target concentration of LNP. This can be achieved by first reaching a target processing concentration, then diafiltering, and then (if necessary) a final concentration step, once the ethanol has been completely removed.

In some embodiments, an LNP of the invention can be prepared using nanoprecipitation, which is the unit operation in which the LNPs are self-assembled from their individual lipid components by way of kinetic mixing and subsequent maturation and continuous dilution. This unit operation includes three individual steps, which are: mixing of the aqueous and organic inputs, maturation of the LNPs, and dilution after a controlled residence time. Due to the continuous nature of these steps, they are considered one unit operation. The unit operation includes the continuous inline combination of three liquid streams with one inline maturation step: mixing of the aqueous buffer with lipid stock solution, maturation via controlled residence time, and dilution of the nanoparticles. The nanoprecipitation itself occurs in the scale-appropriate mixer, which is designed to allow continuous, high-energy, combination of the aqueous solution with the lipid stock solution dissolved in ethanol. The aqueous solution and the lipid stock solution both flow simultaneously into the mixing hardware continuously throughout this operation. The ethanol content, which keeps the lipids dissolved, is abruptly reduced and the lipids all precipitate with each other. The particles are thus self-assembled in the mixing chamber.

One of the objectives of unit operation is to exchange the solution into a fully aqueous buffer, free of ethanol, and to reach a target concentration of LNP. This can be achieved by first reaching a target processing concentration, then diafiltering, and then (if necessary) a final concentration step, once the ethanol has been completely removed.

In some aspects, the present disclosure provides a method of preparing an empty-lipid nanoparticle solution (empty-LNP solution) comprising an empty lipid nanoparticle (empty LNP), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time; and
   i-c) adding a diluting solution to the intermediate empty-LNP solution, thereby forming the empty-LNP solution comprising the empty LNP.

In some aspects, the present disclosure provides a method of preparing an empty-lipid nanoparticle solution (empty-LNP solution) comprising an empty lipid nanoparticle (empty LNP), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution.

In some aspects, the present disclosure provides a method of preparing an empty-lipid nanoparticle solution (empty-LNP solution) comprising an empty lipid nanoparticle (empty LNP), comprising:
ii) processing an empty-LNP solution comprising the empty LNP.

In some aspects, the present disclosure provides a method of preparing a lipid nanoparticle formulation (LNP formulation), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution; and
iii) a loading step, comprising mixing a nucleic acid solution comprising a nucleic acid with the empty-LNP solution, thereby forming a loaded LNP solution comprising a loaded lipid nanoparticle (loaded LNP).

In some aspects, the present disclosure provides a method of preparing a lipid nanoparticle formulation (LNP formulation), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution;
iii) a loading step, comprising mixing a nucleic acid solution comprising a nucleic acid with the empty-LNP solution, thereby forming a loaded LNP solution comprising a loaded lipid nanoparticle (loaded LNP); and
iv) processing the loaded LNP solution, thereby forming the loaded LNP formulation.

In some aspects, the present disclosure provides a method of preparing a lipid nanoparticle formulation (LNP formulation), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution;
iii) a loading step, comprising mixing a nucleic acid solution comprising a nucleic acid with the empty-LNP solution, thereby forming a loaded LNP solution comprising a loaded lipid nanoparticle (loaded LNP);
iv) processing the loaded LNP solution, thereby forming the loaded LNP formulation; and
v) adding a cationic agent.

In some aspects, the present disclosure provides a method of preparing a lipid nanoparticle formulation (LNP formulation), comprising:
iii) a loading step, comprising mixing a nucleic acid solution comprising a nucleic acid with an empty-LNP solution comprising an empty LNP, thereby forming a loaded nanoparticle solution (loaded LNP solution) comprising a loaded lipid nanoparticle (loaded LNP).

In some aspects, the present disclosure provides a method of preparing a lipid nanoparticle formulation (LNP formulation), comprising:
iii) a loading step, comprising mixing a nucleic acid solution comprising a nucleic acid with an empty-LNP solution comprising an empty LNP, thereby forming a loaded nanoparticle solution (loaded LNP solution) comprising a loaded lipid nanoparticle (loaded LNP); and
iv) processing the loaded LNP solution, thereby forming the loaded LNP formulation.

In some aspects, the present disclosure provides a method of preparing a lipid nanoparticle formulation (LNP formulation), comprising:
iii) a loading step, comprising mixing a nucleic acid solution comprising a nucleic acid with an empty-LNP solution comprising an empty LNP, thereby forming a loaded nanoparticle solution (loaded LNP solution) comprising a loaded lipid nanoparticle (loaded LNP)
iv) processing the loaded LNP solution, thereby forming the loaded LNP formulation; and
v) adding a cationic agent.

In some embodiments, steps i-a) to i-c) are performed in separate operation units (e.g., separate reaction devices).

In some embodiments, steps i-a) to i-c) are performed in a single operation unit. In some embodiments, steps i-a) to i-c) are performed in a continuous flow device, such that step i-c) is downstream from step i-b) which is downstream from step i-a).

In some embodiments, in step i-c), the diluting solution is added once.

In some embodiments, in step i-c), the diluting solution is added continuously.

In some aspects, the present disclosure provides a method of producing an empty lipid nanoparticle (empty LNP), the method comprising: i) a mixing step, comprising mixing an ionizable lipid with a first buffering agent, thereby forming the empty LNP, wherein the empty LNP comprises from about 0.1 mol % to about 0.5 mol % of a polymeric lipid (for example, a PEG lipid).

In some aspects, the present disclosure provides a method of preparing an empty-lipid nanoparticle solution (empty-LNP solution) comprising an empty lipid nanoparticle (empty LNP), comprising:
i) a mixing step, comprising mixing a lipid solution comprising an ionizable lipid, a structural lipid, a phospholipid, and a PEG lipid, with an aqueous buffer solution comprising a first buffering agent, thereby forming an empty-lipid nanoparticle solution (empty-LNP solution) comprising the empty LNP.

In some aspects, the present disclosure provides a method of preparing an empty-lipid nanoparticle solution (empty-LNP solution) comprising an empty lipid nanoparticle (empty LNP), comprising:
i) a mixing step, comprising mixing a lipid solution comprising an ionizable lipid, a structural lipid, a phospholipid, and a PEG lipid, with an aqueous buffer solution comprising a first buffering agent, thereby forming an empty-lipid nanoparticle solution (empty-LNP solution) comprising the empty LNP; and
ii) processing the empty-LNP solution.

In some embodiments, the mixing step comprises mixing a lipid solution comprising the ionizable lipid with an aqueous buffer solution comprising the first buffering agent, thereby forming an empty-lipid nanoparticle solution (empty-LNP solution) comprising the empty LNP.

In some aspects, the present disclosure provides a method of preparing a loaded lipid nanoparticle (loaded LNP) associated with a nucleic acid, comprising: ii) a loading step, comprising mixing a nucleic acid with an empty LNP followed by addition of a cationic agent, thereby forming the loaded LNP.

In some embodiments, the loading step comprises mixing the nucleic acid solution comprising the nucleic acid with the empty-LNP solution followed by addition of a cationic agent, thereby forming a loaded lipid nanoparticle solution (loaded-LNP solution) comprising the loaded LNP.

In some embodiments, the empty LNP or the empty-LNP solution is subjected to the loading step without holding or storage.

In some embodiments, the empty LNP or the empty-LNP solution is subjected to the loading step after holding for a period of time.

In some embodiments, the empty LNP or the empty-LNP solution is subjected to the loading step after holding for about 1 minute, about 2 minutes, about 3 minutes, about 4 minutes, about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, or about 24 hours.

In some embodiments, the empty LNP or the empty-LNP solution is subjected to the loading step after storage for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, or about 5 years.

In some embodiments, upon formation, the empty LNP or the empty-LNP solution is subjected to the loading step without storage or holding for a period of time.

In some aspects, the present disclosure provides a method, further comprising: ii) processing the empty-LNP solution.

In some aspects, the present disclosure provides a method, further comprising: iv) processing the loaded-LNP solution, thereby forming a lipid nanoparticle formulation (LNP formulation).

In contrast to other techniques for production (e.g., thin film rehydration/extrusion), ethanol-drop precipitation has been the industry standard for generating nucleic acid lipid nanoparticles. Precipitation reactions are favored due to their continuous nature, scalability, and ease of adoption. Those processes usually use high energy mixers (e.g., T-junction, confined impinging jets, microfluidic mixers, vortex mixers) to introduce lipids (in ethanol) to a suitable anti-solvent (i.e. water) in a controllable fashion, driving liquid supersaturation and spontaneous precipitation into lipid particles. In some embodiments, the vortex mixers used are those described in U.S. Patent Application Nos. 62/799,636 and 62/886,592, which are incorporated herein by reference in their entirety. In some embodiments, the microfluidic mixers used are those described in PCT Application No. WO/2014/172045, which is incorporated herein by reference in their entirety.

In some embodiments, the mixing step is performed with a T-junction, confined impinging jets, microfluidic mixer, or vortex mixer.

In some embodiments, the loading step is performed with a T-junction, confined impinging jets, microfluidic mixer, or vortex mixer.

In some embodiments, the mixing step is performed at a temperature of less than about 30° C., less than about 28° C., less than about 26° C., less than about 24° C., less than about 22° C., less than about 20° C., or less than about ambient temperature.

In some embodiments, the loading step is performed at a temperature of less than about 30° C., less than about 28° C., less than about 26° C., less than about 24° C., less than about 22° C., less than about 20° C., or less than about ambient temperature.

In some embodiments, the step of processing the empty-LNP solution or loaded-LNP solution comprises a first adding step, comprising adding a polyethylene glycol lipid (PEG lipid) to the empty LNP or the loaded LNP.

In some embodiments, the step of processing the empty-LNP solution comprises a first adding step, comprising adding a polyethylene glycol lipid (PEG lipid) to the empty LNP solution.

In some embodiments, the step of processing the empty-LNP solution comprises a first adding step, comprising adding a polyethylene glycol lipid (PEG lipid) to the empty LNP.

In some embodiments, the step of processing the loaded-LNP solution comprises a first adding step, comprising adding a polyethylene glycol lipid (PEG lipid) to the loaded LNP solution.

In some embodiments, the step of processing the loaded-LNP solution comprises a first adding step, comprising adding a polyethylene glycol lipid (PEG lipid) to the loaded LNP.

In some embodiments, the first adding step comprises adding a polyethylene glycol solution (PEG solution) comprising the PEG lipid to the empty-LNP solution or loaded-LNP solution.

In some embodiments, the step of processing the empty-LNP solution or loaded-LNP solution comprises a second adding step, comprising adding a polyethylene glycol lipid (PEG lipid) to the empty LNP or the loaded LNP.

In some embodiments, the step of processing the empty-LNP solution comprises a second adding step, comprising adding a polyethylene glycol lipid (PEG lipid) to the empty LNP solution.

In some embodiments, the step of processing the empty-LNP solution comprises a second adding step, comprising adding a polyethylene glycol lipid (PEG lipid) to the empty LNP.

In some embodiments, the step of processing the loaded-LNP solution comprises a second adding step, comprising adding a polyethylene glycol lipid (PEG lipid) to the loaded LNP solution.

In some embodiments, the step of processing the loaded-LNP solution comprises a second adding step, comprising adding a polyethylene glycol lipid (PEG lipid) to the loaded LNP.

In some embodiments, the second adding step comprises adding a polyethylene glycol solution (PEG solution) comprising the PEG lipid to the empty-LNP solution or loaded-LNP solution.

In some embodiments, first adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty LNP or the loaded LNP.

In some embodiments, the first adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty-LNP or The loaded-LNP. In some embodiments, the first adding step comprises adding about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of PEG lipid (e.g., PEG₂ₖ-DMG).

In some embodiments, the first adding step comprises adding about 1.75±0.5 mol %, about 1.75±0.4 mol %, about 1.75±0.3 mol %, about 1.75±0.2 mol %, or about 1.75±0.1 mol % (e.g., about 1.75 mol %) of PEG lipid (e.g., PEG₂ₖ-DMG).

In some embodiments, after the first adding step, the empty LNP solution (e.g., the empty LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, after the first adding step, the loaded LNP solution (e.g., the loaded LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, the second adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty LNP or the loaded LNP.

In some embodiments, the second adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty LNP or the loaded LNP.

In some embodiments, the second adding step comprises adding about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of PEG lipid (e.g., PEG₂ₖ-DMG).

In some embodiments, the second adding step comprises adding about 1.0±0.5 mol %, about 1.0±0.4 mol %, about 1.0±0.3 mol %, about 1.0±0.2 mol %, or about 1.0±0.1 mol % (e.g., about 1.0 mol %) of PEG lipid (e.g., PEG₂ₖ-DMG).

In some embodiments, the second adding step comprises adding about 1.0 mol % PEG lipid to the empty LNP or the loaded LNP.

In some embodiments, after the second adding step, the empty LNP solution (e.g., the empty LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, after the second adding step, the loaded LNP solution (e.g., the loaded LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, the first adding step is performed at a temperature of less than about 30° C., less than about 28° C., less than about 26° C., less than about 24° C., less than about 22° C., less than about 20° C., or less than about ambient temperature.

In some embodiments, the second adding step is performed at a temperature of less than about 30° C., less than about 28° C., less than about 26° C., less than about 24° C., less than about 22° C., less than about 20° C., or less than about ambient temperature.

In some embodiments, the step of processing the empty-LNP solution or loaded-LNP solution further comprises at least one step selected from filtering, pH adjusting, buffer exchanging, diluting, dialyzing, concentrating, freezing, lyophilizing, storing, and packing.

In some embodiments, the step of processing the empty-LNP solution or loaded-LNP solution further comprises pH adjusting.

In some embodiments, the pH adjusting comprises adding a second buffering agent is selected from the group consisting of an acetate buffer, a citrate buffer, a phosphate buffer, and a tris buffer.

In some embodiments, the first adding step is performed prior to the pH adjusting.

In some embodiments, the first adding step is performed after the pH adjusting.

In some embodiments, the second adding step is performed prior to the pH adjusting.

In some embodiments, the second adding step is performed after the pH adjusting.

In some embodiments, the step of processing the empty-LNP solution or loaded-LNP solution further comprises filtering.

In some embodiments, the filtering is a tangential flow filtration (TFF).

In some embodiments, the filtering removes an organic solvent (e.g., an alcohol or ethanol) from the LNP solution. In some embodiments, upon removal of the organic solvent (e.g. an alcohol or ethanol), the LNP solution is converted to a solution buffered at a neutral pH, pH 6.5 to 7.8, pH 6.8 to pH 7.5, preferably, pH 7.0 to pH 7.2 (e.g., a phosphate or HEPES buffer). In some embodiments, the LNP solution is converted to a solution buffered at a pH of about 7.0 to pH to about 7.2. In some embodiments, the resulting LNP solution is sterilized before storage or use, e.g., by filtration (e.g., through a 0.1-0.5 μm filter).

In some embodiments, the step of processing the empty-LNP solution or loaded-LNP solution further comprises buffer exchanging.

In some embodiments, the buffer exchanging comprises addition of an aqueous buffer solution comprising a third buffering agent.

In some embodiments, the first adding step is performed prior to the buffer exchanging.

In some embodiments, the first adding step is performed after the buffer exchanging.

In some embodiments, the second adding is performed prior to the buffer exchanging.

In some embodiments, the second adding step is performed after the buffer exchanging.

In some embodiments, the step of processing the empty-LNP solution or loaded-LNP solution further comprises diluting.

In some embodiments, the step of processing the empty-LNP solution or loaded-LNP solution further comprises dialyzing.

In some embodiments, the step of processing the empty-LNP solution or loaded-LNP solution further comprises concentrating.

In some embodiments, the step of processing the empty-LNP solution or loaded-LNP solution further comprises freezing.

In some embodiments, the step of processing the empty-LNP solution or loaded-LNP solution further comprises lyophilizing.

In some embodiments, the lyophilizing comprises freezing the loaded-LNP solution at a temperature from about −100° C. to about 0° C., about −80° C. to about −10° C., about −60° C. to about −20° C., about −50° C. to about −25° C., or about −40° C. to about −30° C.

In some embodiments, the lyophilizing further comprises drying the frozen loaded-LNP solution to form a lyophilized empty LNP or lyophilized loaded LNP.

In some embodiments, the drying is performed at a vacuum ranging from about 50 mTorr to about 150 mTorr.

In some embodiments, the drying is performed at about −35° C. to about −15° C.

In some embodiments, the drying is performed at about room temperature to about 25° C.

In some embodiments, the step of processing the empty-LNP solution or loaded-LNP solution further comprises storing.

In some embodiments, the storing comprises storing the empty LNP or the loaded LNP at a temperature of about −80° C., about −78° C., about −76° C., about −74° C., about −72° C., about −70° C., about −65° C., about −60° C., about −55° C., about −50° C., about −45° C., about −40° C., about −35° C., or about −30° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the storing comprises storing the empty LNP or the loaded LNP at a temperature of about −40° C., about −35° C., about −30° C., about −25° C., about −20° C., about −15° C., about −10° C., about −5° C., about 0° C., about 5° C., about 10° C., about 15° C., about 20° C., or about 25° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the storing comprises storing the empty LNP or the loaded LNP at a temperature of about −40° C. to about 0° C., from about −35° C. to about −5° C., from about −30° C. to about −10° C., from about −25° C. to about −15° C., from about −22° C. to about −18° C., or from about −21° C. to about −19° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the storing comprises storing the empty LNP or the loaded LNP at a temperature of about −20° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the step of processing the empty-LNP solution or loaded-LNP solution further comprises packing.

As used herein, “packing” may refer to storing a drug product in its final state or in-process storage of an empty LNP, loaded LNP, or LNP formulation before they are placed into final packaging. Modes of storage and/or packing include, but are not limited to, refrigeration in sterile bags, refrigerated or frozen formulations in vials, lyophilized formulations in vials and syringes, etc.

In some embodiments, the step of processing the empty-LNP solution or loaded-LNP solution comprises: iia) adding a cryoprotectant to the empty-LNP solution or loaded-LNP solution.

In some embodiments, the step of processing the empty-LNP solution or loaded-LNP solution comprises: iib) filtering the empty-LNP solution or loaded-LNP

### Solution

In some embodiments, the step of processing the empty-LNP solution or loaded-LNP solution comprises:
iia) adding a cryoprotectant to the empty-LNP solution or loaded-LNP solution; and
iic) filtering the empty-LNP solution or loaded-LNP solution.

In some embodiments, the step of processing the empty-LNP solution or loaded-LNP solution comprises one or more of the following steps:
iib) adding a cryoprotectant to the empty-LNP solution or loaded-LNP solution;
iic) lyophilizing the empty-LNP solution or loaded-LNP solution, thereby forming a lyophilized LNP composition;
iid) storing the empty-LNP solution or loaded-LNP solution of the lyophilized LNP composition; and
iie) adding a buffering solution to the empty-LNP solution, loaded-LNP solution or the lyophilized LNP composition, thereby forming the LNP formulation.

In some embodiments, the step of processing the empty-LNP solution comprises: iia) adding a cryoprotectant to the empty-LNP solution.

In some embodiments, the step of processing the empty-LNP solution comprises: iib) filtering the empty-LNP solution.

In some embodiments, the step of processing the empty-LNP solution comprises:
iia) adding a cryoprotectant to the empty-LNP solution; and
iic) filtering the empty-LNP solution.

In some embodiments, the cryoprotectant is added to the empty-LNP solution or loaded-LNP solution prior to the lyophilization. In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof. In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, the cryoprotectant comprises a cryoprotective agent present at a concentration from about 10 g/L to about 1000 g/L, from about 25 g/L to about 950 g/L, from about 50 g/L to about 900 g/L, from about 75 g/L to about 850 g/L, from about 100 g/L to about 800 g/L, from about 150 g/L to about 750 g/L, from about 200 g/L to about 700 g/L, from about 250 g/L to about 650 g/L, from about 300 g/L to about 600 g/L, from about 350 g/L to about 550 g/L, from about 400 g/L to about 500 g/L, and from about 450 g/L to about 500 g/L. In some embodiments, the cryoprotectant comprises a cryoprotective agent present at a concentration from about 10 g/L to about 500 g/L, from about 50 g/L to about 450 g/L, from about 100 g/L to about 400 g/L, from about 150 g/L to about 350 g/L, from about 200 g/L to about 300 g/L, and from about 200 g/L to about 250 g/L. In some embodiments, the cryoprotectant comprises a cryoprotective agent present at a concentration of about 10 g/L, about 25 g/L, about 50 g/L, about 75 g/L, about 100 g/L, about 150 g/L, about 200 g/L, about 250 g/L, about 300 g/L, about 300 g/L, about 350 g/L, about 400 g/L, about 450 g/L, about 500 g/L, about 550 g/L, about 600 g/L, about 650 g/L, about 700 g/L, about 750 g/L, about 800 g/L, about 850 g/L, about 900 g/L, about 950 g/L, and about 1000 g/L.

In some embodiments, the cryoprotectant comprises a cryoprotective agent present at a concentration from about 0.1 mM to about 100 mM, from about 0.5 mM to about 90 mM, from about 1 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 25 mM, from about 8 mM to about 20 mM, from about 9 mM to about 15 mM, and from about 10 mM to about 15 mM. In some embodiments, the cryoprotectant comprises a cryoprotective agent present at a concentration from about 0.1 mM to about 10 mM, from about 0.5 mM to about 9 mM, from about 1 mM to about 8 mM, from about 2 mM to about 7 mM, from about 3 mM to about 6 mM, and from about 4 mM to about 5 mM. In some embodiments, the cryoprotectant comprises a cryoprotective agent present at a concentration of about 0.1 mM, about 0.5 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, and about 100 mM.

In some embodiments, the cryoprotectant comprises sucrose.

In some embodiments, the cryoprotectant comprises an aqueous solution comprising sucrose.

In some embodiments, the cryoprotectant comprises an aqueous solution comprising about 700±300 g/L, 700±200 g/L, 700±100 g/L, 700±90 g/L, 700±80 g/L, 700±70 g/L, 700±60 g/L, 700±50 g/L, 700±40 g/L, 700±30 g/L, 700±20 g/L, 700±10 g/L, 700±9 g/L, 700±8 g/L, 700±7 g/L, 700±6 g/L, 700±5 g/L, 700±4 g/L, 700±3 g/L, 700±2 g/L, or 700±1 g/L of sucrose.

In some embodiments, the cryoprotectant comprises an aqueous solution comprising sodium acetate and sucrose.

In some embodiments, the cryoprotectant comprises an aqueous solution comprising:
(a) about 5±1 mM, about 5±0.9 mM, about 5±0.8 mM, about 5±0.5 mM, about 5±0.6 mM, about 5±0.5 mM, about 5±0.4 mM, about 5±0.3 mM, about 5±0.2 mM, or about 5±0.1 mM of sodium acetate; and
(b) about 700±300 g/L, 700±200 g/L, 700±100 g/L, 700±90 g/L, 700±80 g/L, 700±70 g/L, 700±60 g/L, 700±50 g/L, 700±40 g/L, 700±30 g/L, 700±20 g/L, 700±10 g/L, 700±9 g/L, 700±8 g/L, 700±7 g/L, 700±6 g/L, 700±5 g/L, 700±4 g/L, 700±3 g/L, 700±2 g/L, or 700±1 g/L of sucrose.

In some embodiments, the cryoprotectant comprises an aqueous solution comprising sodium acetate and sucrose, wherein the aqueous solution has a pH value of 5.0±2.0, 5.0±1.5, 5.0±1.0, 5.0±0.9, 5.0±0.8, 5.0±0.7, 5.0±0.6, 5.0±0.5, 5.0±0.4, 5.0±0.3, 5.0±0.2, or 5.0±0.1.

In some embodiments, the cryoprotectant comprises an aqueous solution comprising:
(a) about 5±1 mM, about 5±0.9 mM, about 5±0.8 mM, about 5±0.5 mM, about 5±0.6 mM, about 5±0.5 mM, about 5±0.4 mM, about 5±0.3 mM, about 5±0.2 mM, or about 5±0.1 mM of sodium acetate; and
(b) about 700±300 g/L, 700±200 g/L, 700±100 g/L, 700±90 g/L, 700±80 g/L, 700±70 g/L, 700±60 g/L, 700±50 g/L, 700±40 g/L, 700±30 g/L, 700±20 g/L, 700±10 g/L, 700±9 g/L, 700±8 g/L, 700±7 g/L, 7000±6 g/L, 700±5 g/L, 700±4 g/L, 700±3 g/L, 700±2 g/L, or 700±1 g/L of sucrose; and
wherein the aqueous solution has a pH value of 5.0±2.0, 5.0±1.5, 5.0±1.0, 5.0±0.9, 5.0±0.8, 5.0±0.7, 5.0±0.6, 5.0±0.5, 5.0±0.4, 5.0±0.3, 5.0±0.2, or 5.0±0.1.

In some embodiments, the lyophilization is carried out in a suitable glass receptacle (e.g., a 10 mL cylindrical glass vial). In some embodiments, the glass receptacle withstand extreme changes in temperatures between lower than −40° C. and higher than room temperature in short periods of time, and/or be cut in a uniform shape. In some embodiments, the step of lyophilizing comprises freezing the LNP solution at a temperature higher than about −40° C., thereby forming a frozen LNP solution; and drying the frozen LNP solution to form the lyophilized LNP composition. In some embodiments, the step of lyophilizing comprises freezing the LNP solution at a temperature higher than about −40° C. and lower than about −30° C. The freezing step results in a linear decrease in temperature to the final over about 6 minutes, preferably at about 1° C. per minute from 20° C. to −40° C. In some embodiments, the freezing step results in a linear decrease in temperature to the final over about 6 minutes at about 1° C. per minute from 20° C. to −40° C. In some embodiments, sucrose at 12-15% may be used, and the drying step is performed at a vacuum ranging from about 50 mTorr to about 150 mTorr. In some embodiments, sucrose at 12-15% may be used, and the drying step is performed at a vacuum ranging from about 50 mTorr to about 150 mTorr, first at a low temperature ranging from about −35° C. to about −15° C., and then at a higher temperature ranging from room temperature to about 25° C. In some embodiments, sucrose at 12-15% may be used, and the drying step is performed at a vacuum ranging from about 50 mTorr to about 150 mTorr, and the drying step is completed in three to seven days. In some embodiments, sucrose at 12-15% may be used, and the drying step is performed at a vacuum ranging from about 50 mTorr to about 150 mTorr, first at a low temperature ranging from about −35° C. to about −15° C., and then at a higher temperature ranging from room temperature to about 25° C., and the drying step is completed in three to seven days. In some embodiments, the drying step is performed at a vacuum ranging from about 50 mTorr to about 100 mTorr. In some embodiments, the drying step is performed at a vacuum ranging from about 50 mTorr to about 100 mTorr, first at a low temperature ranging from about −15° C. to about 0° C., and then at a higher temperature.

In some embodiments, the empty-LNP solution, loaded-LNP solution, or the lyophilized LNP composition is stored at a pH from about 3.5 to about 8.0, from about 4.0 to about 7.5, from about 4.5 to about 7.0, from about 5.0 to about 6.5, and from about 5.5 to about 6.0. In some embodiments, the empty-LNP solution, loaded-LNP solution, or the lyophilized LNP composition is stored at a pH of about 3.5, about 4.0, about 4.5, about 4.6, about 4.7, about 4.8, about 4.9, about 5.0, about 5.1, about 5.2, about 5.3, about 5.4, about 4.5, about 5.5, about 6.5, about 7.0, about 7.5, and about 8.0.

In some embodiments, the LNP solution, loaded-LNP solution, or the lyophilized LNP composition is stored in a cryoprotectant comprising sucrose and sodium acetate. In some embodiments, the LNP solution, loaded-LNP solution, or the lyophilized LNP composition is stored in a cryoprotectant comprising from about 150 g/L to about 350 g/L sucrose and from about 3 mM to about 6 mM sodium acetate at a pH from about 4.5 to about 7.0. In some embodiments, the LNP solution, loaded-LNP solution, or the lyophilized LNP composition is stored in a cryoprotectant comprising about 200 g/L sucrose and 5 mM sodium acetate at about pH 5.0.

In some embodiments, the empty-LNP solution, loaded-LNP solution, or the lyophilized LNP composition is stored at a temperature of about −80° C., about −78° C., about −76° C., about −74° C., about −72° C., about −70° C., about −65° C., about −60° C., about −55° C., about −50° C., about −45° C., about −40° C., about −35° C., or about −30° C. prior to adding the buffering solution.

In some embodiments, the empty-LNP solution, loaded-LNP solution, or the lyophilized LNP composition is stored at a temperature of about −40° C., about −35° C., about −30° C., about −25° C., about −20° C., about −15° C., about −10° C., about −5° C., about 0° C., about 5° C., about 10° C., about 15° C., about 20° C., or about 25° C. prior to adding the buffering solution.

In some embodiments, the empty-LNP solution, loaded-LNP solution, or the lyophilized LNP composition is stored at a temperature of ranging from about −40° C. to about 0° C., from about −35° C. to about −5° C., from about −30° C. to about −10° C., from about −25° C. to about −15° C., from about −22° C. to about −18° C., or from about −21° C. to about −19° C. prior to adding the buffering solution.

In some embodiments, the empty-LNP solution, loaded-LNP solution, or the lyophilized LNP composition is stored at a temperature of about −20° C. prior to adding the buffering solution.

Certain aspects of the methods are described in PCT Application No. WO/2020/160397 which is incorporated herein by reference in their entirety.

Described herein are also cells comprising a nanoparticle. The cells can be epithelial cells. For example, the cells can be lung cells. The cells can be respiratory epithelial cells. For example, the cells can be lung cells, nasal cells, alveolar epithelial cells, or bronchial epithelial cells. The cells can be human bronchial epithelial (HBE) cells. The cells can be HeLa cells. Such cells can be contacted with LNPs in vitro or in vivo.

### Pharmaceutical Compositions and Formulations

The present disclosure provides pharmaceutical compositions and formulations that comprise any of nanoparticles described herein.

Pharmaceutical compositions or formulations can optionally comprise one or more additional active substances, e.g., therapeutically and/or prophylactically active substances. Pharmaceutical compositions or formulations of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to the nanoparticle comprising the polynucleotides or polypeptide payload to be delivered as described herein.

Formulations and pharmaceutical compositions described herein can be prepared by any method known or hereafter developed in the art of pharmacology. In general, such preparatory methods include the step of associating the nanoparticle with an excipient and/or one or more other accessory ingredients, and then, if necessary and/or desirable, dividing, shaping and/or packaging the product into a desired single- or multi-dose unit.

A pharmaceutical composition or formulation in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals.

A pharmaceutically acceptable excipient, as used herein, includes, but is not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, Md., 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, acorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions are maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present disclosure can include, but are not limited to sucrose, trehalose, mannitol, glycine, lactose, raffinose, and combinations thereof.

The compositions can be in a liquid form or a solid form. In some embodiments, the compositions or formulations are in a liquid form. In some embodiments, the compositions are suitable for inhalation. The compositions can be administered to the pulmonary tract. Aerosolized pharmaceutical formulations can be delivered to the lungs, preferably using a number of commercially available devices.

Compositions can be administered to the respiratory tract by suitable methods such as intranasal instillation, intratracheal instillation, and intratracheal injection. In some embodiments, the compositions or the nanoparticle is administered by intranasal, intrabronchial, or pulmonary administration. For example, the compositions and nanoparticles are administered by nebulizer or inhaler.

In some embodiments, the compositions are delivered into the lungs by inhalation of an aerosolized pharmaceutical formulation. Inhalation can occur through the nose and/or the mouth of the subject. Administration can occur by self-administration of the formulation while inhaling, or by administration of the formulation via a respirator to a subject on a respirator. Exemplary devices for delivering formulations to the lung include, but are not limited to, dry powder inhalers, pressurized metered dose inhalers, nebulizers, and electrohydrodynamic aerosol devices.

Liquid formulations can be administered to the lungs of a patient using a pressurized metered dose inhaler (pMDI). pMDIs generally include at least two components: a canister in which the liquid formulation is held under pressure in combination with one or more propellants, and a receptacle used to hold and actuate the canister. The canister may contain a single or multiple doses of the formulation. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the liquid formulation is atomized, forming an aerosol. pMDIs typically employ one or more propellants to pressurize the contents of the canister and to propel the liquid formulation out of the receptacle outlet, forming an aerosol. Any suitable propellants may be utilized. The propellant may take a variety of forms. For example, the propellant may be a compressed gas or a liquefied gas.

The liquid formulations can also be administered using a nebulizer. Nebulizers are liquid aerosol generators that convert the liquid formulation into mists or clouds of small droplets, preferably having diameters less than 5 microns mass median aerodynamic diameter, which can be inhaled into the lower respiratory tract. This process is called atomization. The droplets carry the one or more active agents into the nose, upper airways or deep lungs when the aerosol cloud is inhaled. Any type of nebulizer may be used to administer the formulation to a patient, including, but not limited to pneumatic (jet) nebulizers and electromechanical nebulizers. Pneumatic (jet) nebulizers use a pressurized gas supply as a driving force for atomization of the liquid formulation. Compressed gas is delivered through a nozzle or jet to create a low pressure field which entrains a surrounding liquid formulation and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir. Electromechanical nebulizers use electrically generated mechanical force to atomize liquid formulations. The electromechanical driving force can be applied, for example, by vibrating the liquid formulation at ultrasonic frequencies, or by forcing the bulk liquid through small holes in a thin film. The forces generate thin liquid films or filament streams which break up into small droplets to form a slow moving aerosol stream which can be entrained in an inspiratory flow. Liquid formulations can also be administered using an electrohydrodynamic (EHD) aerosol device. EHD aerosol devices use electrical energy to aerosolize liquid drug solutions or suspensions.

Dry powder inhalers (DPIs) typically use a mechanism such as a burst of gas to create a cloud of dry powder inside a container, which can then be inhaled by the subject. In a DPI, the dose to be administered is stored in the form of a non-pressurized dry powder and, on actuation of the inhaler, the particles of the powder are inhaled by the subject. In some cases, a compressed gas (i.e., propellant) may be used to dispense the powder, similar to pressurized metered dose inhalers (pMDIs). In some cases, the DPI may be breath actuated, meaning that an aerosol is created in precise response to inspiration. Typically, dry powder inhalers administer a dose of less than a few tens of milligrams per inhalation to avoid provocation of cough. Examples of DPIs include the Turbohaler® inhaler (Astrazeneca, Wilmington, Del.), the Clickhaler® inhaler (Innovata, Ruddington, Nottingham, UKL), the Diskus® inhaler (Glaxo, Greenford, Middlesex, UK), the EasyHaler® (Orion, Expoo, FI), the Exubera® inhaler (Pfizer, New York, N.Y.), the Qdose® inhaler (Microdose, Monmouth Junction, N.J.), and the Spiros® inhaler (Dura, San Diego, Calif.).

The pharmaceutical compositions of the invention are administered in an effective amount to cause a desired biological effect, e.g., a therapeutic or prophylactic effect, e.g., owing to expression of a normal gene product to supplement or replace a defective protein or to reduce expression of an undesired protein, as measured by, in some embodiments, the alleviation of one or more symptoms. The formulations may be administered in an effective amount to deliver LNP to, e.g., the apical membrane of respiratory and non-respiratory epithelial cells to deliver a payload. In some embodiments, the pharmaceutical compositions are administered in an effective amount to induce absent CFTR activity in a patient suffering from CF or augment the existing level of residual CFTR activity in a patient suffering from CF.

The presence of desired biologic activity, e.g., residual CFTR activity at the epithelial surface can be readily detected using methods known in the art, including standard electrophysiological, biochemical, and/or histochemical techniques. Such methods identify and/or quantify CFTR activity using in vivo or ex vivo electrophysiological techniques, measurement of sweat or salivary CT concentrations, or ex vivo biochemical or histochemical techniques to monitor CFTR cell surface density.

### Methods of Use

Described herein are methods of treating or preventing a disease in a patient which disease is associated with airway cell dysfunction. The method comprises administering to the patient a nanoparticle or composition comprising a nucleic acid payload as described herein for treatment or prevention of the disease. For example, in one embodiment, the payload is a nucleic acid molecule, e.g., an mRNA molecule and the disease is ameliorated by expression of a protein or polypeptide in airway epithelial cells. In some embodiment, the disease is cystic fibrosis.

In some embodiments, the nanoparticles described herein are used in methods for reducing cellular sodium levels in a subject in need thereof.

In some embodiments, the nanoparticles described herein are used to reduce the level of a metabolite associated with CF (e.g., the substrate or product), the method comprising administering to the subject an effective amount of a polynucleotide encoding a CFTR polypeptide.

In some embodiments, the administration of an effective amount of the nanoparticles described herein reduces the levels of a biomarker of CF, e.g., intracellular sodium levels. In some embodiments, the administration of the nanoparticles described herein results in reduction in the level of one or more biomarkers of CF, e.g., intracellular sodium levels, within a short period of time after administration of the nanoparticles described herein.

In some embodiments, the administration of the nanoparticles described herein to a subject results in a decrease in intracellular sodium levels in cells to a level at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% lower than the level observed prior to the administration of the composition or formulation.

In some embodiments, provided herein is a method of delivering a polynucleotide or polypeptide payload into a cell, which comprises contacting the cell with a nanoparticle described herein. In some embodiments, the administration of the nanoparticles described herein results in expression of CFTR in cells of the subject. In some embodiments, administering the nanoparticles described herein results in an increase of CFTR enzymatic activity in the subject. For example, the method can result in an increase of CFTR enzymatic activity in at least some cells of a subject.

In some embodiments, the administration of the nanoparticles described herein comprising an mRNA encoding a CFTR polypeptide to a subject results in an increase of CFTR enzymatic activity in cells subject to a level at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% or more of the activity level expected in a normal subject, e.g., a human not suffering from CF.

In some embodiments, the administration of the nanoparticles described herein results in expression of CFTR protein in at least some of the cells of a subject that persists for a period of time sufficient to allow significant chloride channel activity to occur.

In some embodiments, the expression of the encoded polypeptide is increased. In some embodiments, the polynucleotide increases CFTR expression levels in cells when introduced into those cells, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% with respect to the CFTR expression level in the cells before the polypeptide is introduced in the cells.

As will be appreciated by those skilled in the art, the sterol amines disclosed herein have additional uses. For example, sterol amines can be used to treat inflammatory diseases. Sterol amines can also be used as antimicrobial agents.

### Kits and Devices

The present disclosure provides a variety of kits for conveniently and/or effectively using the claimed nanoparticles of the present disclosure. Typically kits will comprise sufficient amounts and/or numbers of components to allow a user to perform multiple treatments of a subject(s) and/or to perform multiple experiments.

In one aspect, the present disclosure provides kits comprising the nanoparticles of the present disclosure.

The kit can further comprise packaging and instructions and/or a delivery agent to form a formulation composition. The delivery agent can comprise a saline, a buffered solution, a lipidoid or any delivery agent disclosed herein. In one embodiment, such a kit further comprises an administration device such as a nebulizer or an inhaler.

### Respiratory Function and Other Test for Improvement in Respiratory Symptoms

In some embodiments, a nanoparticle or pharmaceutical composition comprising an mRNA comprising an open reading frame (ORF) encoding a polypeptide or protein. Such a polypeptide or protein can be tested for improvement to respiratory function or symptoms. For example, in one embodiment, cystic fibrosis transmembrane conductance regulator (CFTR) polypeptide, when administered to a subject in need thereof, is sufficient to improve a measure of at least one respiratory volume by at least 10%, at least 15%, at least 20%, at least 25%, or at least 30% as compared to at least one reference respiratory volume measured in the subject untreated for cystic fibrosis, for at least 24 hours, at least 48 hours, at least 72 hours, at least 96 hours, or at least 120 hours post-administration. Respiratory volumes are the amount of air inhaled, exhaled and stored within the lungs at any given time. Non-limiting examples of various respiratory volumes that may be measured are provided below.

Total lung capacity (TLC) is the volume in the lungs at maximal inflation, the sum of VC and RV. The average total lung capacity is 6000 ml, although this varies with age, height, sex and health.

Tidal volume (TV) is the volume of air moved into or out of the lungs during quiet breathing (TV indicates a subdivision of the lung; when tidal volume is precisely measured, as in gas exchange calculation, the symbol TV or VT is used). The average tidal volume is 500 ml.

Residual volume (RV) is the volume of air remaining in the lungs after a maximal exhalation. Residual volume (RV/TLC %) is expressed as percent of TLC.

Expiratory reserve volume (ERV) is the maximal volume of air that can be exhaled (above tidal volume) during a forceful breath out.

Inspiratory reserve volume (IRV) is the maximal volume that can be inhaled from the end-inspiratory position.

Inspiratory capacity (IC) is the sum of IRV and TV.

Inspiratory vital capacity (IVC) is the maximum volume of air inhaled from the point of maximum expiration.

Vital capacity (VC) is the volume of air breathed out after the deepest inhalation.

Functional residual capacity (FRC) is the volume in the lungs at the end-expiratory position.

Forced vital capacity (FVC) is the determination of the vital capacity from a maximally forced expiratory effort.

Forced expiratory volume (time) (FEVₜ) is a generic term indicating the volume of air exhaled under forced conditions in the first t seconds. FEV₁ is the volume that has been exhaled at the end of the first second of forced expiration. FEFₓ is the forced expiratory flow related to some portion of the FVC curve; modifiers refer to amount of FVC already exhaled. FEFₘₐₓ is the maximum instantaneous flow achieved during a FVC maneuver.

Forced inspiratory flow (FIF) is a specific measurement of the forced inspiratory curve, denoted by nomenclature analogous to that for the forced expiratory curve. For example, maximum inspiratory flow is denoted FIFₘₐₓ. Unless otherwise specified, volume qualifiers indicate the volume inspired from RV at the point of measurement.

Peak expiratory flow (PEF) is the highest forced expiratory flow measured with a peak flow meter.

Maximal voluntary ventilation (MVV) is the volume of air expired in a specified period during repetitive maximal effort.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the schemes below.

The reactions for preparing compounds described herein can be carried out in suitable solvents, which can be readily selected by one of skill in the art of organic synthesis. Suitable solvents can be substantially non-reactive with the starting materials (reactants), the intermediates, or products at the temperatures at which the reactions are carried out, (e.g., temperatures, which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Compounds of Formula A2a can be prepared, e.g., using a process as illustrated in the schemes below:

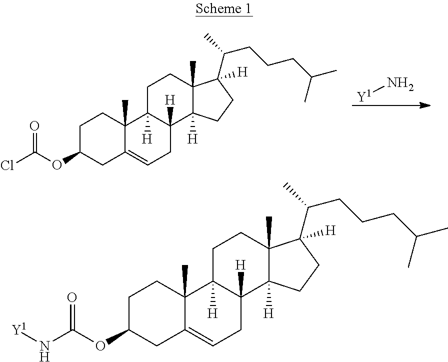

Compounds of Formula A2a can be prepared via the synthetic route outlined in Scheme 1. An appropriate reaction between cholesteryl chloroformate and amines can be carried out under suitable conditions to generate a compound of Formula A2a.

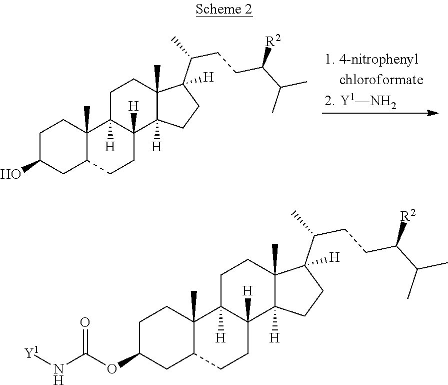

Compounds of Formula A2a can be prepared via the synthetic route outlined in Scheme 2. An appropriate reaction between cholesterol or a cholesterol derivative (such as stigmasterol) and 4-nitrophenyl chloroformate can be carried out under suitable conditions (such as using triethylamine and 4-dimethylaminopyridine). The product of said reaction can be reacted with an amine under suitable conditions (such as using triethylamine) to give a compound of Formula A2a.

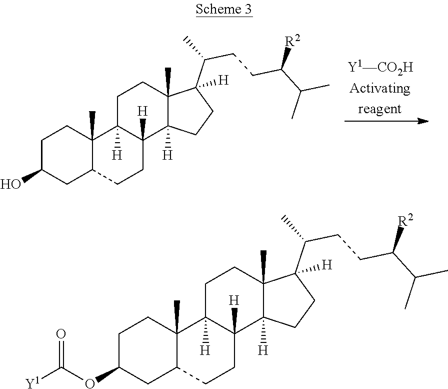

Compounds of Formula A2a can be prepared via the synthetic route outlined in Scheme 3. An appropriate reaction between cholesterol or a cholesterol derivative (such as stigmasterol) and a carboxylic acid can be carried out in the presences of an activating reagent (such as, e.g., EDC-HCl, DMAP, DCC, or pivalic anhydride) in suitable conditions to give compounds of Formula A2a.

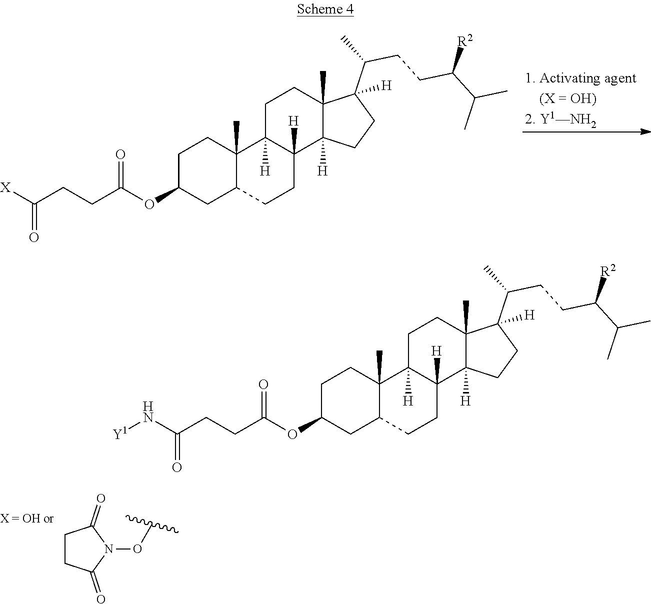

Compounds of Formula A2a can be prepared via the synthetic route outlined in Scheme 4. An appropriate reaction between cholesterol hemisuccinate or a cholesterol derivative hemisuccinate and an activating agent can be carried out under suitable conditions. The product of said reaction can be reacted with an amine under suitable conditions to give compounds of Formula A2a.

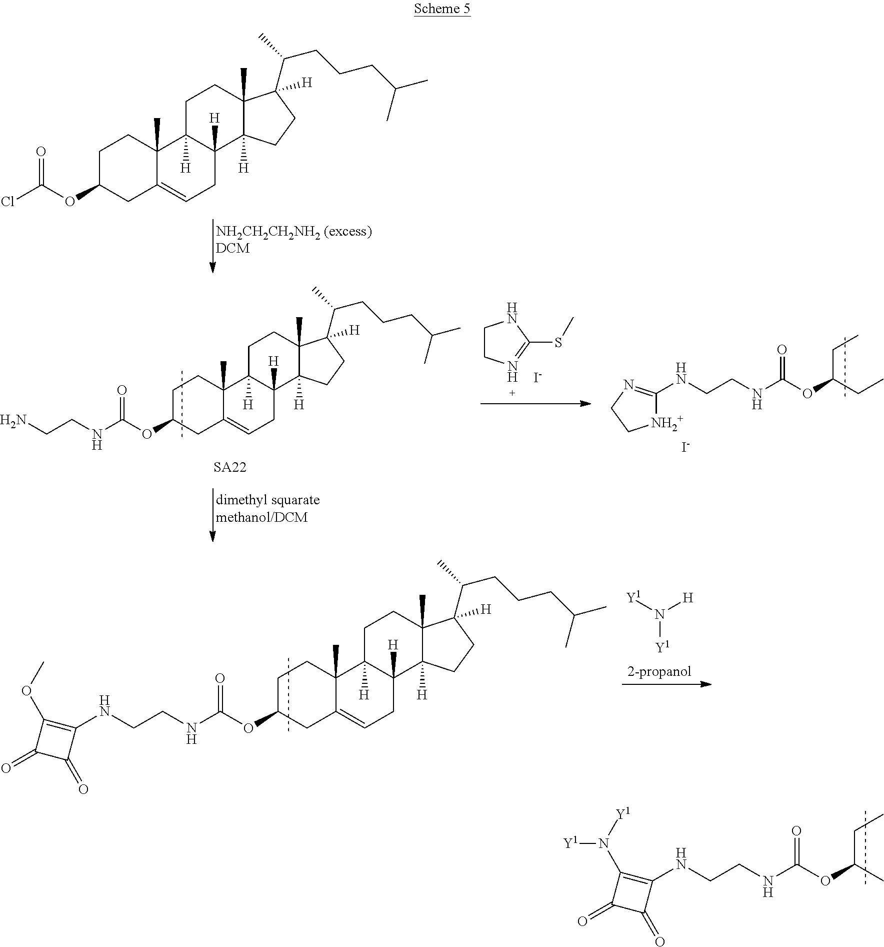

Compounds of Formula A2a can be prepared via the synthetic route outlined in Scheme 5. An appropriate reaction between cholesteryl chloroformate and ethane-1,2-diamine can be carried out under suitable conditions to give a SA22. SA22 can be reacted with 2-(methylthio)-4,5-dihydro-1H-imidazole hydroiodide under suitable conditions to give a compound of Formula A2a. SA22 can also be reacted with dimethyl squarate under suitable conditions, and the product of the reaction can be further reacted with a secondary amine under suitable conditions to give a compound of Formula A2a.

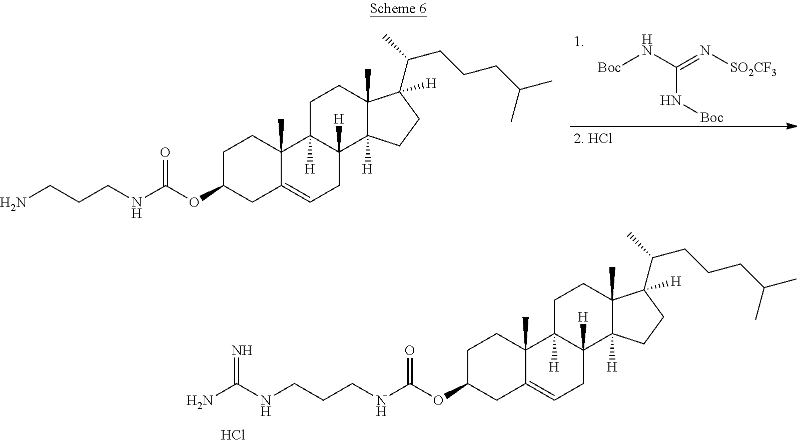

Compounds of Formula A2a can be prepared via the synthetic route outlined in Scheme 6. An appropriate reaction between an aminoalkyl carbamate and a guanidinylation agent can be carried out under suitable conditions. The product of said reaction can be reacted with HCl under suitable conditions to give a compound of Formula A2a.

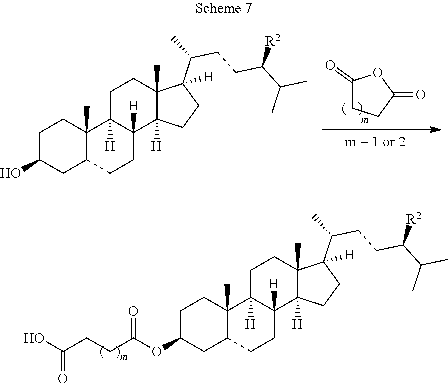

Precursors to compounds of Formula A2a can be prepared via the synthetic route outlined in Scheme 7. An appropriate reaction between cholesterol or a cholesterol derivative (such as stigmasterol) and can be carried out under suitable conditions (such as using triethylamine and 4-dimethylaminopyridine). The product of said reaction can be reacted with an amine under suitable conditions (such as using triethylamine) to give a precursor to a compound of Formula A2a.

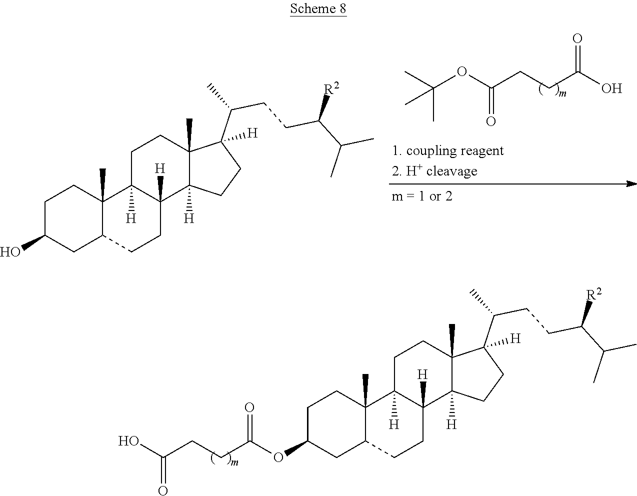

Precursors to compounds of Formula A2a can be prepared via the synthetic route outlined in Scheme 8. An appropriate reaction between cholesterol or a cholesterol derivative (such as stigmasterol) and a boc-hemiester can be carried out under suitable conditions. The product of said reaction can be reacted under suitable conditions to give a precursor to a compound of Formula A2a.

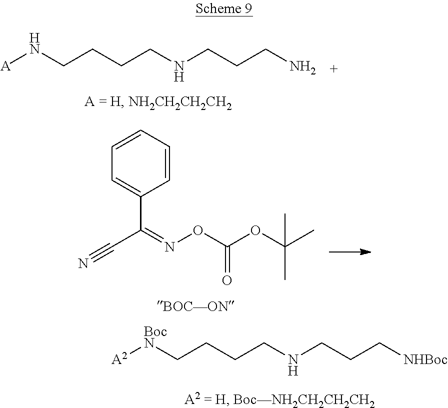

Intermediates for the synthesis of compounds of Formula A2a can be prepared via the synthetic route outlined in Scheme 9. An appropriate reaction between spermidine or spermine and (E)-N-((tert-butoxycarbonyl)oxy)benzimidoyl cyanide (BOC-ON) can be carried out under suitable conditions to give an intermediate for the synthesis of compounds of Formula A2a.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the present disclosure. Where a value is explicitly recited, it is to be understood that values which are about the same quantity or amount as the recited value are also within the scope of the present disclosure. Where a combination is disclosed, each subcombination of the elements of that combination is also specifically disclosed and is within the scope of the present disclosure. Conversely, where different elements or groups of elements are individually disclosed, combinations thereof are also disclosed. Where any element of an present disclosure is disclosed as having a plurality of alternatives, examples of that present disclosure in which each alternative is excluded singly or in any combination with the other alternatives are also hereby disclosed; more than one element of an present disclosure can have such exclusions, and all combinations of elements having such exclusions are hereby disclosed.

About: The term “about” as used in connection with a numerical value throughout the specification and the claims denotes an interval of accuracy, familiar and acceptable to a person skilled in the art. Such interval of accuracy is ±10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

Administered in combination: As used herein, the term “administered in combination” or “combined administration” means that two or more agents are administered to a subject at the same time or within an interval such that there can be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Contacting: As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal can be performed by varied routes of administration (e.g., intravenous, intramuscular, intradermal, and subcutaneous) and can involve varied amounts of nanoparticle compositions. Moreover, more than one mammalian cell can be contacted by a nanoparticle composition. A further example of contacting is between a nanoparticle and a cationic agent. Contacting a nanoparticle and a cationic agent can mean that the surface of the nanoparticle is put in physical connection with the cationic agent so that, the cationic agent can form a non-bonded interaction with the nanoparticle. In some embodiments, contacting a nanoparticle and a cationic agent intercalates the cationic agent into the nanoparticle, for example, starting at the surface of the nanoparticle. In some embodiments, the terms “layering,” “coating,” and “post addition” and “addition” can be used to mean “contacting” in reference to contacting a nanoparticle with a cationic agent

Delivering: As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route). Administration of a nanoparticle composition to a mammal or mammalian cell can involve contacting one or more cells with the nanoparticle composition.

Delivery Agent: As used herein, “delivery agent” refers to any substance that facilitates, at least in part, the in vivo, in vitro, or ex vivo delivery of a polynucleotide to targeted cells.

Diastereomer: As used herein, the term “diastereomer,” means stereoisomers that are not mirror images of one another and are non-superimposable on one another.

Disposed: As used herein, the term “disposed” means that a molecule formed a non-bonding interaction with a nanoparticle after the two were contacted with each other.

Dosing regimen: As used herein, a “dosing regimen” or a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats a protein deficiency (e.g., a CFTR deficiency), an effective amount of an agent is, for example, an amount of mRNA expressing sufficient CFTR to ameliorate, reduce, eliminate, or prevent the signs and symptoms associated with the CFTR deficiency, as compared to the severity of the symptom observed without administration of the agent. The term “effective amount” can be used interchangeably with “effective dose,” “therapeutically effective amount,” or “therapeutically effective dose.”

Enantiomer: As used herein, the term “enantiomer” means each individual optically active form of a compound of the present disclosure, having an optical purity or enantiomeric excess (as determined by methods standard in the art) of at least 80% (i.e., at least 90% of one enantiomer and at most 10% of the other enantiomer), at least 90%, or at least 98%.

Encapsulate: As used herein, the term “encapsulate” means to enclose, surround or encase.

Encapsulation Efficiency: As used herein, “encapsulation efficiency” refers to the amount of a polynucleotide that becomes part of a nanoparticle composition, relative to the initial total amount of polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency can be given as 97%. As used herein, “encapsulation” can refer to complete, substantial, or partial enclosure, confinement, surrounding, or encasement.

Epithelial Cells: As used herein, “epithelial cells” include cells derived from epithelium. Example epithelial cells are respiratory epithelial cells, nasal epithelial cells, alveolar epithelial cells, lung epithelial cells, or bronchial epithelial cells. In some embodiments, the epithelial cells are human bronchial epithelial (HBE) cells. In some embodiments, epithelial cells are in vitro cells. In some embodiments, epithelial cells are in vivo cells.

Expression: As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

Ex Vivo: As used herein, the term “ex vivo” refers to events that occur outside of an organism (e.g., animal, plant, or microbe or cell or tissue thereof). Ex vivo events can take place in an environment minimally altered from a natural (e.g., in vivo) environment.

Helper Lipid: As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In Vivo: As used herein, the term “in vivo” refers to events that occur within an organism (e.g., animal, plant, or microbe or cell or tissue thereof).

Ionizable amino lipid: The term “ionizable amino lipid” includes those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3) and (13Z,165Z)-N,N-dimethyl-3-nonydocosa-13-16-dien-1-amine (L608).

Isomer: As used herein, the term “isomer” means any tautomer, stereoisomer, enantiomer, or diastereomer of any compound of the present disclosure. It is recognized that the compounds of the present disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the present disclosure, the chemical structures depicted herein, and therefore the compounds of the present disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the present disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

Lipid nanoparticle core: As used herein, a lipid nanoparticle core is a lipid nanoparticle to which post addition layers of additional components can be added, such as a cationic agent and/or a PEG-lipid or other lipid. In some embodiments, the lipid nanoparticle core comprises: (i) an ionizable lipid, (ii) a phospholipid, (iii) a structural lipid, and (iv) optionally a PEG-lipid. In further embodiments, the lipid nanoparticle core comprises: (i) an ionizable lipid, (ii) a phospholipid, (iii) a structural lipid, and (iv) a PEG-lipid.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Lung Cells: As used herein, “lung cells” include cells derived from the lungs. Lungs cells can be, for example, lung epithelial cells, airway basal cells, bronchiolar exocrine cells, pulmonary neuroendocrine cells, alveolar cells, or airway epithelial cells. In some embodiments, lung cells are in vitro cells. In some embodiments, lung cells are in vivo cells.

Methods of Administration: As used herein, “methods of administration” can include intravenous, intramuscular, intradermal, subcutaneous, or other methods of delivering a composition to a subject. A method of administration can be selected to target delivery (e.g., to specifically deliver) to a specific region or system of a body.

The term “nucleic acid,” in its broadest sense, includes any compound and/or substance that comprises a polymer of nucleotides. These polymers are often referred to as polynucleotides. Exemplary nucleic acids or polynucleotides of the present disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

Patient: As used herein, “patient” refers to a subject who can seek or be in need of treatment, requires treatment, is receiving treatment, will receive treatment, or a subject who is under care by a trained professional for a particular disease or condition.

CFTR Associated Disease: As use herein the terms “CFTR-associated disease” or “CFTR-associated disorder” refer to diseases or disorders, respectively, which result from aberrant CFTR activity (e.g., decreased activity or increased activity). As a non-limiting example, cystic fibrosis is a CFTR associated disease. Numerous clinical variants of cystic fibrosis are known in the art. See, e.g., www.omim.org/entry/219700.

The terms “CFTR enzymatic activity,” “CFTR activity,” and “cystic fibrosis transmembrane conductance regulator activity” are used interchangeably in the present disclosure and refer to CFTR's ability to transport chloride ions through the cellular membrane. Accordingly, a fragment or variant retaining or having CFTR enzymatic activity or CFTR activity refers to a fragment or variant that has measurable chloride transport across the cell membrane.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “solvate,” as used herein, means a compound of the present disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an infection, disease, disorder and/or condition; partially or completely delaying onset of one or more signs and symptoms, features, or clinical manifestations of a particular infection, disease, disorder, and/or condition; partially or completely delaying onset of one or more signs and symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis” refers to a measure to produce

Salts: In some aspects, the pharmaceutical composition disclosed herein and comprises salts of some of their lipid constituents. The term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Sample: As used herein, the term “sample” or “biological sample” refers to a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). A sample further can include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which can contain cellular components, such as proteins or nucleic acid molecule.

Single unit dose: As used herein, a “single unit dose” is a dose of any therapeutic administered in one dose/at one time/single route/single point of contact, i.e., single administration event.

Split dose: As used herein, a “split dose” is the division of single unit dose or total daily dose into two or more doses.

Stereoisomer: As used herein, the term “stereoisomer” refers to all possible different isomeric as well as conformational forms that a compound can possess (e.g., a compound of any formula described herein), in particular all possible stereochemically and conformationally isomeric forms, all diastereomers, enantiomers and/or conformers of the basic molecular structure. Some compounds of the present disclosure can exist in different tautomeric forms, all of the latter being included within the scope of the present disclosure.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

Substantially: As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

Suffering from: An individual who is “suffering from” a disease, disorder, and/or condition has been diagnosed with or displays one or more signs and symptoms of the disease, disorder, and/or condition.

Susceptible to: An individual who is “susceptible to” a disease, disorder, and/or condition has not been diagnosed with and/or cannot exhibit signs and symptoms of the disease, disorder, and/or condition but harbors a propensity to develop a disease or its signs and symptoms. In some embodiments, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or other molecules of the present disclosure can be chemical or enzymatic.

Therapeutic Agent: The term “therapeutic agent” refers to an agent that, when administered to a subject, has a therapeutic, diagnostic, and/or prophylactic effect and/or elicits a desired biological and/or pharmacological effect. For example, in some embodiments, an mRNA encoding a CFTR polypeptide can be a therapeutic agent.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve signs and symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve signs and symptoms of, diagnose, prevent, and/or delay the onset of the infection, disease, disorder, and/or condition.

Total daily dose: As used herein, a “total daily dose” is an amount given or prescribed in 24 hr. period. The total daily dose can be administered as a single unit dose or a split dose.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more signs and symptoms or features of a disease, e.g., cystic fibrosis. For example, “treating” cystic fibrosis can refer to diminishing signs and symptoms associated with the disease, prolong the lifespan (increase the survival rate) of patients, reducing the severity of the disease, preventing or delaying the onset of the disease, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms).

The notation “C₁₋₁₄ alkyl” means a linear or branched, saturated hydrocarbon including 1-14 carbon atoms. An alkyl group can be optionally substituted.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond.

The notation “C₂₋₁₄ alkenyl” means a linear or branched hydrocarbon including 2-14 carbon atoms and at least one double bond. An alkenyl group can include one, two, three, four, or more double bonds. An alkenyl group can be optionally substituted.

As used herein, the term “carbocycle” or “carbocyclic group” means a mono- or multi-cyclic system including one or more rings of carbon atoms. Rings can be three, four, five, six, seven, eight, nine, ten, eleven, twelve, thirteen, fourteen, or fifteen membered rings.

The notation “C₃₋₆ carbocycle” means a carbocycle including a single ring having 3-6 carbon atoms. Carbocycles can include one or more double bonds and can be aromatic (e.g., aryl groups). Examples of carbocycles include cyclopropyl, cyclopentyl, cyclohexyl, phenyl, naphthyl, and 1,2-dihydronaphthyl groups. Carbocycles can be optionally substituted.

As used herein, the term “heterocycle” or “heterocyclic group” means a mono- or multi-cyclic system including one or more rings, where at least one ring includes at least one heteroatom. Heteroatoms can be, for example, nitrogen, oxygen, or sulfur atoms. Rings can be three, four, five, six, seven, eight, nine, ten, eleven, or twelve membered rings. Heterocycles can include one or more double bonds and can be aromatic (e.g., heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. Heterocycles can be optionally substituted.

As used herein, an “aryl group” is a carbocyclic group including one or more aromatic rings. Examples of aryl groups include phenyl and naphthyl groups.

As used herein, a “heteroaryl group” is a heterocyclic group including one or more aromatic rings. Examples of heteroaryl groups include pyrrolyl, furyl, thiophenyl, imidazolyl, oxazolyl, and thiazolyl. Both aryl and heteroaryl groups can be optionally substituted.

Alkyl, alkenyl, and cyclyl (e.g., carbocyclyl and heterocyclyl) groups can be optionally substituted unless otherwise specified. Optional substituents can be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR or OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)₂R″″, in which each OR are alkoxy groups that can be the same or different and R″″ is an alkyl or alkenyl group), a phosphate (e.g., P(O)₄³), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²), a sulfonyl (e.g., S(O)₂), an amide (e.g., C(O)NR₂, or N(R)C(O)R), an azido (e.g., N₃), a nitro (e.g., NO₂), a cyano (e.g., CN), an isocyano (e.g., NC), an acyloxy (e.g., OC(O)R), an amino (e.g., NR₂, NRH, or NH₂), a carbamoyl (e.g., OC(O)NR₂, OC(O)NRH, or —OC(O)NH₂), a sulfonamide (e.g., S(O)₂NR₂, S(O)₂NRH, S(O)₂NH₂, N(R)S(O)₂R, —N(H)S(O)₂R, N(R)S(O)₂H, or N(H)S(O)₂H), an alkyl group, an alkenyl group, and a cyclyl (e.g., carbocyclyl or heterocyclyl) group. R is an alkyl or alkenyl group, as defined herein.

As used herein, “comprises one to five primary, secondary, or tertiary amines or combination thereof” refers to alkyl, heterocycloalkyl, cycloalkyl, aryl, or heteroaryl groups that comprise, in addition to the other atoms, at least one nitrogen atom. The nitrogen atom is part of a primary, secondary, or tertiary amine group. The amine group can be selected from, but not limited to,

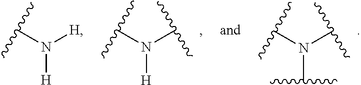

The primary, secondary, or tertiary amine can be part of a larger amine containing functional group selected from, but not limited to, —C(═N—)—N—, —C═C—N—, —C═N—, and —N—C(═N—)—N—.

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the present disclosure (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1

### Synthesis of Compounds According to Formula (I)

### A. General Considerations

All solvents and reagents used were obtained commercially and used as such unless noted otherwise. ¹H NMR spectra were recorded in CDCl₃, at 300 K using a Bruker Ultrashield 300 MHz instrument. Chemical shifts are reported as parts per million (ppm) relative to TMS (0.00) for ¹H. Silica gel chromatographies were performed on ISCO CombiFlash Rf+ Lumen Instruments using ISCO RediSep Rf Gold Flash Cartridges (particle size: 20-40 microns). Reverse phase chromatographies were performed on ISCO CombiFlash Rf+ Lumen Instruments using RediSep Rf Gold C18 High Performance columns. All final compounds were determined to be greater than 85% pure via analysis by reverse phase UPLC-MS (retention times, RT, in minutes) using Waters Acquity UPLC instrument with DAD and ELSD and a ZORBAX Rapid Resolution High Definition (RRHD) SB-C18 LC column, 2.1 mm, 50 mm, 1.8 μm, and a gradient of 65 to 100% acetonitrile in water with 0.1% TFA over 5 minutes at 1.2 mL/min. Injection volume was 5 μL and the column temperature was 80° C. Detection was based on electrospray ionization (ESI) in positive mode using Waters SQD mass spectrometer (Milford, Mass., USA) and evaporative light scattering detector.

The representative procedures described below are useful in the synthesis of Compounds 1-147.

The following abbreviations are employed herein:
THF: Tetrahydrofuran
DMAP: 4-Dimethylaminopyridine
LDA: Lithium Diisopropylamide
rt: Room Temperature
DME: 1,2-Dimethoxyethane
n-BuLi: n-Butyllithium

### B. Compound 2: Heptadecan-9-yl 8-((2-hydroxyethyl)(tetradecyl)amino) octanoate

### Representative Procedure 1

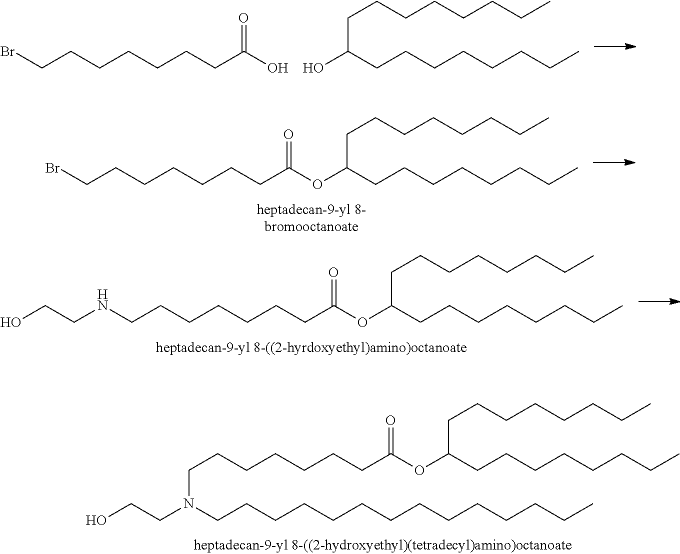

### Heptadecan-9-yl 8-bromooctanoate (Method A)

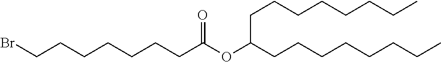 [SMILES: CCCCCCCCC(CCCCCCCC)OC(=O)CCCCCCCBr]

To a solution of 8-bromooctanoic acid (1.04 g, 4.6 mmol) and heptadecan-9-ol (1.5 g, 5.8 mmol) in dichloromethane (20 mL) was added N-(3-dimethylaminopropyl)-N′-ethylcarbodiimide hydrochloride (1.1 g, 5.8 mmol), N,N-diisopropylethylamine (3.3 mL, 18.7 mmol) and DMAP (114 mg, 0.9 mmol). The reaction was allowed to stir at rt for 18 h. The reaction was diluted with dichloromethane and washed with saturated sodium bicarbonate. The organic layer was separated and washed with brine, and dried over MgSO4. The organic layer was filtered and evaporated in vacuo. The residue was purified by silica gel chromatography (0-10% ethyl acetate in hexanes) to obtain heptadecan-9-yl 8-bromooctanoate (875 mg, 1.9 mmol, 41%).

¹H NMR (300 MHz, CDCl₃) δ: ppm 4.89 (m, 1H); 3.42 (m, 2H); 2.31 (m, 2H); 1.89 (m, 2H); 1.73-1.18 (br. m, 36H); 0.88 (m, 6H).

### Heptadecan-9-yl 8-((2-hydroxyethyl)amino)octanoate (Method B)

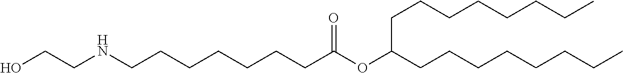 [SMILES: CCCCCCCCC(CCCCCCCC)OC(=O)CCCCCCCNCCO]

A solution of heptadecan-9-yl 8-bromooctanoate (3.8 g, 8.2 mmol) and 2-aminoethan-1-ol (15 mL, 248 mmol) in ethanol (3 mL) was allowed to stir at 62° C. for 18 h. The reaction mixture was concentrated in vacuo and the residue was taken-up in ethyl acetate and water. The organic layer was separated and washed with water, brine and dried over Na2SO₄. The mixture was filtered and evaporated in vacuo. The residue was purified by silica gel chromatography (0-100% (mixture of 1% NH₄OH, 20% MeOH in dichloromethane) in dichloromethane) to obtain heptadecan-9-yl 8-((2-hydroxyethyl)amino)octanoate (3.1 g, 7 mmol, 85%). UPLC/ELSD: RT=2.67 min. MS (ES): m/z (MH⁺) 442.68 for C₂₇H₅₅NO₃

¹H NMR (300 MHz, CDCl₃) δ: ppm 4.89 (p, 1H); 3.67 (t, 2H); 2.81 (t, 2H); 2.65 (t, 2H); 2.30 (t, 2H); 2.05 (br. m, 2H); 1.72-1.41 (br. m, 8H); 1.40-1.20 (br. m, 30H); 0.88 (m, 6H).

### Heptadecan-9-yl 8-((2-hydroxyethyl)(tetradecyl)amino)octanoate (Method C)

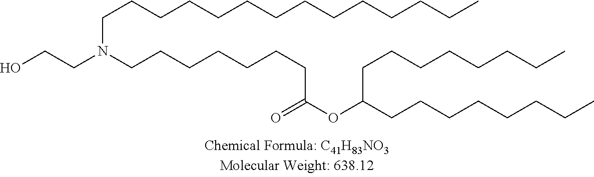 [SMILES: CCCCCCCCCCCCCCN(CCO)CCCCCCCC(=O)OC(CCCCCCCC)CCCCCCCC]

A solution of heptadecan-9-yl 8-((2-hydroxyethyl)amino)octanoate (125 mg, 0.28 mmol), 1-bromotetradecane (94 mg, 0.34 mmol) and N,N-diisopropylethylamine (44 mg, 0.34 mmol) in ethanol was allowed to stir at 65° C. for 18 h. The reaction was cooled to rt and solvents were evaporated in vacuo. The residue was taken-up in ethyl acetate and saturated sodium bicarbonate. The organic layer was separated, dried over Na2SO₄ and evaporated in vacuo. The residue was purified by silica gel chromatography (0-100% (mixture of 1% NH₄OH, 20% MeOH in dichloromethane) in dichloromethane) to obtain heptadecan-9-yl 8-((2-hydroxyethyl)(tetradecyl)amino)octanoate (89 mg, 0.14 mmol, 50%). UPLC/ELSD: RT=3.61 min. MS (ES): m/z (MH⁺) 638.91 for C₄₁H₈₃NO₃. ¹H NMR (300 MHz, CDCl₃) δ: ppm 4.86 (p, 1H); 3.72-3.47 (br. m, 2H); 2.78-2.40 (br. m, 5H); 2.28 (t, 2H); 1.70-1.40 (m, 10H); 1.38-1.17 (br. m, 54H); 0.88 (m, 9H).

### SYNTHESIS OF INTERMEDIATES

### Intermediate A: 2-Octyldecanoic acid

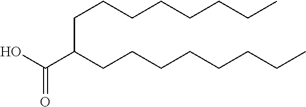 [SMILES: CCCCCCCCC(CCCCCCCC)C(=O)O]

A solution of diisopropylamine (2.92 mL, 20.8 mmol) in THE (10 mL) was cooled to −78° C. and a solution of n-BuLi (7.5 mL, 18.9 mmol, 2.5 M in hexanes) was added. The reaction was allowed to warm to 0° C. To a solution of decanoic acid (2.96 g, 17.2 mmol) and NaH (754 mg, 18.9 mmol, 60% w/w) in THF (20 mL) at 0° C. was added the solution of LDA and the mixture was allowed to stir at rt for 30 min. After this time 1-iodooctane (5 g, 20.8 mmol) was added and the reaction mixture was heated at 45° C. for 6 h. The reaction was quenched with 1N HCl (10 mL). The organic layer was dried over MgSO₄, filtered and evaporated in vacuo. The residue was purified by silica gel chromatography (0-20% ethyl acetate in hexanes) to yield 2-octyldecanoic acid (1.9 g, 6.6 mmol, 38%). ¹H NMR (300 MHz, CDCl₃) δ: ppm 2.38 (br. m, 1H); 1.74-1.03 (br. m, 28H); 0.91 (m, 6H).

### Intermediate B: 7-Bromoheptyl 2-octyldecanoate

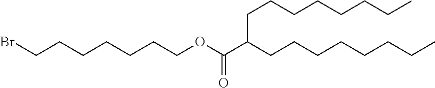 [SMILES: CCCCCCCCC(CCCCCCCC)C(=O)OCCCCCCCBr]

7-bromoheptyl 2-octyldecanoate was synthesized using Method A from 2-octyldecanoic acid and 7-bromoheptan-1-ol. ¹H NMR (300 MHz, CDCl₃) δ: ppm 4.09 (br. m, 2H); 3.43 (br. m, 2H); 2.48-2.25 (br. m, 1H); 1.89 (br. m, 2H); 1.74-1.16 (br. m, 36H); 0.90 (m, 6H).

### Intermediate C: (2-Hexylcyclopropyl)methanol

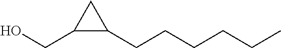

A solution of diethyl zinc (20 mL, 20 mmol, 1 M in hexanes), in dichloromethane (20 mL) was allowed to cool to −40° C. for 5 min. Then a solution of diiodomethane (3.22 mL, 40 mmol) in dichloromethane (10 mL) was added dropwise. After the reaction was allowed to stir for 1 h at −40° C., a solution of trichloro-acetic acid (327 mg, 2 mmol) and DME (1 mL, 9.6 mmol) in dichloromethane (10 mL) was added. The reaction was allowed to warm to −15° C. and stir at this temperature for 1 h. A solution of (Z)-non-2-en-1-ol (1.42 g, 10 mmol) in dichloromethane (10 mL) was then added to the −15° C. solution. The reaction was then slowly allowed to warm to rt and stir for 18 h. After this time saturated NH₄Cl (200 mL) was added and the reaction was extracted with dichloromethane (3×), washed with brine, and dried over Na₂SO₄. The organic layer was filtered, evaporated in vacuo and the residue was purified by silica gel chromatography (0-50% ethyl acetate in hexanes) to yield (2-hexylcyclopropyl)methanol (1.43 g, 9.2 mmol, 92%). ¹H NMR (300 MHz, CDCl₃) δ: ppm 3.64 (m, 2H); 1.57-1.02 (m, 12H); 0.99-0.80 (m, 4H); 0.72 (m, 1H), 0.00 (m, 1H).

### C. Compound 18: Heptadecan-9-yl 8-((2-hydroxyethyl)(8-(nonyloxy)-8-oxooctyl)amino) octanoate

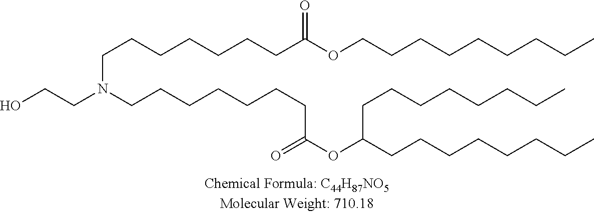 [SMILES: CCCCCCCCCOC(=O)CCCCCCCN(CCO)CCCCCCCC(=O)OC(CCCCCCCC)CCCCCCCC]

Compound 18 was synthesized according to the general procedure and Representative Procedure 1 described above.

UPLC/ELSD: RT=3.59 min. MS (ES): m/z (MH⁺) 710.89 for C₄₄H₈₇NO₅. ¹H NMR (300 MHz, CDCl₃) δ: ppm 4.86 (m, 1H); 4.05 (t, 2H); 3.53 (br. m, 2H); 2.83-2.36 (br. m, 5H); 2.29 (m, 4H); 0.96-1.71 (m, 64H); 0.88 (m, 9H).

### D. Compound 136: Nonyl 8-((2-hydroxyethyl)((9Z,12Z)-octadeca-9,12-dien-1-yl)amino)octanoate

### Representative Procedure 2

### Nonyl 8-bromooctanoate (Method A)

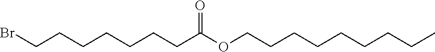 [SMILES: CCCCCCCCCOC(=O)CCCCCCCBr]

To a solution of 8-bromooctanoic acid (5 g, 22 mmol) and nonan-1-ol (6.46 g, 45 mmol) in dichloromethane (100 mL) were added N-(3-Dimethylaminopropyl)-N-ethylcarbodiimide hydrochloride (4.3 g, 22 mmol) and DMAP (547 mg, 4.5 mmol). The reaction was allowed to stir at rt for 18 h. The reaction was diluted with dichloromethane and washed with saturated sodium bicarbonate. The organic layer was separated and washed with brine, dried over MgSO4. The organic layer was filtered and evaporated under vacuum. The residue was purified by silica gel chromatography (0-10% ethyl acetate in hexanes) to obtain nonyl 8-bromooctanoate (6.1 g, 17 mmol, 77%).

¹H NMR (300 MHz, CDCl₃) δ: ppm 4.06 (t, 2H); 3.40 (t, 2H); 2.29 (t, 2H); 1.85 (m, 2H); 1.72-0.97 (m, 22H); 0.88 (m, 3H).

### Nonyl 8-((2-hydroxyethyl)amino)octanoate

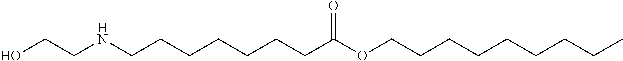 [SMILES: CCCCCCCCCOC(=O)CCCCCCCNCCO]

A solution of nonyl 8-bromooctanoate (1.2 g, 3.4 mmol) and 2-aminoethan-1-ol (5 mL, 83 mmol) in ethanol (2 mL) was allowed to stir at 62° C. for 18 h. The reaction mixture was concentrated in vacuum and the residue was extracted with ethyl acetate and water. The organic layer was separated and washed with water, brine and dried over Na₂SO₄. The organic layer was filtered and evaporated in vacuo. The residue was purified by silica gel chromatography (0-100% (mixture of 1% NH₄OH, 20% MeOH in dichloromethane) in dichloromethane) to obtain nonyl 8-((2-hydroxyethyl)amino)octanoate (295 mg, 0.9 mmol, 26%).

UPLC/ELSD: RT=1.29 min. MS (ES): m/z (MH⁺) 330.42 for C₁₉H₃₉NO₃

¹H NMR (300 MHz, CDCl₃) δ: ppm 4.07 (t, 2H); 3.65 (t, 2H); 2.78 (t, 2H); 2.63 (t, 2H); 2.32-2.19 (m, 4H); 1.73-1.20 (m, 24H); 0.89 (m, 3H)

### Nonyl 8-((2-hydroxyethyl)((9Z,12Z)-octadeca-9,12-dien-1-yl)amino)octanoate

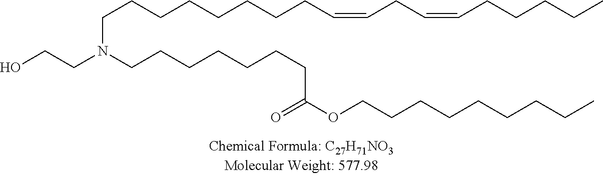 [SMILES: CCCCC/C=C\C/C=C\CCCCCCCCN(CCO)CCCCCCCC(=O)OCCCCCCCCC]

A solution of nonyl 8-((2-hydroxyethyl)amino)octanoate (150 mg, 0.46 mmol), (6Z,9Z)-18-bromooctadeca-6,9-diene (165 mg, 0.5 mmol) and N,N-diisopropylethylamine (65 mg, 0.5 mmol) in ethanol (2 mL) was allowed to stir at reflux for 48 h. The reaction was allowed to cool to rt and solvents were evaporated under vacuum. The residue was purified by silica gel chromatography (0-10% MeOH in dichloromethane) to obtain nonyl 8-((2-hydroxyethyl)((9Z,12Z)-octadeca-9,12-dien-1-yl)amino)octanoate (81 mg, 0.14 mmol, 30%) as a HBr salt.

UPLC/ELSD: RT=3.24 min. MS (ES): m/z (MH⁺) 578.64 for C₃₇H₇₁NO₃

¹H NMR (300 MHz, CDCl₃) δ: ppm 10.71 (br., 1H); 5.36 (br. m, 4H); 4.04 (m, 4H); 3.22-2.96 (br. m, 5H); 2.77 (m, 2H); 2.29 (m, 2H); 2.04 (br. m, 4H); 1.86 (br. m, 4H); 1.66-1.17 (br. m, 40H); 0.89 (m, 6H)

### E. Compound 138: Dinonyl 8,8′-((2-hydroxyethyl)azanediyl)dioctanoate

### Representative Procedure 3

### Dinonyl 8,8′-((2-hydroxyethyl)azanediyl)dioctanoate

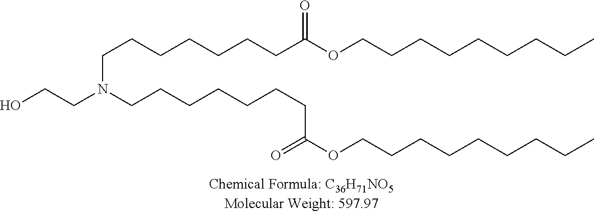 [SMILES: CCCCCCCCCOC(=O)CCCCCCCN(CCO)CCCCCCCC(=O)OCCCCCCCCC]

A solution of nonyl 8-bromooctanoate (200 mg, 0.6 mmol) and 2-aminoethan-1-ol (16 mg, 0.3 mmol) and N, N-diisopropylethylamine (74 mg, 0.6 mmol) in THF/CH₃CN (1:1) (3 mL) was allowed to stir at 63° C. for 72 h. The reaction was cooled to rt and solvents were evaporated under vacuum. The residue was extracted with ethyl acetate and saturated sodium bicarbonate. The organic layer was separated, dried over Na₂SO₄ and evaporated under vacuum. The residue was purified by silica gel chromatography (0-10% MeOH in dichloromethane) to obtain dinonyl 8,8′-((2-hydroxyethyl)azanediyl)dioctanoate (80 mg, 0.13 mmol, 43%).

UPLC/ELSD: RT=3.09 min. MS (ES): m/z (MH⁺) 598.85 for C₃₆H₇₁NO₅

¹H NMR (300 MHz, CDCl₃) δ: ppm 4.05 (m, 4H); 3.57 (br. m, 2H); 2.71-2.38 (br. m, 6H); 2.29 (m, 4H), 1.71-1.01 (br. m, 49H), 0.88 (m, 6H).

All other compounds of Formula (I) of this disclosure can be obtained by a method analogous to Representative Procedures 1-3 as described above.

### Example 2

### Production of Nanoparticle Compositions

Lipid nanoparticle cores were prepared using ethanol drop nanoprecipitation followed by solvent exchange into an aqueous buffer using a desalting chromatography column. An exemplary lipid nanoparticle can be prepared by a process where lipids were dissolved in ethanol at concentration of 15.4 mM and molar ratios of 50:10:38.5:1.5 (ionizable lipid:DSPC:cholesterol:DMG-PEG2K lipid) and mixed with mRNA at a concentration of 0.1515 mg/mL diluted in 25 mM sodium acetate pH 5.0. The N:P ratio was set to 5.8 in each formulation. The lipid solution and mRNA were mixed using a micro-tee mixer at a 1:3 volumetric ratio of lipid:mRNA. Once the nanoparticles were formed, they underwent solvent exchange over a desalting chromatography column preconditioned with 1×PBS buffer at pH 7.0. The elution profile of the nanoparticle was captured by UV, pH, and conductivity detectors. The UV profile was used to collect the solvent-exchanged nanoparticles. The resulting nanoparticle suspension underwent concentration using Amicon ultra-centrifugal filters and was passed through a 0.22 μm syringe filter. The nanoparticles were prepared to a specific concentration.

GL67 was added to the nanoparticle core by dissolving GL67 in macrogol (15)-hydroxy stearate, Kolliphor® HS15 (HS15) and post-added to LNP at a mass ratio of 1.25 (GL67 to mRNA). Specifically, 3HCl-GL67 was dissolved directly in HS15 (1 mg/mL, ˜70 μM, water) to generate initial stock solution at 5 mg/mL (6.92 mM), which could be in micellar form in solution. GL67 at 5 mg/mL was further diluted ([GL67] required for post-addition (PA) at a specific GL67:mRNA weight ratio) with HS15 (1 mg/mL) and added to LNPs (1:1 by volume) at ambient temperature via simple mixing: [mRNA] 0.2 mg/mL, [3HCl-GL67] 0.25 mg/mL, [HS15] 0.5 mg/mL, [PBS] 0.5×. LNPs further diluted with 1×PBS (1:1 by volume):

[mRNA] 0.1 mg/mL, [3HCl-GL67] 0.125 mg/mL, [HS15] 0.5 mg/mL, [PBS] 0.75×.

An example LNP core, designated LNP-1a is as follow:

LNP-1a

| Components | mass (mg) | MW (g/mol) | Core LNP mol % |
|---|---|---|---|
| Compound 18 of | 11.99 | 710.18 | 50.0% |
| Example 1 | | | |
| DSPC | 2.67 | 790.15 | 10.0% |
| Cholesterol | 5.02 | 386.65 | 38.5% |
| DMG-PEG 2k | 1.27 | 2500 | 1.5% |
| mRNA | 1 | — | — |

An example LNP as described, designated LNP-1 is as follows:

LNP-1

| | | MW | Core LNP | LNP-1 | LNP-1 |
|---|---|---|---|---|---|
| Components | mass (mg) | (g/mol) | mol % | mol % | mass % |
| Compound | 11.99 | 710.18 | 50.0% | 47.6% | 51.7% |
| 18 of | | | | | |
| Example 1 | | | | | |
| DSPC | 2.67 | 790.15 | 10.0% | 9.5% | 11.5% |
| Cholesterol | 5.02 | 386.65 | 38.5% | 36.6% | 21.6% |
| DMG-PEG | 1.27 | 2500 | 1.5% | 1.4% | 5.5% |
| 2k | | | | | |
| GL67•3HCl | 1.25 | 724 | — | 4.9% | 5.4% |
| HS 15 | 0.25 | — | — | — | — |
| mRNA | 1 | — | — | — | 4.3% |

HS15 has a MW of 960-1900, with average MW of 1430.

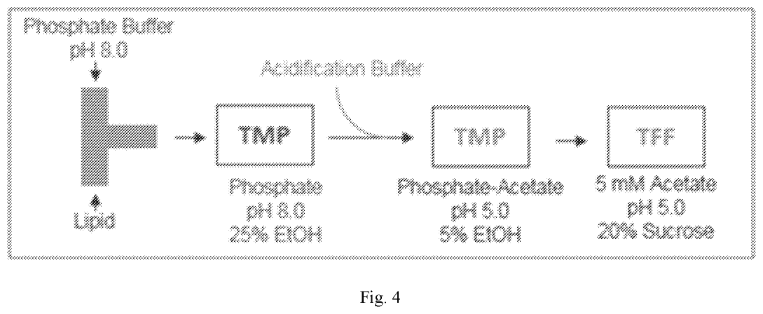

Exemplary LNP (without GL67) can be prepared according to the schematic in FIGS. 1-3. FIG. 1 refers to post-hoc loading (PHL) process of generating an empty lipid nanoparticle and the solution containing nucleic acid is then added to an empty-LNP. FIG. 2 refers to post-insertion/post-addition (PHL-PIPA) process refers to adding PEG lipid to a lipid nanoparticle. FIG. 3 refers to second generation post-hoc loading process, which includes post-insertion/post-addition of PEG steps. FIG. 4 refers to empty lipid nanoparticle prototype (“Neutral assembly”), where the empty LNP is mixed at pH 8.0 and the final formulation is pH 5.0.

### Example 3

### Small-Angle x-Ray Scattering (SAXS)

The x-ray scattering experiments were performed using an in-house small-angle x-ray scattering instrument, SAXS point 2.0, from Anton Paar. The LNPs were typically in the mRNA concentration range from 0.5 to 1 mg/mL which were loaded into a quartz capillary with 1 mm in diameter. X-rays of wavelength of 0.154 nm were generated from a Primux 100 micro x-ray source. The scattered intensity was measured using a two-dimensional (2D) EIGER R series CMOS detector from DECTRIS at a sample to detector distance of 575 mm. The 2D data was then circularly averaged, yielding the one-dimensional (1D) profile q ranging from 0.06 nm⁻¹ to 4 nm⁻¹, where

$$q [ q = \frac{4 π}{λ} sin ( \frac{θ}{2} ) ]$$

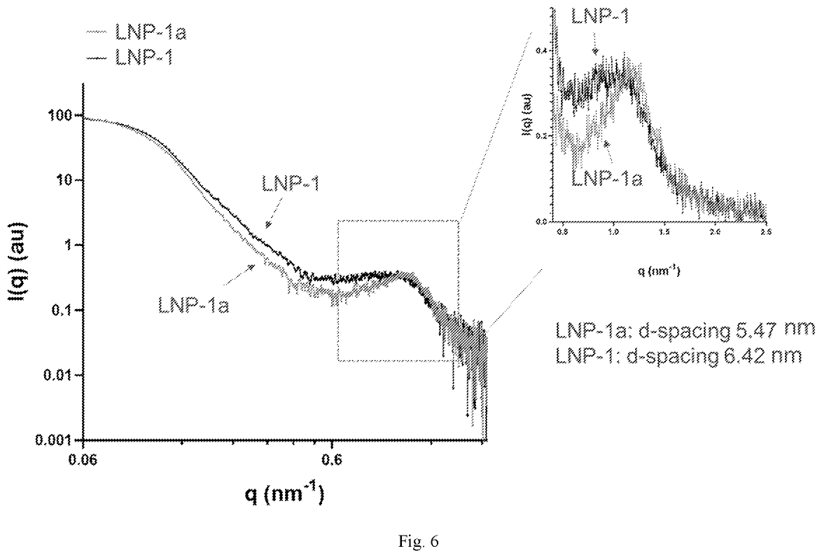

is the wave vector, with λ and θ being the wavelength and scattering angle, respectively. The 1D data was further corrected for sample transmission and buffer background. LNP-1 prepared according to Example 3 has d-spacing of 6.42 nm. LNP-1a has a d-spacing of 5.47 nm. FIG. 6 shows the graph of 1(q) (au) versus q (nm-1) of LNP-1 and LNP-1a.

### Example 4

### Generalized Polarization (GP)

Laurdan (6-dodecanoyl-2-dimethylaminonaphthane) was pre-dissolved in dimethyl sulfoxide (DMSO) at a concentration of 0.075 mg/mL. The Laurdan/DMSO solution was then added to LNP solutions at 0.18 mg/mL lipid concentration at a DMSO to aqueous buffer volume ratio at 1:500. For the control experiments, DMSO instead of Laurdan/DMSO was added to the LNP solution following the same protocol. The Laurdan dyes were allowed to incubate with the LNP solution for three hours. The fluorescence intensities at 435 and 490 nm were collected with an excitation wavelength at 340 nm using the MicroMax 384 Microwell-plate reader that is connected to an in-house fluorescence spectrometer, FluoroMax-4, from Horiba. The generalized polarization (GP) was calculated on the basis of the following equation:

$$G P = \frac{{I}_{435} - {I}_{490}}{{I}_{435} + {I}_{490}}$$

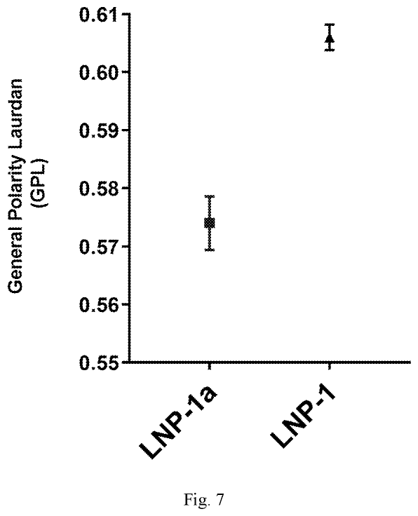

FIG. 7 shows the general polarity laurdan (GPL) values for LNP-1 prepared according to Example 3 and LNP-1a.

### Example 5

### Percent mRNA Encapsulation

Encapsulation efficiency (EE %) was measured using a modified Quant-iT RiboGreen assay. To determine the EE %, nanoparticles (or PBS, blank) were diluted in 1×TE to achieve a concentration of 2-4 μg/mL mRNA per well. These samples were aliquoted and diluted 1:1 in 1×TE or 1×TE with 2.5 mg/mL heparin buffer (measuring free mRNA) or TE buffer with 2% Triton X-100 or 2% Triton with 2.5 mg/mL heparin (measuring total mRNA). Quant-iT RiBogreen reagent was added and fluorescent signal was quantified using a plate reader. Encapsulation efficiency was calculated as follows:

$$EE \% = ( 1 - \frac{Free mRNA}{Total mRNA} ) ⋆ 100$$

Total mRNA: quantification of the total amount of mRNA by dissolving the particles with the detergent Triton (TX) with or without heparin.
Free mRNA: quantification of the amount of mRNA that is not encapsulated by diluting the particles in TE (Tris+EDTA buffer) with or without heparin.
Heparin is an anionic glycosaminoglycan, which competes with the sterol amine for the mRNA, and is used to quantify the amount of mRNA in LNP with a cationic agent such as sterol amine.
LNP-1 prepared according to Example 2 has 98% encapsulated mRNA.

### Example 6

### LNP Cellular Uptake and Protein Expression in Healthy Human Bronchial Epithelial Cell Models

To evaluate LNP cellular uptake and protein expression in healthy human bronchial epithelial cells (HBE), the EpiAirway model from MatTek (Ashland, Mass.) a ready-to-use 3D tissue model is used. The model consists of human-derived tracheal/bronchial epithelial cells from healthy donors.

The cells are plated on 24 mm transwells inserts with a pore size of 0.4 μm, and upon developing a confluent monolayer, media is removed from the apical chamber, with cultures being kept at the air-liquid interface (ALI) for up to 4 weeks to achieve complete cell differentiation and pseudo-stratification. The model recapitulates in vivo phenotypes of mucociliary barriers and exhibits human relevant tissue structure and cellular morphology, with a 3D structure consisting of organized Keratin 5+ basal cells, mucus producing goblet cells, functional tight junctions and beating cilia.

### Accumulation and Expression Assay in Healthy HBE Cells

LNPs incorporating 0.1 mole % Rhodamine-DOPE and encapsulating NPI-Luc reporter mRNA were dosed apically in healthy HBE in Hyclone Phosphate Buffered Saline. The cells were washed with 1 mM DTT in PBS for 10 min prior to LNP addition to remove the mucus accumulated during post-ALI differentiation. The NPI-Luc reporter includes a nuclear localization sequence and multiple V5 tags at N-terminus for enhanced detection sensitivity of expressed protein molecules. LNP transfected cells were incubated 4-72 h, after that the cells were detached from membranes using trypsin EDTA and fixed in suspension with 4% PFA in PBS.

Cells were processed separately for LNP accumulation and protein expression. To quantify LNP accumulation, PFA fixed cells were transferred in 96 well Cell Carrier Ultra plates (PerkinElmer) with optically-clear cyclic olefin bottom for high content analysis, and imaged using Opera Phenix spinning disk confocal microscope (PerkinElmer). Cell were detected using DAPI (405 nm channel), and LNP accumulation was detected using the Rhodamine-DOPE (561 nm channel). Image analysis was performed in Harmony 4.8, using spot segmentation in the 561 nm channel to quantify LNP accumulation in endocytic organelles, and to derive % cells positive for LNP uptake as wells as LNP accumulation per cell.

To quantify protein expression, PFA fixed cells were transferred in 96 well v-bottom plates and processed for immunofluorescence (IF) using an anti-V5 rabbit monoclonal antibody. Briefly, the cells were permeabilized with 0.5% TX-100 for 5 min, blocked with 1% bovine serum albumin (BSA) in PBS for 30 min, followed by incubation with anti-V5 primary antibody for 1 h at room temperature, and Alexa 488 conjugated secondary antibody for 30 min. Between the different incubation steps the cells were spun down and washed by resuspension in PBS. Following anti-V5 IF staining, the cells were transferred in 96 well Cell Carrier Ultra plates for imaging with the Opera Phenix, NPI-Luc expression was detected was using the 488 nm channel. Image analysis was performed in Harmony 4.8, with mean nuclear intensity in the 488 nm channel being used to derive % cells positive for protein expression and protein expression per cell.

### Example 7

### Protein Expression in Human Cervical Cancer Epithelial Cell (HeLa) Model

To evaluate protein expression In Vitro, HeLa cells from ATCC.org (ATCC CCL-2) are used. The cells are cultured in complete Minimum Essential Medium (MEM) and are plated in 96 well Cell Carrier Ultra plate with PDL coated surface (PerkinElmer) prior to running an experiment.

### Expression Assay in HeLa Cells

LNPs encapsulating NPI-Luc mRNA were dosed with MEM media in the absence of serum. LNP transfected cell were incubated for 5 h post LNP transfection, the cells were imaged live using Opera Phoenix spinning disk confocal microscope (PerkinElmer). Cells were detected using DAPI (405 nm channel), and image analysis was performed in Harmony 4.9, to quantify the number of cells. After imaging the cells were processed with One-Glo Luciferase assay (Promega) to quantify protein expression. Results were reported in relative luminescence units (RLU) normalized to cells counts.

### Example 8

### Production of Nanoparticle Compositions Using a Post-Hoc Approach

Exemplary empty lipid nanoparticles can be prepared by a process where lipids were dissolved in ethanol at concentration of 40 mM and molar ratios of 50.5:10.1:38.9:0.5 (ionizable lipid:DSPC:cholesterol:DMG-PEG2K lipid) and mixed with 7.15 mM sodium acetate pH 5.0. The lipid solution and buffer were mixed using a multi-inlet vortex mixer at a 3:7 volumetric ratio of lipid:buffer. After a 5 second residence time, the eLNPs were mixed with 5 mM sodium acetate pH 5.0 at a volumetric ratio of 5:7 of eLNP:buffer. The dilute eLNPs were then buffer exchanged and concentrated using tangential flow filtration into a final buffer containing 5 mM sodium acetate pH 5.0 and a sucrose solution was subsequently added to complete the storage matrix. mRNA loading into the eLNP took place using the PHL process. An exemplary mRNA-loaded nanoparticle can be prepared by mixing eLNP at a lipid concentration of 2.85 mg/mL with mRNA at a concentration of 0.25 mg/mL in 42.5 mM sodium acetate pH 5.0. The N:P ratio was set to 4.93 in each formulation. The eLNP solution and mRNA were mixed using a multi-inlet vortex mixer at a 3:2 volumetric ratio of eLNP:mRNA. Once the eLNP were loaded with mRNA, they underwent a 30 s-60 s residence time prior to mixing in-line with a buffer containing 120 mM TRIS pH 8.12 at a volumetric ratio of 5:1 of nanoparticle:buffer. After this addition step, the nanoparticle formulation was mixed in-line with a buffer containing 20 mM TRIS, 0.352 mg/mL DMG-PEG2k, 0.625 mg/mL GL-67, pH 7.5 at a volumetric ratio of 6:1 of nanoparticle:buffer. The resulting nanoparticle suspension underwent concentration using tangential flow filtration and was diluted with a salt solution to a final buffer matrix containing 70 mM NaCl. The resulting nanoparticle suspension was filtered through a 0.8/0.2 μm capsule filter and filled into glass vials a mRNA strength of 0.5-2 mg/mL.

### Example 9

### Synthesis of Sterol Amines

### A. Compound SA1: (3S,8S,9S,10R,13R,14S,17R)—N,N,10,13-tetramethyl-17-((R)-6-methylheptan-2-yl)-2,3,4,7,8,9,10,11,12,13,14,15,16,17-tetradecahydro-1H-cyclopenta[a]phenanthren-3-amine

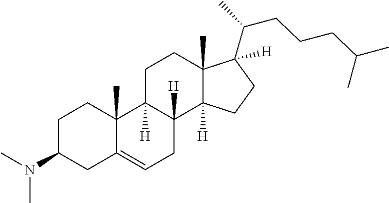

SA1 was prepared as described in Justus Liebigs Annalen der Chemie, 663, 135-49 (1963)

### B. Compound SA2: 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-N,N-dimethylethan-1-amine

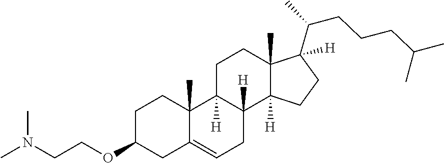

SA2 was prepared as described in Biochem. and Biophys. Res. Communications, 6, 359 (1961).

### C. Compound SA3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate, trihydrochloride salt

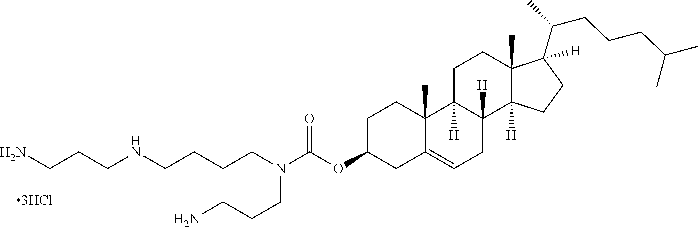

SA3 was purchased from Avanti Polar Lipids, Inc. (Alabaster, Ala.).

### D. Compound SA4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(dimethylamino)ethyl)carbamate, hydrochloride salt

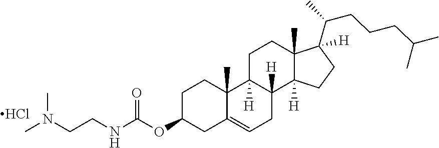

SA4 was purchased from Avanti Polar Lipids, Inc. (Alabaster, Ala.).

### E. Compound SA5: 4-((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)methyl)-1H-imidazole

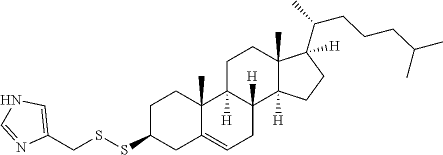

SA5 was prepared as described in US Patent Application 20140288160 (“Cleavable Lipids”).

### F. Compound SA6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate

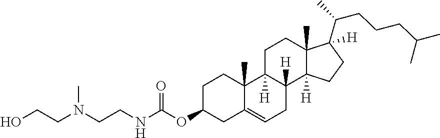

To a stirred solution of 2-[(2-aminoethyl)(methyl)amino]ethanol (53 mg, 0.44 mmol) in 1 mL dry DCM under dry nitrogen at 0° C. was added a solution of cholesteryl chloroformate (100 mg, 0.22 mmol) in 2 mL dry DCM dropwise over five minutes. The reaction was allowed to slowly warm to room temp and stirred overnight after which no starting material remained by LCMS. The white mixture was concentrated, and the residue purified by silica gel chromatography (100% DCM going to 20% DCM/80% DCM/MeOH/NH₄OH (80:20:1)) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate (57 mg, 0.11 mmol, 48%) as a white solid. MS (ES): m/z (MH⁺) 531.5 for C₃₆H₆₄N₂O₂. ¹H NMR (300 MHz, CDCl₃) δ: ppm 5.36 (d, 1H, J=5.1 Hz); 5.02 (t, 1H, J=5.3 Hz); 4.48 (m, 1H); 3.61 (t, 2H, J=5.3 Hz); 3.27 (q, 2H, J=5.5 Hz, 11.2 Hz); 2.69 (br. s, 4H); 2.55 (m, 4H); 2.42-2.16 (m, 5H); 2.10-1.71 (m, 5H); 1.63-1.22 (m, 11H); 1.21-0.95 (m, 13H); 0.90 (d, 3H, J=6.5 Hz); 0.88 (dd, 6H, J=1.2 Hz, 6.6 Hz); 0.66 (s, 3H).

### G. Compound SA7: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(4-methylpiperazin-1-yl)ethyl)carbamate

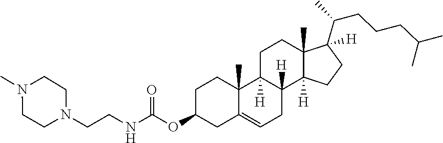

SA7 was prepared in the same manner as SA6 but using 2-(4-methylpiperazin-1-yl)ethanamine instead of 2-[(2-aminoethyl)(methyl)amino]ethanol to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(4-methylpiperazin-1-yl)ethyl)carbamate (115 mg, 0.21 mmol, 93%) as a white solid. MS (ES): m/z (MH⁺) 556.5 for C₃₅H₆₁N₃O₂. ¹H NMR (300 MHz, CDCl₃) δ: ppm 5.37 (d, 1H, J=5.0 Hz); 5.08 (m, 1H); 4.48 (m, 1H); 3.59 (t, 2H, J=6.4 Hz); 3.27 (d, 2H, J=5.3 Hz); 2.71 (br. s, 4H); 2.57-2.45 (m, 8H); 2.42-2.18 (m, 3H); 2.05-1.72 (m, 5H); 1.70-1.23 (m, 11H); 1.22-0.95 (m, 13H); 0.91 (d, 3H, J=6.5 Hz); 0.86 (dd, 6H, J=1.1 Hz, 6.6 Hz); 0.67 (s, 3H).

### H. Compound SA8: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(4-(2-hydroxyethyl)piperazin-1-yl)ethyl)carbamate

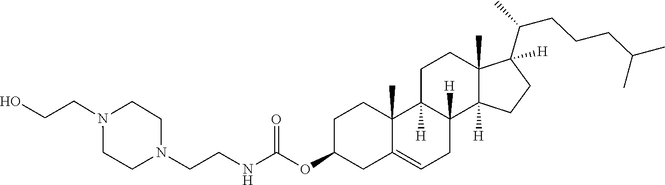

SA8 was prepared in the same manner as SA6 but using 2-[4-(2-aminoethyl)piperazin-1-yl]ethanol instead of 2-[(2-aminoethyl)(methyl)amino]ethanol to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(4-(2-hydroxyethyl)piperazin-1-yl)ethyl)carbamate (85 mg, 0.15 mmol, 87%) as a white solid. MS (ES): m/z (MH⁺) 589.9 for C₃₆H₆₃N₃O₃. ¹H NMR (300 MHz, CDCl₃) δ: ppm 5.37 (d, 1H, J=5.0 Hz); 5.08 (m, 1H); 4.50 (m, 1H); 3.61 (t, 2H, J=6.4 Hz); 3.27 (d, 2H, J=5.3 Hz); 2.69 (br. s, 4H); 2.57-2.45 (m, 11H); 2.42-2.18 (m, 3H); 2.05-1.72 (m, 5H); 1.70-1.23 (m, 11H); 1.22-0.95 (m, 13H); 0.91 (d, 3H, J=6.5 Hz); 0.86 (dd, 6H, J=1.1 Hz, 6.6 Hz); 0.67 (s, 3H).

### I. Compound SA9: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((2-hydroxyethyl)(methyl)amino)propyl)carbamate

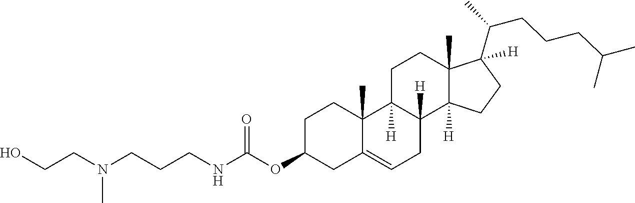

SA9 was prepared in the same manner as SA6 but using 2-[(3-aminopropyl)(ethyl)amino]ethanol instead of 2-[(2-aminoethyl)(methyl)amino]ethanol to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((2-hydroxyethyl)(methyl)amino)propyl)carbamate (97 mg, 0.18 mmol, 80%) as a colorless oil. MS (ES): m/z (MH⁺) 545.5 for C₃₄H₆₀N₂O₃. ¹H NMR (300 MHz, CDCl₃) δ: ppm 5.35 (d, 1H, J=5.1 Hz); 5.19 (m, 1H); 4.45 (m, 1H); 3.60 (t, 2H, J=5.3 Hz); 3.21 (m, 2H); 2.51 (t, 2H, J=5.2 Hz); 2.44 (t, 2H, J=6.9 Hz); 2.39-1.73 (m, 10H); 1.71-1.25 (m, 14H); 1.24-0.95 (m, 12H); 0.89 (d, 3H, J=6.4 Hz); 0.84 (dd, 6H, J=1.2 Hz, 6.6 Hz); 0.65 (s, 3H).

### J. Compound SA10: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate

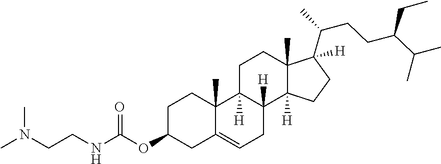

### Step 1: β-Sitosterol 4-nitrophenyl carbonate

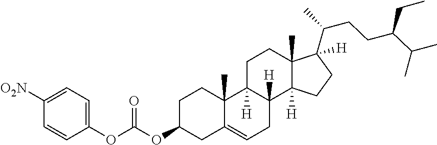

A stirred solution of β-sitosterol (8.00 g, 19.3 mmol), triethylamine (5.4 mL, 39 mmol), and 4-dimethylaminopyridine (0.471 g, 3.86 mmol) in DCM (80 mL) was cooled to 0° C. in an ice bath. 4-Nitrophenyl chloroformate (4.277 g, 21.22 mmol) was added portion wise over 2 min. The reaction mixture was allowed to slowly come to rt overnight and was monitored by LCMS. At 21 h, the reaction mixture was cooled to 0° C. in an ice bath, then triethylamine (2.7 mL) and 4-nitrophenyl chloroformate (3.00 g) were added. The reaction mixture was allowed to come to rt. At 40 h, the reaction mixture was filtered, and the filtrate was added dropwise over 1 h to a flask of stirred ACN (250 mL) cooled to 0° C. in an ice bath. Solids were collected by vacuum filtration, rinsing with ACN to afford β-sitosterol 4-nitrophenyl carbonate (9.15 g, 15.8 mmol, 81.8%) as an off white solid. UPLC/ELSD: RT=3.38 min. ¹H NMR (300 MHz, CDCl₃): δ 8.28 (m, 2H), 7.39 (m, 2H), 5.37-5.49 (m, 1H), 4.54-4.69 (m, 1H), 2.36-2.57 (m, 2H), 0.86-2.10 (br. m, 27H), 1.05 (s, 3H), 0.93 (d, 3H, J=6.4 Hz), 0.78-0.88 (m, 9H), 0.69 (s, 3H).

### Step 2a: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate

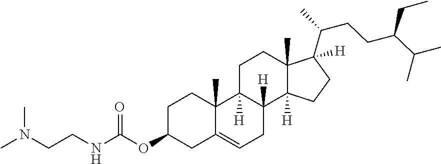

(2-Aminoethyl)dimethylamine (0.11 mL, 1.0 mmol) was added to a stirred solution of β-sitosterol 4-nitrophenyl carbonate (0.487 g, 0.840 mmol) and triethylamine (0.18 mL, 1.3 mmol) in DCM (8.4 mL). The reaction mixture stirred at 40° C. and was monitored by LCMS. At 2 h, the reaction mixture was allowed to cool to rt. The reaction mixture was diluted with DCM and washed with water. The aqueous mixture was extracted with DCM. The combined organic layers were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate (0.296 g, 0.56 mmol, 66.6%) as an off-white solid. UPLC/ELSD: RT=2.43 min. MS (ES): m/z=529.3 [M+H]⁺ for C₃₄H₆₀N₂O₂; ¹H NMR (300 MHz, CDCl₃): δ 5.34-5.41 (m, 1H), 5.01-5.71 (m, 1H), 4.42-4.58 (m, 1H), 3.24 (dt, 2H, J=5.6, 5.4 Hz), 2.19-2.44 (m, 2H), 2.39 (t, 2H, J=6.0 Hz), 2.22 (s, 6H), 1.76-2.05 (br. m, 5H), 0.88-1.72 (br. m, 22H), 1.01 (s, 3H), 0.92 (d, 3H, J=6.4 Hz), 0.77-0.88 (m, 9H), 0.68 (s, 3H).

### Step 2b: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate hydrochloride

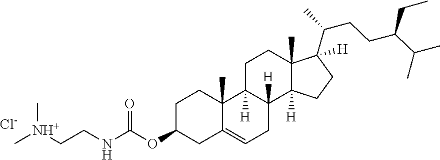

To a stirred solution of β-sitosterol 4-nitrophenyl carbonate (2.50 g, 4.31 mmol) and CHCl₃ (40 mL) was added (2-aminoethyl)dimethylamine (0.56 mL, 5.2 mmol) and triethylamine (0.91 mL, 6.5 mmol). The reaction mixture was stirred at 40° C. and was monitored by LCMS. At 26 h, the reaction mixture was cooled to rt, washed with water (3×), passed through a hydrophobic frit, and then concentrated. The residue was dissolved in iPrOH (15 mL) and DCM (10 mL) to give a yellow solution. To the yellow solution was added 5-6 N HCl in iPrOH (1.0 mL) dropwise, and the reaction mixture stirred for 15 min at rt. The solution was concentrated to remove DCM, and then ACN (10 mL) was added. The mixture was stirred at 0° C. in an ice bath for 15 min, and then solids were collected by vacuum filtration rinsing with 1:1 ACN: iPrOH to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate hydrochloride (1.984 g, 3.344 mmol, 77.6%) as a white solid. UPLC/ELSD: RT=2.43 min. MS (ES): m/z=529.3 [M+H]⁺ for C₃₄H₆₀N₂O₂; ¹H NMR (300 MHz, CDCl₃): δ 12.49 (br. s, 1H), 6.25-6.37 (m, 1H), 5.33-5.40 (m, 1H), 4.40-4.55 (m, 1H), 3.66 (dt, 2H, J=5.4, 5.3 Hz), 3.19 (dt, 2H, J=5.4, 5.3 Hz), 2.86 (d, 6H, J=4.9 Hz), 2.24-2.42 (m, 2H), 1.75-2.07 (br. m, 5H), 0.88-1.72 (br. m, 22H), 1.00 (s, 3H), 0.92 (d, 3H, J=6.4 Hz), 0.77-0.88 (m, 9H), 0.67 (s, 3H).

### K. Compound SA11: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-5-yl)propanoate, hydrochloride salt

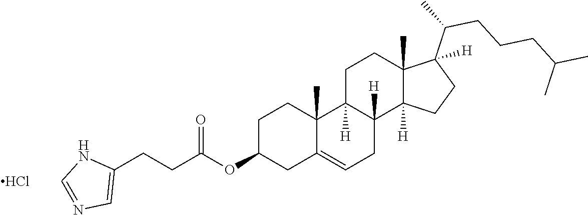

SA11 was prepared as described in WO 2011/068810 (“Delivery of mRNA for the augmentation of proteins and enzymes in human genetic diseases”) and then converted to the hydrochloride salt with 2.5 equivalents of 2M hydrogen chloride in diethyl ether. The resulting precipitate was washed with additional ether, air-dried, and then dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-5-yl)propanoate, hydrochloride salt (29 mg, 0.05 mmol, 77%) as a white solid. MS (ES): m/z (MH⁺) 509.8 for C₃₃H₅₂N₂O₂. ¹H NMR (300 MHz, CDCl₃) δ: ppm 7.54 (s, 1H); 6.80 (s, 1H); 5.36 (d, 1H, J=4.0 Hz); 4.62 (m, 1H); 2.91 (t, 2H, J=6.8 Hz); 2.64 (d, 2H, J=6.9 Hz); 2.30 (d, 1H, J=7.8 Hz); 2.07-1.72 (m, 6H); 1.70-0.94 (m, 28H); 0.91 (d, 3H, J=6.4 Hz); 0.86 (dd, 6H, J=1.0 Hz, 6.5 Hz); 0.67 (s, 3H).

### L. Compound SA12: (3S,8R,9S,10S,13R,14S,17R)-10,13-dimethyl-17-((R)-6-methylheptan-2-yl)hexadecahydro-1H-cyclopenta[a]phenanthren-3-yl (2-(dimethylamino)ethyl)carbamate

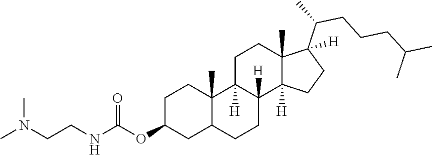

A stirred solution of (3S,8R,9S,10S,13R,14S,17R)-10,13-dimethyl-17-((R)-6-methylheptan-2-yl)hexadecahydro-1H-cyclopenta[a]phenanthren-3-ol (280 mg, 0.72 mmol) in 5 mL dry DCM under dry nitrogen was cooled to 0° C. and triethylamine (60 μL 1.1 mmol) was added, and then a solution of para-nitrophenyl chloroformate (220 mg, 1.1 mmol) in 2 mL dry DCM was added dropwise over five minutes. After ten minutes, the cooling bath was removed, and the mixture stirred at room temp. for three hours, after which no starting material remained by TLC. To the reaction was added N,N-dimethylethylenediamine (neat; 90 μL, 0.8 mmol) dropwise. The reaction mixture stirred at room temp for 30 min. The reaction mixture was then diluted with DCM and washed twice with an aq. 1N NaOH solution. The organics were dried (MgSO4) and filtered. The filtrate was concentrated to a pale yellow solid. This was purified via silica gel chromatography (0-20% (5% conc. aq. NH₄OH in MeOH) in DCM) to give (3S,8R,9S,10S,13R,14S,17R)-10,13-dimethyl-17-((R)-6-methylheptan-2-yl)hexadecahydro-1H-cyclopenta[a]phenanthren-3-yl (2-(dimethylamino)ethyl)carbamate (270 mg, 0.54 mmol, 75%) as a white solid. MS (ES): m/z (MH⁺) 503.9 for C₃₂H₅₈N₂O₂. ¹H NMR (300 MHz, CDCl₃) δ: ppm 5.12 (m, 1H); 4.56 (m, 1H); 3.25 (d, 2H, J=5.4 Hz); 2.41 (t, 2H, J=5.8 Hz); 2.23 (s, 6H); 2.15-1.90 (m, 1H); 1.89-1.41 (m, 11H): 1.40-0.94 (m, 18H); 0.89 (d, 3H, J=6.6 Hz); 0.86 (dd, 6H, J=1.2 Hz, 6.6 Hz); 0.80 (s, 3H); 0.64 (m, 4H).

### M. Compound SA13: (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate

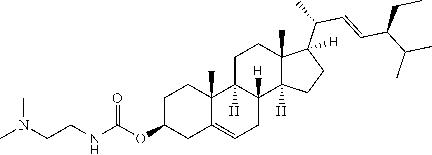

SA13 was prepared in the same manner as SA12 but using (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-ol instead of (3S,8R,9S,10S,13R,14S,17R)-10,13-dimethyl-17-((R)-6-methylheptan-2-yl)hexadecahydro-1H-cyclopenta[a]phenanthren-3-ol to give (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl (2-(dimethylamino)ethyl)carbamate (210 mg, 0.40 mmol, 77%) as a white solid. MS (ES): m/z (MH⁺) 527.9 for C₃₄H₅₈N₂O₂. ¹H NMR (300 MHz, CDCl₃) δ: ppm 5.36 (d, 1H, J=5.1 Hz); 5.15 (dd, 2H, J=8.4 Hz, 15.1 Hz), 5.01 (dd, 1H, J=8.4 Hz, 15.1 Hz), 4.48 (m, 1H), 3.25 (q, 2H, J=5.4 Hz, 11 Hz); 2.40 (t, 2H, J=6.1 Hz); 2.36-2.13 (m, 8H); 2.12-1.77 (m, 6H); 1.76-1.39 (m, 9H); 1.37-1.07 (m, 6H); 1.06-0.90 (m, 8H); 0.89-0.74 (m, 9H), 0.69 (s, 3H).

### N. Compound SA14: (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate

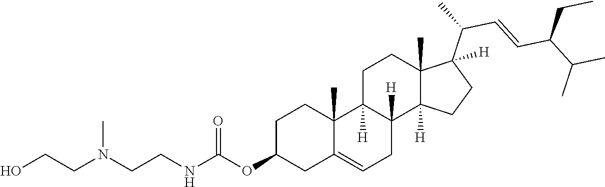

Compound SA14 was prepared in the same manner as SA12 but using (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-ol instead of (3S,8R,9S,10S,13R,14S,17R)-10,13-dimethyl-17-((R)-6-methylheptan-2-yl)hexadecahydro-1H-cyclopenta[a]phenanthren-3-ol and 2-[(2-aminoethyl)(methyl)amino]ethanol instead of N,N-dimethylethylenediamine to give (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate (92 mg, 0.16 mmol, 93%) as a white solid. MS (ES): m/z (MH⁺) 557.46 for C₃₅H₆₀N₂O₃. ¹H NMR (300 MHz, CDCl₃) δ: ppm 5.36 (d, 1H, J=5.1 Hz); 5.19-5.11 (m, 1H); 5.04-4.97 (m, 1H); 4.49 (m, 1H), 3.62 (t, 2H, J=5.2 Hz); 3.28 (d, 2H, J=5.6 Hz); 2.57 (m, 4H); 2.45-2.18 (m, 5H); 2.16-1.62 (m, 8H); 1.60-1.08 (m, 15H); 1.07-0.89 (m, 8H); 0.88-0.74 (m, 9H), 0.69 (s, 3H).

### O. Compound SA15: (3S,8R,9S,10S,13R,14S,17R)-10,13-dimethyl-17-((R)-6-methylheptan-2-yl)hexadecahydro-1H-cyclopenta[a]phenanthren-3-yl (2-((2-hydroxyethyl)(methyl)amino)ethyl)carbamate

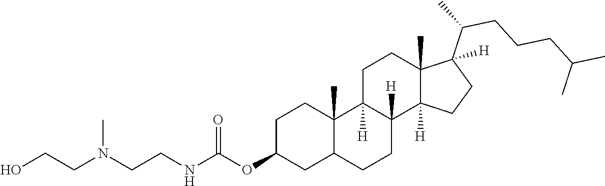

Compound SA14 was prepared in the same manner as SA12 but using 2-[(2-aminoethyl)(methyl)amino]ethanol instead of N,N-dimethylethylenediamine to give (3S,8R,9S,10S,13R,14S,17R)-10,13-dimethyl-17-((R)-6-methylheptan-2-yl)hexadecahydro-1H-cyclopenta[a]phenanthren-3-yl (2-((2-hydroxyethyl)(methyl)amino)ethyl)carbamate (79 mg, 0.15 mmol, 82%) as a white solid. MS (ES): m/z (MH⁺) 532.9 for C₃₃H₆₀N₂O₃. ¹H NMR (300 MHz, CDCl₃) δ: ppm 5.19 (m, 1H); 4.53 (m, 1H); 3.59 (t, 2H, J=5.2 Hz); 3.24 (d, 2H, J=5.4 Hz); 2.53 (m, 4H); 2.25 (s, 3H); 2.02-1.38 (m, 10H); 1.37-0.91 (m, 19H); 0.86 (d, 3H, J=6.6 Hz); 0.83 (dd, 6H, J=1.2 Hz, 6.6 Hz); 0.77 (s, 3H); 0.61 (m, 4H).

### P. Compound SA16: 3-(Dimethylamino)-N-(((1R,4aS,10aR)-7-isopropyl-1,4a-dimethyl-1,2,3,4,4a,9,10,10a-octahydrophenanthren-1-yl)methyl)propenamide

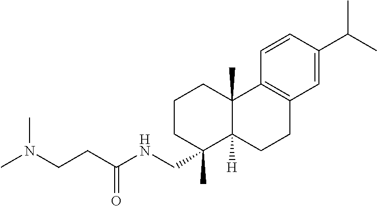 [SMILES: CC(C)c1ccc2c(c1)CC[C@H]1[C@](C)(CNC(=O)CCN(C)C)CCC[C@]21C]

A solution of 3-(dimethylamino)propanoic acid (1.067 g, 9.108 mmol) in thionyl chloride (5.1 mL, 70.1 mmol) was refluxed for 30 min. The reaction mixture was concentrated under vac, and the residue was dissolved in DCM and ((1R,4aS,10aR)-7-isopropyl-1,4a-dimethyl-1,2,3,4,4a,9,10,10a-octahydrophenanthren-1-yl)methanamine (dehydroabietylamine; Combi-Blocks, Inc., San Diego, Calif.) (2 g, 7 mmol) was added followed by triethylamine (2.9 mL, 21.0 mmol). The reaction mixture was stirred at rt for 2 h. Then the reaction mixture was quenched with water and extracted with DCM. The organic layer was diluted with DCM and washed with sat. aq NaHCO₃. The organic layer was separated, washed with brine, dried with Na₂SO₄, filtered, and evaporated under vacuum. The residue was purified by silica gel flash chromatography (0-20% MeOH in DCM) to afford 3-(dimethylamino)-N-(((1R,4aS,10aR)-7-isopropyl-1,4a-dimethyl-1,2,3,4,4a,9,10,10a-octahydrophenanthren-1-yl)methyl)propenamide (0.441 g, 1.147 mmol, Yield 16.4%). UPLC/ELSD: RT=1.19 min. MS (ES): m/z (MH⁺) 385.46 for C₂₅H₄₀N₂O; ¹H NMR (300 MHz, CDCl₃) δ: ppm 8.42 (bs, 1H); 7.18 (m, 1H); 6.99 (m, 1H); 6.90 (m; 1H); 3.17 (m, 2H); 3.01-2.73 (m, 3H); 2.71-2.53 (m, 2H); 2.44 (m, 2H); 2.37-2.16 (m, 7H); 1.95-1.61 (m, 4H); 1.51-1.18 (m, 13H); 0.96 (s, 3H).

### Q. Compound SA17: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-methyl-1H-imidazol-5-yl)ethyl)carbamate

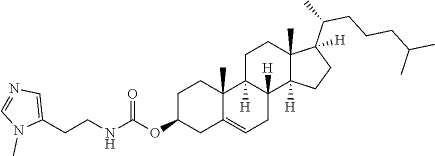

A solution of 3-methylhistamine dihydrochloride (265 mg, 1.27 mmol) in 20 mL of a 1:1 mixture of dry DCM and dry 2-propanol under dry nitrogen was cooled to 0° C. with stirring to give a white slurry. To this was added triethylamine (520 μL, 3.7 mmol) followed by a solution of cholesteryl chloroformate (500 mg, 1.06 mmol) in 10 mL dry THE dropwise over ten minutes. The resulting mixture was stirred at 0° C. for two hours after which no starting material remained by LCMS. The mixture was reduced under vacuum. The residue diluted with a saturated aqueous sodium bicarbonate solution and extracted twice with DCM. The organic layers were combined, dried (MgSO4), and filtered. The filtrate was conc to a pale yellow solid. This was purified by silica gel chromatography (100% DCM going to 90% DCM/10% MeOH) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-methyl-1H-imidazol-5-yl)ethyl)carbamate (530 mg, 0.98 mmol, 93%) as a white solid. UPLC/ELSD: RT=2.85 min. MS (ES): m/z (MH⁺) 538.79 for C₃₄H₅₅N₃O₂. ¹H NMR (300 MHz, CDCl₃) δ: ppm 7.42 (s, 1H); 6.83 (s, 1H); 5.36 (d, 1H, J=5.1 Hz); 4.82 (m, 1H); 4.48 (m, 1H); 3.58 (s, 3H); 3.39 (q, 2H, J=6.7 Hz, 13.2 Hz); 2.42-2.18 (m, 2H); 2.16-1.91 (m, 3H); 1.90-1.73 (m, 3H); 1.65-1.44 (m, 6H); 1.43-1.22 (m, 4H); 1.21-1.06 (m, 6H); 1.05-0.94 (m, 6H); 0.91 (d, 3H, J=6.5 Hz); 0.86 (d, 6H, J=6.5 Hz); 0.67 (s, 3H).

### R. Compound SA18: (1R,4aS,10aR)—N-(2-(Dimethylamino)ethyl)-6-hydroxy-1,4a-dimethyl-1,2,3,4,4a,9,10,10a-octahydrophenanthrene-1-carboxamide

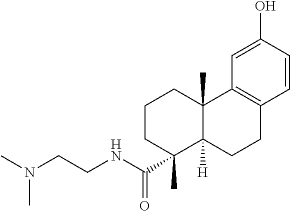 [SMILES: CN(C)CCNC(=O)[C@]1(C)CCC[C@]2(C)c3cc(O)ccc3CC[C@@H]12]

### Step 1: (1R,4aS,10aR)-6-((tert-Butyldimethylsilyl)oxy)-1,4a-dimethyl-1,2,3,4,4a,9,10,10a-octahydrophenanthrene-1-carboxylic acid

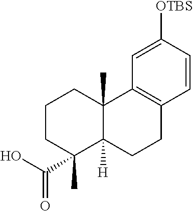 [SMILES: CC(C)(C)[Si](C)(C)Oc1ccc2c(c1)[C@@]1(C)CCC[C@@](C)(C(=O)O)[C@@H]1CC2]

To a stirred solution of (1R,4aS,10aR)-6-hydroxy-1,4a-dimethyl-2,3,4,9,10,10a-hexahydrophenanthrene-1-carboxylic acid (podocarpic acid; Sigma-Aldrich, Inc., St. Louis, Mo.) (0.5 g, 1.8 mmol) in DMF (9.1 mL, 0.2 M) was added imidazole (0.496 g, 7.29 mmol) and t-butyldimethylchlorosilane (0.275 g, 1.822 mmol). The reaction was stirred overnight at room temperature. The reaction mixture was diluted with water and extracted with DCM. The organics were washed with water, dried with sodium sulfate, filtered, and evaporated under vacuum. The residue was purified by silica gel flash chromatography (0-20% MeOH in DCM) to afford (1R,4aS,10aR)-6-[(tert-butyldimethylsilyl)oxy]-1,4a-dimethyl-2,3,4,9,10,10a-hexahydrophenanthrene-1-carboxylic acid (0.471 g, 1.212 mmol, yield 67%). UPLC/ELSD: RT=3.55 min. MS (ES): m/z (MH⁺) 389.56 for C₂₃H₃₆O₃Si; H NMR (300 MHz, CDCl₃) δ: ppm 6.80 (m, 1H); 6.63 (m, 1H); 6.48 (m, 1H); 2.81-2.51 (m, 2H); 2.18-2.00 (m, 3H); 2.00-1.75 (m, 2H); 1.57-1.21 (m, 3H); 1.17 (s, 3H); 1.05-0.77 (m, 14H); 0.15 (m, 6H).

### Step 2: (1R,4aS,10aR)—N-(2-(Dimethylamino)ethyl)-6-hydroxy-1,4a-dimethyl-1,2,3,4,4a,9,10,10a-octahydrophenanthrene-1-carboxamide

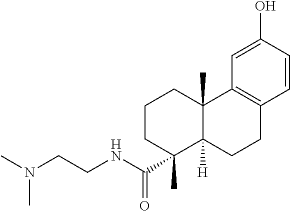 [SMILES: CN(C)CCNC(=O)[C@]1(C)CCC[C@]2(C)c3cc(O)ccc3CC[C@@H]12]

To a solution of (1R,4aS,10aR)-6-[(tert-butyldimethylsilyl)oxy]-1,4a-dimethyl-2,3,4,9,10,10a-hexahydrophenanthrene-1-carboxylic acid (0.213 g, 0.548 mmol) in DCM (2.7 mL) was added (1-chloro-2-methylprop-1-en-1-yl)dimethylamine (0.16 mL, 1.206 mmol). The reaction was stirred at rt for 1 h and evaporated under vacuum. The residue was redissolved in DCM (2.7 mL) and (2-aminoethyl)dimethylamine (0.081 mL, 0.822 mmol) added followed by triethylamine (0.2 mL, 1.6 mmol). The reaction was stirred at rt for 16 h, diluted with DCM, and washed with sat. sodium bicarbonate. The organic layer was separated, washed with brine, dried with Na₂SO₄, filtered, and evaporated under vacuum. The residue was purified by silica gel flash chromatography (0-20% MeOH in DCM) to afford (1R,4aS,10aR)-N-[2-(dimethylamino)ethyl]-6-hydroxy-1,4a-dimethyl-2,3,4,9,10,10a-hexahydrophenanthrene-1-carboxamide (0.095 g, 0.276 mmol, yield 50%). UPLC/ELSD: RT=1.53 min. MS (ES): m/z (MH⁺) 345.37 for C₂₁H₃₂N₂O₂; ¹H NMR (300 MHz, CDCl₃) δ: ppm 6.92 (m, 1H); 6.77 (m, 1H); 6.74-6.57 (m, 2H); 3.44 (m, 2H); 2.94-2.58 (m, 4H); 2.42 (bs, 6H); 2.32-2.16 (m, 3H); 2.14-1.92 (m, 2H); 1.74-1.61 (m, 1H); 1.59-1.07 (m, 10H).

### S. Compound SA19: 2-(Dimethylamino)ethyl (1R,4aS,10aR)-6-hydroxy-1,4a-dimethyl-1,2,3,4,4a,9,10,10a-octahydrophenanthrene-1-carboxylate

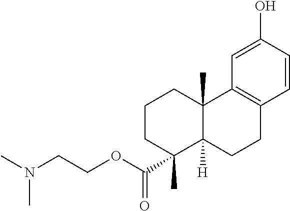 [SMILES: CN(C)CCOC(=O)[C@]1(C)CCC[C@]2(C)c3cc(O)ccc3CC[C@@H]12]

### Step 1: Methyl (1R,4aS,10aR)-6-(benzyloxy)-1,4a-dimethyl-1,2,3,4,4a,9,10,10a-octahydrophenanthrene-1-carboxylate

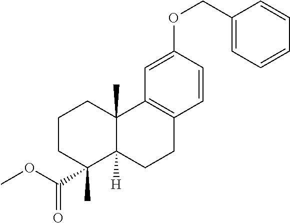 [SMILES: COC(=O)[C@]1(C)CCC[C@]2(C)c3cc(OCc4ccccc4)ccc3CC[C@@H]12]

To a stirred solution of (1R,4aS,10aR)-6-hydroxy-1,4a-dimethyl-2,3,4,9,10,10a-hexahydrophenanthrene-1-carboxylic acid (podocarpic acid; Sigma-Aldrich, Inc., St. Louis, Mo.) (1.9 g, 6.925 mmol) in MeOH (9 mL) and toluene (18 mL) was added trimethylsilyldiazomethane [4.9 mL (2 M solution in hexanes), 9.695 mmol] dropwise over 5 min. The solution was allowed to stir for 1 h at rt. Excess trimethylsilyldiazomethane was quenched with AcOH, and the reaction mixture was evaporated under vacuum. The residue was dissolved in DMF (35 mL) and added Cesium carbonate (9.03 g, 27.74 mmol) and benzyl bromide (1.3 mL, 10.4 mmol). The solution was allowed to stir for 1 h. The reaction was diluted with water and extracted with DCM. The organic layer washed with water and brine. The organic layer was separated, washed with brine, dried with Na₂SO₄, filtered, and evaporated under vacuum. The residue was purified by silica gel flash chromatography (0-100% ethyl acetate in hexanes) to afford methyl (1R,4aS,10aR)-6-(benzyloxy)-1,4a-dimethyl-2,3,4,9,10,10a-hexahydrophenanthrene-1-carboxylate (2.12 g, 5.60 mmol, yield 81%). UPLC/ELSD: RT=3.53 min. MS (ES): m/z (MH⁺) 379.48 for C₂₅H₃₀O₃; ¹H NMR (300 MHz, CDCl₃) δ: ppm 7.38-7.17 (m, 5H); 6.86 (m, 1H); 6.78 (m, 1H); 6.64 (m, 1H); 4.92 (s, 2H); 3.56 (s, 3H); 2.82-2.55 (m, 2H); 2.23-2.02 (m, 3H); 1.96-1.74 (m, 2H); 1.56-1.14 (m, 6H); 1.05-0.89 (m, 4H).

### Step 2: 2-(Dimethylamino)ethyl (1R,4aS,10aR)-6-(benzyloxy)-1,4a-dimethyl-1,2,3,4,4a,9,10,10a-octahydrophenanthrene-1-carboxylate

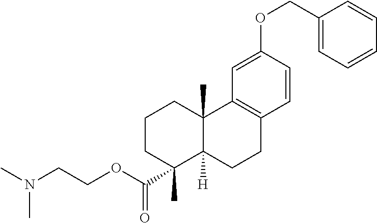 [SMILES: CN(C)CCOC(=O)[C@]1(C)CCC[C@]2(C)c3cc(OCc4ccccc4)ccc3CC[C@@H]12]

To a stirred solution of methyl (1R,4aS,10aR)-6-(benzyloxy)-1,4a-dimethyl-2,3,4,9,10,10a-hexahydrophenanthrene-1-carboxylate (2.1 g, 5.548 mmol) in DMSO (28 mL) was added potassium tert butoxide (9.34 g, 83.22 mmol). The solution was allowed to stir for 1 h. Then, the reaction mixture was poured into ice water, acidified with 2N HCl aqueous solution, and extracted with ethyl acetate. The combined organic layers were washed with water and brine, dried over Na₂SO₄, filtered and the solvent was evaporated off under reduced pressure. The residue was dissolved in DCM (65 mL), and oxalyl chloride (2.2 mL, 25.8 mmol) was added followed by DMF (0.01 mL). The solution was allowed to stir for 1 h. The volatiles were evaporated, and the residue was dissolved in DCM (7 mL). Dimethylaminoethanol (0.21 mL, 2.06 mmol) and 4-(dimethylamino)pyridine (0.033 g, 0.274 mmol) were added followed by triethylamine (0.6 mL, 4.1 mmol). The solution was allowed to stir for 1 h. The residue was diluted with DCM and washed with sat aq NaHCO₃. The organic layer was separated, washed with brine, dried with Na₂SO₄, filtered, and evaporated under vacuum. The residue was purified by silica gel flash chromatography (0-20% MeOH in DCM) to afford the 2-(dimethylamino)ethyl (1R,4aS,10aR)-6-(benzyloxy)-1,4a-dimethyl-2,3,4,9,10,10a-hexahydrophenanthrene-1-carboxylate (0.323 g, 0.741 mmol, yield 54%). UPLC/ELSD: RT=3.13 min. MS (ES): m/z (MH⁺) 436.14 for C₂₈H₃₇NO₃; H NMR (300 MHz, CDCl₃) δ: ppm 7.51-7.30 (m, 5H); 6.99 (m, 1H); 6.91 (m, 1H); 6.76 (m, 1H); 5.04 (s, 2H); 4.23 (m, 2H); 2.93-2.60 (m, 4H); 2.44-2.13 (m, 9H); 2.10-1.91 (m, 2H); 1.71-1.51 (m, 2H); 1.48-1.35 (m, 1H); 1.31 (s, 3H); 1.17-1.03 (m, 4H).

### Step 3: 2-(Dimethylamino)ethyl (1R,4aS,10aR)-6-hydroxy-1,4a-dimethyl-1,2,3,4,4a,9,10,10a-octahydrophenanthrene-1-carboxylate

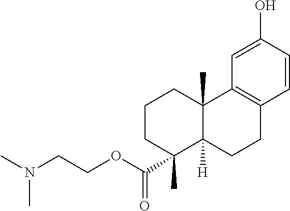 [SMILES: CN(C)CCOC(=O)[C@]1(C)CCC[C@]2(C)c3cc(O)ccc3CC[C@@H]12]

To a flask containing palladium hydroxide (0.07 g) under N2 was added a solution of 2-(dimethylamino)ethyl (1R,4aS,10aR)-6-(benzyloxy)-1,4a-dimethyl-2,3,4,9,10,10a-hexahydrophenanthrene-1-carboxylate (0.31 g, 0.71 mmol) in ethanol (4 mL). The reaction was stirred at rt over the weekend under a hydrogen balloon. The reaction was filtered through a plug of Celite, and the filtrate was evaporated under vacuum. The residue was purified by silica gel flash chromatography (0-20% MeOH in DCM) to afford 2-(dimethylamino)ethyl (1R,4aS,10aR)-6-hydroxy-1,4a-dimethyl-2,3,4,9,10,10a-hexahydrophenanthrene-1-carboxylate (0.14 g, 0.41 mmol, yield 57%). UPLC/ELSD: RT=2.04 min. MS (ES): m/z (MH⁺) 346.11 for C₂₁H₃₁NO₃; ¹H NMR (300 MHz, CDCl₃) δ: ppm 6.92 (m, 1H); 6.75 (m, 1H); 6.60 (m, 1H); 4.21 (m, 2H); 2.90-2.60 (m, 4H); 2.42-2.12 (m, 9H); 2.08-1.90 (m, 2H); 1.69-1.51 (m, 2H); 1.48-1.34 (m, 1H); 1.30 (s, 3H); 1.17-1.02 (m, 4H).

### T. Compound SA20: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(pyridin-4-yl)propanoate

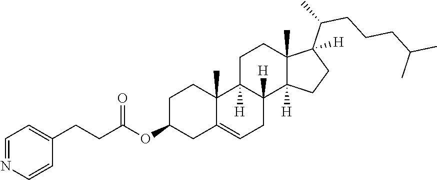

To a stirred solution of 3-(pyridin-4-yl)propanoic acid (196 mg, 1.27 mmol) and cholesterol (415 mg, 1.06 mmol) in 10 mL dry DCM with under dry nitrogen was added EDC-HCl (320 mg, 1.57 mmol) and DMAP (65 mg, 0.53 mmol) followed by DIEA (560 μL, 3.2 mmol), and the resulting mixture stirred at room temp. overnight. No starting material remained by LCMS, so the reaction was diluted with a saturated aqueous sodium bicarbonate solution and extracted twice with DCM. The organic layers were combined, dried (MgSO4), filtered, and concentrated. The residue was purified by silica gel chromatography (0-10% MeOH in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(pyridin-4-yl)propanoate (370 mg, 0.71 mmol, 67%) as a white solid. UPLC/ELSD: RT=3.09 min. MS (ES): m/z (MH⁺) 520.59 for C₃₅H₅₃NO₂. ¹H NMR (300 MHz, CDCl₃) δ: ppm 8.52 (d, 2H, J=6.1 Hz); 7.20 (d, 2H, J=6.0 Hz); 5.37 (d, 1H, J=4.5 Hz); 4.60 (m, 1H); 2.97 (t, 2H, J=7.4 Hz); 2.64 (t, 2H, J=7.4 Hz); 2.27 (d, 2H, J=7.9 Hz); 2.08-1.91 (m, 2H); 1.90-1.74 (m, 3H); 1.64-1.22 (m, 11H); 1.21-1.05 (m, 7H); 1.04-0.95 (m, 6H); 0.91 (d, 3H, J=6.5 Hz); 0.86 (dd, 6H, J=1.2 Hz, 6.5 Hz); 0.67 (s, 3H).

### U. Compound SA21: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(6-aminopyridin-3-yl)propanoate

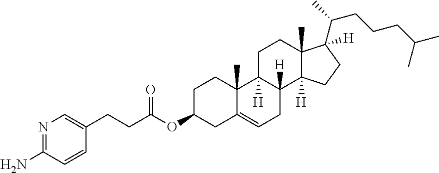

To a stirred solution of 3-(6-aminopyridin-3-yl)propanoic (270 mg, 1.54 mmol) and cholesterol (500 mg, 1.28 mmol) in 10 mL dry DCM under dry nitrogen was added EDC-HCl (390 mg, 1.9 mmol) and DMAP (79 mg, 0.64 mmol) followed by DIEA (680 μL, 3.8 mmol), and the resulting mixture stirred at room temp. overnight. No starting material remained by LCMS, so the reaction mixture was diluted with a saturated aqueous sodium bicarbonate solution and extracted twice with DCM. The organic layers were combined, dried (MgSO4), and filtered, and the filtrate concentrated to a pale yellow solid. This was purified by silica gel chromatography (0-10% MeOH in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(6-aminopyridin-3-yl)propanoate (281 mg, 0.52 mmol, 41%) as a white solid. UPLC/ELSD: RT=3.10 min. MS (ES): m/z (MH⁺) 535.68 for C₃₅H₅₄N₂O₂. ¹H NMR (300 MHz, CDCl₃) δ: ppm 7.88 (d, 1H, J=2.0 Hz); 7.36 (dd, 1H, J=2.3 Hz, 8.5 Hz); 6.50 (d, 1H, J=8.5 Hz); 5.36 (d, 1H, J=4.4 Hz); 4.75-4.54 (m, 3H); 2.81 (t, 2H, J=7.4 Hz); 2.54 (t, 2H, J=7.6 Hz); 2.28 (d, 2H, J=7.7 Hz); 2.08-1.91 (m, 3H); 1.90-1.74 (m, 3H); 1.65-1.23 (m, 10H); 1.22-0.94 (m, 13H); 1.04-0.95 (m, 6H); 0.91 (d, 3H, J=6.5 Hz); 0.86 (dd, 6H, J=0.9 Hz, 6.6 Hz); 0.67 (s, 3H).

### V. Compound SA22: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-aminoethyl)carbamate

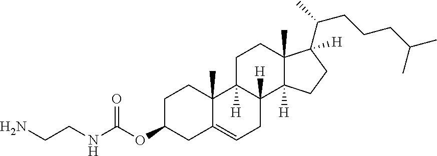

A stirred solution of ethylenediamine (4.6 mL, 64.8 mmol) in 20 mL dry DCM under dry nitrogen was cooled to 0° C., and a solution of cholesteryl chloroformate (2.0 g, 4.3 mmol) in 20 mL dry DCM was added dropwise over twenty minutes. The resulting mixture was allowed to warm to room temp with stirring overnight. The reaction mixture was diluted with DCM, washed three times with water, dried (MgSO4), and filtered, and the filtrate concentrated to a white solid. This was dissolved in hot ethanol and passed through a cotton plug. The filtrate was diluted with acetonitrile until material began to precipitate. The mixture was placed at 4° C. overnight. The resulting solids were isolated via filtration and washed with acetonitrile. The filtrate was concentrated, triturated with acetonitrile, and filtered. The filtered solids were washed with acetonitrile, air-dried, and then dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-aminoethyl)carbamate (1.56 g, 3.3 mmol, 76%) as a white solid. UPLC/ELSD: RT=2.70 min. MS (ES): m/z (MH⁺) 473.55 for C₃₀H₅₂N₂O₂. ¹H NMR (300 MHz, CDCl₃) δ: ppm 5.36 (d, 1H, J=4.6 Hz); 4.96 (m, 1H); 4.50 (m, 1H); 3.23 (q, 2H, J=5.6 Hz, 11.6 Hz); 2.83 (t, 2H, J=5.8 Hz); 2.42-2.19 (m, 2H); 2.06-1.75 (m, 5H); 1.65-1.05 (m, 19H); 1.04-0.94 (m, 6H); 0.91 (d, 3H, J=6.5 Hz); 0.86 (d, 6H, J=6.5 Hz); 0.67 (s, 3H).

### W. Compound SA23: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-guanidinoethyl)carbamate, hydrochloride salt

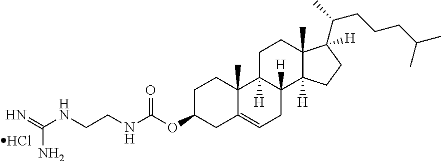

### Step 1

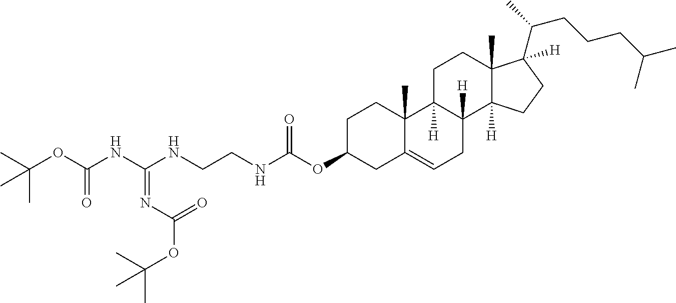

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-aminoethyl)carbamate SA22 (300 mg, 0.63 mmol) and N¹, N²-bis-Boc-guanidine-N³-triflate (250 mg, 0.63 mmol) in 5 mL dry DCM was added triethylamine (92 μL, 0.66 mmol), and the mixture stirred at room temp overnight after which no starting material remained by LCMS. The mixture was diluted with DCM, washed twice with a saturated aqueous sodium bicarbonate solution, dried (MgSO₄), and filtered. The filtrate was concentrated to a pale yellow syrup. This was purified by silica gel chromatography (0-30% EtOAc in hexanes) to give the product (384 mg, 0.53 mmol, 85%) as a white solid. UPLC/ELSD: RT=3.35 min. MS (ES): m/z (MH⁺) 715.66 for C₄₁H₇₀N₄O₆. ¹H NMR (300 MHz, CDCl₃) δ: ppm 11.45 (s, 1H); 8.59 (br. s, 1H); 5.56 (s, 1H); 5.36 (d, 1H, J=4.9 Hz); 4.49 (m, 1H); 3.60 (s, 2H); 3.37 (d, 2H, J=8.8 Hz); 2.42-2.19 (m, 2H); 2.06-1.75 (m, 5H); 1.67-1.44 (m, 23H); 1.43-1.22 (m, 4H); 1.21-0.94 (m, 11H); 0.91 (d, 3H, J=6.5 Hz); 0.86 (dd, 6H, J=1.1 Hz, 6.6 Hz); 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-guanidinoethyl)carbamate, hydrochloride salt

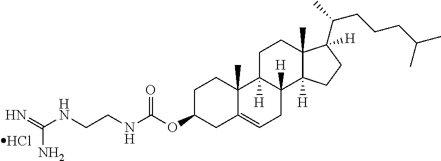

To a stirred solution of the product of step 1 (384 mg, 0.53 mmol) in 5 mL dry DCM was added a 2M solution of HCl in diethyl ether (1.33 mL, 2.66 mmol). The reaction vessel was tightly sealed. The reaction mixture was heated to 40° C. and stirred overnight. Additional 2M HCl in ether (5 mL, 10 mmol) was added. The vial was sealed, and the reaction was heated to 40° C. overnight. No starting material remained by LCMS, so the mixture was concentrated in a stream of nitrogen. The white residue was triturated with diethyl ether and filtered. The filter solids were washed with diethyl ether and air-dried, and then dried under vacuum to give (1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl N-(2-{[(Z)-[(tert-butoxycarbonyl)amino][(tert-butoxycarbonyl)imino]methyl]amino}ethyl)carbamate hydrochloride salt (235 mg, 0.42 mmol, 79%) as a white solid. UPLC/ELSD: RT=2.71 min. MS (ES): m/z (MH⁺) 515.73 for C₃₁H₅₄N₄O₂. ¹H NMR (300 MHz, CDCl₃) δ: ppm 7.88 (br. s, 1H); 7.16 (br. s, 4H); 5.96 (br. s, 1H); 5.36 (s, 1H); 4.42 (m, 1H); 3.55-3.18 (m, 4H); 2.31 (s, 2H); 2.08-1.65 (m, 7H); 1.64-1.22 (m, 11H); 1.23-0.94 (m, 14H); 0.92 (d, 3H, J=5.9 Hz); 0.86 (d, 6H, J=6.6 Hz); 0.68 (s, 3H).

### X. Compound SA24: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate dihydrochloride

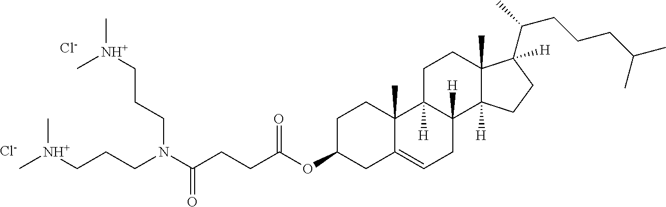

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate

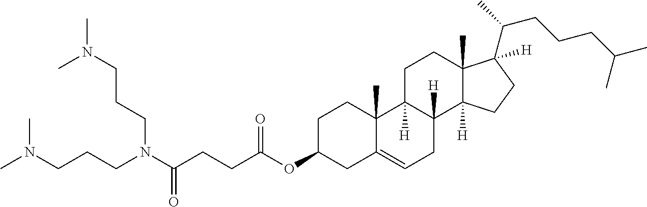

(−)-Cholesterol NHS succinate (517 mg, 0.886 mmol) and 3,3′-iminobis(N,N-dimethylpropylamine) (0.40 mL, 1.8 mmol) were combined in THE (6.0 mL). The reaction mixture stirred at rt and was monitored by TLC. At 20 h, the reaction mixture was diluted with DCM and then washed with water. The aqueous layer was extracted with DCM (2×). The combined organics were passed through a hydrophobic frit, dried over Na₂SO₄, and concentrated. The crude material was purified via silica gel chromatography (0-20% (10% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate (425 mg, 0.607 mmol, 68.5%) as a clear, viscous oil. UPLC/ELSD: RT=2.11 min. MS (ES): m/z=656.6 [M+H]⁺ for C₄₁H₇₃N₃O₃; ¹H NMR (300 MHz, CDCl₃): δ 5.32-5.39 (m, 1H), 4.54-4.68 (m, 1H), 3.29-3.40 (m, 4H), 2.64 (s, 4H), 2.18-2.35 (m, 5H), 2.21 (s, 12H), 0.82-2.05 (br. m, 31H), 1.01 (s, 3H), 0.91 (d, 3H, J=6.5 Hz), 0.86 (d, 3H, J=6.6 Hz), 0.86 (d, 3H, J=6.6 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate dihydrochloride

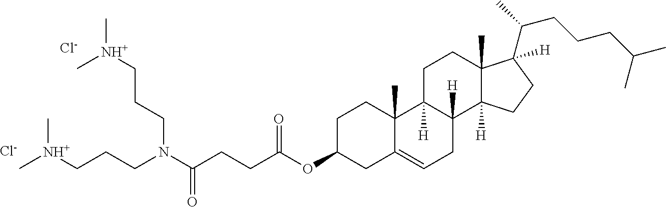

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate (0.135 g, 0.206 mmol) in a mixture of DCM (6.8 mL) and iPrOH (2.7 mL) was added 5-6 N HCl in iPrOH (0.10 mL) dropwise. The reaction mixture stirred at rt for 15 min and then was concentrated. ACN (5 mL) was added, and the mixture was stirred in an ice bath at 0° C. ACN (5 mL) was added. The mixture was concentrated. ACN (3 mL) was added, and the mixture was sonicated. Solids were collected by vacuum filtration and rinsed with cold ACN. Solids were suspended in 1:1 ACN/iPrOH and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate dihydrochloride (0.079 g, 0.10 mmol, 49.1%) as an off-white solid. UPLC/ELSD: RT=1.94 min. MS (ES): m/z=656.3 [M+H]⁺ for C₄₁H₇₃N₃O₃; ¹H NMR (300 MHz, CDCl₃): δ 12.33 (br. s, 1H), 12.14 (br. s, 1H), 5.28-5.38 (m, 1H), 4.45-4.61 (m, 1H), 3.51-3.72 (m, 4H), 3.13-3.26 (m, 2H), 2.97-3.09 (m, 2H), 2.90 (d, 6H, J=4.8 Hz), 2.81 (d, 6H, J=4.7 Hz), 2.55-2.72 (m, 4H), 2.25-2.43 (m, 4H), 2.10-2.24 (m, 2H), 1.75-2.06 (br. m, 5H), 0.92-1.71 (br. m, 21H), 1.01 (s, 3H), 0.91 (d, 3H, 6.4 Hz), 0.86 (d, 3H, J=6.6 Hz), 0.86 (d, 3H, J=6.5 Hz), 0.67 (s, 3H).

### Y. Compound SA25: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((2-(dimethylamino)ethyl)amino)-3,4-dioxocyclobut-1-en-1-yl)amino)ethyl)carbamate, hydrochloride salt

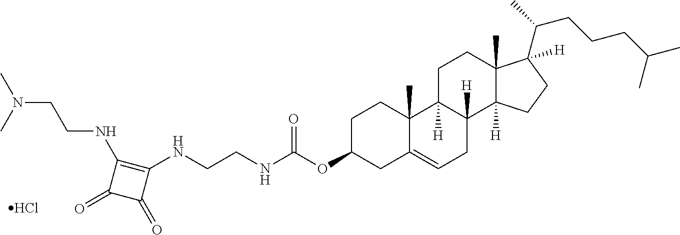

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-methoxy-3,4-dioxocyclobut-1-en-1-yl)amino)ethyl)carbamate

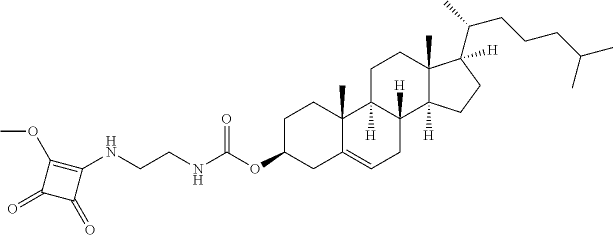

A stirred mixture of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-aminoethyl)carbamate (SA22, 1 g, 2.1 mmol) and 3,4-dimethoxy-3-cyclobutene-1,2-dione (600 mg, 4.2 mmol) in 20 mL of a 1:1 DCM/MeOH mixture was warmed to 35° C. The mixture clarified, and the resulting colorless solution was stirred at room temp overnight after which no starting material remained by LCMS. The mixture was diluted with DCM, washed twice with a saturated aqueous sodium bicarbonate solution, dried (MgSO4), and filtered. The filtrate concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-methoxy-3,4-dioxocyclobut-1-en-1-yl)amino)ethyl)carbamate (1.06 g, 1.8 mmol, 86%) as a white solid. UPLC/ELSD: RT=3.27 min. MS (ES): m/z (MH⁺) 583.68 for C₃₅H₅₄N₂O₅. ¹H NMR (300 MHz, CDCl₃) δ: ppm 6.30 (br. s, 0.5H); 5.99 (br. s, 0.5H); 5.37 (d, 1H, J=5.5 Hz); 4.95 (m, 1H); 4.48 (m, 1H); 4.38 (d, 3H, J=3.5 Hz); 3.79 (m, 1H); 3.56 (m, 1H); 3.40 (q, 2H, J=5.6 Hz, 10.6 Hz); 2.40-2.19 (m, 2H); 2.07-1.75 (m, 5H); 1.68-1.23 (m, 11H); 1.22-0.94 (m, 12H); 0.91 (d, 3H, J=6.5 Hz); 0.86 (dd, 6H, J=1.1 Hz, 6.6 Hz); 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((2-(dimethylamino)ethyl)amino)-3,4-dioxocyclobut-1-en-1-yl)amino)ethyl)carbamate, hydrochloride salt

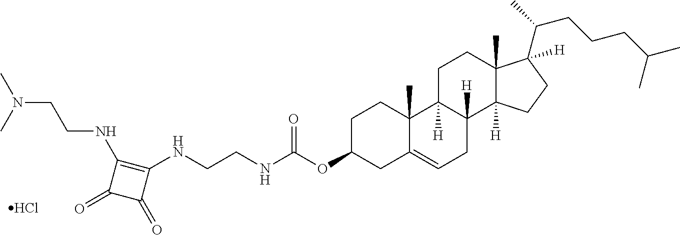

To a stirred suspension of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-methoxy-3,4-dioxocyclobut-1-en-1-yl)amino)ethyl)carbamate (200 mg, 0.34 mmol) in 5 mL methanol was added N,N-dimethylethylenediamine (60 μL, 0.51 mmol). The mixture heated to 45° C. and stirred overnight after which no starting material remained by LCMS. The opaque white mixture was allowed to cool to room temp. and filtered. The filter solids were washed with methanol and then with acetonitrile. The solids were air-dried to give a brittle yellow solid. This was pulverized, dried under vacuum, and suspended in 10 mL of a 1:1 mixture of DCM/methanol. The mixture was heated to give almost complete dissolution and filtered. To the filtrate was added a 2M HCl solution in diethyl ether (1.0 mL, 2 mmol) with stirring. The resulting solution was concentrated in a stream of nitrogen, and the resulting solids were dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((2-(dimethylamino)ethyl)amino)-3,4-dioxocyclobut-1-en-1-yl)amino)ethyl)carbamate hydrochloride (150 mg, 0.22 mmol, 65%) as a white solid. UPLC/ELSD: RT=2.57 min. MS (ES): m/z (MH⁺) 639.56 for C₃₈H₆₂N₄O₄. ¹H NMR (300 MHz, DMSO-d₆) δ: ppm 10.00 (br. s, 1H); 7.94 (m, 2H); 7.16 (t, 1H, J=5.5 Hz); 5.32 (d, 1H, J=3.6 Hz); 4.29 (m, 1H); 3.83 (d, 2H, J=5.7 Hz); 3.50 (br. s, 2H); 3.37 (br. s, 2H); 3.26 (br. s, 2H); 3.13 (q, 2H, J=5.2 Hz, 11.1 Hz); 2.80 (s, 6H); 2.33-2.10 (m, 2H); 2.04-1.68 (m, 5H); 1.63-1.23 (m, 10H); 1.22-0.97 (m, 11H); 0.89 (d, 3H, J=6.4 Hz); 0.83 (dd, 6H, J=1.2 Hz, 6.5 Hz); 0.64 (s, 3H).

### Z. Compound SA26: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((3-(dimethylamino)propyl)amino)-3,4-dioxocyclobut-1-en-1-yl)amino)ethyl)carbamate, hydrochloride salt

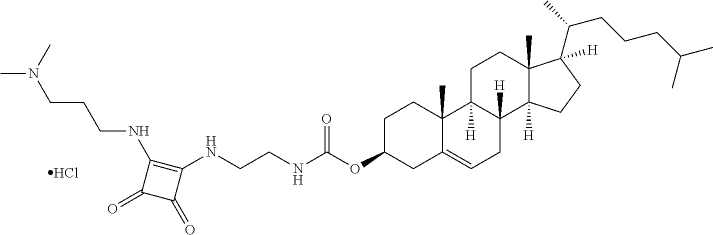

SA26 was prepared in the same manner as SA25 but using N,N-dimethylpropylenediamine in step 2 in place of N,N-dimethylethylenediamine. A similar conversion to the HCl salt was performed to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((3-(dimethylamino)propyl)amino)-3,4-dioxocyclobut-1-en-1-yl)amino)ethyl)carbamate, hydrochloride salt (168 mg, 0.24 mmol, 71%) as a white solid. UPLC/ELSD: RT=2.60 min. MS (ES): m/z (MH⁺) 653.74 for C₃₉H₆₄N₄O₄. ¹H NMR (300 MHz, DMSO-d₆) δ: ppm 10.00 (br. s, 1H); 7.95 (m, 2H); 7.15 (m, 1H); 5.32 (d, 1H, J=2.7 Hz); 4.29 (m, 1H); 3.60-3.43 (m, 4H); 3.19-3.00 (m, 4H); 2.80 (s, 6H); 2.35-2.10 (m, 2H); 2.04-1.68 (m, 5H); 1.56-1.23 (m, 10H); 1.22-0.97 (m, 11H); 0.88 (d, 3H, J=6.4 Hz); 0.83 (dd, 6H, J=0.9 Hz, 6.6 Hz); 0.64 (s, 3H).

### AA. Compound SA27: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((4-(dimethylamino)butyl)amino)-3,4-dioxocyclobut-1-en-1-yl)amino)ethyl)carbamate, hydrochloride salt

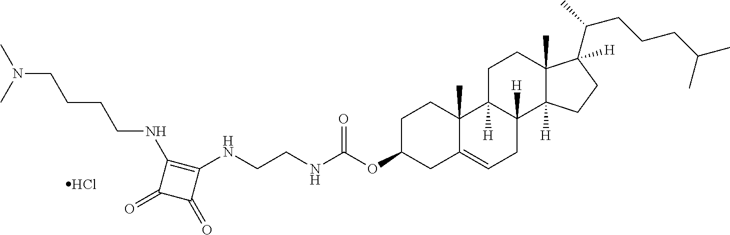

SA27 was prepared in the same manner as SA25 but using N,N-dimethylbutanediamine in step 2 in place of N,N-dimethylethylenediamine. A similar conversion to the HCl salt was performed to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-((4-(dimethylamino)butyl)amino)-3,4-dioxocyclobut-1-en-1-yl)amino)ethyl)carbamate, hydrochloride salt (145 mg, 0.34 mmol, 60%) as a white solid. UPLC/ELSD: RT=2.62 min. MS (ES): m/z (MH⁺) 667.69 for C₃₉H₆₄N₄O₄. ¹H NMR (300 MHz, DMSO-d₆) δ: ppm 10.01 (br. s, 1H); 8.04 (m, 2H); 7.14 (t, 1H, J=5.4 Hz); 5.32 (d, 1H, J=2.9 Hz); 4.29 (m, 1H); 3.95 (m, 2H); 3.49 (br. d, 4H, J=4.8 Hz); 3.19-2.96 (m, 4H); 2.72 (d, 6H, J=4.9 Hz); 2.34-2.08 (m, 2H); 2.04-1.60 (m, 5H); 1.59-1.23 (m, 10H); 1.22-0.97 (m, 11H); 0.88 (d, 3H, J=6.4 Hz); 0.83 (dd, 6H, J=1.0 Hz, 6.6 Hz); 0.64 (s, 3H).

### AB. Compound SA28: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-aminopropyl)amino)-4-oxobutanoate dihydrochloride

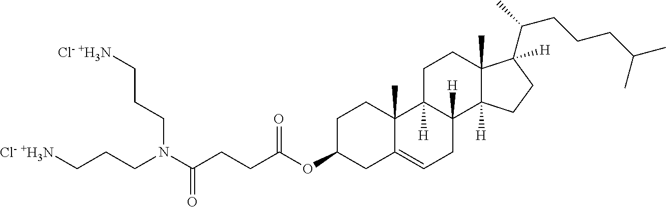

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)propyl)amino)-4-oxobutanoate

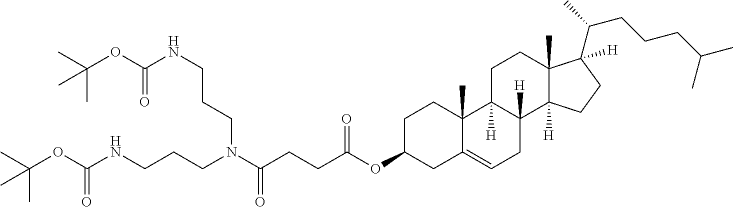

To a solution of (−)-cholesterol NHS succinate (0.100 g, 0.172 mmol) in THE (0.86 mL) was added a solution of tert-butyl N-[3-({3-[(tert-butoxycarbonyl)amino]propyl}amino)propyl]carbamate (0.074 g, 0.22 mmol) in THE (0.40 mL). The reaction mixture stirred at rt and was monitored by LCMS. At 5 h, tert-butyl N-[3-({3-[(tert-butoxycarbonyl)amino]propyl}amino)propyl]carbamate (40 mg) was added. At 21 h, the reaction mixture was diluted with DCM and washed with water. The aqueous layer was extracted with DCM (2×10 mL). The combined organics were passed through a hydrophobic frit, dried over Na₂SO₄, and concentrated. The crude material was purified via silica gel chromatography (30-75% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)propyl)amino)-4-oxobutanoate (quant.) as a clear oil. UPLC/ELSD: RT=3.98 min. MS (ES): m/z=700.7 [(M+H)−(CH₃)₂C═CH₂−CO₂]⁺ for C₄₇H₈₁N₃O₇; ¹H NMR (300 MHz, CDCl₃): δ 5.32-5.40 (m, 1H), 5.29 (br. s, 1H), 4.50-4.73 (m, 2H), 3.40 (t, 2H, J=6.2 Hz), 3.32 (t, 2H, J=6.8 Hz), 3.15 (dt, 2H, J=6.0, 6.4 Hz), 3.04 (dt, 2H, J=5.6, 5.7 Hz), 2.54-2.70 (m, 4H), 2.28-2.37 (m, 2H), 1.75-2.07 (br. m, 5H), 0.94-1.71 (br. m, 25H), 1.44 (s, 9H), 1.43 (s, 9H), 1.01 (s, 3H), 0.91 (d, 3H, J=6.4 Hz), 0.86 (d, 6H, J=6.5 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-aminopropyl)amino)-4-oxobutanoate dihydrochloride

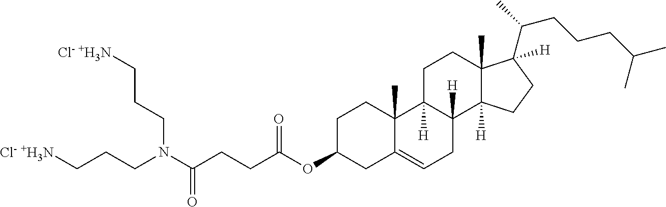

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)propyl)amino)-4-oxobutanoate (110 mg, 0.137 mmol) in DCM (2.0 mL) cooled to 0° C. in an ice bath was added 4 N HCl in dioxane (0.17 mL) dropwise. The reaction mixture was allowed to slowly warm to rt while stirring and was monitored by LCMS. At 3 h, the suspension was diluted with Et₂O. The solids were collected by vacuum filtration, rinsing with Et₂O, to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-aminopropyl)amino)-4-oxobutanoate dihydrochloride (55 mg, 0.081 mmol, 58.7%) as a white solid. UPLC/ELSD: RT=2.13 min. MS (ES): m/z=600.5 [M+H]⁺ for C₃₇H₆₅N₃O₃; ¹H NMR (300 MHz, DMSO-d6): δ 8.07 (br. s, 3H), 7.90 (br. s, 3H), 5.30-5.37 (m, 1H), 4.36-4.51 (m, 1H), 3.34-3.46 (m, 4H), 2.64-2.91 (m, 4H), 2.47-2.63 (m, 2H), 2.19-2.32 (m, 2H), 1.69-2.05 (br. m, 7H), 0.91-1.64 (br. m, 25H), 0.98, (s, 3H), 0.90 (d, 3H, J=6.4 Hz), 0.84 (d, 3H, J=6.6 Hz), 0.84 (d, 3H, J=6.6 Hz), 0.65 (s, 3H).

### AC. Compound SA29: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride

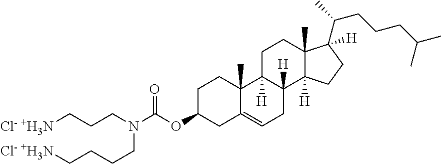

A stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate (prepared as described in Hum. Gene Ther., 7(14), 1701-1717 (1996)) (190 mg, 0.251 mmol) in DCM (3.4 mL) was cooled to 0° C. in an ice bath. Four N HCl in dioxane (0.31 mL) was added dropwise, and the reaction mixture was allowed to slowly warm to rt while stirring. The reaction was monitored by LCMS. At 4 h, 4 N HCl in dioxane (0.10 mL) was added. At 9 h, the reaction mixture was diluted to ca. 20 mL with Et₂O. The suspension was filtered, rinsing with Et₂O. The solids were suspended in heptane, and the suspension was concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride (147 mg, 0.228 mmol, 91.0%) as a white solid. UPLC/ELSD: RT=2.05 min. MS (ES): m/z=558.5 [M+H]⁺ for C₃₅H₆₃N₃O₂; ¹H NMR (300 MHz, DMSO-d6): δ 7.93 (br. s, 6H), 5.29-5.38 (m, 1H), 4.26-4.40 (m, 1H), 3.10-3.30 (m, 4H), 2.68-2.86 (m, 4H), 2.20-2.35 (m, 2H), 1.70-2.03 (br. m, 7H), 0.91-1.64 (br. m, 25H), 0.99 (s, 3H), 0.89 (d, 3H, J=6.4 Hz), 0.85 (d, 3H, J=6.6 Hz), 0.84 (d, 3H, J=6.6 Hz), 0.65 (s, 3H).

### AD. Compound SA30: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (3-((4-aminobutyl)amino)propyl)carbamate dihydrochloride

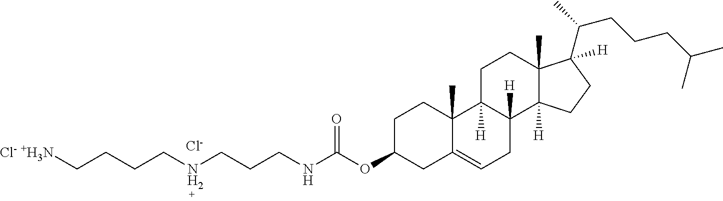

A stirred solution of tert-butyl (4-((tert-butoxycarbonyl)amino)butyl)(3-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)propyl)carbamate (prepared as described in Hum. Gene Ther., 7(14), 1701-1717 (1996)) (215 mg, 0.284 mmol) in DCM (3.9 mL) was cooled to 0° C. in an ice bath. Four N HCl in dioxane (0.35 mL) was added dropwise, and the reaction mixture was allowed to slowly warm to rt while stirring. The reaction was monitored by LCMS. At 4 h, additional 4 N HCl in dioxane (0.10 mL) was added. At 9 h, the reaction mixture was diluted to ca. 20 mL with Et₂O. Solids were collected by vacuum filtration, rinsing with Et₂O. The solids were suspended in heptane and then concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((4-aminobutyl)amino)propyl)carbamate dihydrochloride (142 mg, 0.216 mmol, 76.1%) as a semi-transparent solid. UPLC/ELSD: RT=2.16 min. MS (ES): m/z=558.5 [M+H]⁺ for C₃₅H₆₃N₃O₂; ¹H NMR (300 MHz, DMSO-d₆): δ 8.97 (br. s, 2H), 8.03 (br. s, 3H), 7.20 (t, 1H, J=5.7 Hz), 5.26-5.43 (m, 1H), 4.23-4.39 (m, 1H), 2.97-3.12 (dt, 2H, J=6.1, 6.3 Hz), 2.70-2.95 (m, 6H), 2.13-2.36 (m, 2H), 0.91-2.05 (br. m, 32H), 0.97 (s, 3H), 0.89 (d, 3H, J=6.1 Hz), 0.84 (d, 3H, J=6.6 Hz), 0.84 (d, 3H, J=6.6 Hz), 0.65 (s, 3H).

### AE. Compound SA31: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((4-aminobutyl)(3-aminopropyl)amino)-4-oxobutanoate dihydrochloride

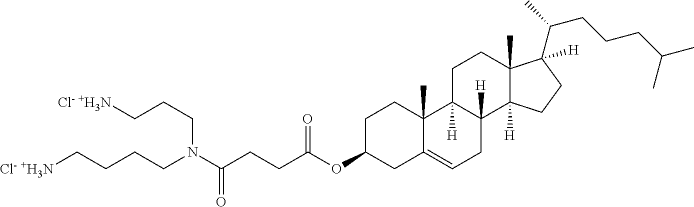

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((4-((tert-butoxycarbonyl)amino)butyl)(3-((tert-butoxycarbonyl)amino)propyl)amino)-4-oxobutanoate

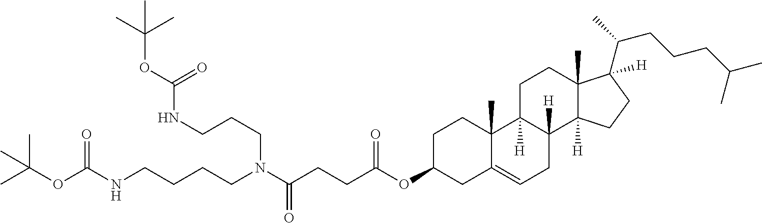

To a solution of (−)-cholesterol NHS succinate (201 mg, 0.344 mmol) in THE (1.7 mL) was added tert-butyl N-[3-({4-[(tert-butoxycarbonyl)amino]butyl}amino)propyl]carbamate (0.178 g, 0.516 mmol) in THE (1.0 mL). The reaction mixture stirred at rt and was monitored by LCMS. At 19 h, the reaction mixture was diluted with DCM (30 mL) and then washed with water. The aqueous layer was extracted with DCM (10 mL). The combined organics were passed through a hydrophobic frit, dried over Na₂SO₄, and concentrated. The crude material was purified by silica gel chromatography (20-65% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((4-((tert-butoxycarbonyl)amino)butyl)(3-((tert-butoxycarbonyl)amino)propyl)amino)-4-oxobutanoate (241 mg, 0.296 mmol, 86.1%) as a white foam. UPLC/ELSD: RT=3.98 min. MS (ES): m/z=814.7 [M+H]⁺ for C₄₈H₈₃N₃O₇; ¹H NMR (300 MHz, CDCl₃): δ 5.21-5.43 (m, 2H), 4.49-4.82 (m, 2H), 3.33 (t, 2H, J=6.4 Hz), 3.22-3.38 (m, 2H), 2.97-3.21 (m, 4H), 2.52-2.72 (m, 4H), 2.24-2.38 (m, 2H), 1.73-2.07 (br. m, 5H), 0.75-1.64 (br. m, 27H), 1.44 (s, 9H), 1.42 (s, 9H), 1.01 (s, 3H), 0.91 (d, 3H, J=6.4 Hz), 0.86 (d, 3H, J=6.6 Hz), 0.86 (d, 3H, J=6.6 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((4-aminobutyl)(3-aminopropyl)amino)-4-oxobutanoate dihydrochloride

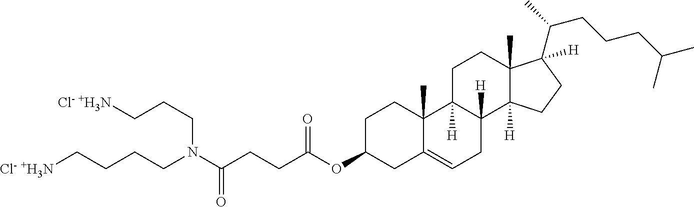

A solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((4-((tert-butoxycarbonyl)amino)butyl)(3-((tert-butoxycarbonyl)amino)propyl)amino)-4-oxobutanoate (0.230 g, 0.282 mmol) in DCM (4.1 mL) was cooled to 0° C. in an ice bath. Four N HCl in dioxane (0.35 mL) was added dropwise, and the reaction mixture was allowed to slowly warm to rt while stirring. The reaction was monitored by LCMS. At 4 h, additional 4 N HCl in dioxane (0.10 mL) was added. At 9 h, the reaction mixture was diluted to ca. 20 mL with Et₂O. Solids were collected by vacuum filtration and were rinsed with Et₂O. The solids were suspended in heptane, concentrated, and further dried under high vacuum to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((4-aminobutyl)(3-aminopropyl)amino)-4-oxobutanoate dihydrochloride (171 mg, 0.247 mmol, 87.4%) as a white solid. UPLC/ELSD: RT=2.22 min. MS (ES): m/z=614.3 [M+H]⁺ for C₃₈H₆₇N₃O₃; ¹H NMR (300 MHz, DMSO-d6): δ 7.73-8.14 (m, 6H), 5.29-5.37 (m, 1H), 4.36-4.51 (m, 1H), 3.15-3.43 (m, 4H), 2.64-2.88 (m, 4H), 2.47-2.63 (m, 2H), 2.21-2.29 (m, 2H), 1.69-2.03 (br. m, 7H), 0.80-1.64 (br. m, 27H), 0.98, (s, 3H), 0.90 (d, 3H, J=6.4 Hz), 0.84 (d, 3H, J=6.5 Hz), 0.84 (d, 3H, J=6.6 Hz), 0.65 (s, 3H).

### AF. Compound SA32: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-(quinuclidin-3-yl)acetate

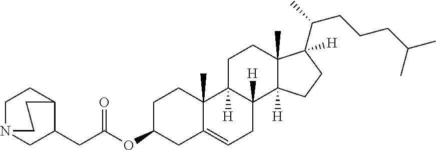

To a solution of 3-(carboxymethyl)-1-azabicyclo[2.2.2]octan-1-ium chloride (AstaTech, Inc., Bristol, Pa.) (0.100 g, 0.486 mmol) in DCM (3.2 mL) was added cholesterol (470 mg, 1.22 mmol), 4-(dimethylamino)pyridine (30 mg, 0.24 mmol), and 1-ethyl-3-(3-dimethylaminopropyl)carbodiimide hydrochloride (0.103 g, 0.535 mmol). THE (3.5 mL) was added. The reaction mixture stirred at rt and was monitored by LCMS. At 16.5 h, the reaction mixture was heated at 50° C. At 21 h, the reaction mixture was cooled to rt. The crude material was diluted with 3:1 CHCl₃/iPrOH (ca. 40 mL) and washed with water. The organics were passed through a hydrophobic frit, dried over Na₂SO₄, and concentrated. The crude material was purified via silica gel chromatography (5-20% (10% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-(quinuclidin-3-yl)acetate (0.193 g, 0.357 mmol, 73.5%) as a clear oil. UPLC/ELSD: RT=3.02 min. MS (ES): m/z=538.5 [M+H]⁺ for C₃₆H₅₉NO₂; ¹H NMR (300 MHz, CD₃OD): δ 5.32-5.43 (m, 1H), 4.47-4.61 (m, 1H), 3.06-3.18 (m, 1H), 2.73-2.92 (m, 4H), 2.25-2.50 (br. m, 5H), 0.91-2.23 (br. m, 32H), 1.05 (s, 3H), 0.95 (d, 3H, J=6.5 Hz), 0.88 (d, 3H, J=6.5 Hz), 0.88 (d, 3H, J=6.6 Hz), 0.73 (s, 3H).

### AG. Compound SA33: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl quinuclidine-3-carboxylate

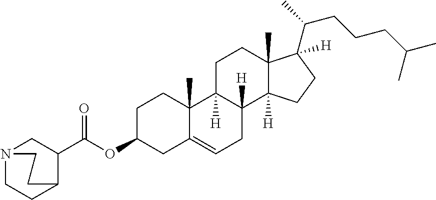

To a suspension of cholesterol (504 mg, 1.30 mmol), 1-azabicyclo[2.2.2]octane-3-carboxylic acid hydrochloride (Enamine, Monmouth Junction, N.J.) (100 mg, 0.522 mmol), and 4-(dimethylamino)pyridine (0.032 g, 0.261 mmol) in THE (3.5 mL) was added 1-ethyl-3-(3-dimethylaminopropyl)carbodiimide hydrochloride (0.110 g, 0.574 mmol). The reaction mixture stirred at rt and was monitored by LCMS. At 16 h, the reaction mixture was stirred at 50° C. At 40 h, the reaction mixture was cooled to rt and diluted with 3:1 CHCl₃:iPrOH (ca. 40 mL). The organics were washed with water, passed through a hydrophobic frit, dried over Na₂SO₄, and concentrated. The crude material was purified via silica gel chromatography (0-10% (10% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl quinuclidine-3-carboxylate (0.121 g, 0.226 mmol, 43.4%) as white solid. UPLC/ELSD: RT=3.06 min. MS (ES): m/z=524.5 [M+H]⁺ for C₃₅H₅₇NO₂; ¹H NMR (300 MHz, CDCl₃): δ 5.34-5.43 (m, 1H), 4.58-4.72 (m, 1H), 3.29 (ddd, 1H, J=13.9, 6.0, 1.7 Hz), 2.72-3.05 (br. m, 5H), 2.46-2.56 (m, 1H), 2.22-2.40 (m, 2H), 2.12-2.19 (m, 1H), 1.75-2.09 (br. m, 5H), 0.92-1.70 (br. m, 25H), 1.02 (s, 3H), 0.91 (d, 3H, J=6.5 Hz), 0.86 (d, 3H, J=6.5 Hz), 0.86 (d, 3H, J=6.6 Hz), 0.68 (s, 3H).

### AH. Compound SA34: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((4,5-dihydro-1H-imidazol-2-yl)amino)ethyl)carbamate, hydrogen iodide salt

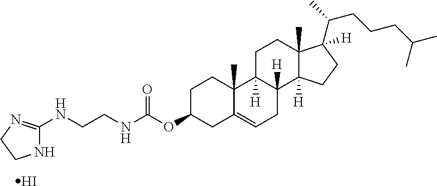

A solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-aminoethyl)carbamate (SA22, 200 mg, 0.42 mmol) and 2-(methylthio)-4,5-dihydro-1H-imidazole hydroiodide (94 mg, 0.38 mmol) in 5 mL dry THE was heated to 40° C. and stirred overnight after which it became a white suspension. No starting material remained by LCMS, so the solution was allowed to cool to room temp. and concentrated (stench!). The resulting solids were triturated with diethyl ether. The mixture was filtered, and the filter solids were washed with ether, air-dried, and then dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((4,5-dihydro-1H-imidazol-2-yl)amino)ethyl)carbamate hydroiodide (77 mg, 0.11 mmol, 30%) as a hygroscopic white solid. UPLC/ELSD: RT=2.76 min. MS (ES): m/z (MH⁺) 541.63 for C₃₃H₅₆N₄O₂. ¹H NMR (300 MHz, CDCl₃) δ: ppm 7.97 (br. s, 2H); 7.29 (br. s, 1H); 5.61 (br. s, 1H); 5.36 (d, 1H, J=3.2 Hz); 4.43 (br. d, 1H, J=8.7 Hz); 3.78 (s, 4H); 3.61-3.21 (m, 4H); 2.31 (s, 2H); 2.30 (d, 2H, J=6.7 Hz); 2.14-1.70 (m, 5H); 1.69-1.22 (m, 11H); 1.21-0.94 (m, 13H); 0.90 (d, 3H, J=6.2 Hz); 0.85 (d, 6H, J=6.4 Hz); 0.66 (s, 3H).

### AI. Compound SA35: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-(bis(3-(dimethylamino)propyl)amino)-3,4-dioxocyclobut-1-en-1-yl)amino)ethyl)carbamate

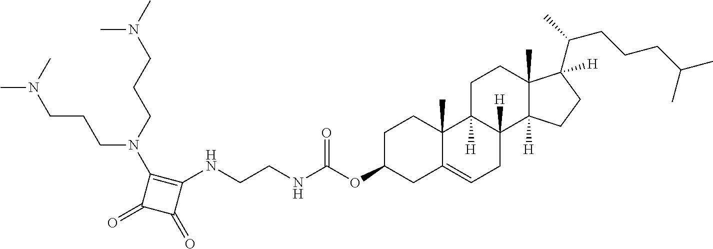

SA35 was prepared in the same manner as SA25 but using 3,3′-iminobis(N,N-dimethylpropylamine) in step 2 in place of N,N-dimethylethylenediamine. Upon completion of the reaction, the opaque white mixture was allowed to cool to room temp. and filtered. The filter solids were washed with methanol and the filtrate concentrated to a pale yellow film. This was purified by silica gel chromatography (0-25% MeOH in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-((2-(bis(3-(dimethylamino)propyl)amino)-3,4-dioxocyclobut-1-en-1-yl)amino)ethyl)carbamate (45 mg, 0.06 mmol, 14%) as a white solid. UPLC/ELSD: RT=2.12 min. MS (ES): m/z (MH⁺) 738.60 for C₄₄H₇₅N₅O₄. ¹H NMR (300 MHz, CDCl₃) δ: ppm 8.85 (br. s, 1H); 5.34 (d, 1H, J=4.7 Hz); 5.19 (t, 1H, J=6.2 Hz); 4.44 (m, 1H); 3.76 (q, 2H, J=5.8 Hz, 11.8 Hz); 3.70-3.47 (m, 3H); 3.39 (q, 2H, J=5.8 Hz, 11.4 Hz); 2.85-2.33 (m, 15H); 2.27 (d, 2H, J=7.1 Hz); 2.10-1.70 (m, 9H); 1.66-1.22 (m, 11H); 1.21-0.95 (m, 12H); 0.91 (d, 3H, J=6.4 Hz); 0.86 (dd, 6H, J=1.1 Hz, 6.6 Hz); 0.67 (s, 3H).

### AJ. Compound SA36: (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride

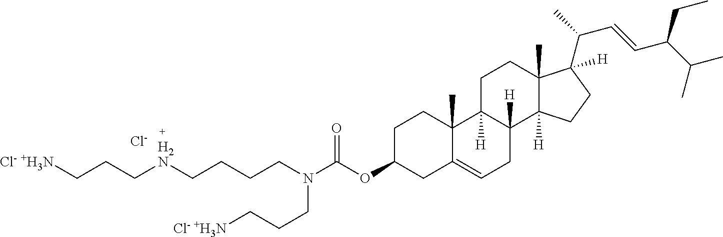

### Step 1: Stigmasterol 4-nitrophenyl carbonate

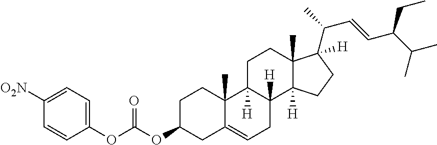

A stirred suspension of stigmasterol (0.600 g, 1.45 mmol), triethylamine (0.41 mL, 2.9 mmol), and 4-dimethylaminopyridine (0.036 g, 0.29 mmol) in DCM (6.0 mL) was cooled in an ice bath to 0° C. Then, 4-nitrophenyl chloroformate (0.322 g, 1.60 mmol) was added. The reaction mixture was allowed to slowly come to rt overnight and was monitored by LCMS. At 21 h, the reaction mixture was filtered. The filtrate was added dropwise to a flask of stirred ACN (30 mL). A solid precipitated to give a yellow suspension. Solids were collected by vacuum filtration, rinsing with CAN, to afford stigmasterol 4-nitrophenyl carbonate (0.526 g, 0.910 mmol, 62.6%). Additional solid precipitated out of the mother liquor. The mother liquor was partially concentrated to remove a majority of the DCM. Once cooled to rt, solids were collected by vacuum filtration rinsing with ACN to afford stigmasterol 4-nitrophenyl carbonate (0.162 g, 0.280 mmol, 19.3%). Combined yield=81.9%. ¹H NMR (300 MHz, CDCl₃): δ 8.28 (m, 2H), 7.39 (m, 2H), 5.40-5.47 (m, 1H), 5.15 (dd, 1H, J=15.1, 8.4 Hz), 5.02 (dd, 1H, J=15.1, 8.4 Hz), 4.55-4.68 (m, 1H), 2.41-2.55 (m, 2H), 1.89-2.13 (br. m, 5H), 0.88-1.84 (br. m, 18H), 1.05 (s, 3H), 1.03 (d, 3H, J=6.6 Hz), 0.76-0.87 (m, 9H), 0.71 (s, 3H).

### Step 2: tert-Butyl ((3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl) butane-1,4-diylbis((3-((tert-butoxycarbonyl)amino)propyl)carbamate)

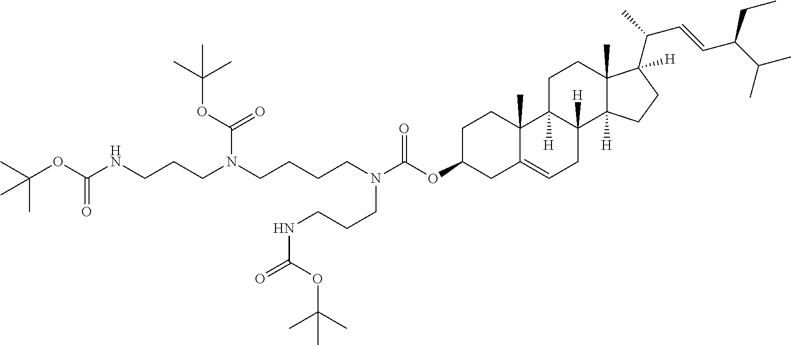

tert-Butyl N-{3-[(tert-butoxycarbonyl)amino]propyl}-N-[4-({3-[(tert-butoxycarbonyl)amino]propyl}amino)butyl]carbamate (0.566 g, 1.12 mmol), stigmasterol 4-nitrophenyl carbonate (0.500 g, 0.865 mmol), and triethylamine (0.36 mL, 2.6 mmol) were combined in toluene (5.0 mL). The reaction mixture stirred at 90° C. and was monitored by LCMS. At 24 h, the reaction mixture was cooled to rt. The reaction mixture was washed with water (3×5 mL) and then concentrated. The crude material was purified via silica gel chromatography (20-60% EtOAc in hexanes) to afford tert-butyl ((3 S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl) butane-1,4-diylbis((3-((tert-butoxycarbonyl)amino)propyl)carbamate) (0.703 g, 0.747 mmol, 86.3%) as a white foam. UPLC/ELSD: RT=3.45 min. MS (ES): m/z=841.8 [(M+H)−(CH₃)₂C═CH₂−CO₂]+ for C₅₅H₉₆N₄O₈; ¹H NMR (300 MHz, CDCl₃): δ 5.33-5.41 (m, 1H), 5.28 (br. s, 1H), 5.16 (dd, 1H, J=15.1, 8.4 Hz), 5.01 (dd, 1H, J=15.1, 8.4 Hz), 4.79 (br. s, 1H), 4.42-4.59 (m, 1H), 2.97-3.48 (br. m, 12H), 2.20-2.43 (m, 2H), 1.79-2.12 (br. m, 5H), 0.88-1.77 (br. m, 26H), 1.45 (s, 9H), 1.43 (s, 18H), 1.02 (s, 3H), 1.02 (d, 3H, J=6.4 Hz), 0.75-0.88 (m, 9H), 0.69 (s, 3H).

### Step 3: (3S,8S,9S,10R,13R,14S,17R)-17-((2R,5S,E)-5-Ethyl-6-methylhept-3-en-2-yl)-10,13-dimethyl-2,3,4,7,8,9,10,11,12,13,14,15,16,17-tetradecahydro-JH-cyclopenta[a]phenanthren-3-yl (3-aminopropyl)(4-((3-aminopropyl)amino)butyl)carbamate trihydrochloride

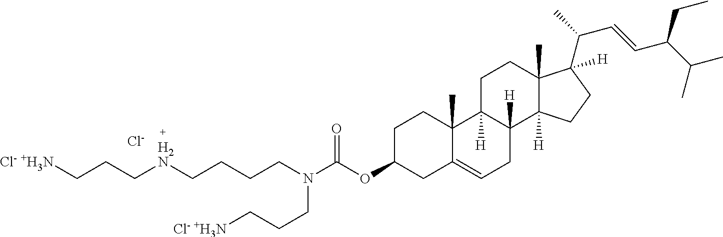

A solution of tert-butyl ((3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl) butane-1,4-diylbis((3-((tert-butoxycarbonyl)amino)propyl)carbamate) (0.700 g, 0.744 mmol) in iPrOH (3.2 mL) was stirred at 30° C. Five to six N HCl in iPrOH (1.5 mL) was added dropwise. The reaction mixture stirred at 40° C. and was monitored by LCMS. At 18.5 h, ACN (3.2 mL) was added, the slurry was sonicated, and then stirred at rt for 1 h. After this time, solids were collected by vacuum filtration, rinsing with 3:1 ACN:iPrOH and then ACN to afford (3S,8S,9S,10R,13R,14S,17R)-17-((2R,5S,E)-5-ethyl-6-methylhept-3-en-2-yl)-10,13-dimethyl-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride (0.509 g, 0.636 mmol, 85.5%) as a white solid. UPLC/ELSD: RT=1.56 min. MS (ES): m/z=641.4 [M+H]⁺ for C₄₀H₇₂N₄O₂; ¹H NMR (300 MHz, CDCl₃): δ 5.38-5.45 (m, 1H), 5.20 (dd, 1H, J=15.1, 8.4 Hz), 5.07 (dd, 1H, J=15.1, 8.4 Hz), 4.40-4.54 (m, 1H), 3.29-3.46 (m, 4H), 3.05-3.20 (m, 6H), 2.92-3.02 (m, 2H), 2.34-2.43 (m, 2H), 1.87-2.20 (m, 9H), 0.92-1.83 (br. m, 22H), 1.09 (s, 3H), 1.07 (d, 3H, 6.6 Hz), 0.80-0.92 (m, 9H), 0.77 (s, 3H).

### AK. Compound SA37: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride

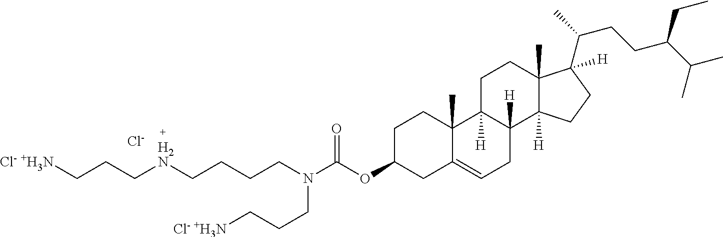

### Step 1: β-Sitosterol 4-nitrophenyl carbonate

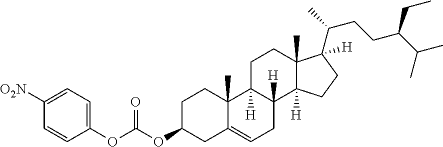

A stirred solution of β-sitosterol (8.00 g, 19.3 mmol), triethylamine (5.4 mL, 39 mmol), and 4-dimethylaminopyridine (0.471 g, 3.86 mmol) in DCM (80 mL) was cooled to 0° C. in an ice bath. To the solution was added 4-Nitrophenyl chloroformate (4.277 g, 21.22 mmol) portion wise over 2 min. The reaction mixture was allowed to slowly come to rt overnight and was monitored by LCMS. At 21 h, the reaction mixture was cooled to 0° C. in an ice bath and then triethylamine (2.7 mL) and 4-nitrophenyl chloroformate (3.00 g) were added. The reaction mixture was allowed to come to rt. At 40 h, the reaction mixture was filtered, and the filtrate was added dropwise over 1 h to a flask of stirred ACN (250 mL) cooled to 0° C. in an ice bath. Solids were collected by vacuum filtration, rinsing with CAN, to afford β-sitosterol 4-nitrophenyl carbonate (9.15 g, 15.8 mmol, 81.8%) as an off white solid. UPLC/ELSD: RT=3.38 min. ¹H NMR (300 MHz, CDCl₃): δ 8.28 (m, 2H), 7.39 (m, 2H), 5.37-5.49 (m, 1H), 4.54-4.69 (m, 1H), 2.36-2.57 (m, 2H), 0.86-2.10 (br. m, 27H), 1.05 (s, 3H), 0.93 (d, 3H, J=6.4 Hz), 0.78-0.88 (m, 9H), 0.69 (s, 3H).

### Step 2: tert-Butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) butane-1,4-diylbis((3-((tert-butoxycarbonyl)amino)propyl)carbamate)

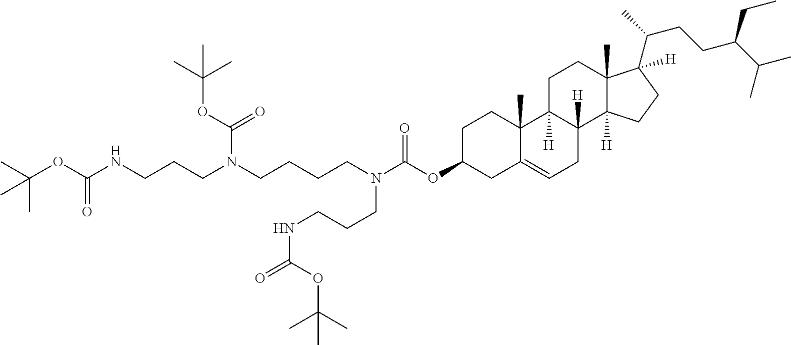

tert-Butyl N-{3-[(tert-butoxycarbonyl)amino]propyl}-N-[4-({3-[(tert-butoxycarbonyl)amino]propyl}amino)butyl]carbamate(0.564 g, 1.12 mmol), β-sitosterol 4-nitrophenyl carbonate (0.500 g, 0.862 mmol), and triethylamine (0.36 mL, 2.6 mmol) were combined in PhMe (5.0 mL). The reaction mixture stirred at 90° C. and was monitored by TLC. At 44 h, the reaction mixture was cooled to rt. The reaction mixture was washed with water (3×5 mL) and then concentrated. The crude material was purified via silica gel chromatography (20-60% EtOAc in hexanes) to afford tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) butane-1,4-diylbis((3-((tert-butoxycarbonyl)amino)propyl)carbamate) (0.704 g, 0.746 mmol, 86.5%) as a white foam.

¹H NMR (300 MHz, CDCl₃): δ 5.32-5.44 (m, 1H), 5.28 (br. s, 1H), 4.78 (br. s, 1H), 4.43-4.58 (m, 1H), 2.97-3.45 (br. m, 12H), 2.20-2.43 (m, 2H), 1.76-2.06 (br. m, 5H), 0.87-1.73 (br. m, 30H), 1.45 (s, 9H), 1.43 (s, 18H), 1.02 (s, 3H), 0.92 (d, 3H, J=6.4 Hz), 0.77-0.87 (m, 9H), 0.68 (s, 3H). UPLC/ELSD: RT=3.51 min. MS (ES): m/z=843.9 [(M+H)−(CH₃)₂C═CH₂−CO₂]⁺ for C₅₅H₉₈N₄O₈.

### Step 3: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride

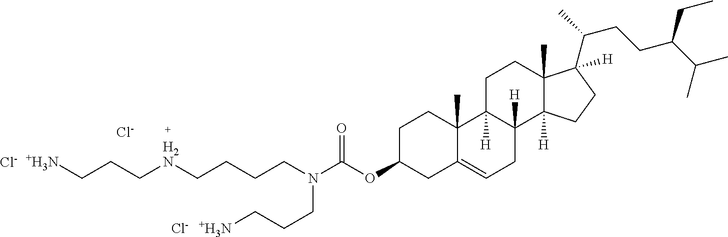

A stirred solution of tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) butane-1,4-diylbis((3-((tert-butoxycarbonyl)amino)propyl)carbamate) (0.700 g, 0.742 mmol) in iPrOH (3.2 mL) was heated at 30° C. To the solution was added 5-6 N HCl in iPrOH (1.5 mL). The reaction mixture stirred at 40° C. and was monitored by LCMS. At 18.5 h, ACN (3.2 mL) was added. The mixture was sonicated and then stirred at rt for 1 h. After this time, solids were collected by vacuum filtration, rinsing with 3:1 ACN:iPrOH and then ACN, to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride (0.498 g, 0.639 mmol, 86.1%) as a white solid. UPLC/ELSD: RT=1.63 min. MS (ES): m/z=643.4 [M+H]⁺ for C₄₀H₇₄N₄O₂; ¹H NMR (300 MHz, CD₃OD): δ 5.39-5.45 (m, 1H), 4.40-4.54 (m, 1H), 3.29-3.48 (m, 4H), 3.05-3.19 (m, 6H), 2.91-3.02 (m, 2H), 2.34-2.43 (m, 2H), 1.83-2.20 (br. m, 9H), 0.92-1.82 (br. m, 26H), 1.08 (s, 3H), 0.97 (d, 3H, J=6.4 Hz), 0.80-0.93 (m, 9H), 0.75 (s, 3H).

### AL. Compound SA38: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminopropyl)(4-((3-aminopropyl)amino)butyl)amino)-4-oxobutanoate trihydrochloride

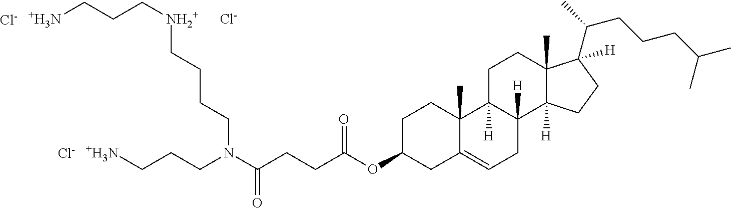

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaoctadecan-18-oate

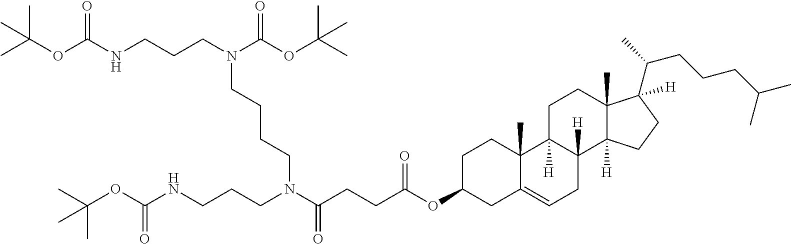

To a stirred solution of tert-butyl N-{3-[(tert-butoxycarbonyl)amino]propyl}-N-[4-({3-[(tert-butoxycarbonyl)amino]propyl}amino)butyl]carbamate (0.341 g, 0.678 mmol) and cholesteryl hemisuccinate (0.300 g, 0.616 mmol) in DCM (6.0 mL) was added 1-ethyl-3-(3-dimethylaminopropyl)carbodiimide hydrochloride (0.177 g, 0.925 mmol). The reaction mixture stirred at rt and was monitored by TLC. At 18 h, water (10 mL) was added, and the reaction mixture stirred at rt for 10 min. After this time, the layers were separated. The aqueous was extracted with DCM (2×10 mL). The combined organics were passed through a hydrophobic frit, dried over Na₂SO₄, and concentrated. The crude material was purified via silica gel chromatography (30-70% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaoctadecan-18-oate (371 mg, 0.382 mmol, 62.0%) as a white foam. UPLC/ELSD: RT=3.37 min. MS (ES): m/z=994.2 [M+Na]⁺ for C₅₆H₉₈N₄O₉; ¹H NMR (300 MHz, CDCl₃): δ 5.31-5.49 (m, 1H), 5.25 (br. s, 1H), 4.74 (br. s, 1H), 4.51-4.67 (m, 1H), 2.95-3.53 (br. m, 12H), 2.52-2.71 (m, 4H), 2.23-2.38 (m, 2H), 1.73-2.08 (br. m, 5H), 0.93-1.72 (br. m, 29H), 1.46 (s, 9H), 1.44 (s, 9H), 1.42 (s, 9H), 1.01 (s, 3H), 0.91 (d, 3H, J=6.5 Hz), 0.86 (d, 6H, J=6.2 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminopropyl)(4-((3-aminopropyl)amino)butyl)amino)-4-oxobutanoate trihydrochloride

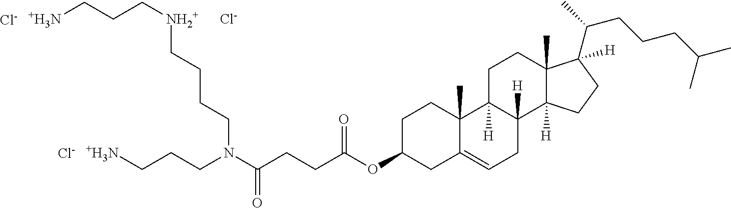

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaoctadecan-18-oate (363 mg, 0.374 mmol) in iPrOH (2.5 mL) was added 5-6 N HCl in iPrOH (0.78 mL). The reaction mixture stirred at 40° C. and was monitored by LCMS. At 16.5 h, additional 5-6 N HCl in iPrOH (0.20 mL) was added, and the reaction mixture stirred at rt. At 22.5 h, ACN (7.5 mL) was added, and the reaction mixture stirred at rt for 10 min. After this time, solids were collected by vacuum filtration and rinsed with ACN to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminopropyl)(4-((3-aminopropyl)amino)butyl)amino)-4-oxobutanoate trihydrochloride (0.240 g, 0.285 mmol, 76.3%) as a white solid. UPLC/ELSD: RT=1.84 min. MS (ES): m/z=671.9 [M+H]⁺ C₄₁H₇₄N₄O₃; ¹H NMR (300 MHz, CD₃OD): δ 5.35-5.41 (m, 1H), 4.46-4.59 (m, 1H), 3.36-3.58 (m, 4H), 2.99-3.19 (m, 6H), 2.86-2.94 (m, 2H), 2.58-2.75 (m, 4H), 2.25-2.41 (m, 2H), 0.96-2.19 (br. m, 34H), 1.05 (s, 3H), 0.95 (d, 3H, J=6.4 Hz), 0.88 (m, 6H, J=6.6 Hz), 0.73 (s, 3H).

### AM. Compound SA39: N-(3-Aminopropyl)-N-(4-((3-aminopropyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

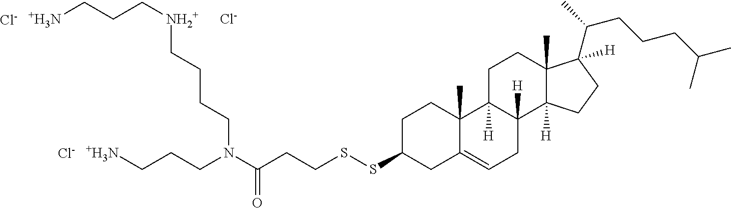

To a stirred solution of tert-butyl (3-((tert-butoxycarbonyl)amino)propyl)(4-(N-(3-((tert-butoxycarbonyl)amino)propyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butyl)carbamate (prepared as described in WO 98/50417 “Cationic Amphiphiles containing a Disulphide Linker for Cell Transfections”) (0.460 g, 0.464 mmol) in DCM (9.2 mL) was added 4 N HCl in dioxane (0.81 mL). The reaction mixture stirred at rt and was monitored by LCMS. At 5 h the reaction mixture was diluted with MTBE to 35 mL and then centrifuged (5000 RPM, 30 min). The supernatant was decanted. The solids were suspended in heptane and then concentrated to afford N-(3-aminopropyl)-N-(4-((3-aminopropyl)amino)butyl)-3-(((3S,8S,9S,10R,13R,14S,17R)-10,3-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride (0.288 g, 0.347 mmol, 74.9%) as a white solid. UPLC/ELSD: RT=1.78 min. MS (ES): m/z=691.2 [M+H]⁺ for C₄₀H₇₄N₄OS₂; ¹H NMR (300 MHz, DMSO-d₆): δ 9.20 (br. s, 1H), 9.11 (br. s, 1H), 8.10 (br. s, 4H), 7.94 (br. s, 2H), 5.30-5.40 (m, 1H), 3.20-3.51 (br. m, 6H), 2.61-3.03 (br. m, 13H), 2.20-2.35 (m, 2H), 0.91-2.07 (br. m, 29H), 0.96 (s, 3H), 0.89 (d, 3H, J=6.4 Hz), 0.84 (d, 3H, J=6.7 Hz), 0.84 (d, 3H, J=6.6 Hz), 0.65 (s, 3H).

### AN. Compound SA40: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)carbamate, hydrochloride salt

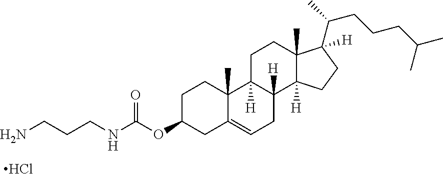

### Step 1: tert-Butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) propane-1,3-diyldicarbamate

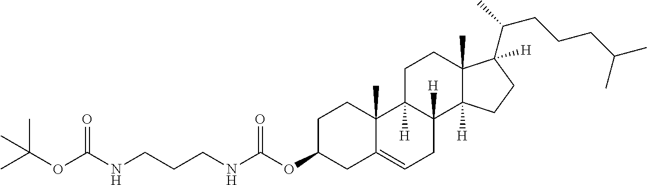

A stirred solution of cholesteryl chloroformate (5 g, 10.8 mmol) in 90 mL dry DCM under dry nitrogen was cooled to 0° C. Triethylamine (3 mL, 21.6 mmol) added. A solution of N-Boc-1,3-diaminopropane (2.3 g, 12.9 mmol) in 10 mL dry DCM was added dropwise over 15 minutes. The resulting colorless solution was stirred at room temp overnight, diluted with DCM, washed twice with 50% saturated brine, washed twice with an aqueous 1N HCl solution, dried (Na₂SO₄), and filtered. The filtrate was concentrated to a colorless oil which began slowly solidifying. This was diluted with a small amount of DCM (˜10 mL) with swirling to solubilize most of the material, and the mixture was diluted with ca. 100 mL hexanes to give a white precipitate. The solids were pulverized. The mixture were stirred vigorously at room temp for 60 minutes and filtered. The filter solids were washed with hexanes and air-dried and then dried under vacuum to give tert-butyl ((3 S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) propane-1,3-diyldicarbamate (5.45 g, 9.3 mmol, 86%) as a white solid. Material was sufficiently pure to use without further purification. UPLC/ELSD: RT=3.37 min. MS (ES): m/z (MH⁺) 587.26 for C₃₆H₆₂N₂O₄. ¹H NMR (300 MHz, CDCl₃) δ: ppm 5.37 (d, 1H, J=5.2 Hz); 5.00 (br. s, 1H); 4.84 (br. s, 1H); 4.49 (m, 1H); 3.19 (m, 4H); 2.43-2.20 (m, 2H); 2.10-1.74 (m, 5H); 1.69-1.39 (m, 18H); 1.38-0.94 (m, 16H); 0.91 (d, 3H, J=6.5 Hz); 0.86 (dd, 6H, J=1.2 Hz, 6.6 Hz); 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)carbamate, hydrochloride salt

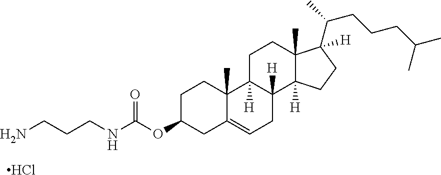

To a stirred solution of tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) propane-1,3-diyldicarbamate (2 g, 3.37 mmol) in 25 mL dry DCM was added a 2M solution of HCl in diethyl ether (8.4 mL, 16.8 mmol). The reaction vessel was tightly sealed, heated to 40° C., and stirred overnight. No starting material remained by LCMS, so the mixture was concentrated in a stream of nitrogen. The white residue was triturated with diethyl ether and filtered. The filter solids were washed with diethyl ether, air-dried, and then dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)carbamate, hydrochloride salt (1.65 g, 3.12 mmol, 93%) as a white solid. UPLC/ELSD: RT=2.35 min. MS (ES): m/z (MH⁺) 487.12 for C₃₁H₅₄N₂O₂. ¹H NMR (300 MHz, CD₃OD) δ: ppm 5.38 (d, 1H, J=4.3 Hz); 4.40 (m, 1H); 3.20 (t, 2H, J=6.6 Hz); 2.96 (t, 2H, J=7.4 Hz); 2.32 (d, 2H, J=7.2 Hz); 2.13-1.72 (m, 7H); 1.71-1.26 (m, 11H); 1.26-0.98 (m, 13H); 0.95 (d, 3H, J=6.5 Hz); 0.88 (dd, 6H, J=0.8 Hz, 6.6 Hz); 0.72 (s, 3H).

### AO. Compound SA41: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-guanidinopropyl)carbamate, hydrochloride salt

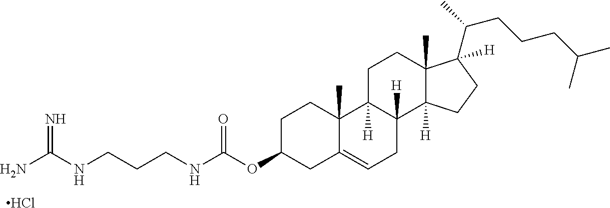

### Step 1

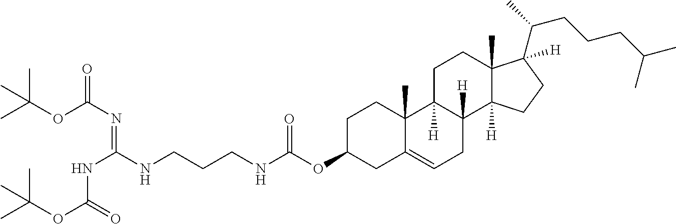

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)carbamate, hydrochloride salt (SA40, 600 mg, 1.13 mmol) and N¹,N²-bis-Boc-guanidine-N³-triflate (450 mg, 1.13 mmol) in 15 mL dry DCM was added triethylamine (330 μL, 2.32 mmol), and the mixture stirred at room temp for 96 hours after which no starting material remained by LCMS. The mixture was diluted with DCM, washed once with an aqueous 1N HCl solution, washed once with a saturated aqueous sodium bicarbonate solution, dried (Na₂SO₄), and filtered. The filtrate was concentrated to a colorless oil. This was purified by silica gel chromatography (0-30% EtOAc in hexanes) to give the product (521 mg, 0.71 mmol, 62%) as a white solid.

¹H NMR (300 MHz, CDCl₃) δ: ppm 11.39 (s, 1H); 8.44 (br. s, 1H); 5.98 (s, 1H); 5.36 (d, 1H, J=3.2 Hz); 4.50 (m, 1H); 3.53 (d, 2H, J=6.9 Hz); 3.20 (s, 2H); 2.43-2.120 (m, 2H); 2.08-1.63 (m, 7H); 1.62-1.22 (m, 29H); 1.20-0.95 (m, 11H); 0.91 (d, 3H, J=6.5 Hz); 0.86 (dd, 6H, J=1.2 Hz, 6.6 Hz); 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-guanidinopropyl)carbamate, hydrochloride salt

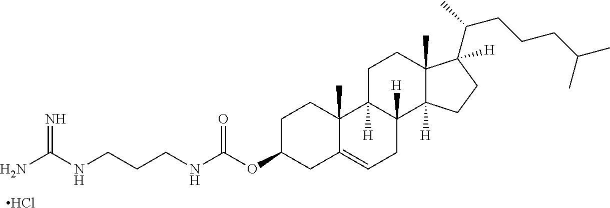

SA41 was prepared in the same manner as SA23 using the product from step 1 to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-guanidinopropyl)carbamate, hydrochloride salt (45 mg, 0.08 mmol, 10%) as a white solid. UPLC/ELSD: RT=2.38 min. MS (ES): m/z (MH⁺) 529.30 for C₃₂H₅₆N₄O₂. ¹H NMR (300 MHz, CD₃OD) δ: ppm 7.88 (br. s, 1H); 6.90 (t, 1H, J=6.2 Hz); 5.39 (d, 1H, J=4.9 Hz); 4.39 (m, 1H); 3.18 (m, 4H); 2.31 (d, 2H, J=7.1 Hz); 2.12-1.68 (m, 5H); 1.66-1.27 (m, 9H); 1.26-0.97 (m, 14H); 0.95 (d, 3H, J=6.5 Hz); 0.88 (dd, 6H, J=0.8 Hz, 6.6 Hz); 0.72 (s, 3H).

### AP. Compound SA42: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-(diisopropylamino)propyl)carbamate

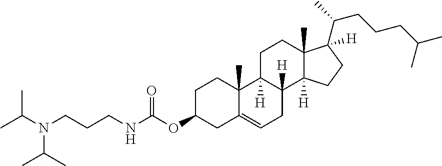

To a stirred solution of 3-(diisopropylamino)propylamine (175 mg, 1.09 mmol) in 5 mL dry DCM under dry nitrogen at 0° C. was added a solution of cholesteryl chloroformate (500 mg, 1.09 mmol) in 5 mL dry DCM dropwise over five minutes. The reaction was allowed to slowly warm to room temp and stirred for two hours after which no starting material remained by LCMS. The solution was diluted with DCM, washed once with a saturated aqueous sodium bicarbonate solution, dried (Na₂SO₄), and filtered. The filtrate was concentrated to a pale yellow oil. This was purified by silica gel chromatography (100% DCM going to 100% DCM/MeOH/NH₄OH (80:20:1)) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-(diisopropylamino)propyl)carbamate (392 mg, 0.66 mmol, 60%) as a colorless syrup which solidified on standing. UPLC/ELSD: RT=2.55 min. MS (ES): m/z (MH⁺) 571.48 for C₃₇H₆₆N₂O₂. ¹H NMR (300 MHz, CDCl₃) δ: ppm 6.00 (br. s, 1H); 5.36 (d, 1H, J=5.1 Hz); 4.48 (m, 1H); 3.23 (d, 2H, J=5.6 Hz); 3.04 (t, 2H, J=6.2 Hz); 2.52 (s, 2H); 2.42-2.16 (m, 2H); 2.07-1.74 (m, 5H); 1.72-1.06 (m, 22H); 1.05-0.94 (m, 16H); 0.91 (d, 3H, J=6.5 Hz); 0.86 (dd, 6H, J=1.3 Hz, 6.6 Hz); 0.67 (s, 3H).

### AQ. Compound SA43: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(diisopropylamino)ethyl)carbamate

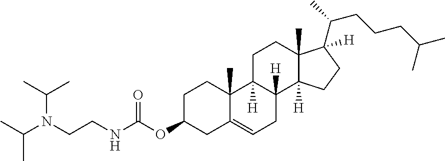

To a stirred solution of 3-(diisopropylamino)ethylamine (210 μL, 1.15 mmol) in 5 mL dry DCM under dry nitrogen at 0° C. was added a solution of cholesteryl chloroformate (500 mg, 1.09 mmol) in 5 mL dry DCM dropwise over five minutes. The reaction was allowed to slowly warm to room temp and stirred for two hours after which no starting material remained by LCMS. The solution was diluted with DCM, washed once with a saturated aqueous sodium bicarbonate solution, dried (Na₂SO₄), and filtered. The filtrate was concentrated to a pale yellow oil. This was purified by silica gel chromatography (100% DCM going to 25% DCM/75% DCM/MeOH/NH₄OH (80:20:1)) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(diisopropylamino)ethyl)carbamate (300 mg, 0.52 mmol, 47%) as a white solid. UPLC/ELSD: RT=2.62 min. MS (ES): m/z (MH⁺) 557.42 for C₃₆H₆₄N₂O₂. ¹H NMR (300 MHz, CDCl₃) δ: ppm 5.36 (d, 1H, J=5.1 Hz); 5.06 (br. s, 1H); 4.50 (m, 1H); 3.12 (q, 2H, J=5.6 Hz, 11.3 Hz); 2.99 (m, 2H); 2.54 (t, 2H, J=6.3 Hz); 2.42-2.17 (m, 2H); 2.07-1.72 (m, 5H); 1.64-1.25 (m, 10H); 1.24-1.04 (m, 8H); 1.03-0.94 (m, 18H); 0.91 (d, 3H, J=6.5 Hz); 0.86 (dd, 6H, J=1.2 Hz, 6.6 Hz); 0.67 (s, 3H).

### Example 10

### Protein Expression Data in Human Cervical Cancer Epithelial Cell (HeLa) Model

LNPs were prepared according to Example 2 using NPI-Luc as the mRNA construct. NPI-Luc is a dual read reporter made by adding a 5xV5 tag and a C-myc nuclear localization sequence at the N-terminus of Firefly Luciferase to enhance the signal to noise ratio. Protein expression can be detected using OneGLo assays with luminescence readout or by immunofluorescence with anti-V5 antibodies. Protein expression was evaluated according to the procedure outlined in Example 7. The LNPs are dosed in 4 wells and the average response was reported. For the HeLa assay the luminescence read (RLU) were normalized to cell counts. The results are shown in Table 10a.

TABLE 10a

| | | Average protein | Standard |
|---|---|---|---|
| | | expression per | deviation protein |
| | | cell | expression |
| Sterol Amine | LNP Core | (RLU per cell) | per cell |
| | | 0.4* | 0.0* |
| SA3 | Compound 18, DSPC, | 111.0 | 11.1 |
| | Cholesterol, and DMG- | | |
| | PEG 2K | | |
| SA4 | Compound 18, DSPC, | 248.5 | 80.5 |
| | Cholesterol, and | | |
| | Compound 428 | | |
| SA6 | Compound 18, DSPC, | 196.2 | 30.3 |
| | Cholesterol, and | | |
| | Compound 428 | | |
| SA10 | Compound 18, DSPC, | 236.6 | 81.3 |
| | Cholesterol, and | | |
| | Compound 428 | | |
| SA11 | Compound 18, DSPC, | 5.3 | 2.9 |
| | Cholesterol, and DMG- | | |
| | PEG 2K | | |
| SA16 | Compound 18, DSPC, | 14.5 | 0.8 |
| | Cholesterol, and | | |
| | Compound 428 | | |
| SA23 | Compound 18, DSPC, β- | 868.8 | 72.8 |
| | sitosterol, Cholesterol, and | | |
| | DMG-PEG 2K | | |
| SA24 | Compound 18, DSPC, | 457.2 | 45.2 |
| | Cholesterol, and DMG- | | |
| | PEG 2K | | |
| SA26 | Compound 18, DSPC, | 84.3 | 35.3 |
| | Cholesterol, and DMG- | | |
| | PEG 2K | | |
| SA29 | Compound 18, DSPC, β- | 550.3 | 91.6 |
| | sitosterol, Cholesterol, and | | |
| | DMG-PEG 2K | | |
| SA30 | Compound 18, DSPC, β- | 648.3 | 157.5 |
| | sitosterol, Cholesterol, and | | |
| | DMG-PEG 2K | | |
| SA31 | Compound 18, DSPC, ß- | 643.6 | 69.5 |
| | sitosterol, Cholesterol, and | | |
| | DMG-PEG 2K | | |
| SA39 | Compound 18, DSPC, | 201.9 | 6.0 |
| | Cholesterol, and DMG- | | |
| | PEG 2K | | |
| SA32 | Compound 18, DSPC, | 301.8 | 44.5 |
| | Cholesterol, and DMG- | | |
| | PEG 2K | | |

*Data taken in phosphate buffered saline solution

### Example 11

### LNP Cellular Uptake and Protein Expression Data in Healthy HBE Cells

LNPs were prepared according to Example 2 using NPI-Luc as the mRNA construct. NPI-Luc is a dual read reporter made by adding a 5xV5 tag and a C-myc nuclear localization sequence at the N-terminus of Firefly Luciferase to enhance the signal to noise ratio. Protein expression can be detected using OneGLo assays with luminescence readout or by immunofluorescence with anti-V5 antibodies. LNP cellular uptake and protein expression was evaluated according to the procedure outlined in Example 6. The results are shown in Table 11a.

TABLE 11a

| | | | | | Standard |
|---|---|---|---|---|---|
| | | | | Average | deviation |
| | | | | protein | protein |
| | | | Standard | expression | expression |
| | | Average | deviation | in | in |
| | | accumulation | accumulation | healthy | healthy |
| | | of LNP in | of LNP in | HBE (% | HBE (% |
| | | healthy HBE | healthy HBE | V5 | V5 |
| Sterol | | (% positive | (% positive | positive | positive |
| Amine | LNP Core | cells) | cells) | cells) | cells) |
| | | 1.6* | 0.2* | 0.1* | 0.0* |
| SA3 | Compound | 43.7 | 1.9 | 9.4 | 1.2 |
| | 18, DSPC, | | | | |
| | Cholesterol, | | | | |
| | and | | | | |
| | DMG-PEG | | | | |
| | 2K | | | | |
| SA23 | Compound | 34.3 | 2.2 | 9.1 | 1.4 |
| | 18, DSPC, | | | | |
| | ß-sitosterol, | | | | |
| | Cholesterol, | | | | |
| | and | | | | |
| | DMG-PEG | | | | |
| | 2K | | | | |
| SA29 | Compound | 56.2 | 3.7 | 12.1 | 3.5 |
| | 18, DSPC, | | | | |
| | ß-sitosterol, | | | | |
| | Cholesterol, | | | | |
| | and | | | | |
| | DMG-PEG | | | | |
| | 2K | | | | |
| SA30 | Compound | 49.9 | 2.0 | 11.9 | 3.8 |
| | 18, DSPC, | | | | |
| | ß-sitosterol, | | | | |
| | Cholesterol, | | | | |
| | and | | | | |
| | DMG-PEG | | | | |
| | 2K | | | | |
| SA31 | Compound | 43.2 | 3.8 | 11.1 | 2.5 |
| | 18, DSPC, | | | | |
| | ß-sitosterol, | | | | |
| | Cholesterol, | | | | |
| | and | | | | |
| | DMG-PEG | | | | |
| | 2K | | | | |

*Data taken in phosphate buffered saline solution

### Example 12

### Nanoparticle Zeta Potential

LNPs were prepared according to Example 2. Zeta potential was measured by diluting LNPs to [mRNA] 0.01 mg/mL in 0.1×PBS on a Malvern Zetasizer (Nano ZS). The results are shown in Table 12a.

TABLE 12a

| Sterol Amine | Core LNP | Zeta Potential (mV) |
|---|---|---|
| SA3 | Compound 18, DSPC, Cholesterol, | 10.6 |
| | and DMG-PEG 2K | |
| SA23 | Compound 18, DSPC, ß-sitosterol, | 12.7 |
| | Cholesterol, and DMG-PEG 2K | |
| SA29 | Compound 18, DSPC, ß-sitosterol, | 8.3 |
| | Cholesterol, and DMG-PEG 2K | |
| SA30 | Compound 18, DSPC, ß-sitosterol, | 8.7 |
| | Cholesterol, and DMG-PEG 2K | |
| SA31 | Compound 18, DSPC, ß-sitosterol, | 8.6 |
| | Cholesterol, and DMG-PEG 2K | |

### Example 13

### In Vivo Studies

Dosing Procedure A: Intratracheal mRNA Delivery

Animals are anesthetized under isoflurane. The tongue is displaced and a small diameter cannula is inserted into the trachea (oropharyngeal route). The cannula tip is passed through the vocal chords, down the trachea so that the tip is very near, but not touching, the carina. Upon placement, 50 μL (mouse) or 200 μL (rat) of formulation is infused into the lungs. After 30 seconds upright, animals are released into a recovery cage and returned to their respective cages once recovered.

### Dosing Procedure B: Nose-Only Aerosol Exposure

Aerosol is generated using a vibrating mesh nebulizer and a defined inlet air flow rate. Aerosol is introduced into the rodent nose-only directed flow exposure chamber by first passing through a mixing chamber before flowing into the exposure tier. Animals are exposed to fresh aerosol at each nose port, which is then exhausted out of the system.

Animals were trained to the nose-only dosing cones for three days prior to initiation of the study. On study day, animals were placed into the dosing cones that were then attached to the aerosol exposure chamber for designated exposure times of 60, 120 or 240 minutes per group for lung doses of 0.4, 0.6 and 1.1 mpk. Animals were monitored continuously throughout the entire exposure and subsequently for any observable adverse reactions. Aerosol concentration (mRNA) and aerodynamic particle size distribution were monitored at the dosing port before and after each dosing occasion to evaluate achieved dose levels and respirable aerosol particle size targets (1-4 μm for rat) respectively.

### Sample Collection and Assays Procedure A: Tissue Collection for Histology

Trachea, lungs and for the aerosol study nasal cavities, nasopharynx and larynx are collected for analysis. Lungs are inflated with 10% NBF fixative and trachea tied off to maintain inflation. Lungs are removed en bloc with attached trachea, bronchi and lobes. Whole lungs en bloc are fixed in 10% NBF at room temperature for at least 24 hours with a maximum of 48 hours and then removed from fixative and placed in PBS. Samples are immediately sent to be processed for paraffin 5-micron sections and H&E staining.

For the aerosol study, nasal cavities, nasopharynx and larynx were also collected in addition to trachea and lungs.

### Sample Collection and Assays Procedure B: Immunohistochemistry (IHC)

IHC was performed on FFPE sections using the Leica Bond RX autostainer. NPI-Luc protein expression was detected by anti-V5 tag antibody at a 1:100 dilution. V5 antibody was detected with the Bond Polymer Refine Detection kit followed by hematoxylin and bluing reagent counterstain. Images were imaged at 20× magnification with the Panoramic 250 Flash III whole slide scanner. Image analysis was completed with Indica Labs HALO image analysis software. Trachea, lung and/or nasal cavity images were analyzed to capture total tracheal, bronchial or nasal epithelial cells and data expressed when appropriate as p % V5 positive epithelial cells per total epithelial cells per animal.

LNP Protein Expression Data in Mouse after Single Dose of mRNA-LNP by Intratracheal Delivery

LNPs were prepared according to Example 2 using NPI-Luc as the mRNA construct. LNPs were delivered to mice by intratracheal instillation for a dose of ˜0.7 mpk. LNP protein expression in respiratory epithelium was evaluated according to sample collection and assay procedures A and B. The results are shown in Table 13a. LNPs with cationic agent disposed primarily on the outer surface demonstrated positive respiratory epithelium protein expression in the trachea and bronchi.

TABLE 13a

| | | | Average | Standard |
|---|---|---|---|---|
| | | | % V5 | deviation |
| | | | positive | % V5 positive |
| | | | cells/total | cells/total |
| Sterol Amine | LNP Core | Tissue | cells | cells |
| | | Mouse Trachea | 0.09* | 0.04* |
| SA3 | Compound 18, | | 40.7 | 0.39 |
| | DSPC, | | | |
| | Cholesterol, and | | | |
| | DMG-PEG 2K | | | |
| SA23 | Compound 18, | | 0.8 | 0.66 |
| | DSPC, β- | | | |
| | sitosterol, | | | |
| | Cholesterol, and | | | |
| | DMG-PEG 2K | | | |
| SA31 | Compound 18, | | 27.07 | 8.96 |
| | DSPC, β- | | | |
| | sitosterol, | | | |
| | Cholesterol, and | | | |
| | DMG-PEG 2K | | | |
| | | | 0.08* | 0.05* |
| SA3 | Compound 18, | Mouse Bronchi | 9.33 | 4.93 |
| | DSPC, | (left lung) | | |
| | Cholesterol, and | | | |
| | DMG-PEG 2K | | | |
| SA23 | Compound 18, | | 0.98 | 0.42 |
| | DSPC, β- | | | |
| | sitosterol, | | | |
| | Cholesterol, and | | | |
| | DMG-PEG 2K | | | |
| SA31 | Compound 18, | | 10.52 | 6.59 |
| | DSPC, β- | | | |
| | sitosterol, | | | |
| | Cholesterol, and | | | |
| | DMG-PEG 2K | | | |

*Data taken in phosphate buffered saline solution

LNP Protein Expression Data in Rat after Single Dose of mRNA-LNP by Intratracheal Delivery

LNPs were prepared according to Example 2 using NPI-Luc as the mRNA construct. LNPs were delivered to rats by intratracheal instillation for a dose of ˜1.2 mpk. LNP protein expression in respiratory epithelium was evaluated according to sample collection and assay procedures A and B. The results are shown in Table 13b. LNPs with cationic agent disposed primarily on the outer surface demonstrated positive respiratory epithelium protein expression in the trachea and bronchi.

TABLE 13b

| | | | Average | Standard |
|---|---|---|---|---|
| | | | % V5 | deviation % V5 |
| Sterol | | | positive cells/ | positive cells/ |
| Amine | LNP Core | Tissue | total cells | total cells |
| | | Rat | 0.03* | 0.03* |
| SA3 | Compound 18, | Trachea | 2.97 | 2.14 |
| | DSPC, Cholesterol, | | | |
| | and DMG-PEG 2K | | | |
| SA23 | Compound 18, | | 0.83 | 0.58 |
| | DSPC, ß-sitosterol, | | | |
| | Cholesterol, and | | | |
| | DMG-PEG 2K | | | |
| | | Rat | 0.11* | 0.11* |
| SA3 | Compound 18, | Bronchi | 4.95 | 4.38 |
| | DSPC, Cholesterol, | (left and | | |
| | and DMG-PEG 2K | right lung) | | |
| SA23 | Compound 18, | | 1.38 | 0.95 |
| | DSPC, ß-sitosterol, | | | |
| | Cholesterol, and | | | |
| | DMG-PEG 2K | | | |

*Data taken in phosphate buffered saline solution

LNP Protein Expression Data in Rat after Single Dose of mRNA-LNP by Aerosol Delivery

LNPs were prepared according to Example 8 using NPI-Luc as the mRNA construct. LNPs were delivered to rats by aerosol delivery using a nose-only aerosol dosing system. LNP protein expression in respiratory epithelium was evaluated according to sample collection and assay procedures A and B. The results are shown in Table 13c. Respiratory epithelium in the nasal cavity, trachea and bronchi were positive for protein expression after aerosol delivery of LNPs.

TABLE 13c

| | | | | | Standard |
|---|---|---|---|---|---|
| | | | | Average | deviation |
| | | | | % V5 | % V5 |
| | | | | positive | positive |
| Sterol | | | | cells/total | cells/total |
| Amine | LNP Core | Tissue | Dose (mpk) | cells | cells |
| | | Rat | | 0.20* | 0.19* |
| SA3 | Compound 18, | Trachea | 0.4 | 0.29 | 0.16 |
| | DSPC, | | 0.6 | 0.16 | 0.15 |
| | Cholesterol, | | 1.1 | 0.65 | 0.39 |
| | and DMG- | | | | |
| | PEG 2K | | | | |
| | | Rat | | 0.03* | 0.02* |
| SA3 | Compound 18, | Bronchi | 0.4 | 0.13 | 0.07 |
| | DSPC, | (left and | 0.6 | 0.13 | 0.03 |
| | Cholesterol, | right | 1.1 | 0.46 | 0.17 |
| | and DMG- | lung) | | | |
| | PEG 2K | | | | |
| | | Rat Nasal | | 0.18* | 0.16* |
| SA3 | Compound 18, | Cavity | 0.4 | 0.64 | 0.76 |
| | DSPC, | | 0.6 | 1.20 | 1.08 |
| | Cholesterol, | | 1.1 | 2.84 | 3.05 |
| | and DMG- | | | | |
| | PEG 2K | | | | |

*Data taken in Tris based buffered solution

All publications, patent applications, patents, and other references mentioned herein are incorporated by reference in their entirety. In case of conflict, the present specification, including definitions, will control.

1. A nanoparticle comprising:
(a) a lipid nanoparticle core,
(b) a polynucleotide or polypeptide payload encapsulated within the core for delivery into a cell, and
(c) a cationic agent disposed primarily on the outer surface of the core,
wherein the nanoparticle has a greater than neutral zeta potential at physiologic pH.

2. A nanoparticle comprising:
(a) a lipid nanoparticle core comprising:
(i) an ionizable lipid,
(ii) a phospholipid,
(iii) a structural lipid, and
(iv) a PEG-lipid, and
(b) a polynucleotide or polypeptide payload encapsulated within the core for delivery into a cell, and
(c) a cationic agent.

3. A nanoparticle comprising:
(a) a lipid nanoparticle core,
(b) a polynucleotide or polypeptide payload encapsulated within the core for delivery into a cell, and
(c) a cationic agent,
wherein the nanoparticle exhibits a cellular accumulation of at least about 20% in epithelial cells and exhibits about 5% or greater expression in epithelial cells.

4. A nanoparticle comprising:
(a) a lipid nanoparticle core,
(b) a polynucleotide or polypeptide payload encapsulated within the core for delivery into a cell, and
(c) a cationic agent,
wherein the nanoparticle exhibits protein expression of about 0.5% to 50% in cells, wherein the cells are in vivo.

5. The nanoparticle of claim 1, wherein a weight ratio of the cationic agent to polynucleotide payload is about 1:1 to about 4:1, about 1.25:1 to about 3.75:1, about 1.25:1, about 2.5:1, or about 3.75:1.

6. The nanoparticle of claim 1, wherein the nanoparticle has a zeta potential of about 5 mV to about 20 mV, about 5 mV to about 20 mV, about 5 mV to about 15 mV, or about 5 mV to about 10 mV.

7. The nanoparticle of claim 1, wherein the lipid nanoparticle core has a neutral charge at a neutral pH.

8. The nanoparticle of claim 1, wherein greater than about 80%, greater than 90%, greater than 95%, or greater than 95% of the cationic agent is on the surface on the nanoparticle.

9. The nanoparticle of claim 1, wherein at least about 50%, at least about 75%, at least about 90%, or at least about 95% of the polynucleotide or polypeptide payload is encapsulated within the core.

10. The nanoparticle of claim 1, wherein the nanoparticle has a polydispersity value of less than about 0.4, less than about 0.3, or less than about 0.2.

11. The nanoparticle of claim 1, wherein the nanoparticle has a mean diameter of about 40 nm to about 150 nm, about 50 nm to about 100 nm, about 60 nm to about 120 nm, about 60 nm to about 100 nm, or about 60 nm to about 80 nm.

12. The nanoparticle of claim 1, wherein a general polarization of laurdan (GPL) of the nanoparticle is greater than or equal to about 0.6.

13. The nanoparticle of claim 1, wherein the nanoparticle has a d-spacing of greater than about 6 nm or greater than about 7 nm.

14. The nanoparticle of claim 1, wherein at least 50%, at least 75%, at least 90%, or at least 95% of the nanoparticles have a surface fluidity value of greater than a threshold polarization level.

15. The nanoparticle of claim 1, wherein about 10% or greater, about 15% or greater, or about 20% or greater of cell population has accumulated the nanoparticle when the nanoparticle is contacted with a population of cells.

16. The nanoparticle of claim 1, wherein about 5% or greater, or about 10% or greater, of cell expresses the polynucleotide or polypeptide when the nanoparticle is contacted with a population of cells.

17. The nanoparticle of claim 1, wherein the cell population is an epithelial cell population, a respiratory epithelial cell population, a nasal cell population, an alveolar epithelial cell population, a lung cell population, a bronchial epithelial cell population, or HBE population.

18-37. (canceled)

38. The nanoparticle of claim 1, wherein:
the cationic agent is a cationic lipid;
the cationic agent is a cationic lipid, wherein the cationic lipid is a sterol amine comprising a hydrophobic moiety and a hydrophilic moiety; or
the cationic agent is a cationic lipid, wherein the cationic lipid is a sterol amine having a compound of Formula (A1):
A-L-B (A1)
or a salt thereof, wherein:
A is an amine group, L is an optional linker, and B is a sterol.

39. The nanoparticle of claim 38, wherein the sterol amine has Formula A2a: 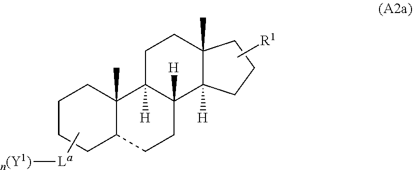
or a salt thereof, wherein:
---- is a single or double bond
R¹ is C₁₋₁₄ alkyl or C₁₋₁₄ alkenyl;
L^{a} is absent, —O—, —S—S—, —OC(═O), —C(═O)N—, —OC(═O)N—, CH₂—NH—C(O)—, —C(═O)O—, —OC(═O)—CH₂—CH₂—C(═O)N—, —S—S—CH₂, —SS—CH₂—CH₂—C(═O)N—, or a group of formula (a): 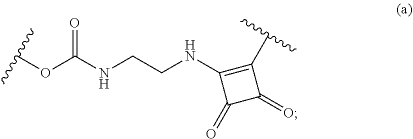 [SMILES: *OC(=O)NCCNc1c(*)c(=O)c1=O]
Y¹ is C₁₋₁₀ alkyl, 3 to 8-membered heterocycloalkyl, 5 to 6-membered heteroaryl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof,
and wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents independently selected from C₁₋₆ alkyl, halo, OH, —O(C₁₋₆ alkyl), —C₁₋₆ alkyl-OH, NH₂, —NH(C₁₋₆ alkyl), N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH-(3 to 8-membered heterocycloalkyl), and —NH(5 to 6-membered heteroaryl); and
n is 1 or 2.

40-41. (canceled)

42. The nanoparticle of claim 39, wherein L^{a} is —OC(═O), —OC(═O)N—, or —OC(═O)—CH₂—CH₂—C(═O)N—.

43-46. (canceled)

47. The nanoparticle of claim 39, wherein R¹ is

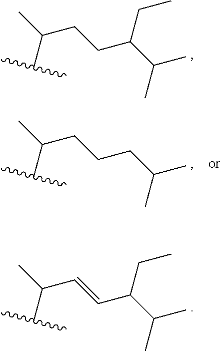

48. The nanoparticle of claim 39, wherein Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, OH, —C₁₋₆ alkyl-OH, or NH₂.

49. The nanoparticle of claim 39, wherein Y¹ is selected from:

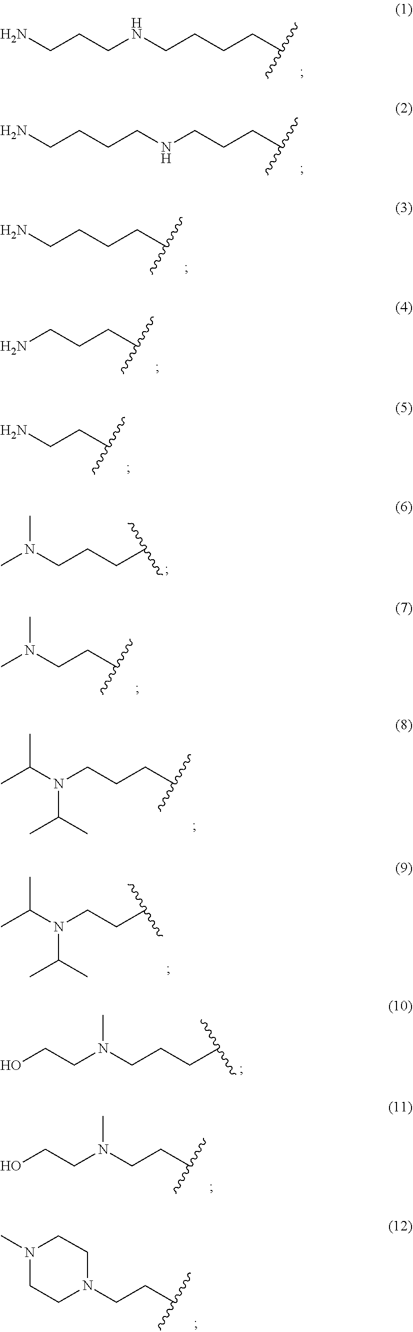

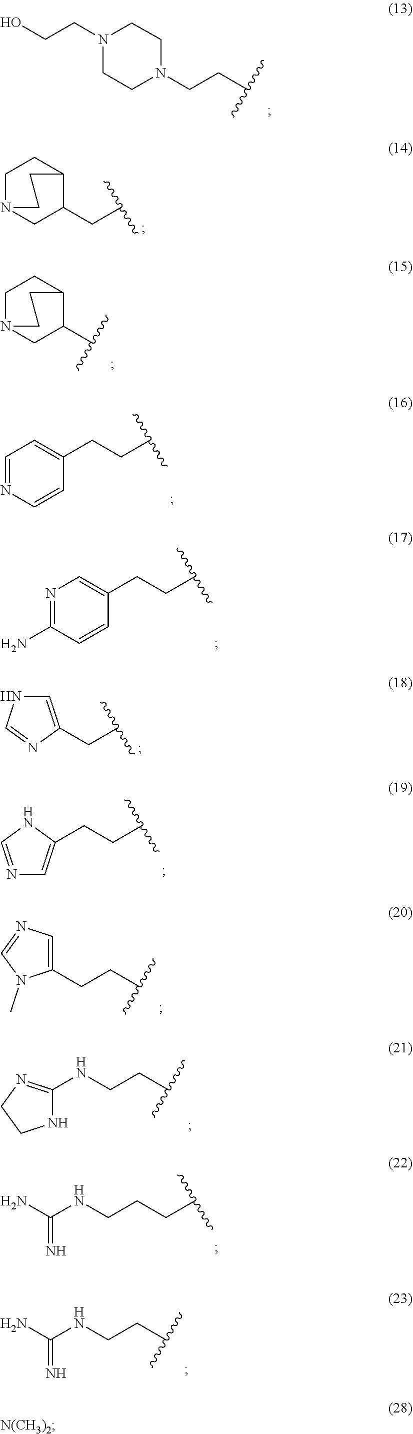

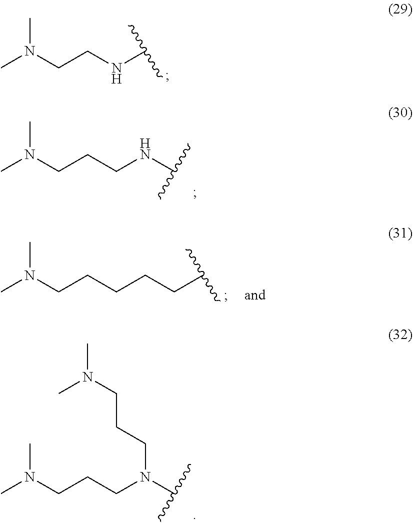

50. The nanoparticle of claim 38, wherein the sterol amine has Formula A4 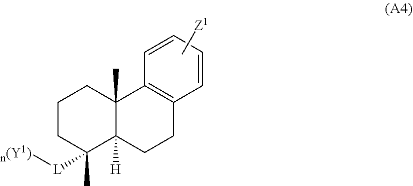
or a salt thereof, wherein:
Z¹ is OH or C₃₋₆ alkyl;
L is absent, —O—, —S—S—, —OC(═O), —C(═O)N—, —OC(═O)N—, —CH₂—NH—C(═O)—, —C(═O)O—, —OC(═O)—CH₂—CH₂—C(═O)N—, —S—S—CH₂, or —SS—CH₂—CH₂—C(O)N—;
Y¹ is C₁₋₁₀ alkyl, 3 to 8-membered heterocycloalkyl, 5 to 6-membered heteroaryl, —C₁₋₆ alkyl-(3 to 8 membered heterocycloalkyl), or —C₁₋₆ alkyl-(5 to 6 membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof,
and wherein the C₁₋₁₀ alkyl, 3 to 8-membered heterocycloalkyl, 5 to 6 membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents selected from C₁₋₆ alkyl, halo, OH, —O(C₁₋₆ alkyl), —C₁₋₆ alkyl-OH, NH₂, —NH(C₁₋₆ alkyl), —N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH(3 to 8-membered heterocycloalkyl), and —NH(5 to 6-membered heteroaryl); and
n is 1 or 2.

51. The nanoparticle of claim 50, wherein Z¹ is OH.

52. The nanoparticle of claim 50, wherein Z¹ is C₃₋₆ alkyl.

53. The nanoparticle of claim 50, wherein L is —C(═O)N—, —CH₂—NH—C(═O)—, or —C(═O)O—.

54. The nanoparticle of claim 50, wherein Y¹ is C₁₋₁₀ alkyl comprising one to five primary, secondary, or tertiary amines or combination thereof.

55. The nanoparticle of claim 50, wherein Y¹ is

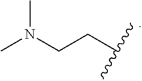 [SMILES: *CCN(C)C]

56-57. (canceled)

58. The nanoparticle of claim 38, wherein the sterol amine is selected from:

| Sterol amine no. | Structure |
|---|---|
| SA1 | 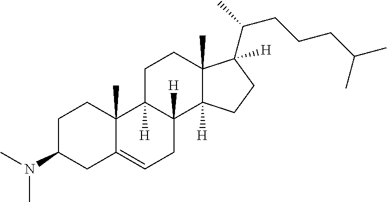 |
| SA2 | 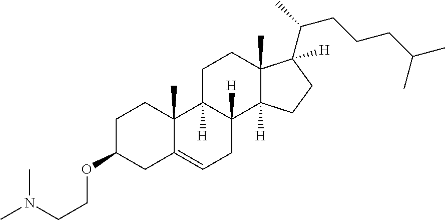 |
| SA3 | 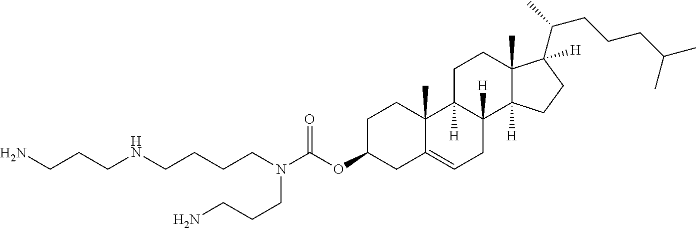 |
| SA4 | 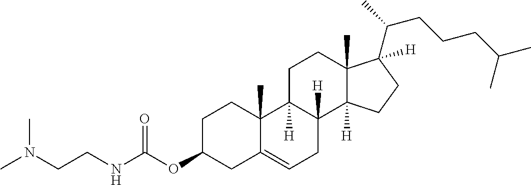 |
| SA5 | 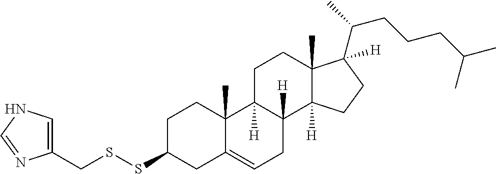 |
| SA6 | 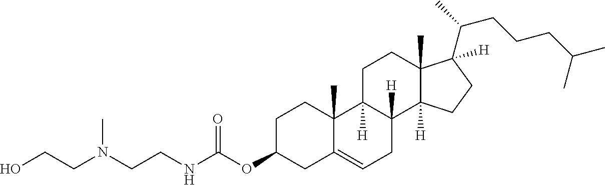 |
| SA7 | 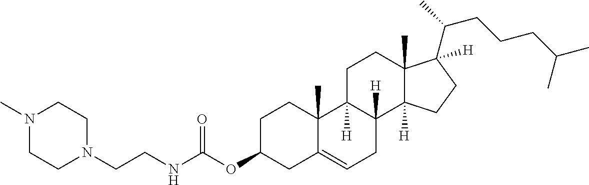 |
| SA8 | 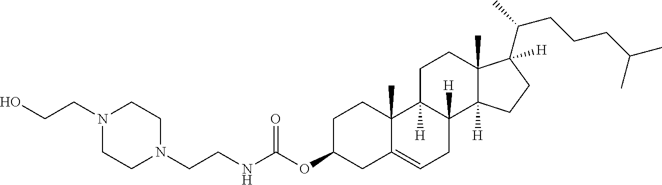 |
| SA9 | 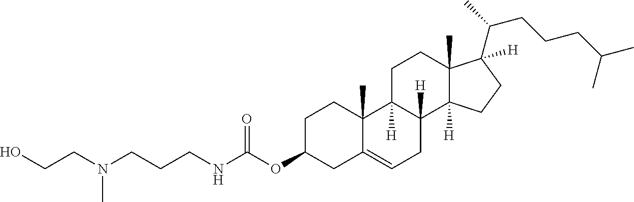 |
| SA10 | 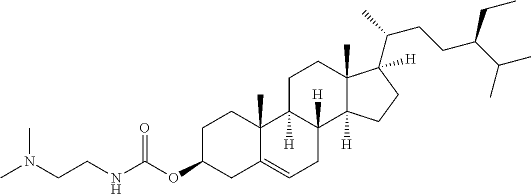 |
| SA11 | 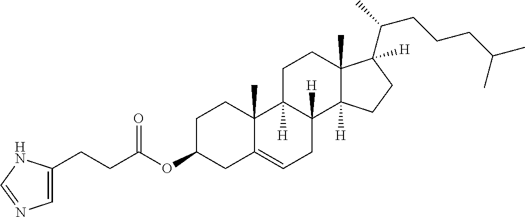 |
| SA12 | 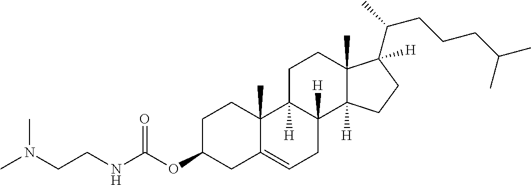 |
| SA13 | 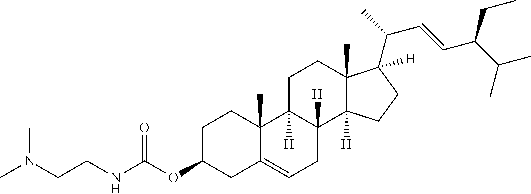 |
| SA14 | 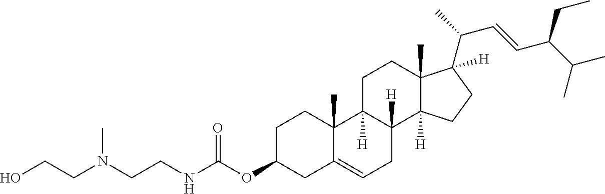 |
| SA15 | 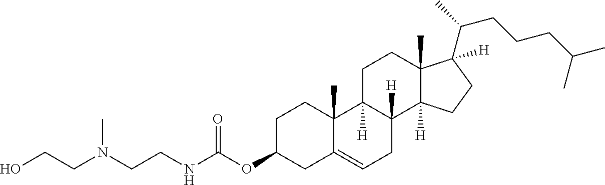 |
| SA16 | 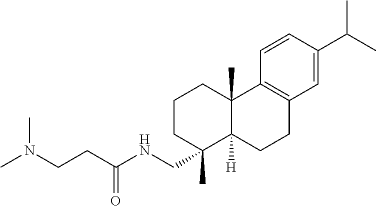 [SMILES: CC(C)c1ccc2c(c1)CC[C@H]1[C@](C)(CNC(=O)CCN(C)C)CCC[C@]21C] |
| SA17 | 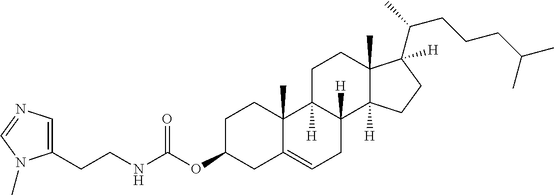 |
| SA18 | 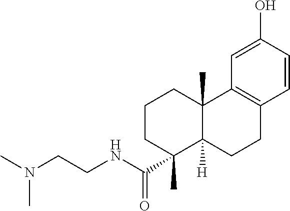 [SMILES: CN(C)CCNC(=O)[C@]1(C)CCC[C@]2(C)c3cc(O)ccc3CC[C@@H]12] |
| SA19 | 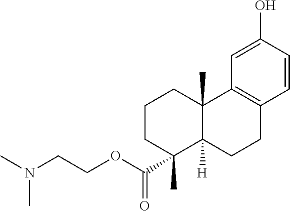 [SMILES: CN(C)CCOC(=O)[C@]1(C)CCC[C@]2(C)c3cc(O)ccc3CC[C@@H]12] |
| SA20 | 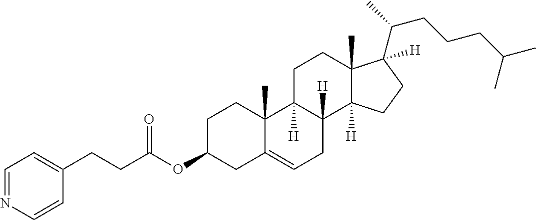 |
| SA21 | 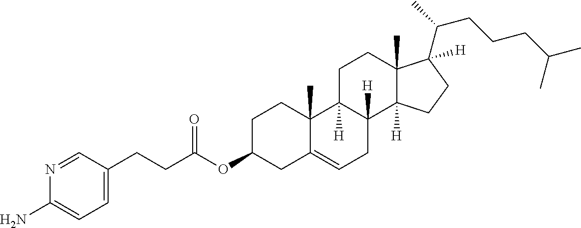 |
| SA22 | 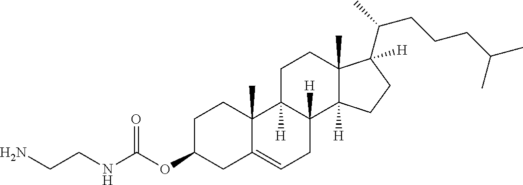 |
| SA23 | 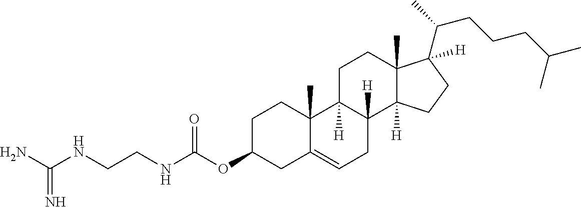 |
| SA24 | 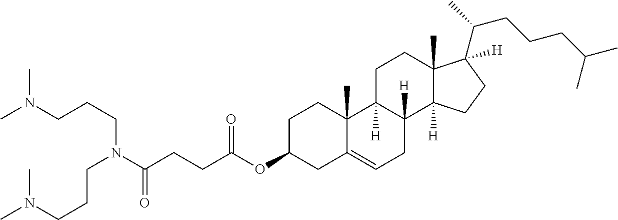 |
| SA25 | 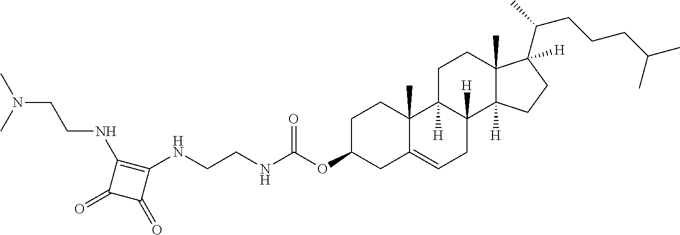 |
| SA26 | 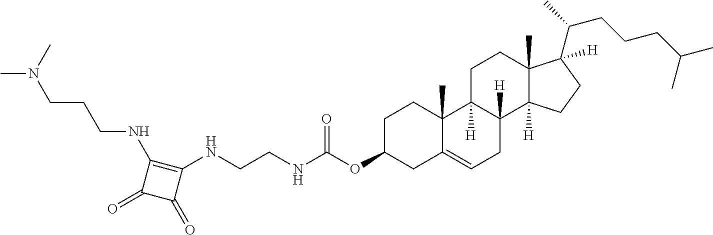 |
| SA27 | 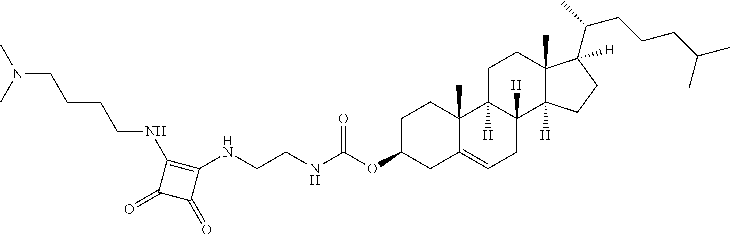 |
| SA28 | 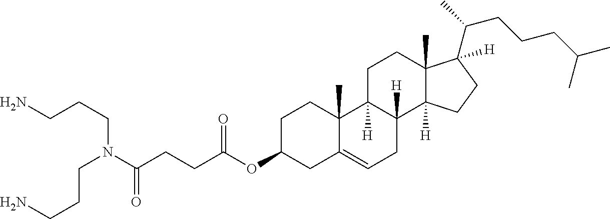 |
| SA29 | 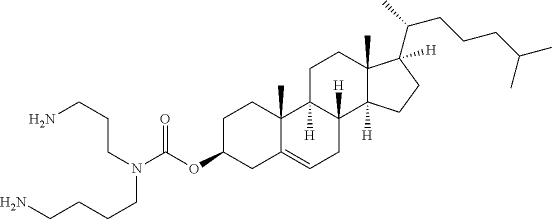 |
| SA30 | 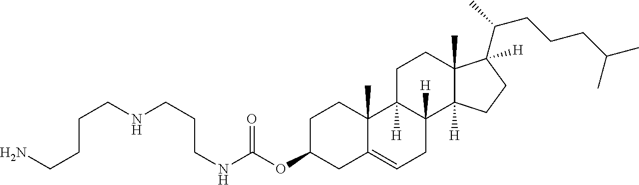 |
| SA31 | 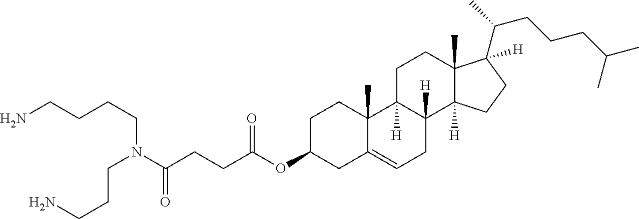 |
| SA32 | 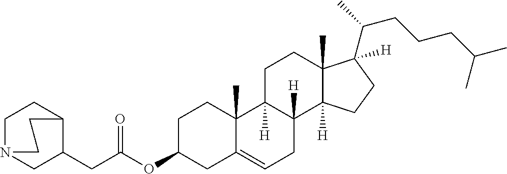 |
| SA33 | 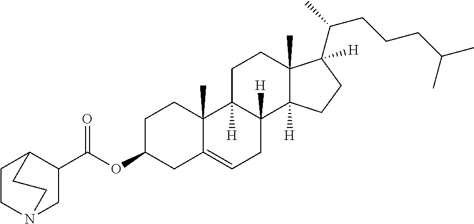 |
| SA34 | 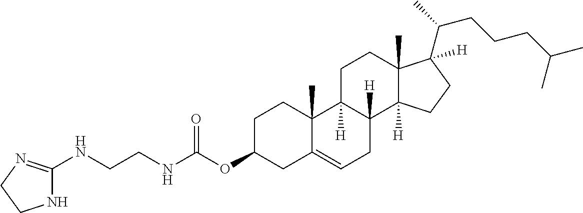 |
| SA35 | 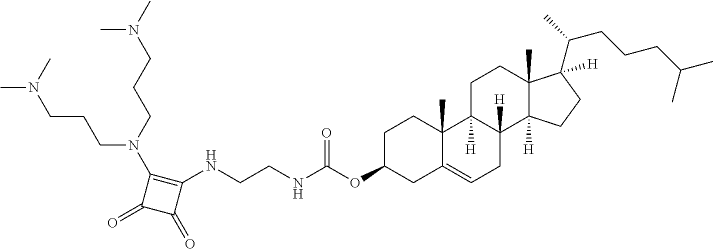 |
| SA36 | 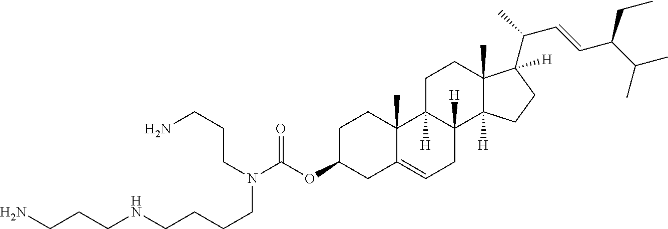 |
| SA37 | 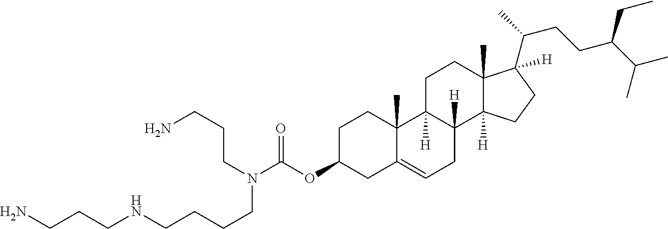 |
| SA38 | 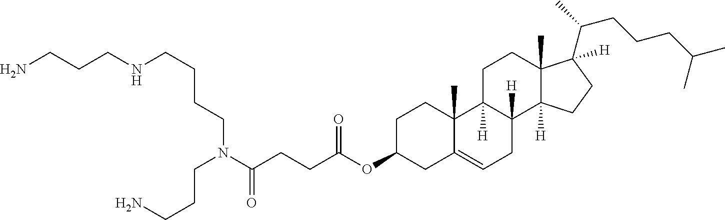 |
| SA39 | 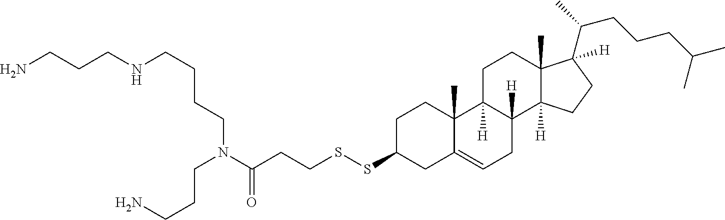 |
| SA40 | 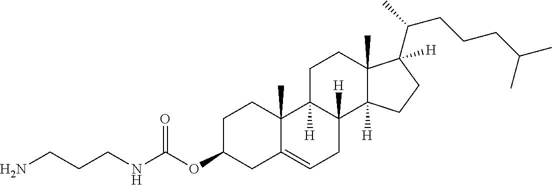 |
| SA41 | 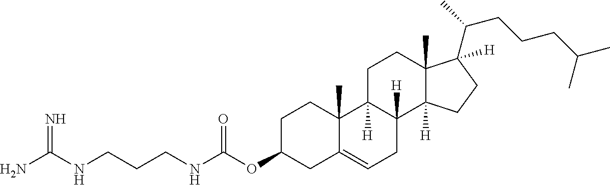 |
| SA42 | 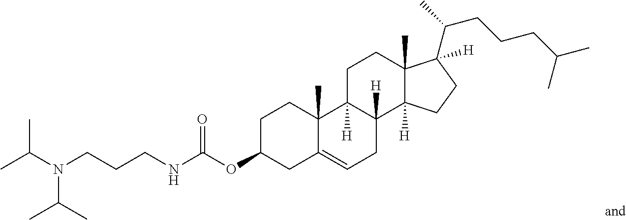 |
| SA43 | 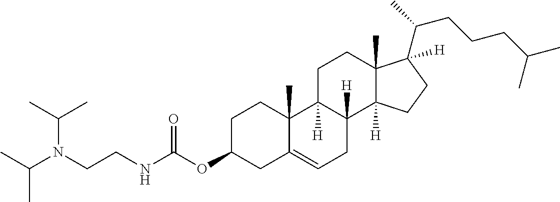 |

or a salt thereof.

59-61. (canceled)

62. The nanoparticle of claim 1, wherein the nanoparticle comprises about 30 mol % to about 60 mol % or about 40 mol % to about 50 mol % of ionizable lipid.

63. The nanoparticle of claim 1, wherein ionizable lipid is compound 18: 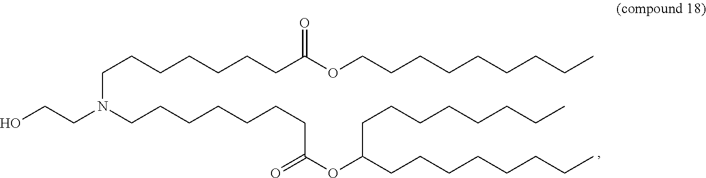 [SMILES: CCCCCCCCCOC(=O)CCCCCCCN(CCO)CCCCCCCC(=O)OC(CCCCCCCC)CCCCCCCC]
or a salt thereof.

64. The nanoparticle of claim 1, wherein the nanoparticle comprises about 5 mol % to about 15 mol %, about 8 mol % to about 13 mol %, or about 10 mol % to about 12 mol % of phospholipid.

65. (canceled)

66. The nanoparticle of claim 1, wherein the nanoparticle comprises about 20 mol % to about 60 mol %, about 30 mol % to about 50 mol %, about 35 mol %, or about 40 mol % structural lipid.

67-73. (canceled)

74. The nanoparticle of claim 1, wherein the nanoparticle comprises about 1 mol % to about 5 mol % of PEG-lipid, or about 1 mol % to about 2.5 mol % of PEG-lipid.

75. A cell comprising the nanoparticle of claim 1.

76-82. (canceled)

83. A pharmaceutical composition comprising the nanoparticle of claim 1.

84. (canceled)

85. The pharmaceutical composition of claim 83, wherein the composition is suitable for inhalation.

86. A process of preparing a nanoparticle comprising contacting a lipid nanoparticle with a cationic agent, wherein the lipid nanoparticle comprises:
(a) a lipid nanoparticle core comprising:
(i) an ionizable lipid,
(ii) a phospholipid,
(iii) a structural lipid, and
(iv) a PEG-lipid, and
(b) a polynucleotide or polypeptide payload encapsulated within the core for delivery into a cell.

87-91. (canceled)

92. A method of delivering a polynucleotide or polypeptide payload into a cell comprising contacting the cell with a nanoparticle of claim 1.

93. A method of treating or preventing a disease in a patient comprising administering to the patient a nanoparticle of claim 1 comprising a payload for treatment or prevention of the disease.

94-96. (canceled)

97. A compound of Formula A2a: 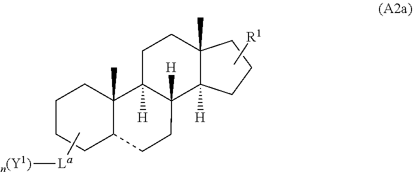
or a salt thereof, wherein:
---- is a single or double bond
R₁ is C₁₋₁₄ alkyl or C₁₋₁₄ alkenyl;
L^{a} is absent, —O—, —S—S—, —OC(═O), —C(═O)N—, —OC(═O)N—, CH₂—NH—C(O)—, —C(O)O—, —OC(O)—CH₂—CH₂—C(═O)N—, —S—S—CH₂, —SS—CH₂—CH₂—C(O)N—, or a group of formula (a): 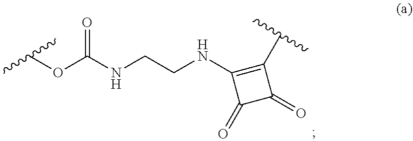 [SMILES: *OC(=O)NCCNc1c(*)c(=O)c1=O]
Y¹ is C₁₋₁₀ alkyl, 3 to 8-membered heterocycloalkyl, 5 to 6-membered heteroaryl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof,
and wherein the C₁₋₁₀ alkyl, 3 to 8-membered heterocycloalkyl, 5 to 6-membered heteroaryl, C₁₋₆ alkyl-(3 to 8-membered heterocycloalkyl), and C₁₋₆ alkyl-(5 to 6-membered heteroaryl) are each optionally substituted with 1, 2, 3, or 4 substituents independently selected from C₁₋₆ alkyl, halo, OH, —O(C₁₋₆ alkyl), —C₁₋₆ alkyl-OH, NH₂, NH(C₁₋₆ alkyl), N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH-(3 to 8-membered heterocycloalkyl), and —NH(5 to 6-membered heteroaryl); and
n is 1 or 2;
with the proviso that the compound of Formula A2a is other than:

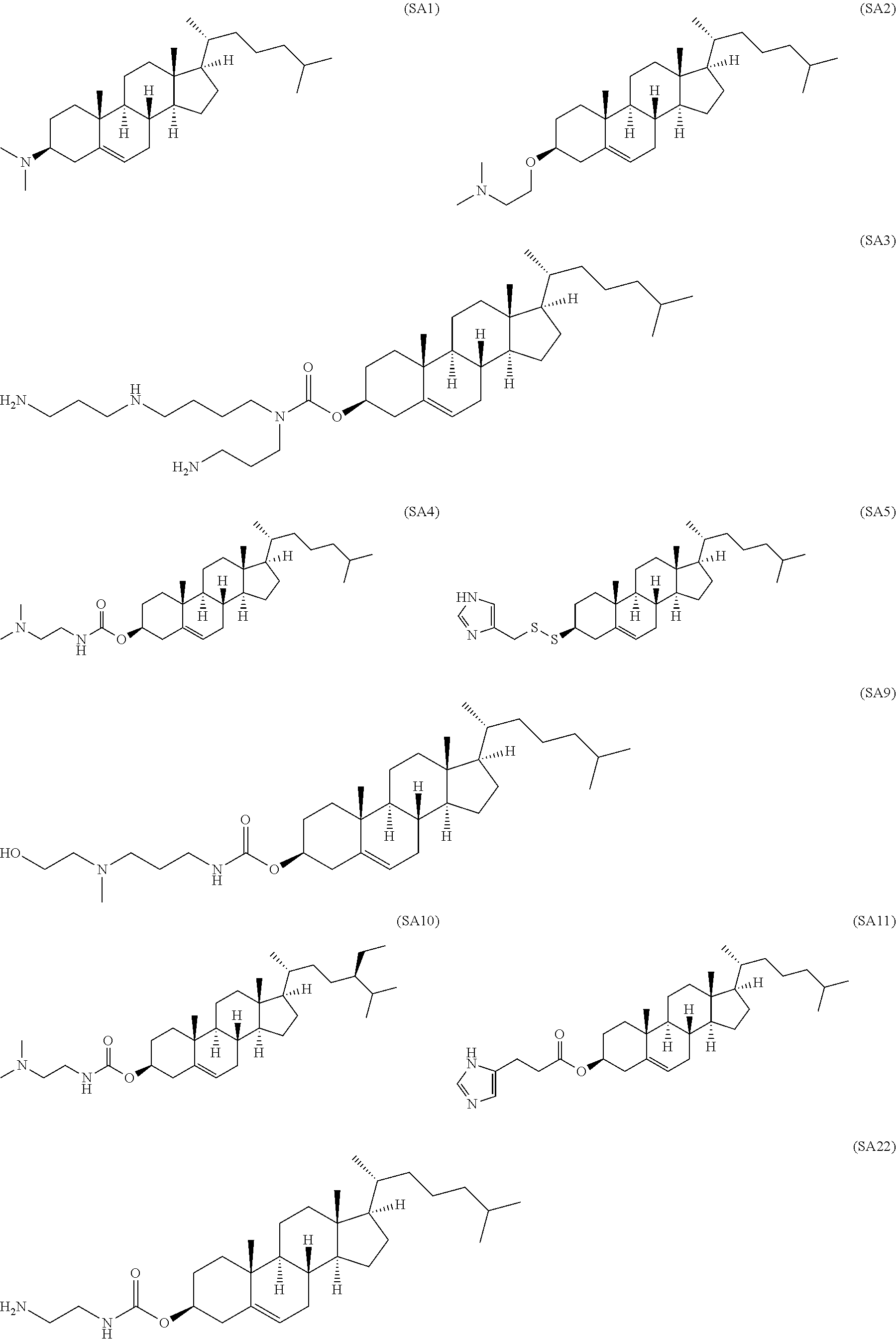

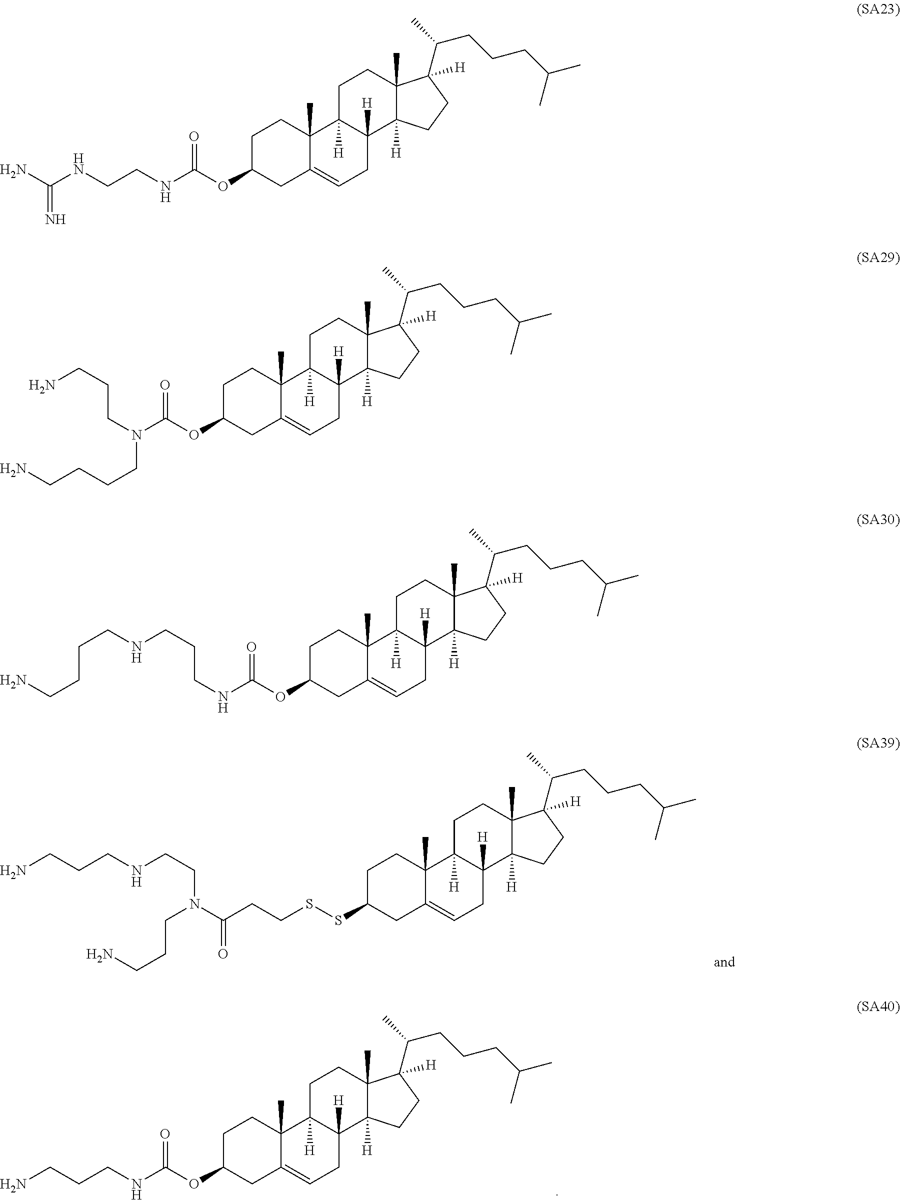

98-119. (canceled)
